(19) United States
(12) Patent Application Publication
(10) Pub. No.: US 2025/0295801 A1
(43) Pub. Date: Sep. 25, 2025

(54) CD25 ANTIBODIES, ANTIBODY DRUG CONJUGATES, AND USES THEREOF

(71) Applicant: Seagen Inc., New York, NY (US)

(72) Inventors: Matthew Levengood, Bothell, WA (US); Shyra Gardai, Monroe, WA (US); Sherif Abdelhamed, Marysville, WA (US); Paromita Raha, Kirkland, WA (US)

(73) Assignee: Seagen Inc., New York, NY (US)

(21) Appl. No.: 19/082,873

(22) Filed: Mar. 18, 2025

Related U.S. Application Data
(60) Provisional application No. 63/568,066, filed on Mar. 21, 2024.
(60) Provisional application No. 63/704,737, filed on Oct. 8, 2024.
(60) Provisional application No. 63/760,457, filed on Feb. 19, 2025.

(51) Int. Cl. A61K 47/68; A61P 35/00; C07K 16/28

(52) U.S. Cl. CPC A61K 47/6849; A61K 47/68031; A61P 35/00; C07K 16/2866; C07K 2317/92

(57) ABSTRACT

Antigen binding proteins such as antibodies and fragments thereof that bind CD25 are provided. Nucleic acids encoding such antigen binding proteins and vectors and cells useful in preparing such antigen binding proteins are also provided. Also provided are antibody-drug conjugates comprising such antigen binding proteins. The antigen binding proteins and antibody-drug conjugates are useful in a variety of methods, including the treatment of CD25 expressing and non-CD25 expressing tumors.

### CROSS-REFERENCE TO RELATED APPLICATIONS

This application claims the benefit of U.S. Provisional Application No. 63/568,066, filed on Mar. 21, 2024, U.S. Provisional Application No. 63/704,737, filed on Oct. 8, 2024, and U.S. Provisional Application No. 63/760,457, filed on Feb. 19, 2025, the entire contents of each of which are incorporated herein by reference for all purposes.

### REFERENCE TO AN ELECTRONIC SEQUENCE LISTING

The instant application contains a Sequence Listing which has been submitted electronically in .xml format and is hereby incorporated by reference in its entirety. Said .xml file, created on Mar. 4, 2025, is named “PC040982A Sequence Listing ST26.xml,” having a size of 49,395 bytes.

### BACKGROUND

CD25 is expressed on T cells within a tumor microenvironment, including regulatory T cells (Tregs). Tregs are known to suppress anti-tumor immune responses and their presence in the tumor microenvironment is associated with cancer progression. Depletion of Tregs is a promising strategy to enhance cancer immunotherapy. CD25, the alpha chain of the IL-2 receptor, is highly expressed on intratumoral Tregs, making it an attractive target for selective depletion. Agents targeting CD25 have been devised but these agents may cause adverse effects such as Guillain-Barre Syndrome, an auto-immune disorder of the peripheral nervous system. Thus, there is a need for providing therapeutic agents that can target intratumoral Tregs.

### SUMMARY

Provided herein is an antigen binding protein that binds CD25, wherein the antigen binding protein comprises: (a) a heavy chain comprising a complementarity determining region-heavy 1 (CDR-H1), a CDR-H2, a CDR-H3, or any combination thereof, wherein the CDR-H1 comprises an amino acid sequence selected from SEQ ID NO: 1 and SEQ ID NO: 25, the CDR-H2 comprises an amino acid sequence selected from SEQ ID NO: 2 and SEQ ID NO: 26, and the CDR-H3 comprises an amino acid sequence selected from SEQ ID NO: 3, SEQ ID NO: 21, and SEQ ID NO: 27; and (b) a light chain comprising a complementarity determining region-light 1 (CDR-L1), a CDR-L2, a CDR-L3, or any combination thereof, wherein the CDR-L1 comprises an amino acid sequence selected from SEQ ID NO: 4 and SEQ ID NO: 28, the CDR-L2 comprises an amino acid sequence selected from SEQ ID NO: 5 and SEQ ID NO: 29, and the CDR-L3 comprises an amino acid sequence selected from SEQ ID NO: 6 and SEQ ID NO: 30.

In some aspects, the antigen binding protein comprises a heavy chain variable region (VH) that comprises a CDR-H1 comprising the amino acid sequence of SEQ ID NO: 1, a CDR-H2 comprising the amino acid sequence of SEQ ID NO: 2, and a CDR-H3 comprising an amino acid sequence selected from SEQ ID NO: 3 and SEQ ID NO: 21.

In some aspects, the VH further comprises framework (FR) sequences between the CDRs according to the formula: (HC-FR1)-(CDR-H1)-(HC-FR2)-(CDR-H2)-(HC-FR3)-(CDR-H3)-(HC-FR4), wherein the framework sequences are optionally human sequences.

In some aspects, the VH framework sequences comprise 1, 2, 3 or 4 of the framework sequences as follows: a HC-FR1 comprising the amino acid sequence of SEQ ID NO: 9; a HC-FR2 comprising the amino acid sequence of SEQ ID NO: 10; a HC-FR3 comprising the amino acid sequence of SEQ ID NO: 11; and a HC-FR4 comprising the amino acid sequence of SEQ ID NO: 12.

In some aspects, the antigen binding protein comprises a light chain variable region (VL) that comprises a CDR-L1 comprising the amino acid sequence of SEQ ID NO: 4, a CDR-L2 comprising the amino acid sequence of SEQ ID NO: 5, and a CDR-L3 comprising the amino acid sequence of SEQ ID NO: 6.

In some aspects, the VL further comprises framework sequences between the CDRs according to the formula: (LC-FR1)-(CDR-L1)-(LC-FR2)-(CDR-L2)-(LC-FR3)-(LVR-H3)-(LC-FR4), wherein the framework sequences are optionally human sequences.

In some aspects, the framework sequences comprise 1, 2, 3 or 4 of the framework sequences as follows: a LC-FR1 comprising the amino acid sequence of SEQ ID NO: 13; a LC-FR2 comprising the amino acid sequence of SEQ ID NO: 14; a LC-FR3 comprising the amino acid sequence of SEQ ID NO: 15; and a LC-FR4 comprising the amino acid sequence of SEQ ID NO: 16.

In some aspects, the antigen binding protein comprises a VH and a VL, wherein the VH comprises a CDR-H1 of SEQ ID NO: 1, a CDR-H2 of SEQ ID NO: 2, and a CDR-H3 selected from SEQ ID NO: 3 and SEQ ID NO: 21.

In some aspects, the antigen binding protein comprises a VH and a VL, wherein the VH comprises an amino acid sequence that has at least 80%, 85%, 90%, 95% or 99% amino acid sequence identity to the amino acid sequence of SEQ ID NO: 7; or at least 80%, 85%, 90%, 95% or 99% amino acid sequence identity to the amino acid sequence of SEQ ID NO: 22.

In some aspects, the antigen binding protein comprises a VH and a VL, wherein the VL comprises a CDR-L1 of SEQ ID NO: 4, a CDR-L2 of SEQ ID NO: 5, and a CDR-L3 of SEQ ID NO: 6.

In some aspects, the antigen binding protein comprises a VH and a VL, wherein the VL comprises an amino acid sequence that has at least 80%, 85%, 90%, 95% or 99% amino acid sequence identity to the amino acid sequence of SEQ ID NO: 8.

In some aspects, the antigen binding protein comprises the six CDRs as described, and wherein the amino acid modifications in the CDRs collectively total at most 1, 2 or 3 conservative amino acid modifications.

In some aspects, the antigen binding protein comprises the following 6 CDRs: a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 3; a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6.

In some aspects, the antigen binding protein comprises the following 6 CDRs: a CDR-H1 comprising the amino acid sequence of SEQ ID NO: 1; a CDR-H2 comprising the amino acid sequence of SEQ ID NO: 2; a CDR-H3 comprising the amino acid sequence of SEQ ID NO: 21; a CDR-L1 comprising the amino acid sequence of SEQ ID NO: 4; a CDR-L2 comprising the amino acid sequence of SEQ ID NO: 5; and a CDR-L3 comprising the amino acid sequence of SEQ ID NO: 6.

In some aspects, the antigen binding protein comprises the following 6 CDRs: a CDR-H1 comprising the amino acid sequence of SEQ ID NO: 25; a CDR-H2 comprising the amino acid sequence of SEQ ID NO: 26; a CDR-H3 comprising the amino acid sequence of SEQ ID NO: 27; a CDR-L1 comprising the amino acid sequence of SEQ ID NO: 28; a CDR-L2 comprising the amino acid sequence of SEQ ID NO: 29; and a CDR-L3 comprising the amino acid sequence of SEQ ID NO: 30.

In some aspects, the VH of the antigen binding protein thereof comprises the amino acid sequence that has at least 80%, 85%, 90%, 95% or 99% amino acid sequence identity to the amino acid sequence of SEQ ID NO: 22.

In some aspects, the VH of the antigen binding protein comprises the amino acid sequence that has at least 80%, 85%, 90%, 95% or 99% amino acid sequence identity to the amino acid sequence of SEQ ID NO: 31.

In some aspects, the VL of the antigen binding protein comprises the amino acid sequence that has at least 80%, 85%, 90%, 95% or 99% amino acid sequence identity to the amino acid sequence of SEQ ID NO: 32.

In some aspects, the VH of the antigen binding protein comprises the amino acid sequence that has at least 80%, 85%, 90%, 95% or 99% amino acid sequence identity to the amino acid sequence of SEQ ID NO: 22, and the VL comprises the amino acid sequence that has at least 80%, 85%, 90%, 95% or 99% amino acid sequence identity to the amino acid sequence of SEQ ID NO: 8.

In some aspects, the VH of the antigen binding protein comprises the amino acid sequence that has at least 80%, 85%, 90%, 95% or 99% amino acid sequence identity to the amino acid sequence of SEQ ID NO: 31, and the VL comprises the amino acid sequence that has at least 80%, 85%, 90%, 95% or 99% amino acid sequence identity to the amino acid sequence of SEQ ID NO: 32.

In some aspects, the antigen binding protein comprises a heavy chain (HC) comprising the amino acid sequence that has at least 80%, 85%, 90%, 95% or 99% amino acid sequence identity to the amino acid sequence of SEQ ID NO: 45.

In some aspects, the antigen binding protein comprises a light chain (LC) comprising the amino acid sequence that has at least 80%, 85%, 90%, 95% or 99% amino acid sequence identity to the amino acid sequence of SEQ ID NO: 47.

In some aspects, the antigen binding protein comprises a HC comprising the amino acid sequence that has at least 80%, 85%, 90%, 95% or 99% amino acid sequence identity to the amino acid sequence of SEQ ID NO: 45 and a LC comprising the amino acid sequence that has at least 80%, 85%, 90%, 95% or 99% amino acid sequence identity to the amino acid sequence of SEQ ID NO: 47.

In some aspects, the antigen binding protein comprises a HC comprising the amino acid sequence that has at least 80%, 85%, 90%, 95% or 99% amino acid sequence identity to the amino acid sequence of SEQ ID NO: 46.

In some aspects, the antigen binding protein comprises a LC comprising the amino acid sequence that has at least 80%, 85%, 90%, 95% or 99% amino acid sequence identity to the amino acid sequence of SEQ ID NO: 47.

In some aspects, the antigen binding protein comprises a HC comprising the amino acid sequence that has at least 80%, 85%, 90%, 95% or 99% amino acid sequence identity to the amino acid sequence of SEQ ID NO: 46 and a LC comprising the amino acid sequence that has at least 80%, 85%, 90%, 95% or 99% amino acid sequence identity to the amino acid sequence of SEQ ID NO: 47.

In some aspects, the antigen binding protein is a monoclonal antibody or antigen binding fragment thereof. In some aspects, the antigen binding protein is a chimeric antibody or antigen binding fragment thereof. In some aspects, the antigen binding protein is a humanized antibody or antigen binding fragment thereof. In some aspects, the antigen binding protein is a human antibody or antigen binding fragment thereof. In some aspects, antigen binding protein is selected from a Fab, Fab′, Fv, scFv or (Fab′)₂ fragment.

Further provided is an antibody-drug conjugate (ADC) comprising an antigen binding protein as described herein conjugated to cytotoxic or cytostatic agent.

In some aspects, the cytotoxic or cytostatic agent is conjugated to the antigen binding protein using a linker, and, optionally, a spacer. In some aspects, the ADC comprises a spacer. In some aspects, the spacer is para-aminobenzylcarbamate. In some aspects, the linker is a cleavable linker, a non-cleavable linker, or a hydrophilic linker. In some aspects, the cleavable linker comprises an enzyme-cleavable linker. In some aspects, the linker comprises a valine-citrulline dipeptide.

In some aspects, the ADC comprises a linker-spacer of formula (I):

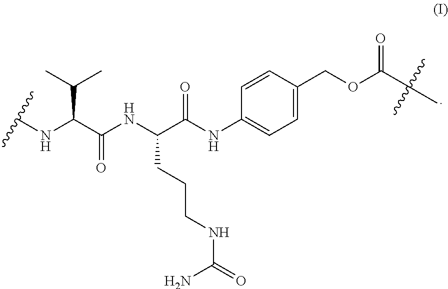 [SMILES: *N[C@H](C(=O)N[C@@H](CCCNC(N)=O)C(=O)Nc1ccc(COC(*)=O)cc1)C(C)C]

In some aspects, the ADC further comprises a maleimide-caproic acid attachment group.

In some aspects, the cytotoxic or cytostatic agent is an auristatin.

In some aspects, the cytotoxic or cytostatic agent is a peptide analogue selected from the group consisting of monomethyl auristatin E (MMAE), and dolostatin 10/auristatin.

In some aspects, the cytotoxic agent is MMAE of formula (II)

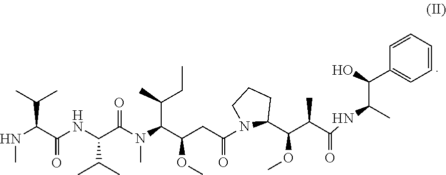 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In some aspects, the ADC comprises 2 to 10 molecules of MMAE.

In some aspects, the ADC comprises formula (III):

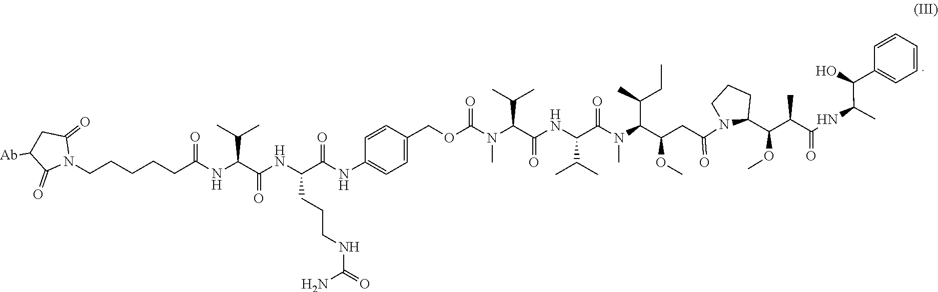

In some aspects, the ADC comprises:

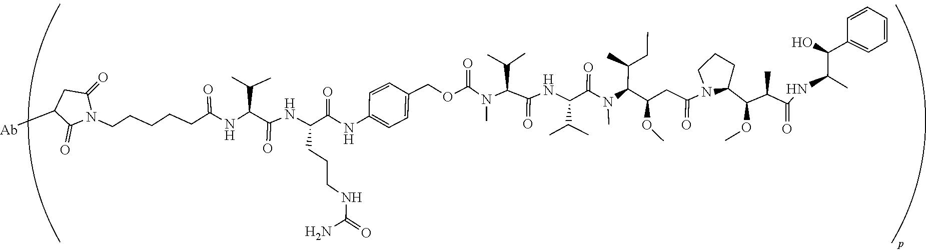

wherein the Ab is an antigen binding protein disclosed herein, and p ranges from 1 to 20, preferably from 1 to 8, and in some preferred aspects, when p represents the average drug loading, p ranges from about 2 to about 5, and in some aspects, p is about 4. In some embodiments, the antibody-drug conjugate (ADC) is an anti-CD25 monoclonal antibody and the drug is MMAE, wherein the antibody comprises a CDR-H1, CDR-H2, and CDR-H3 having amino acid sequences SEQ ID NOs: 1, 2 and 21, respectively, and a CDR-L1, CDR-L2, and CDR-L3 having amino acid sequences SEQ ID NO: 4, 5 and 6, respectively. In some embodiments, the antibody comprises a VH that comprises the amino acid sequence of SEQ ID NO: 22 and a VL that comprises the amino acid sequence of SEQ ID NO: 8. In some embodiments, the antibody comprises a HC that comprises the amino acid sequence of SEQ ID NO: 46 and a LC that comprises the amino acid sequence of SEQ ID NO: 47. In some embodiments, the antibody is linked to the drug by maleimidocaproyl valine citrulline p-amino-benzyloxy (mc-ve-pAB).

In some embodiments, the ADC is represented by formula Ab-(L-U)n, wherein Ab is an antigen binding protein disclosed herein, L is a linker between the cytotoxic molecule and the antigen binding protein, U is the conjugated cytotoxic molecule, and n is an integer from 1 to 8 (for example, from 2 to 6, or about 2, about 3, about 4, about 5, or about 6), representing the number of cytotoxic molecules bound to the antibody.

Further provided is an isolated nucleic acid encoding the antigen binding protein described herein.

In some aspects, the isolated nucleic acid further comprising a regulatory nucleic acid sequence that controls expression of the antigen binding protein or antigen binding fragment thereof in a host cell. In some aspects, the nucleic acid is codon-optimized for expression in a host cell. In some aspects, the host cell is a bacterial, yeast, insect, or mammalian cell.

Further provided is a vector comprising the nucleic acid as described herein. In some aspects, the host cell comprises the vector. In some aspects, the host cell is a bacterial, yeast, insect, or mammalian cell. In some aspects, the mammalian cell is a Chinese hamster ovary (CHO) cell.

Provided is a method of producing an antigen binding protein (e.g., an antibody or antigen binding fragment thereof) that binds to CD25, wherein the method comprises: a) culturing the host cell described herein under conditions suitable for expression of the polynucleotide encoding the antigen binding protein; and b) isolating the antigen binding protein. Further provided is an antigen binding protein produced by the method described herein.

Also provided is a method of producing an ADC comprising an antibody or antigen binding fragment thereof that binds to CD25, wherein the method comprises: a) culturing the host cell described herein under conditions suitable for expression of the polynucleotide encoding the antibody or antigen binding fragment thereof; b) isolating the antibody or antigen binding fragment thereof; and c) conjugating the antibody or antigen binding fragment thereof to a cytotoxic or cytostatic agent, wherein each unit of the cytotoxic or cytostatic agent is conjugated via a linker. Further provided is an ADC produced by the method described herein.

Provided is a pharmaceutical composition comprising an antigen binding protein described herein, or an ADC described herein and a pharmaceutically acceptable carrier.

Further provided is a method of inhibiting regulatory T (Treg) cell functioning comprising contacting a Treg cell with an effective amount of the antigen binding protein or antigen binding fragment thereof described herein, the ADC described herein, or the pharmaceutical composition described herein. In some aspects, the Treg cells are intratumoral infiltrating Treg cells.

Provided is further a method of increasing immune surveillance of aberrantly proliferating cells in a subject comprising administering to the subject an effective amount of an antigen binding protein or antigen binding fragment thereof described herein, an ADC described herein, or a pharmaceutical composition described herein.

Also provided is a method of increasing the ratio of effector T cells to regulatory T cells in a tumor of a subject in need thereof, the method comprising administering to the subject an effective amount of an antigen binding protein described herein, an ADC described herein, or a pharmaceutical composition described herein.

Provided is a method of inhibiting tumor cell growth in a subject in need thereof comprising administering to the subject an effective amount of an antigen binding protein described herein, an ADC described herein, or a pharmaceutical composition described herein.

Further provided is a method of treating cancer in a subject in need thereof comprising administering to the subject an effective amount of an antigen binding protein described herein, an ADC described herein, or a pharmaceutical composition described herein. In some aspects, the subject is a human. In some aspects, the subject has a solid tumor. In some aspects, the solid tumor is a bladder, bone, brain, breast, colon, esophageal, gastrointestinal, gum, kidney, liver, lung, nasopharynx, head and neck, ovarian, prostate, skin, stomach, testicular, tongue, or uterine tumor.

In some aspects, the cancer is a leukemia or lymphoma. In some aspects, the leukemia is chronic lymphocytic leukemia, chronic myeloid leukemia, acute lymphocytic leukemia or acute myeloid leukemia.

In some aspects, the lymphoma is non-Hodgkin's lymphoma. In some aspects, the lymphoma is peripheral T-cell lymphoma, diffuse large B-cell lymphoma, or classical Hodgkin lymphoma.

In some aspects, the cancer is a non-small cell lung, head and neck squamous cell carcinoma, melanoma, gastric cancer, gastroesophageal junction cancer, triple-negative breast cancer, or colorectal cancer. In some aspects, the non-small cell lung cancer is a squamous cell carcinoma, adenocarcinoma or large cell carcinoma. In some aspects, the colorectal cancer is a microsatellite instability-high colorectal cancer.

In some aspects, the method further comprises administration of ad additional therapy, such as radiation or a chemotherapeutic agent.

In some aspects, the method comprises administration of a PD-1 inhibitor. In some aspects, the PD-1 inhibitor is pembrolizumab. In some aspects, the PD-1 inhibitor is nivolumab. In some aspects, the PD-1 inhibitor is sasanlimab.

In some aspects, the method comprises administration of a PD-L1 inhibitor. In some aspects, the PD-L1 inhibitor is atezolizumab. In some aspects, the PD-L1 inhibitor is avelumab. In some aspects, the PD-L1 inhibitor is durvalumab.

In some aspects, the method comprises administration of a CTLA-1 inhibitor. In some aspects, the CTLA-1 inhibitor is ipilimumab. In some aspects, the CTLA-1 inhibitor is tremelimumab.

In some aspects, the administration is sequential or simultaneous and the additional therapy and the antigen binding protein, ADC, or pharmaceutical composition are administered via the same route. In some aspects, the administration is sequential or simultaneous and the additional therapy and the antigen binding protein, ADC, or pharmaceutical composition are administered via different routes.

In some aspects, one or more of the administrations is intravenous, intratumoral, intranodular, intraventricular, intrathecal, intraperitoneal, intramuscular, intradermal, transdermal, or subcutaneous.

Further provided is a use of an antigen binding protein thereof described herein, an ADC described herein, or a pharmaceutical composition described herein for treating a tumor or a cancer.

Also provided is a use of an antigen binding protein described herein, ADC described herein, or a pharmaceutical composition described herein in the manufacture of a medicament for treating a tumor or a cancer.

Provided is an article of manufacture comprising an antigen binding protein described herein, an ADC described herein, or a pharmaceutical composition described herein.

Further provided is a kit comprising an antigen binding protein described herein, an ADC described herein, or a pharmaceutical composition described herein and optional instructions for use. In some aspects, the kit further comprises an additional therapeutic agent.

Also provided herein is a method of treating cancer in a subject in need thereof comprising administering to the subject a dose of about 0.1 mg/kg to about 0.3 mg/kg of an antigen binding protein or antibody-drug conjugate described herein. In some embodiments, the method further comprises administering sasanlimab. In some embodiments, the cancer is a lymphoma or solid tumor.

### BRIEF DESCRIPTION OF THE DRAWINGS

FIG. 1A shows the binding of CD25 antibody SG25Ab-9 to human and cynomolgus CD25. FIG. 1B shows the binding of nine CD25 antibodies to cynomolgus CD25. FIG. 1C shows the binding of nine CD25 antibodies to CD25 expressing Karpas-299 cells. FIG. 1D shows the binding of nine CD25 antibodies to CD25 expressing L540cy cells.

FIG. 1E shows the internalization of CD25 antibody SG25Ab-4, SG25Ab-9, Daclizumab and CD30 antibody clone cAC10 into CD25 and CD30 expressing L82 cells.

FIG. 2A shows ADCC activity of CD25 antibody SG25Ab-9 IgG1 and CD25 antibody SG25Ab-9 non-fucosylated (NF). FIG. 2B shows depletion of Treg cells from peripheral blood mononuclear cells (PBMC) in the presence of non-fucosylated CD25 antibody SG25Ab-9 ADC and non-fucosylated IgG1 control.

FIG. 3A shows in vitro cytotoxicity of CD25 antibody SG25Ab-9 MMAE ADC and CD30 antibody cAC10 MMAE ADC on L540cy cells compared to IgG1 control ADC. FIG. 3B shows in vitro cytotoxicity of nine CD25 antibody MMAE ADCs on L540cy cells. FIG. 3C shows in vitro cytotoxicity of nine CD25 antibody MMAE ADCs on L82 cells. FIG. 3D shows in vitro cytotoxicity of nine CD25 antibody MMAE ADCs on DEL cells.

FIG. 4A shows in vivo anti-tumor activity of CD25 antibody SG25Ab-9 MMAE ADC and CD30 antibody cAC10 MMAE ADC in a mouse L540cy cell tumor model compared to control IgG1 ADC. FIG. 4B shows in vivo anti-tumor activity of CD25 antibody SG25Ab-9 MMAE ADC and CD30 antibody cAC10 MMAE ADC in a mouse L82 cell tumor model compared to IgG1 control ADC. FIG. 4C shows in vivo anti-tumor activity of CD25 antibody SG25Ab-9 Camptothecin ADC and CD30 antibody cAC10 Camptothecin ADC in a mouse L540cy cell tumor model compared to control IgG Camptothecin ADC. FIG. 4D shows in vivo anti-tumor activity of CD25 antibody SG25Ab-9 and its MMAE ADC, detuned CD25 antibody SG25Ab-9 YH98A and its MMAE ADC (“SG25Ab-9 YH98A MMAE ADC”), CD30 antibody cAC10 and its MMAE ADC in a mouse L540cy cell tumor model compared to untreated and control IgG MMAE ADC.

FIG. 5A shows percent of Treg cells in human peripheral blood cells treated with IgG1 control MMAE ADC and remaining after treatment with CD25 antibody SG25Ab-4 MMAE ADC and SG25Ab-9 MMAE ADC. FIG. 5B shows percent of CD8 T cells in human peripheral blood cells treated with IgG1 control MMAE ADC and remaining after treatment with CD25 antibody SG25Ab-4 MMAE ADC and SG25Ab-9 MMAE ADC. FIG. 5C shows percent of Treg cells in human peripheral blood cells treated with IgG1 control Camptothecin ADC and remaining after treatment with CD25 antibody SG25Ab-4 Camptothecin ADC and SG25Ab-9 Camptothecin ADC. FIG. 5D shows percent of CD8 T cells in human peripheral blood cells treated with IgG1 control Camptothecin ADC and remaining after treatment with CD25 antibody SG25Ab-4 Camptothecin ADC and SG25Ab-9 Camptothecin ADC.

FIG. 6A shows a kinetic binding analysis of CD25 antibody SG25Ab-9 binding to recombinant human CD25. FIG. 6B shows a kinetic binding analysis of detuned CD25 antibody SG25Ab-9 YH98A binding to recombinant human CD25.

FIG. 7 shows binding of CD25 antibody SG25Ab-9 and detuned CD25 antibody SG25Ab-9 YH98A to human and cynomolgus CD25.

FIG. 8A shows binding of CD25 antibody SG25Ab-9 and eight detuned SG25Ab-9 variants to human CD25. FIG. 8B shows binding of CD25 antibody SG25Ab-9 and seven detuned SG25Ab-9 variants to human CD25. FIG. 8C shows binding of CD25 antibody SG25Ab-9 and eight detuned SG25Ab-9 variants to L540cy cells. FIG. 8D shows binding of CD25 antibody SG25Ab-9 and seven detuned SG25Ab-9 variants to L540cy cells. FIG. 8E shows binding of CD25 antibody SG25Ab-9, CD25 antibody SG25Ab-9 MMAE ADC, and detuned CD25 antibody SG25Ab-9 YH98A MMAE ADC to L540cy cells.

FIG. 9 shows ADCC activity of CD25 antibody SG25Ab-9 and CD25 antibody SG25Ab-9 YH98A, both fucosylated and non-fucosylated, towards L540cy target cells compared to IgG1 control.

FIG. 10A shows in vitro cytotoxicity of CD25 antibody SG25Ab-9 MMAE ADC and five detuned SG25Ab-9 CD25 antibody MMAE ADCs on L540cy cells. FIG. 10B shows in vitro cytotoxicity of CD25 antibody SG25Ab-9 MMAE ADC and five detuned SG25Ab-9 CD25 antibody MMAE ADCs on L82 cells. FIG. 10C shows in vitro cytotoxicity of CD25 antibody SG25Ab-9 MMAE ADC and detuned CD25 antibody SG25Ab-9 YH98A MMAE ADC on SUDHL1 cells compared to control MMAE ADC. FIG. 10D shows in vitro cytotoxicity of detuned CD25 antibody SG25Ab-9 YH98A MMAE ADC on DEL, Karpas-299 and L540cy cells. FIG. 10E shows internalization of detuned CD25 antibody SG25Ab-9 YH98A MMAE ADC into DEL, Karpas-299 and L540cy cells.

FIG. 11A shows in vivo anti-tumor activity of CD25 antibody SG25Ab-9 MMAE ADC and five detuned SG25Ab-9 CD25 antibody MMAE ADCs (1.2 mg/kg) in a L82 cell xenograft tumor model compared to untreated and control IgG1 MMAE ADC. FIG. 11B shows in vivo anti-tumor activity of CD25 antibody SG25Ab-9 MMAE ADC (0.2, 0.6 mg/kg) and detuned CD25 antibody SG25Ab-9 YH98A MMAE ADC (0.2, 0.6 mg/kg) in at in a DEL cell xenograft tumor model compared to untreated and control IgG1 MMAE ADC (0.6 mg/kg). FIG. 11C shows in vivo anti-tumor activity of detuned CD25 antibody SG25Ab-9 YH98A MMAE ADC (0.6, 1.8 mg/kg) in a DEL cell xenograft tumor model compared to untreated and control IgG1 MMAE ADC (1.8 mg/kg). FIG. 11D shows in vivo anti-tumor activity of detuned CD25 antibody SG25Ab-9 YH98A MMAE ADC (0.6, 1.8 mg/kg) in a L540cy cell xenograft tumor model compared to untreated and control IgG1 MMAE ADC (1.8 mg/kg).

FIG. 12 shows depletion of Treg cells from peripheral blood mononuclear cells (PBMC) in the presence of fucosylated and non-fucosylated CD25 antibody SG25Ab-9 and non-fucosylated detuned CD25 antibody SG25Ab-9 YH98A compared to non-fucosylated control IgG1.

FIG. 13A shows in vitro cytotoxicity of CD25 antibody SG25Ab-9 tesirine ADC, CD25 antibody SG25Ab-9 MMAE ADC, and detuned CD25 antibody SG25Ab-9 YH98A MMAE ADC towards purified human Treg cells. FIG. 13B shows in vitro cytotoxicity of CD25 antibody SG25Ab-9 tesirine ADC, CD25 antibody SG25Ab-9 MMAE ADC, and detuned CD25 antibody SG25Ab-9 YH98A MMAE ADC towards purified human CD8 T cells.

FIG. 14A shows the frequency of CD4+ cells in spleens of human CD25 transgenic mice untreated or treated with non-fucosylated CD25 antibody SG25Ab-9, non-fucosylated CD25 antibody SG25Ab-9 YH98A, and non-fucosylated control IgG1 antibody after 72 hours. FIG. 14B shows the frequency of CD4+ huCD25+ cells and Treg cells in spleens of human CD25 transgenic mice untreated or treated with non-fucosylated CD25 antibody SG25Ab-9, non-fucosylated detuned CD25 antibody SG25Ab-9 YH98A and non-fucosylated control IgG1 antibody after 72 hours.

FIG. 15 shows anti-tumor activity of CD25 antibody SG25Ab-9 MMAE ADC, detuned CD25 antibody SG25Ab-9 YH98A MMAE ADC, control IgG1 MMAE ADC, and an anti-PD1 antibody in a colon cancer xenograft (MC38) model in human CD25 transgenic mice.

FIG. 16A shows in vivo cytotoxicity of CD25 antibody SG25Ab-9 MMAE ADC and control IgG1 MMAE ADC towards Treg cells in PBMC in a xenograft colon cancer (MC38) model in human CD25 transgenic mice. FIG. 16B shows in vivo cytotoxicity of CD25 antibody SG25Ab-9 MMAE ADC and control IgG1 MMAE ADC towards Treg cells in splenocytes in an MC38 xenograft model in human CD25 transgenic mice. FIG. 16C shows in vivo cytotoxicity of CD25 antibody SG25Ab-9 MMAE ADC and control IgG1 MMAE ADC towards Treg cells in an MC38 xenograft model in human CD25 transgenic mice. FIG. 16D shows hCD25 expression as mean fluorescence intensity on Tregs or CD8+ T cells in blood or tumor samples derived from an MC38 xenograft model in human CD25 transgenic mice. Samples were compared by one-way ANOVA followed by Tukey's multiple comparisons test. N=6 mice per group. Significance from selected pairs is shown. ****P<0.0001; ***P<0.001. FIG. 16E, FIG. 16F and FIG. 16G shows frequency of intratumoral Tregs, peripheral Tregs, and intratumoral CD8+ T cells, respectively, as a percent of total CD45+ cells from MC38 tumor-bearing hCD25-expressing transgenic mice treated with detuned CD25 antibody SG25Ab-9 YH98A MMAE ADC (3 or 6 mg/kg) or an isotype control ADC versus untreated group. N=6 mice per group. Frequencies were compared by one-way ANOVA followed by Tukey's pairwise comparisons test between each group. Error bars represent mean and SEM. ***P<0.001; **P<0.01; *P<0.05; ns, not significant.

FIG. 17A shows Treg cell depletion as analyzed by flow cytometry showing Treg frequency as a percent of pre-dose baseline in non-human primates (cynomolgus monkeys) treated with CD25 antibody SG25Ab-9 MMAE ADC, detuned CD25 antibody SG25Ab-9 YH98A MMAE ADC, and control IgG1 MMAE ADC intravenously q3wx3 at 6 mg/kg/dose. FIG. 17B shows flow cytometry analysis of Treg frequency as a percent of pre-dose baseline in cynomolgus monkeys from the 3-month GLP-compliant toxicity study treated intravenously with detuned CD25 antibody SG25Ab-9 YH98A MMAE ADC by q2wx7 at 3 or 5 mg/kg/dose versus vehicle-treated controls. One way ANOVA followed by Dunnett's pairwise test for every group versus the untreated group was performed and did not show a statistically significant difference.

FIG. 18A shows the effect of treating a Treg CD8+ co-culture with detuned CD25 antibody SG25Ab-9 YH98A MMAE ADC on normalized Treg counts (shown a percent of isotype control) and proliferating CSFElo CD8+ T effector cells (shown as a percent of total CD8+ Teff cells). FIG. 18B shows the effect of treating a Treg CD8+ co-culture with detuned CD25 antibody SG25Ab-9 YH98A MMAE ADC and CD25 antibody SG25Ab-9 MMAE ADC at 1, 3 or 10 g/mL on CD25hi Tregs and CD25lo Tregs counts. A non-binding isotype control ADC (10 mg/mL) was used as a control. Asterisks represent select comparisons from Dunnett's posthoc pairwise test versus isotype control. N=2 biological repeats with 2 technical repeats each. Error bars represent SD. FIG. 18C shows the effect of treating PBMC with detuned CD25 antibody SG25Ab-9 YH98A MMAE ADC and CD25 antibody SG25Ab-9 MMAE ADC at 1 μg/mL on CD25hi Tregs and CD25lo Tregs counts.

FIG. 19 shows binding of PC61 IgG2a and its variant, PC61 mIgG2a FH100BA, to mouse CD25 positive Yac-1 mouse lymphoma cells.

FIG. 20 shows T cell CD25 expression profiling by flow cytometry in tumor and blood from Renca tumor-bearing BALB/c mice. CD25 mean fluorescence intensity (MFI) in tumor or peripheral Tregs or CD8+ T cells in Renca mouse model. N=3 mice. ****P<0.0001; ***P<0.001, ns, not significant.

FIG. 21 shows PC61 mIgG2a FH100BA val-cit-PABC-MMAE(4) mediates antitumor activity in the Renca syngeneic tumor model in BALB/c mice. Mean tumor growth of Renca syngeneic tumors treated q3dx3. Error bars show SEM.

FIG. 22 shows PC61 mIgG2a FH100BA val-cit-PABC-MMAE(4) depletes Tregs in Renca syngeneic tumors. Tumoral Treg frequency as a fraction of total tumoral CD45+ cells. Error bars show mean and SD.

FIG. 23 shows different doses of PC61 mIgG2a FH100BA MC-val-cit-PABC-MMAE(4) in the Renca syngeneic tumor model. ADCs were dosed q3dx3, IV. Mean tumor growth of Renca syngeneic tumors treated q3dx3.

FIG. 24 shows a combination of anti-PD-1 and PC61 mIgG2a FH100BA MC-val-cit-PABC-MMAE(4) results in improved antitumor activity in the Renca syngeneic tumor model. ADCs were dosed q3dx3, IV. Mean tumor growth of Renca syngeneic tumors treated q3dx3.

FIG. 25 shows an analysis of CD8 T cell activation by PC61 mIgG2a FH100BA MC-val-cit-PABC-MMAE (4) (0.3 or 1 mg/kg), anti-PD-1, or the combination of both in the Renca syngeneic tumor model. Tumor-derived Ki67+ CD8+ T cells as a percentage of total CD8+ cells. N=5 mice per group. Error bars represent mean and SD. Select comparisons are shown following one-way ANOVA and Dunnett's pairwise comparisons test for each group versus the untreated group. ****P<0.0001; **P<0.01; *P<0.05; ns, not significant.

FIG. 26A shows increased the tumor ratio of Ki67+CD8+ T cells to Tregs and FIG. 26B shows the blood ratio of Ki67+CD8+ T cells to Tregs, in the Renca syngeneic tumor model after treatment with anti-mCD25V (PC61 mIgG2a FH100BA MC-val-cit-PABC-MMAE (4), 0.3 or 1 mg/kg), anti-PD-1, the combination of both anti-mCD25V and anti-PD-1, or anti-CD8.

FIG. 27 shows the schema for a phase 1 study to evaluate detuned CD25 antibody SG25Ab-9 YH98A MMAE ADC as a monotherapy and part of a combination therapy in subjects with advanced malignancies. A2* limited additional tumor types permitted in the protocol; 1L=first line; cHL=classical Hodgkin's lymphoma; DL=dose level; DLBCL=diffuse large B-cell lymphoma; HNSCC=head and neck squamous cell carcinoma; MTD=maximum tolerated dose; NSCLC=non-small cell lung cancer; PD=pharmacodynamics; PD-1=programmed cell death protein 1; PK=pharmacokinetics; PTCL=peripheral t cell lymphoma; RDEC=recommended combination dose for expansion; RDEM=recommended dose for monotherapy; R/R=relapsed/refractory.

### DETAILED DESCRIPTION

The section headings used herein are for organizational purposes only and are not to be construed as limiting the subject matter described. Each of the references disclosed herein is incorporated herein by reference in its entirety.

### I. Definitions

Unless defined otherwise, all technical and scientific terms used herein have the same meaning as commonly understood by one of ordinary skill in the art to which this disclosure is related.

Unless otherwise required by context or expressly indicated, singular terms shall include pluralities and plural terms shall include the singular.

It is understood that aspect and aspects of the invention described herein include “comprising,” “consisting,” and/or “consisting essentially of” aspects and aspects.

As used herein, the singular form “a”, “an”, and “the” should be understood to refer to “one or more” of any recited or enumerated component unless indicated otherwise.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The term “about” refers to a value or composition that is within an acceptable error range for the particular value or composition as determined by one of ordinary skill in the art, which will depend in part on how the value or composition is measured or determined, i.e., the limitations of the measurement system. As is understood by one skilled in the art, reference to “about” a value or parameter herein includes (and describes) aspects that are directed to that value or parameter per se. For example, description referring to “about X” includes description of “X”.

As described herein, any concentration range, percentage range, ratio range, or integer range is to be understood to include the value of any integer within the recited range and, when appropriate, fractions thereof (such as one tenth and one hundredth of an integer), unless otherwise indicated.

When a trade name is used herein, reference to the trade name also refers to the product formulation, the generic drug, and the active pharmaceutical ingredient(s) of the trade name product, unless otherwise indicated by context.

The terms “CD25,” “Interleukin 2 receptor subunit alpha,” “IL-2RA,” “IL2R,” “IDDM10,” “IMD41,” “TCGFR,” “TAC antigen,” and “p55” are used interchangeably herein, and, unless otherwise specified, include any naturally occurring variants (e.g., splice variants, allelic variants), isoforms, and vertebrate species homologs of human CD25. The term encompasses “full length,” unprocessed CD25 as well as any form of CD25 that results from processing within a cell. The amino acid sequence of an exemplary human CD25 is provided in GenBank NM_000417, Gene ID: 3559, UNIPROT P01589. The amino acid sequence of one specific example of a mature human CD25 protein is set forth in SEQ ID NO: 22. CD25 (IL-2 receptor α) is part of the IL-2 receptor complex that further comprises IL2Rβ (CD132) and IL-2γ (CD122). CD25 is present on many types of T cells and is expressed at high levels on regulatory T cells (Tregs). In fact, CD25 expression is elevated on tumor infiltrating T cells compared to peripheral blood mononuclear cells and CD25 expression is highest on intratumoral Tregs compared to other T cells. It has been postulated that high affinity IL-2 receptor complexes on Tregs serve as a sink for IL-2, e.g., in a tumor microenvironment.

The terms “polypeptide” and “protein” are used interchangeably to refer to a polymer of amino acid residues and are not limited to a minimum length. Such polymers of amino acid residues can contain natural or non-natural amino acid residues, and include, but are not limited to, dimers, trimers, peptides, oligopeptides, and multimers of amino acid residues. Both full-length proteins and fragments thereof are encompassed by the definition. The terms also include post-expression modifications of the polypeptide, for example, glycosylation, sialylation, acetylation, phosphorylation, and the like. The term “polypeptide” also refers to a protein which includes modifications, such as deletions, additions, and substitutions (generally conservative in nature), to the native sequence, as long as the protein maintains the desired activity. The terms “polypeptide” and “protein” encompass CD25 antigen binding proteins, including antibodies, antibody fragments, or sequences that have deletions from, additions to, and/or substitutions of one or more amino acids of the antigen binding protein.

A “native sequence” or a “naturally-occurring” polypeptide comprises a polypeptide having the same amino acid sequence as a polypeptide found in nature. Thus, a native sequence polypeptide can have the amino acid sequence of a naturally-occurring polypeptide from any mammal. Such native sequence polypeptide can be isolated from nature or can be produced by recombinant or synthetic means. The term “native sequence” polypeptide specifically encompasses naturally-occurring truncated or secreted forms of the polypeptide (e.g., an extracellular domain sequence), naturally-occurring variant forms (e.g., alternatively spliced forms) and naturally-occurring allelic variants of the polypeptide.

A polypeptide “variant” means a biologically active polypeptide (e.g., an antigen binding protein or antibody) having at least about 70%, 72%, 75%, 77%, 80%, 82%, 85%, 87%, 90%, 92%, 95%, 97%, or 99% amino acid sequence identity with the native or a reference sequence polypeptide after aligning the sequences and introducing gaps, if necessary, to achieve the maximum percent sequence identity, and not considering any conservative substitutions as part of the sequence identity. Such variants include, for instance, polypeptides wherein one or more amino acid residues are added, or deleted, at the N- or C-terminus of the polypeptide. In some aspects, a variant will have at least about 80% amino acid sequence identity. In some aspects, a variant will have at least about 90% amino acid sequence identity. In some aspects, a variant will have at least about 95% amino acid sequence identity with the native sequence polypeptide.

As used herein, “percent (%) amino acid sequence identity” and “homology” with respect to a peptide, polypeptide or antigen binding protein (e.g., antibody) sequence are defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared. For example, the % sequence identity of a given amino acid sequence A to, with, or against a given amino acid sequence B (which can alternatively be phrased as a given amino acid sequence A that has or comprises a certain % sequence identity to, with, or against a given amino acid sequence B) is calculated as follows:

100 times the fraction X/Y

where X is the number of amino acid residues scored as identical matches by the sequence in that program's alignment of A and B, and where Y is the total number of amino acid residues in B. Unless specifically stated otherwise, all % amino acid sequence identity values used herein are calculated according to this formula using the ALIGN-2 computer program. It will be appreciated that where the length of amino acid sequence A is not equal to the length of amino acid sequence B, the % sequence identity of A to B will not equal the % sequence identity of B to A.

The term “leader sequence” refers to a sequence of amino acid residues located at the N-terminus of a polypeptide that facilitates secretion of a polypeptide from a mammalian cell. A leader sequence may be cleaved upon export of the polypeptide from the mammalian cell, forming a mature protein. Leader sequences can be natural or synthetic, and they can be heterologous or homologous to the protein to which they are attached.

An “antigen binding protein” as used herein means any protein that binds a specified target antigen. In the instant application, the specified target antigen is CD25 or a fragment of CD25. An antigen binding protein includes proteins that include at least one antigen binding region or domain (e.g., at least one hypervariable region (HVR) or complementarity determining region (CDR) as defined herein). In some aspects, an antigen binding protein comprises a scaffold, such as a polypeptide or polypeptides, into which one or more (e.g., 1, 2, 3, 4, 5 or 6) HVR(s) or CDR(s), as described herein, are embedded and/or joined. In some antigen binding proteins, the HVRs or CDRs are embedded into a “framework” region, which orients the HVR(s) or CDR(s) such that the proper antigen binding properties of the CDR(s) are achieved. For some antigen binding proteins, the scaffold is the immunoglobulin heavy and/or light chain(s) from an antibody or a fragment thereof. Additional examples of scaffolds include, but are not limited to, human fibronectin (e.g., the 10^{th} extracellular domain of human fibronectin III), neocarzinostatin CBM4-2, anticalins derived from lipocalins, designed ankyrin repeat domains (DARPins), protein-A domain (protein Z), Kunitz domains, Im9, TPR proteins, zinc finger domains, pVIII, GC4, transferrin, B-domain of SPA, Sac7d, A-domain, SH3 domain of Fyn kinase, and C-type lectin-like domains (see, e.g., Gebauer and Skerra (2009) Curr. Opin. Chem. Biol., 13:245-255; Binz et al. (2005) Nat. Biotech. 23:1257-1268; and Yu et al. (2017) Annu Rev Anal Chem 10:293-320, each of which is incorporated herein by reference in its entirety).

Accordingly, antigen binding proteins include, but are not limited to, monoclonal antibodies, bispecific antibodies, minibodies, domain antibodies such as Nanobodies®, synthetic antibodies (sometimes referred to herein as “antibody mimetics”), chimeric antibodies, humanized antibodies, human antibodies, antibody fusions, and antigen binding portions or fragments of each, respectively. In some aspects, the term “antigen binding protein” includes derivatives, for example an antigen binding protein that has been chemically modified, for example an antigen binding protein that is joined to another agent such as a label or a cytotoxic or cytostatic agent (e.g., an antigen binding protein conjugate such as an ADC).

The terms “immunoglobulin” and “antibody” refer to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See, for instance, Fundamental Immunology (Paul, W., ed., 7^{th} ed. Raven Press, N.Y. (2013)). Briefly, each heavy chain typically is comprised of a heavy chain variable region (abbreviated herein as V_{H} or VH) and a heavy chain constant region (C_{H} or CH). The heavy chain constant region typically is comprised of three domains, C_{H}1, C_{H}2, and C_{H}3. The heavy chains are generally inter-connected via disulfide bonds in the so-called “hinge region.” Each light chain typically is comprised of a light chain variable region (abbreviated herein as V_{L} or VL) and a light chain constant region (C_{L} or CL). The light chain constant region typically is comprised of one domain, C_{L}. The CL can be of κ (kappa) or λ (lambda) isotype. The terms “constant domain” and “constant region” are used interchangeably herein. An immunoglobulin or antibody can derive from any of the commonly known classes, including but not limited to IgA, secretory IgA, IgE, IgD, IgG, and IgM. IgG subclasses are also well known to those in the art and include but are not limited to human IgG1, IgG2, IgG3 and IgG4. “Isotype” refers to an immunoglobulin or antibody class or subclass (e.g., IgM or IgG1) that is encoded by the heavy chain constant region genes. The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies include, for example, monoclonal antibodies (including full length or intact monoclonal antibodies), antibodies with polyepitopic or monoepitopic specificity, polyclonal or monovalent antibodies, multivalent antibodies, multispecific antibodies (e.g., bispecific antibodies so long as they exhibit the desired biological activity), single chain antibodies. An antibody can be human, humanized, chimeric and/or affinity matured, as well as an antibody from other species, for example, mouse and rabbit, etc. The term “antibody” thus includes, for instance, a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). The term “antibody” also includes, but is not limited to, synthetic antibodies, recombinantly produced antibodies, camelized antibodies, intrabodies, and anti-idiotypic (anti-Id) antibodies.

An “antibody fragment” as used herein refers to one or more fragments of an antibody, regardless of how obtained or synthesized, that retain the ability to specifically bind to the antigen bound by the whole antibody. In particular, antibodies provided herein include antibody molecules and immunologically active portions of antibody molecules, for example, antigen-binding domains or molecules that contain an antigen-binding site that binds to an antigen (e.g., one or more CDRs of an antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Plückthun and Skerra, 1989, Meth. Enzymol. 178:497-515; and Day, Advanced Immunochemistry (2d ed. 1990). Functional fragment (e.g., antigen-binding fragment) of an antibody refers to a portion of an antibody heavy and/or light chain polypeptide that retains some or all of the binding activity of the antibody from which the fragment was derived. Non-limiting examples of functional fragments (e.g., antibody fragments) include single-chain Fvs (scFv) (e.g., including monospecific, bispecific, etc.), Fab fragments, F(ab′) fragments, Fab′-SH; F(ab)₂ fragments, F(ab′)₂ fragments, disulfide-linked Fvs (dsFv), Fd fragments, Fv fragments, diabody, triabodies, tetrabodies, peptibodies, minibodies, and multispecific antibodies formed from antibody fragments. A “Fv” fragment includes a non-covalently-linked dimer of one heavy chain variable domain and one light chain variable domain. A “Fab” fragment includes, the constant domain of the light chain and the first constant domain (C_{H1}) of the heavy chain, in addition to the heavy and light chain variable domains of the Fv fragment. A “F(ab′)₂” fragment includes two Fab fragments joined, near the hinge region, by disulfide bonds.

The term “hypervariable region” or “HVR,” as used herein, refers to each of the regions of an antibody variable domain that are hypervariable in sequence. HVRs can form structurally defined loops (“hypervariable loops”). Generally, native four-chain antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, e.g., Xu et al., Immunity 13:37-45 (2000); Johnson and Wu, in Methods in Molecular Biology 248:1-25 (Lo, ed., Human Press, Totowa, NJ, 2003). Indeed, naturally-occurring camelid antibodies consisting of a heavy chain only are functional and stable in the absence of light chain. See, e.g., Hamers-Casterman et al., Nature 363:446-448 (1993); Sheriff et al., Nature Struct. Biol. 3:733-736 (1996).

HVRs generally comprise amino acid residues from the hypervariable loops and/or from the “complementary determining regions” (CDRs), CDRs being of highest sequence variability and/or involved in antigen recognition. A variety of schemes for defining the boundaries of a given CDR are known in the art. For example, the Kabat Complementarity Determining Regions (CDRs) are based on sequence variability and are the most commonly used (Kabat et at., Sequences of Proteins of Immunological Interest, 5th Ed. Public Health Service, National Institutes of Health, Bethesda, MD. (1991)). Chothia refers instead to the location of the structural loops (Chothia and Lesk, J. Mol. Biol. 196:901-917 (1987)). The AbM CDRs represent a compromise between the Kabat CDRs and Chothia structural loops and are used by Oxford Molecular's AbM antibody modeling software. The “contact” CDRs are based on an analysis of the available complex crystal structures. Additional details on the foregoing schemes as well as other numbering conventions are provided in the following references: Al-Lazikani et al., (1997) J. Mol. Biol. 273: 927-948 (“Chothia” numbering scheme); MacCallum et al., (1996) J. Mol. Biol. 262:732-745 (1996), (Contact” numbering scheme); Lefranc M-P., et al., (2003) Dev. Comp. Immunol. 27:55-77 (“IMGT” numbering scheme); and Honegger A. & Pluckthun A. (2001) J. Mol/Biol. 309:657-70, (AHo numbering scheme).

In some aspects, the HVR regions and associated sequences are the same as the CDR regions and associated sequences based upon one of the foregoing numbering conventions. As such, residues for exemplary HVRs and/or CDRs are summarized in Table 1 below.

TABLE 1
Summary of Different CDR Numbering Schemes

| Loop | IMGT | Kabat | AbM | Chothia | Contact |
|---|---|---|---|---|---|
| CDR-H1 | 27-38 | 31-35 | 26-35 | 26-32 | 30-35 |
| CDR-H2 | 56-65 | 50-65 | 50-58 | 52-56 | 47-58 |
| CDR-H3 | 105-117 | 95-102 | 95-102 | 95-102 | 93-101 |
| CDR-L1 | 27-38 | 24-34 | 24-34 | 24-34 | 30-36 |
| CDR-L2 | 56-65 | 50-56 | 50-56 | 50-56 | 46-55 |
| CDR-L3 | 105-117 | 89-97 | 89-97 | 89-97 | 89-96 |

In some aspects, CDRs can comprise extended CDRs as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et at., supra, for each of these definitions.

Unless otherwise specified, the terms “CDR” and “complementary determining region” of a given antibody or region thereof, such as a variable region, as well as individual CDRs (e.g., “CDR-H1, CDR-H2) of the antibody or region thereof, should be understood to encompass the complementary determining region as defined by any of the known schemes described herein above. In some instances, the scheme for identification of a particular CDR or CDRs is specified, such as the CDR as defined by the IMGT, Kabat, AbM, Chothia, or Contact method. In other instances, the particular amino acid sequence of a CDR is given.

Thus, in some aspects, the antigen binding protein comprises CDRs and/or HVRs as defined by the IMGT system. In other aspects, the antigen binding protein comprises CDRs or HVRs as defined by the Kabat system. In still other aspects, the antigen binding protein comprises CDRs or HVRs as defined by the AbM system. In further aspects, the antigen binding protein comprises CDRs or HVRs as defined by the Chothia system. In yet other aspects, the antigen binding protein comprises CDRs or HVRs as defined by the IMGT system. In some aspects, the antigen binding proteins comprise the HVR and/or CDR residues as identified in Tables 2 and 3 or as set forth elsewhere herein.

The term “variable region” or “variable domain” refers to the domain of an antigen binding protein (e.g., an antibody) heavy or light chain that is involved in binding the antigen binding protein (e.g., antibody) to antigen. The variable regions or domains of the heavy chain and light chain (VH and VL, respectively) of an antigen binding protein such as an antibody can be further subdivided into regions of hypervariability (or hypervariable regions, which may be hypervariable in sequence and/or form of structurally defined loops), such as hypervariable regions (HVRs) or complementarity-determining regions (CDRs), interspersed with regions that are more conserved, termed framework regions (FRs). In general, there are three HVRs (HVR-H1, HVR-H2, HVR-H3) or CDRs (CDR-H1, CDR-H2, CDR-H3) in each heavy chain variable region, and three HVRs (HVR-L1, HVR-L2, HVR-L3) or CDRs in (CDR-L1, CDR-L2, CDR-L3) in each light chain variable region. “Framework regions” and “FR” are known in the art to refer to the non-HVR or non-CDR portions of the variable regions of the heavy and light chains. In general, there are four FRs in each full-length heavy chain variable region (FR-H1, FR-H2, FR-H3, and FR-H4), and four FRs in each full-length light chain variable region (FR-L1, FR-L2, FR-L3, and FR-L4). Within each VH and VL, three HVRs or CDRs and four FRs are typically arranged from amino-terminus to carboxy-terminus in the following order: FR1, HVR1, FR2, HVR2, FR3, HVR3, FR4 in the case of HVRs, or FR1, CDR1, FR2, CDR2, FR3, CDR3, FR4 in the case of CDRs (See also Chothia and Lesk J Mot. Biol., 195, 901-917 (1987)). A single VH or VL domain can be sufficient to confer antigen-binding specificity. In addition, antibodies that bind a particular antigen can be isolated using a VH or VL domain from an antibody that binds the antigen to screen a library of complementary VL or VH domains, respectively. See, e.g., Portolano et al. J. Immunol. 150:880-887 (1993); Clarkson et al., Nature 352:624-628 (1991).

The term “heavy chain variable region” (VH) as used herein refers to a region comprising heavy chain HVR-H1, FR-H2, HVR-H2, FR-H3, and HVR-H3. For example, a heavy chain variable region may comprise heavy chain CDR-H1, FR-H2, CDR-H2, FR-H3, and CDR-H3. In some aspects, a heavy chain variable region also comprises a FR-H1 or at least a portion of an FR-H1 and/or a FR-H4 or at least a portion of an FR-H4.

The term “heavy chain constant region” as used herein refers to a region comprising at least three heavy chain constant domains, C_{H}1, C_{H}2, and C_{H}3. Nonlimiting exemplary heavy chain constant regions include γ, δ, and α. Nonlimiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, and an antibody comprising an α constant region is an IgA antibody. Further, an antibody comprising a μ constant region is an IgM antibody, and an antibody comprising an E constant region is an IgE antibody. Certain isotypes can be further subdivided into subclasses. For example, IgG antibodies include, but are not limited to, IgG1 (comprising a γ₁ constant region), IgG2 (comprising a γ₂ constant region), IgG3 (comprising a γ₃ constant region), and IgG4 (comprising a γ₄ constant region) antibodies; IgA antibodies include, but are not limited to, IgA1 (comprising an ai constant region) and IgA2 (comprising an α₂ constant region) antibodies; and IgM antibodies include, but are not limited to, IgM1 and IgM2.

The term “heavy chain” (HC) as used herein refers to a polypeptide comprising at least a heavy chain variable region, with or without a leader sequence. In some aspects, a heavy chain comprises at least a portion of a heavy chain constant region. The term “full-length heavy chain” as used herein refers to a polypeptide comprising a heavy chain variable region and a heavy chain constant region, with or without a leader sequence.

The term “light chain variable region” (VL) as used herein refers to a region comprising light chain HVR-L1, FR-L2, HVR-L2, FR-L3, and HVR-L3. In some aspects, the light chain variable region comprises light chain CDR-L1, FR-L2, CDR-L2, FR-L3, and CDR-L3. In some aspects, a light chain variable region also comprises an FR-L1 or at least a portion of a FR-L1 and/or an FR-L4 or at least a portion of a FR-L4.

The term “light chain constant region” as used herein refers to a region comprising a light chain constant domain, CL. Nonlimiting exemplary light chain constant regions include λ and κ.

The term “light chain” (LC) as used herein refers to a polypeptide comprising at least a light chain variable region, with or without a leader sequence. In some aspects, a light chain comprises at least a portion of a light chain constant region. The term “full-length light chain” as used herein refers to a polypeptide comprising a light chain variable region and a light chain constant region, with or without a leader sequence.

The “EU numbering system” or “EU index” is generally used when referring to a residue in an immunoglobulin heavy chain constant region (e.g., the EU index reported in Kabat et al., Sequences of Proteins of Immunological Interest, 5th Ed. Public Health Service, National Institutes of Health, Bethesda, Md., 1991). The “EU index as in Kabat” refers to the residue numbering of the human IgG1 EU antibody. Unless stated otherwise herein, references to residue numbers in the constant domain of antibodies means residue numbering by the EU numbering system.

The term “monoclonal antibody” refers to an antibody obtained from a population of substantially homogeneous antibodies, that is, the individual antibodies comprising the population are identical except for possible naturally occurring mutations that can be present in minor amounts. Monoclonal antibodies are highly specific, being directed against a single antigenic site. In contrast to polyclonal antibody preparations, which can include different antibodies directed against different determinants (epitopes), each monoclonal antibody is directed against a single determinant on the antigen.

A “bispecific” antibody as used herein refers to an antibody, having binding specificities for at least two different antigenic epitopes. In some aspects, the epitopes are from the same antigen. In other aspects, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al., Nature 305:537-39 (1983). Alternatively, bispecific antibodies can be prepared using chemical linkage. See, e.g., Brennan, et al., Science 229:81 (1985). Bispecific antibodies include bispecific antibody fragments. See, e.g., Hollinger, et al., Proc. Natl. Acad. Sci. U.S.A. 90:6444-48 (1993), Gruber, et al., J. Immunol. 152:5368 (1994).

A “dual variable domain immunoglobulin” or “DVD-Ig” refers to multivalent and multispecific binding proteins as described, e.g., in DiGiammarino et al., Methods Mol. Biol. 899:145-156, 2012; Jakob et al., MABs 5:358-363, 2013; and U.S. Pat. Nos. 7,612,181; 8,258,268; 8,586,714; 8,716,450; 8,722,855; 8,735,546; and 8,822,645, each of which is incorporated by reference in its entirety.

A “dual-affinity re-targeting protein” or a “DART” is a form of a bispecific antibody in which the heavy variable domain from one antibody is linked with the light variable domain of another, and the two chains associate, and are described in, e.g., Garber, Nature Reviews Drug Discovery 13:799-801, 2014.

A “Bispecific T-cell Engager” or “BiTE®”, is the genetic fusion of two scFv fragments resulting in tandem scFv molecules, and are described, e.g., in Baeuerle et al., Cancer Res. 69: 4941-4944, 2009.

A “chimeric antibody” as used herein refers to an antibody in which a portion of the heavy and/or light chain is derived from a particular source or species, while the remainder of the heavy and/or light chain is derived from a different source or species. In some aspects, a chimeric antibody refers to an antibody comprising at least one variable region from a first species (such as mouse, rat, cynomolgus monkey, etc.) and at least one constant region from a second species (such as human, cynomolgus monkey, etc.). In some aspects, a chimeric antibody comprises at least one mouse variable region and at least one human constant region. In some aspects, a chimeric antibody comprises at least one cynomolgus variable region and at least one human constant region. In some aspects, all of the variable regions of a chimeric antibody are from a first species and all of the constant regions of the chimeric antibody are from a second species.

The term “humanized antibody” as used herein, refers to a genetically engineered non-human antibody, which contains human antibody constant domains and non-human variable domains modified to contain a high level of sequence homology to human variable domains. Humanized antibodies can be prepared by grafting the six non-human antibody complementarity-determining regions (CDRs), onto a homologous human acceptor framework region (FR) (see WO92/22653 and EP0629240). In order to fully reconstitute the binding affinity and specificity of the parental antibody, the substitution of framework residues from the parental antibody (i.e. the non-human antibody) into the human framework regions (back-mutations) may be required. Structural homology modeling may help to identify the amino acid residues in the framework regions that are important for the binding properties of the antibody. Thus, a humanized antibody may comprise non-human CDR sequences, primarily human framework regions optionally comprising one or more amino acid back-mutations to the non-human amino acid sequence, and fully human constant regions. Optionally, additional amino acid modifications, which are not necessarily back-mutations, may be applied to obtain a humanized antibody with preferred characteristics, such as affinity and biochemical properties.

“Human antibodies” as used herein refer to antibodies produced in humans, antibodies produced in non-human animals that comprise human immunoglobulin genes, such as XenoMouse®, and antibodies selected using in vitro methods, such as phage display, wherein the antibody repertoire is based on a human immunoglobulin sequence. A “human antibody” is one having variable regions in which both the FRs and CDRs are derived from human germline immunoglobulin sequences. Furthermore, if the antibody contains a constant region, the constant region also is derived from human germline immunoglobulin sequences. The human antibodies of the disclosure can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human antibodies” and “fully human antibodies” are used synonymously.

As used herein, a human antibody is “derived from” a particular germline sequence if the antibody is obtained from a system using human immunoglobulin sequences, for instance by immunizing a transgenic mouse carrying human immunoglobulin genes or by screening a human immunoglobulin gene library, and wherein the selected human antibody variable domain sequence is at least 90%, at least 95%, at least 96%, at least 97%, at least 98%, or at least 99% identical in amino acid variable domain sequence to the amino acid sequence encoded by the germline immunoglobulin gene. Typically, outside the heavy chain CDR3, a human antibody derived from a particular human germline sequence will display no more than 20 amino acid differences, e.g. no more than 10 amino acid differences, such as no more than 9, 8, 7, 6 or 5, for instance no more than 4, 3, 2, or 1 amino acid difference from the amino acid sequence encoded by the germline immunoglobulin gene.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework derived from a human immunoglobulin framework or a human consensus framework can comprise the same amino acid sequence thereof, or it can contain amino acid sequence changes. In some aspects, the number of amino acid changes are 10 or less, 9 or less, 8 or less, 7 or less, 6 or less, 5 or less, 4 or less, 3 or less, or 2 or less. In some aspects, the VL acceptor human framework is identical in sequence to the VL human immunoglobulin framework sequence or human consensus framework sequence.

An “affinity matured” antibody refers to an antibody with one or more alterations in one or more hypervariable regions (HVRs) compared to a parent antibody which does not possess such alterations, such alterations resulting in an improvement in the affinity of the antibody for antigen. In some examples, an affinity matured antibody refers to an antibody with one or more alterations in one or more complementarity determining regions (CDRs) compared to a parent antibody which does not possess such alterations, such alterations resulting in an improvement in the affinity of the antibody for antigen.

The term “derivative” refers to a molecule (e.g., an antigen binding protein such as an antibody or fragment thereof) that includes a chemical modification other than an insertion, deletion, or substitution of amino acids (or nucleic acids). In certain aspects, derivatives comprise covalent modifications, including, but not limited to, chemical bonding with polymers, lipids, or other organic or inorganic moieties. In certain aspects, a derivative of a particular antigen binding protein can have a greater circulating half-life than an antigen binding protein that is not chemically modified. In certain aspects, a derivative can have improved targeting capacity for desired cells, tissues, and/or organs. In some aspects, a derivative of an antigen binding protein is covalently modified to include one or more polymers, including, but not limited to, monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of such polymers. See, e.g., U.S. Pat. Nos. 4,640,835, 4,496,689, 4,301,144, 4,670,417, 4,791,192 and 4,179,337.

As used herein, the term “epitope” refers to a site on an antigen (e.g., CD25), to which an antigen binding protein (e.g., an antibody or fragments thereof) that targets that antigen binds. Epitopes often consist of a chemically active surface grouping of molecules such as amino acids, polypeptides, sugar side chains, phosphoryl or sulfonyl groups, and have specific three-dimensional structural characteristics as well as specific charge characteristics. Epitopes can be formed both from contiguous or noncontiguous amino acids of the antigen that are juxtaposed by tertiary folding. Epitopes formed from contiguous residues typically are retained on exposure to denaturing solvents, whereas epitopes formed by tertiary folding typically are lost on treatment with denaturing solvents. In certain aspects, an epitope can include, but is not limited to, at least 3, at least 4, at least 5, at least 6, at least 7, amino acids in a unique spatial arrangement. In some aspects, the epitope refers to 3-5, 4-6, or 8-10 amino acids in a particular spatial conformation. In further aspects, an epitope is less than 20 amino acids in length, less than 15 amino acids or less than 12 amino acids, less than 10 amino acids, or less than 8 amino acids in length. The epitope can comprise amino acid residues directly involved in the binding (also called immunodominant component of the epitope) and other amino acid residues that are not directly involved in the binding, including amino acid residues that are effectively blocked or covered by the antigen binding molecule (i.e., the amino acids are within the footprint of the antigen binding molecule). Methods of determining spatial conformation of epitopes include, for example, x-ray crystallography, two-dimensional nuclear magnetic resonance, and HDX-MS (see, e.g., Epitope Mapping Protocols in Methods in Molecular Biology, Vol. 66, G. E. Morris, Ed. (1996)). Once a desired epitope of an antigen is determined, antigen binding proteins (e.g., antibodies or fragments thereof) to that epitope can be generated using established techniques. It is then possible to screen the resulting antigen binding proteins in competition assays to identify antigen binding proteins that bind the same or overlapping epitopes. Methods for binning antibodies based upon cross-competition studies are described in WO 03/48731.

A “nonlinear epitope” or “conformational epitope” comprises noncontiguous polypeptides, amino acids, and/or sugars within the antigenic protein to which an antibody specific to the epitope binds.

A “linear epitope” comprises contiguous polypeptides, amino acids, and/or sugars within the antigenic protein to which an antigen binding protein (e.g., an antibody or fragment thereof) specific to the epitope binds.

A “paratope” or “antigen binding site” is the site on the antigen binding protein (e.g., antibody or fragment thereof) that binds the epitope and typically includes the amino acids that are in close proximity to the epitope once the antibody is bound (see, e.g., Sela-Culang et al., 2013, Front Immunol. 4:302).

The term “compete” when used in the context of antigen binding proteins (e.g., antibodies or fragments thereof) that compete for the same epitope means competition between antigen binding proteins as determined by an assay in which the antigen binding protein (e.g., an antibody or fragment thereof) being tested (e.g., a test antibody) prevents or inhibits (partially or completely) specific binding of a reference antigen binding protein (e.g., a reference antibody) to a common antigen (e.g., CD25 or a fragment thereof). Numerous types of competitive binding assays can be used to determine if one antigen binding protein competes with another, including various label-free biosensor approaches such as surface plasmon resonance (SPR) analysis (see, e.g., Abdiche, et al., 2009, Anal. Biochem. 386:172-180; Abdiche, et al., 2012, J. Immunol Methods 382:101-116; and Abdiche, et al., 2014 PLoS One 9:e92451. Other assays that can be used include: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J Immunol. 137:3614-3619) solid phase direct labeled assay, solid phase direct labeled sandwich assay (see, e.g., Harlow and Lane, 1988, Antibodies, A Laboratory Manual, Cold Spring Harbor Press); solid phase direct label RIA using I-125 label (see, e.g., Morel et al., 1988, Mol. Immunol. 25:7-15); solid phase direct biotin-avidin EIA (see, e.g., Cheung, et al., 1990, Virology 176:546-552); direct labeled RIA (Moldenhauer et al., 1990, Scand. J. Immunol. 32:77-82). Typically, the test antigen binding protein is present in excess (e.g., at least 2×, 5×, 10×, 20× or 100×). Usually, when a competing antigen binding protein is present in excess, it will inhibit specific binding of a reference antigen binding protein to a common antigen by at least 40%, 50%, 60%, 70%, 80%, 90%, 95% or 100%. In instances in in which each antigen binding protein (e.g., an antibody or fragment thereof) detectably inhibits the binding of the other antigen binding protein with its cognate epitope, whether to the same, greater, or lesser extent, the antigen binding proteins are said to “cross-compete” with each other for binding of their respective epitope(s) or to “cross-block” one another. Typically, such cross-competition studies are done using the conditions and methods described above for competition studies and the extent of blocking is at least 30%, at least 40%, or at least 50% each way. “ ”

“Affinity” refers to the strength of the sum total of noncovalent interactions between a single binding site of a molecule (e.g., an antibody) and its binding partner (e.g., an antigen). The affinity of a molecule X for its partner Y can generally be represented by the dissociation constant (K_{d}). Affinity can be measured by common methods known in the art, including those described herein.

An “affinity matured” antibody refers to an antibody with one or more alterations in one or more hypervariable regions (HVRs) compared to a parent antibody which does not possess such alterations, such alterations resulting in an improvement in the affinity of the antibody for antigen. In some examples, an affinity matured antibody refers to an antibody with one or more alterations in one or more complementarity determining regions (CDRs) compared to a parent antibody which does not possess such alterations, such alterations resulting in an improvement in the affinity of the antibody for antigen.

A “detuned” antibody refers to an antibody with one or more alterations in one or more hypervariable regions (HVRs) compared to a parent antibody which does not possess such alterations, wherein such one or more alterations results in a reduction in the affinity of the antibody for an antigen. In some examples, a detuned antibody refers to an antibody with one or more alterations in one or more complementarity determining regions (CDRs) compared to a parent antibody which does not possess such alterations, wherein such one or more alterations result in a reduction in the affinity of the antibody for an antigen.

As used herein, the term “specifically binds”, “binding” or simply “binds” or other related terms in the context of the binding of an antigen binding protein to its target antigen means that the antigen binding protein exhibits essentially background binding to non-target molecules. An antigen binding protein that specifically binds the target antigen (e.g., CD25) may, however, cross-react with CD25 proteins from different species. Typically, a CD25 antigen binding protein specifically binds human CD25 when the dissociation constant (K_{D}) is between about 10⁻¹¹ M and about 10⁻⁶ M; or about 5×10⁻⁶ M, about 10⁻⁶ M, about 10⁻⁷ M, about 5×10⁻⁸ M, about 10⁻⁸ M, about 5×10⁻⁹ M, about 10⁻⁹ M, about 5×10⁻¹⁰ M, about 10⁻¹⁰ M, about 5×10⁻¹¹ or about 10⁻¹¹ M; about 10⁻⁶ M or less, about 10⁻⁷ M or less, about 10⁻⁸ M or less, about 10⁻¹⁰ M or less, about 10⁻¹⁰ M or less, or about 10⁻¹¹ M or even less as measured via a surface plasma resonance (SPR) technique (e.g., BIACore, GE-Healthcare Uppsala, Sweden) using the antibody as the ligand and the antigen as the analyte.

The term “K_{D}” (M), as used herein, refers to the dissociation equilibrium constant of a particular antigen binding protein-antigen interaction (e.g., antibody-antigen interaction). Affinity, as used herein, and K_{D} are inversely related, such that higher affinity is intended to refer to lower K_{D}, and lower affinity is intended to refer to higher K_{D}.

An “antibody-drug conjugate” or simply “ADC” refers to an antigen binding protein (e.g., antibody) conjugated to a cytotoxic agent or cytostatic agent. An ADC typically binds to the target antigen (e.g., CD25) on a cell surface followed by internalization of the ADC into the cell where the drug is released. The ADC can include MMAE, camptothecin, tesirine, or anthracycline.

A “cytotoxic effect” refers to the depletion, elimination and/or killing of a target cell.

A “cytotoxic agent” refers to an agent that has a cytotoxic effect on a cell. A cytotoxic agent can be conjugated to an antibody or administered in combination with an antibody.

A “cytostatic effect” refers to the inhibition of cell proliferation.

A “cytostatic agent” refers to an agent that has a cytostatic effect on a cell, thereby inhibiting the growth of and/or expansion of a specific subset of cells. Cytostatic agents can be conjugated to an antibody or administered in combination with an antibody.

The term “Fc region” herein is used to define a C-terminal region of an immunoglobulin heavy chain that contains at least a portion of the constant region. The term includes native sequence Fc regions and variant Fc regions. In one aspects, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “functional Fc region” possesses an “effector function” of a native sequence Fc region. Exemplary “effector functions” include Fc receptor binding; C1q binding; complement dependent cytotoxicity (CDC); antibody-dependent cell-mediated cytotoxicity (ADCC); antibody-dependent cellular phagocytosis (ADCP); down regulation of cell surface receptors (e.g. B cell receptor; BCR), etc. Such effector functions generally require the Fc region to be combined with a binding domain (e.g., an antibody variable domain) and can be assessed using various assays.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

A “variant Fc region” comprises an amino acid sequence which differs from that of a native sequence Fc region by virtue of at least one amino acid modification.

“Fc receptor” or “FcR” describes a receptor that binds to the Fc region of an antibody. In some aspects, an FcγR is a native human FcR. In some aspects, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term “Fc receptor” or “FcR” also includes the neonatal receptor, FcRn, which is responsible for the transfer of maternal IgGs to the fetus (Guyer et al., J Immunol. 117:587 (1976) and Kim et al., J Immunol. 24:249 (1994)) and regulation of homeostasis of immunoglobulins. Methods of measuring binding to FcRn are known (see, e.g., Ghetie and Ward, Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.).

“Effector functions” refer to biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); antibody-dependent cellular phagocytosis (ADCP); down regulation of cell surface receptors (e.g. B cell receptor); and B cell activation. Such functions can be affected by, for example, binding of an Fc effector domain(s) to an Fc receptor on an immune cell with phagocytic or lytic activity or by binding of an Fc effector domain(s) to components of the complement system. Typically, the effect(s) mediated by the Fc-binding cells or complement components result in inhibition and/or depletion of the targeted cell. Fc regions of antibodies can recruit Fc receptor (FcR)-expressing cells and juxtapose them with antibody-coated target cells. Cells expressing surface FcR for IgGs including FcγRIII (CD16), FcγRII (CD32) and FcγRIII (CD64) can act as effector cells for the destruction of IgG-coated cells. Such effector cells include monocytes, macrophages, natural killer (NK) cells, neutrophils and eosinophils. Engagement of FcγR by IgG activates antibody-dependent cellular cytotoxicity (ADCC) or antibody-dependent cellular phagocytosis (ADCP). ADCC is mediated by CD16′ effector cells through the secretion of membrane pore-forming proteins and proteases, while phagocytosis is mediated by CD32+ and CD64⁺ effector cells (see, e.g., Fundamental Immunology, 4^{th} ed., Paul ed., Lippincott-Raven, N.Y., 1997, Chapters 3, 17 and 30; Uchida et al., 2004, J. Exp. Med. 199:1659-69; Akewanlop et al., 2001, Cancer Res. 61:4061-65; Watanabe et al., 1999, Breast Cancer Res. Treat. 53:199-207.

“Human effector cells” are leukocytes, which express one or more FcRs and perform effector functions. In certain aspects, the cells express at least FcγRIII and perform ADCC effector function(s). Examples of human leukocytes, which mediate ADCC include peripheral blood mononuclear cells (PBMC), natural killer (NK) cells, monocytes, cytotoxic T cells, and neutrophils. The effector cells may be isolated from a native source, e.g., from blood.

“Antibody-dependent cell-mediated cytotoxicity” or “ADCC” refers to a mechanism of cytotoxicity in which the Fc region of antibodies bound to antigen on the cell surface of target cells interact with Fc receptors (FcRs) present on certain cytotoxic effector cells (e.g. NK cells, neutrophils, and macrophages). This interaction enables these cytotoxic effector cells to subsequently kill the target cell with cytotoxins. The primary cells for mediating ADCC, NK cells, express FcγRIII only, whereas monocytes express FcγRI, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or 6,737,056 (Presta), can be performed. Useful effector cells for such assays include PBMC and NK cells. ADCC activity of the molecule of interest can also be assessed in vivo, e.g., in an animal model such as that disclosed in Clynes et al. Proc. Natl. Acad. Sci. (USA) 95:652-656 (1998). Additional polypeptide variants with altered Fc region amino acid sequences (polypeptides with a variant Fc region) and increased or decreased ADCC activity are described, e.g., in U.S. Pat. Nos. 7,923,538, and 7,994,290.

“Complement dependent cytotoxicity” or “CDC” refers to the lysis of a target cell in the presence of complement. Activation of the classical complement pathway is initiated by the binding of the first component of the complement system (C1q) to the Fc region of antibodies (of the appropriate subclass), which are bound to their cognate antigen on a target cell. This binding activates a series of enzymatic reactions culminating in the formation of holes in the target cell membrane and subsequent cell death. Activation of complement may also result in deposition of complement components on the target cell surface that facilitate ADCC by binding complement receptors (e.g., CR3) on leukocytes. To assess complement activation, a CDC assay, e.g., as described in Gazzano-Santoro et al., J. Immunol. Methods 202:163 (1996), can be performed. Polypeptide variants with altered Fc region amino acid sequences (polypeptides such as antibodies with variant Fc regions) and increased or decreased C1q binding capability are described, e.g., in U.S. Pat. No. 6,194,551 B1, U.S. Pat. Nos. 7,923,538, 7,994,290 and WO 1999/51642. See also, e.g., Idusogie et al., J Immunol. 164: 4178-4184 (2000).

The term “antibody-dependent cellular phagocytosis”, or simply “ADCP”, refers to the process by which antibody-coated cells are internalized, either in whole or in part, by phagocytic immune cells (e.g., macrophages, neutrophils and dendritic cells) that bind to an Fc region of Ig.

A polypeptide variant with “altered” FcR binding affinity or ADCC activity (e.g., an antibody) is one, which has either enhanced or diminished FcR binding activity and/or ADCC activity compared to a parent polypeptide or to a polypeptide comprising a native sequence Fc region. The polypeptide variant which “displays increased binding” to an FcR binds at least one FcR with better affinity than the parent polypeptide. The polypeptide variant which “displays decreased binding” to an FcR, binds at least one FcR with lower affinity than a parent polypeptide. In some aspects, such variants which display decreased binding to an FcR may possess little or no appreciable binding to an FcR, e.g., 0-20% binding to the FcR compared to a native sequence IgG Fc region.

The term “substantially similar” or “substantially the same,” as used herein, denotes a sufficiently high degree of similarity between two or more numeric values such that one of skill in the art would consider the difference between the two or more values to be of little or no biological and/or statistical significance within the context of the biological characteristic measured by said value. In some aspects, the two or more substantially similar values differ by no more than about any one of 5%, 10%, or 15%.

The phrase “substantially different,” as used herein, denotes a sufficiently high degree of difference between two numeric values such that one of skill in the art would consider the difference between the two values to be of statistical significance within the context of the biological characteristic measured by said values. In some aspects, the two substantially different numeric values differ by greater than about any one of 20%, 25%, 30%, 35%, 40%, 45%, 50%, 60%, 70%, 80%, 90%, or 100%.

The phrase “substantially reduced,” as used herein, denotes a sufficiently high degree of reduction between a numeric value and a reference numeric value such that one of skill in the art would consider the difference between the two values to be of statistical significance within the context of the biological characteristic measured by said values. In some aspects, the substantially reduced numeric values is reduced by greater than about any one of 20%, 25%, 30%, 35%, 40%, 45%, 50%, 60%, 70%, 80%, 90%, or 100% compared to the reference value. In some aspects, “substantially reduced” can mean reduced by about 2-fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105-fold, about 110-fold, about 115-fold, about 120-fold, about 125-fold, about 130-fold, about 135-fold, about 140-fold, about 145-fold, about 150-fold, about 155-fold, about 160-fold, about 165-fold, about 170-fold, about 175-fold, about 180-fold, about 185-fold, about 190-fold, about 195-fold, about 200-fold, about 500-fold, about 600-fold, about 700-fold, about 800-fold, about 900-fold, about 1000-fold, about 1100-fold, about 1200-fold, or more.

The phrase “substantially increased,” as used herein, denotes a sufficiently high degree of increase between a numeric value and a reference numeric value such that one of skill in the art would consider the difference between the two values to be of statistical significance within the context of the biological characteristic measured by said values. In some aspects, the substantially increased numeric values is increased by greater than about any one of 20%, 25%, 30%, 35%, 40%, 45%, 50%, 60%, 70%, 80%, 90%, or 100% compared to the reference value. In some aspects, “substantially increased” can mean increased by about 2-fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, about 105-fold, about 110-fold, about 115-fold, about 120-fold, about 125-fold, about 130-fold, about 135-fold, about 140-fold, about 145-fold, about 150-fold, about 155-fold, about 160-fold, about 165-fold, about 170-fold, about 175-fold, about 180-fold, about 185-fold, about 190-fold, about 195-fold, about 200-fold, about 500-fold, about 600-fold, about 700-fold, about 800-fold, about 900-fold, about 1000-fold, about 1100-fold, about 1200-fold, or more.

The terms “nucleic acid molecule”, “nucleic acid” and “polynucleotide” are used interchangeably herein and refer to a polymer of nucleotides of any length. Such polymers of nucleotides can contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

The term “vector” means any molecule or entity (e.g., nucleic acid, plasmid, bacteriophage or virus) used to transfer a nucleic acid molecule into a host cell. A vector typically includes a nucleic acid molecule engineered to contain a cloned polynucleotide or polynucleotides encoding a polypeptide or polypeptides of interest that can be propagated in a host cell. Examples of vectors include, but are not limited to, plasmids, viral vectors, and expression vectors, for example, recombinant expression vectors. A vector may include one or more of the following elements: an origin of replication, one or more regulatory sequences (such as, for example, promoters and/or enhancers) that regulate the expression of the polypeptide of interest, and/or one or more selectable marker genes. The term includes vectors, which are self-replicating nucleic acid molecules as well as vectors incorporated into the genome of a host cell into which it has been introduced.

The term “expression vector” refers to a vector that is suitable for transformation of a host cell and that can be used to express a polypeptide of interest in a host cell.

The terms “host cell” or “host cell line” are used interchangeably herein and refer to a cell or population of cells that may be or has been a recipient of a vector or isolated polynucleotide. Host cells can be prokaryotic cells or eukaryotic cells. Exemplary eukaryotic cells include mammalian cells, such as primate or non-primate animal cells; fungal cells, such as yeast; plant cells; and insect cells. Nonlimiting exemplary mammalian cells include, but are not limited to, NSO cells, PER.C6® cells (Crucell), 293 and CHO cells, and their derivatives, such as 293-6E and DG44 cells, respectively. Such terms refer not only to the original cell, but also to the progeny of such a cell. Certain modifications may occur in succeeding generations due to, for example, mutation or environmental influences. Such progeny are also encompassed by the terms so long as the cells have the same function or biological activity as the original cells.

The term “control sequence” refers to a polynucleotide sequence that can affect the expression and processing of coding sequences to which it is operably linked. The nature of such control sequences can depend upon the host organism. In particular aspects, control sequences for prokaryotes can include a promoter, a ribosomal binding site, and a transcription termination sequence. Control sequences for eukaryotes can include, for example, promoters comprising one or a plurality of recognition sites for transcription factors, transcription enhancer sequences, and transcription termination sequence. “Control sequences” can include leader sequences and/or fusion partner sequences.

As used herein, “operably linked” means that the components to which the term is applied are in a relationship that allows them to carry out their inherent functions under suitable conditions. For example, a control sequence in a vector that is “operably linked” to a protein coding sequence is ligated thereto such that expression of the protein coding sequence is achieved under conditions compatible with the transcriptional activity of the control sequence. In the case in which two encoding sequences are operably linked, the phrase means that the two DNA fragments or encoding sequences are joined such that the amino acid sequences encoded by the two fragments remain in-frame.

The term “transfection” means the uptake of foreign or exogenous DNA by a cell, and a cell has been “transfected” when the exogenous DNA has been introduced inside the cell membrane. A number of transfection techniques are well known in the art and are disclosed herein. See, e.g., Graham et al., 1973, Virology 52:456; Sambrook et al., 2001, Molecular Cloning: A Laboratory Manual, supra; Davis et al., 1986, Basic Methods in Molecular Biology, Elsevier; Chu et al., 1981, Gene 13:197. Such techniques can be used to introduce one or more exogenous DNA moieties into suitable host cells.

The term “transformation” refers to a change in a cell's genetic characteristics, and a cell has been transformed when it has been modified to contain new DNA or RNA. For example, a cell is transformed where it is genetically modified from its native state by introducing new genetic material via transfection, transduction, or other techniques. Following transfection or transduction, the transforming DNA can recombine with that of the cell by physically integrating into a chromosome of the cell, or can be maintained transiently as an episomal element without being replicated, or can replicate independently as a plasmid. A cell is considered to have been “stably transformed” when the transforming DNA is replicated with the division of the cell.

The term “isolated” as used herein refers to a molecule that has been separated from at least some of the components with which it is typically found in nature or produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated”.

The terms “individual”, “subject”, or “patient” are used interchangeably herein to refer to an animal, for example, a mammal. In some aspects, methods of treating mammals, including, but not limited to, humans, rodents, simians, felines, canines, equines, bovines, porcines, ovines, caprines, mammalian laboratory animals, mammalian farm animals, mammalian sport animals, and mammalian pets, are provided. In some instances, the “individual” or “subject” is a human. In some examples, an “individual” or “subject” refers to an individual or subject (e.g., a human) in need of treatment for a disease or disorder.

A “disease” or “disorder” as used herein refers to a condition where treatment is needed.

“Cancer” and “tumor,” as used herein, are interchangeable terms that refer to any abnormal cell or tissue growth or proliferation in an animal. As used herein, the terms “cancer” and “tumor” encompass solid and hematological/lymphatic cancers and also encompass malignant, pre-malignant, and benign growths, such as dysplasias. A solid tumor is an abnormal growth or mass of tissue that usually does not contain cysts or liquid areas. Examples of cancer include but are not limited to, carcinoma, lymphoma, blastoma, sarcoma, and leukemia and include cancers of the head and neck, e.g., cancers of the nasal cavity, paranasal sinuses, nasopharynx, oral cavity, oropharynx, middle ear, larynx, hypopharynx, salivary glands; cancers of the lung, e.g., non-small cell lung cancer (NSCLC) (squamous cell carcinoma, spindle cell carcinoma, adenocarcinoma, large cell carcinoma, clear cell carcinoma, bronchioalveolar), small cell lung cancer (SCLC) (oat cell cancer, intermediate cell cancer, combined oat cell cancer); neoplasms of the mediastinum, e.g., neurogenic tumors (including neurofibroma, neurilemoma, malignant schwannoma, neurosarcoma, ganglioneuroblastoma, anglioneuroma, neuroblastoma, pheochromocytoma, paraganglioma), germ cell tumors (including seminoma, teratoma, non-seminoma), thymic tumors (including thymoma, thymolipoma, thymic carcinoma, thymic carcinoid), mesenchymal tumors (including fibroma, fibrosarcoma, lipoma, liposarcoma, myxoma, mesothelioma, leiomyoma, leiomyosarcoma, rhabdomyosarcoma, xanthogranuloma, mesenchymoma, hemangioma, hemangioendothelioma, hemangiopericytoma, lymphangioma, lymphangiopericytoma, lymphangiomyoma); cancers of the gastrointestinal (Gl) tract, e.g., cancers of the esophagus, stomach (gastric cancer), pancreas, liver, biliary tree, gall bladder, small intestine (including duodenum, jejunum, ileum), large intestine (including cecum, colon, rectum, anus), colorectal cancer, gastrointestinal stroma tumor, hepatocellular carcinoma (HCC), hepatoblastoma, cholangiocarcinoma, cholangiocellular carcinoma, hepatic cystadenocarcinoma, angiosarcoma, hemangioendothelioma, leiomyosarcoma, malignant Schwannoma, fibrosarcoma; cancer of the genitourinary system (including kidney, e.g. renal pelvis, renal cell carcinoma (RCC), nephroblastoma (Wilms' tumor), hypernephroma, cancer of the ureter; urinary bladder, urethra, penis, testis; gynecologic cancer, e.g., cancer of the ovary, fallopian tube, peritoneum, cervix, vulva, vagina, uterine body; cancers of the breast, e.g., mammary carcinoma (infiltrating ductal, colloid, lobular invasive, tubular, adenocystic, papillary, medullary, mucinous), hormone receptor positive breast cancer (estrogen receptor positive breast cancer, progesterone receptor positive breast cancer), Her2 positive breast cancer, triple negative breast cancer, Paget's disease of the breast; cancers of the endocrine system, e.g., cancers of the endocrine glands, thyroid gland (thyroid carcinomas/tumors; papillary, follicular, anaplastic, medullary), parathyroid gland (parathyroid carcinoma), adrenal cortex (adrenal cortical carcinoma/tumors), pituitary gland (including prolactinoma, craniopharyngioma), thymus, adrenal glands, pineal gland, carotid body, islet cell tumors, paraganglion, pancreatic endocrine tumors (PET; non-functional PET, gastrinoma, insulinoma, glucagonoma, somatostatinoma, carcinoid tumors; sarcomas of the soft tissues, e.g., fibrosarcoma, fibrous histiocytoma, liposarcoma, leiomyosarcoma, rhabdomyosarcoma, angiosarcoma, lymphangiosarcoma, Kaposi's sarcoma, glomus tumor, hemangiopericytoma, synovial sarcoma, giant cell tumor of tendon sheath, solitary fibrous tumor of pleura and peritoneum, diffuse mesothelioma, malignant peripheral nerve sheath tumor (MPNST), granular cell tumor, clear cell sarcoma, melanocytic schwannoma, plexosarcoma, neuroblastoma, ganglioneuroblastoma, neuroepithelioma, extraskeletal Ewing's sarcoma, paraganglioma, extraskeletal chondrosarcoma, extraskeletal osteosarcoma, mesenchymoma, alveolar soft part sarcoma epithelioid sarcoma, extrarenal rhabdoid tumor, desmoplastic small cell tumor; sarcomas of the bone, e.g., myeloma, reticulum cell sarcoma, chondrosarcoma (including central, peripheral, clear cell, mesenchymal chondrosarcoma), osteosarcoma, Ewing's tumor, malignant giant cell tumor, histiocytoma, fibrosarcoma, chordoma, hemangioendothelioma, hemangiopericytoma, osteochondroma, osteoid osteoma, osteoblastoma, eosinophilic granuloma, chondroblastoma; mesothelioma: e.g. pleural mesothelioma, peritoneal mesothelioma; cancers of the skin, e.g. basal cell carcinoma, squamous cell carcinoma, Merkel's cell carcinoma, melanoma (including cutaneous, superficial spreading, lentigo maligna, acral lentiginous, nodular, intraocular melanoma), actinic keratosis, eyelid cancer; neoplasms of the central nervous system and brain, e.g., astrocytoma (cerebral, cerebellar, diffuse, fibrillary, anaplastic, pilocytic, protoplasmic, gemistocytary), glioblastoma, gliomas, oligodendrogliomas, oligoastrocytomas, ependymomas, ependymoblastomas, choroid plexus tumors, medulloblastomas, meningiomas, schwannomas, hemangioblastomas, hemangiomas, hemangiopericytomas, neuromas, ganglioneuromas, neuroblastomas, retinoblastomas, neurinomas (e.g. acoustic), spinal axis tumors; lymphomas and leukemias, e.g., B-cell non-Hodgkin lymphomas (NHL) including small lymphocytic lymphoma (SLL), lymphoplasmacytoid lymphoma (LPL), mantle cell lymphoma (MCL), follicular lymphoma (FL), diffuse large cell lymphoma (DLCL), Burkitt's lymphoma (BL)), T-cell non-Hodgkin lymphomas (including anaplastic large cell lymphoma (ALCL), adult T-cell leukemia/lymphoma (ATLL), cutaneous T-cell lymphoma (CTCL), peripheral T cell lymphoma (PTCL)), lymphoblastic T-cell lymphoma (T-LBL), adult T-cell lymphoma, lymphoblastic B-cell lymphoma (B-LBL), immunocytoma, chronic B-cell lymphocytic leukemia (B-CLL), chronic T-cell lymphocytic leukemia (T-CLL) B-cell small lymphocytic lymphoma (B-SLL), cutaneous T-cell lymphoma (CTLC), primary central nervous system lymphoma (PCNSL), immunoblastoma, Hodgkin's disease (HD) (including nodular lymphocyte predominance HD (NLPHD), nodular sclerosis HD (NSHD), mixed-cellularity HD (MCHD), lymphocyte-rich classic HD, lymphocyte-depleted HD (LDHD)), large granular lymphocyte leukemia (LGL), chronic myelogenous leukemia (CML), acute myelogenous/myeloid leukemia (AML), acute lymphatic/lymphoblastic leukemia (ALL), acute promyelocytic leukemia (APL), chronic lymphocytic/lymphatic leukemia (CLL), prolymphocytic leukemia (PLL), hairy cell leukemia, chronic myelogenous/myeloid leukemia (CML), myeloma, plasmacytoma, multiple myeloma (MM), plasmacytoma, myelodysplastic syndromes (MDS), chronic myelomonocytic leukemia (CMML); or a cancer of unknown primary site (CUP). The cancers enumerated are meant to include the primary tumors and any metastatic tumors derived therefrom.

“Tumor burden” also referred to as “tumor load,” which refers to the total amount of tumor material distributed throughout the body. Tumor burden refers to the total number of cancer cells or the total size of tumor(s) throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The terms “metastatic cancer” and “metastatic disease” mean cancers that have spread from the site of origin to another part of the body, e.g., to regional lymph nodes or to distant sites.

The terms “advanced cancer”, “locally advanced cancer”, “advanced disease” and “locally advanced disease” mean cancers that have extended, e.g., through a relevant tissue capsule or a basement membrane. Surgery is typically not recommended for patients with locally advanced disease, and these patients have substantially less favorable outcomes compared to patients having clinically localized (organ-confined) cancer.

As used herein, “treatment” is an approach for obtaining beneficial or desired clinical results. “Treatment” as used herein, covers any administration or application of a therapeutic for disease in a mammal, including a human. Beneficial or desired clinical results include, but are not limited to, any one or more of: alleviation of one or more symptoms, diminishment of extent of disease, prevention or delay of the spread (e.g., metastasis, for example metastasis to the lung or to the lymph node) of disease, prevention or delay of the recurrence of disease, delay or slowing of disease progression, amelioration of the disease state, inhibition of the disease or progression of the disease, inhibition or slowing the disease or its progression, arrest of its development, and remission (whether partial or total). Also encompassed by “treatment” is a reduction of pathological consequence of a proliferative disease.

In the context of cancer, the term “treating” includes any or all of: inhibiting growth of cancer cells, inhibiting replication of cancer cells, reducing the number of cancer cells, reducing the rate of cancer cell infiltration into peripheral organs, reducing the rate or extent of tumor metastasis, lessening of overall tumor burden, and ameliorating one or more symptoms associated with the cancer.

In the context of an autoimmune disease, the term “treating” includes any or all of: preventing replication of cells associated with an autoimmune disease state including, but not limited to, cells capable of producing an autoimmune antibody, lessening the autoimmune-antibody burden and ameliorating one or more symptoms of an autoimmune disease.

The terms “inhibition” or “inhibit” refer to a decrease or cessation of any phenotypic characteristic or to a decrease or cessation in the incidence, degree, or likelihood of that characteristic. To “reduce” or “inhibit” is to decrease, reduce or arrest an activity, function, and/or amount as compared to a reference. In certain aspects, by “reduce” or “inhibit” is meant the ability to cause an overall decrease of 20% or greater. In another aspects, by “reduce” or “inhibit” is meant the ability to cause an overall decrease of 50% or greater. In yet another aspects, “reduce” or “inhibit” is meant the ability to cause an overall decrease of 70%, 75%, 80%, 85%, 90%, 95%, 98%, or greater.

A “reference” as used herein, refers to any sample, standard, or level that is used for comparison purposes. A reference can be obtained from a healthy and/or non-diseased sample. In some examples, a reference can be obtained from an untreated sample. In some examples, a reference is obtained from a non-diseased or non-treated sample of a subject individual. In some examples, a reference is obtained from one or more healthy individuals who are not the subject or patient.

A “reference antibody” as used herein, refers to an antibody that binds an antigen that is similar or identical to the antigen bound by an antibody of interest but the reference antibody comprises at least one difference compared to the antibody of interest, which at least one difference is located outside the antigen binding site.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease.

As used herein, to “suppress” a function or activity is to reduce the function or activity when compared to otherwise same conditions except for a condition or parameter of interest, or alternatively, as compared to another condition. For example, an antibody which suppresses tumor growth reduces the rate of growth of the tumor compared to the rate of growth of the tumor in the absence of the antibody.

An “effective amount” or “therapeutically effective amount” or “therapeutically effective dosage” of a drug or therapeutic agent is any amount of the drug or agent that, when used alone or in combination with another therapeutic agent provides a treatment effect, such as protecting a subject against the onset of a disease or promoting disease regression as evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

By way of example for the treatment of tumors, in some aspects a therapeutically effective amount of an anti-tumor agent inhibits cell growth or tumor growth by at least about 10%, by at least about 20%, by at least about 30%, by at least about 40%, by at least about 50%, by at least about 60%, by at least about 70%, or by at least about 80%, by at least about 90%, by at least about 95%, by at least about 96%, by at least about 97%, by at least about 98%, by at least about 99%, or up to 100% in a treated subject(s) (e.g., one or more treated subjects) relative to an untreated subject(s) (e.g., one or more untreated subjects). In some aspects, a therapeutically effective amount of an anti-tumor agent inhibits cell growth or tumor growth by 100% in a treated subject(s) (e.g., one or more treated subjects) relative to an untreated subject(s) (e.g., one or more untreated subjects). In other aspects of the disclosure, tumor regression can be observed and continue for a period of at least about 20 days, at least about 30 days, at least about 40 days, at least about 50 days, at least about 60 days, at least 3 months, at least 4 months, at least 5 months, at least 6 months, at least 7 months, at least 8 months, at least 9 months, at least 10 months, at least 11 months, at least 12 months, at least 18 months, at least 24 months, at least 36 months, at least 48 months or at least 60 months.

A therapeutically effective amount of a drug includes a “prophylactically effective amount,” which is any amount of the drug that, when administered alone or in combination with an anti-cancer agent to a subject at risk of developing a cancer (e.g., a subject having a pre-malignant condition) or of suffering a recurrence of cancer, inhibits the development or recurrence of the cancer. In some aspects, the prophylactically effective amount prevents the development or recurrence of the cancer entirely. “Inhibiting” the development or recurrence of a cancer means either lessening the likelihood of the cancer's development or recurrence, or preventing the development or recurrence of the cancer entirely.

As used herein, “subtherapeutic dose” means a dose of a therapeutic compound that is lower than the usual or typical dose of the therapeutic compound when administered alone for the treatment of a hyperproliferative disease (e.g., cancer).

“Administering” or “administration” refer to the physical introduction of a therapeutic agent to a subject, using any of the various methods and delivery systems known to those skilled in the art. Exemplary routes of administration include intravenous, intramuscular, subcutaneous, intradermal, intranasal, intraperitoneal, intraarterial, intracranial, intrathecal, subarachnoidal, intraorbital, intracapsular, subcapsular, intracardiac, intrahepatic, intraarticular, intrasynovial, intraspinal, epidural, intrasternal, intralesional or combinations thereof, wherein administration by each route can be, e.g., by injection or infusion. Administration can also be performed, for example, once, a plurality of times, and/or over one or more extended periods.

The term “monotherapy” as used herein means that the CD25 antigen binding protein is the only anti-cancer agent administered to the subject during the treatment cycle. Other therapeutic agents, however, can be administered to the subject. For example, anti-inflammatory agents or other agents administered to a subject with cancer to treat symptoms associated with cancer, but not the underlying cancer itself, including, for example inflammation, pain, weight loss, and general malaise, can be administered during the period of monotherapy.

Administration “in combination with” one or more further therapeutic agents includes simultaneous (concurrent) and consecutive or sequential administration in any order.

The term “concurrently” is used herein to refer to administration of two or more therapeutic agents, where at least part of the administration overlaps in time or where the administration of one therapeutic agent falls within a short period of time relative to administration of the other therapeutic agent. For example, the two or more therapeutic agents are administered simultaneously or with a time separation of no more than about any of 20, 15, 10, 5, or 1 minutes.

The term “sequentially” is used herein to refer to administration of two or more therapeutic agents where the administration of one or more agent(s) occurs after discontinuing the administration of one or more other agent(s). For example, administration of the two or more therapeutic agents are administered with a time separation of more than 20 minutes, such as about 21, about 22, about 23 about 24 about 25, about 26, about 27, about 28, about 29, about 30, or about 31 minutes, such as about any of 35, 40, 50, or 60 minutes, 1 day, 2 days, 3 days, 1 week, 2 weeks, or 1 month, or longer.

The term “chemotherapeutic agent” refers to all chemical compounds that are effective in inhibiting tumor growth. Non-limiting examples of chemotherapeutic agents include alkylating agents (e.g., nitrogen mustards, ethyleneimine compounds, alkyl sulphonates, thiotepa and cyclosphosphamide); antimetabolites (e.g., folic acid, purine or pyrimidine antagonists); mitotic inhibitors (e.g., anti-tubulin agents such as vinca alkaloids, auristatins and derivatives of podophyllotoxin); cytotoxic antibiotics (e.g. anthracyclins such as doxorubicin, doxil (pegylated liposomal doxorubicin hydrochloride, myocet (non-pegylated liposomal doxorubicin), daunorubicin, epirubicin and idarubicin, mitomycin-C, bleomycin, dactinomycin, plicamycin, streptozocin); compounds that damage or interfere with DNA expression or replication (e.g., DNA minor groove binders); inhibitors of growth factors and/or of their corresponding receptors (growth factors such as for example platelet derived growth factor (PDGF), fibroblast growth factor (FGF), vascular endothelial growth factor (VEGF), epidermal growth factor (EGF), insuline-like growth factors (IGF), human epidermal growth factor (HER, e.g. HER2, HER3, HER4) and hepatocyte growth factor (HGF) and/or their corresponding receptors); hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide); aromatase inhibitors (e.g. anastrozole, letrozole, liarozole, vorozole, exemestane, atamestane); LHRH agonists and antagonists (e.g. goserelin acetate, luprolide); and cytotoxic or cytostatic agents.

The phrase “other additional cancer therapies” includes any cancer therapy used to treat a cancer and known to the person of skill in the art, such as e.g., radiation therapy, surgery, chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy. In some aspects, the additional cancer therapy is the administration of a small molecule enzymatic inhibitor or anti-metastatic agent. In some aspects, the additional cancer therapy is a therapy targeting the PBK/AKT/mTOR pathway, a HSP90 inhibitor, a tubulin inhibitor, an apoptosis inhibitor, and/or a chemopreventative agent. The additional cancer therapy may be one or more of the chemotherapeutic agents known in the art. A wide variety of chemotherapeutic agents may be used in accordance with the present ADC. The term “chemotherapy” refers to the use of drugs to treat cancer. A chemotherapeutic agent is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Examples of chemotherapeutic agents include, but are not limited to, alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins including bullatacin and bullatacinone; a camptothecin including the synthetic analogue topotecan; bryostatin; callystatin; CC-1065 including its adozelesin, carzelesin and bizelesin synthetic analogues; cryptophycins including cryptophycin 1 and cryptophycin 8; dolastatin; duocarmycin including the synthetic analogues, KW-2189 and CB 1-TMl; eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and raninmustine; antibiotics, such as the enediyne antibiotics, e.g., calicheamicin, including calicheamicin gammall and calicheamicin omegall; dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin including morpholinodoxorubicin, cyanomorpholino-doxorubicin, 2-pyrrolinodoxorubicin and deoxydoxorubicin; epirubicin; esorubicin; idarubicin; marcellomycin; mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; itoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes including T-2 toxin, verracurin A, roridin A and anguidine; urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan, e.g., CPT-11; topoisomerase inhibitor RFS 2000; difluorometlhylomithine (DMFO); retinoids, such as retinoic acid; capecitabine; carboplatin, procarbazine, plicomycin, gemcitabien, navelbine, famesylprotein tansferase inhibitors, transplatinum, and pharmaceutically acceptable salts, acids, or derivatives of any of the above. The dosing regimen of a combination therapy of an anti-CD25-drug conjugate and an additional cancer therapy can be determined based on the overall health of the subject to be treated and standard dosing guidelines for cancer therapy and/or combination therapies comprising antibody therapy and additional antibody or non-antibody cancer therapy.

“Sustained response” refers to the sustained effect on reducing tumor growth after cessation of a treatment. For example, the tumor size may remain the same or be reduced in size compared to the size at the beginning of the administration phase. In some aspects, the sustained response has a duration that is at least the same as the treatment duration, or at least 1.5, 2.0, 2.5, or 3 times longer than the treatment duration.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for progressive disease (PD), taking as reference the smallest SLD since the treatment started.

As used herein, “progressive disease” or “PD” refers to a disease (e.g., cancer) that is getting worse or is spreading. For example, progressive disease refers to the growing and/or spreading of a cancer.

As used herein, “progression free survival” or “PFS” refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” or “ORR” refers to the sum of complete response (CR) rate and partial response (PR) rate.

As used herein, “overall survival” or “OS” refers to the percentage of individuals in a group who are likely to be alive after a particular duration of time.

The phrase “pharmaceutically acceptable” indicates that the substance or composition is compatible chemically and/or toxicologically with the other ingredients comprising a formulation, and/or the subject being treated therewith.

The terms “pharmaceutical formulation” and “pharmaceutical composition” refer to a preparation which is in such form as to permit the biological activity of the active ingredient(s) to be effective, and which contains no additional components which are unacceptably toxic to a subject to which the formulation would be administered. Such formulations may be sterile.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, pamoate (i.e., 4,4′-methylene-bis-(2-hydroxy-3-naphthoate)) salts, alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

A “sterile” formulation is aseptic or essentially free from living microorganisms and their spores.

The terms “baseline” or “baseline value” used interchangeably herein can refer to a measurement or characterization of a symptom before the administration of the therapy or at the beginning of administration of the therapy. The baseline value can be compared to a reference value in order to determine the reduction or improvement of a symptom of a disease contemplated herein (e.g., cancer). The terms “reference” or “reference value” used interchangeably herein can refer to a measurement or characterization of a symptom after administration of the therapy. The reference value can be measured one or more times during a dosage regimen or treatment cycle or at the completion of the dosage regimen or treatment cycle. A “reference value” can be an absolute value; a relative value; a value that has an upper and/or lower limit; a range of values; an average value; a median value: a mean value; or a value as compared to a baseline value.

Similarly, a “baseline value” can be an absolute value; a relative value; a value that has an upper and/or lower limit; a range of values; an average value; a median value; a mean value; or a value as compared to a reference value. The reference value and/or baseline value can be obtained from one individual, from two different individuals or from a group of individuals (e.g., a group of two, three, four, five or more individuals).

An “adverse event” (AE) as used herein is any unfavorable and generally unintended or undesirable sign (including an abnormal laboratory finding), symptom, or disease associated with the use of a medical treatment. A medical treatment can have one or more associated AEs and each AE can have the same or different level of severity. Reference to methods capable of “altering adverse events” means a treatment regime that decreases the incidence and/or severity of one or more AEs associated with the use of a different treatment regime. A “serious adverse event” or “SAE” as used herein is an adverse event that meets one of the following criteria:
Is fatal or life-threatening (as used in the definition of a serious adverse event, “life-threatening” refers to an event in which the patient was at risk of death at the time of the event;
It does not refer to an event, which hypothetically might have caused death if it was more severe;
Results in persistent or significant disability/incapacity;
Constitutes a congenital anomaly/birth defect;
Is medically significant, i.e., defined as an event that jeopardizes the patient or may require medical or surgical intervention to prevent one of the outcomes listed above. Medical and scientific judgment must be exercised in deciding whether an AE is “medically significant;”
Requires inpatient hospitalization or prolongation of existing hospitalization, excluding the following: 1) routine treatment or monitoring of the underlying disease, not associated with any deterioration in condition; 2) elective or pre-planned treatment for a pre-existing condition that is unrelated to the indication under study and has not worsened since signing the informed consent; and 3) social reasons and respite care in the absence of any deterioration in the patient's general condition.

The term “package insert” is used to refer to instructions customarily included in commercial packages of therapeutic products, that contain information about the indications, usage, dosage, administration, combination therapy, contraindications and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker described herein. In certain aspects, the manufacture or kit is promoted, distributed, or sold as a unit for performing the methods described herein.

Various aspects of the disclosure are described in further detail in the following sections.

### II. Overview

Antigen binding proteins, including antibodies and antibody fragments that bind CD25 are provided herein. The antibody and antibody fragments contain an antigen binding domain that specifically binds to CD25, including to human CD25 (e.g., SEQ ID NO. 42).

In some aspects, the antigen binding proteins provided enable ADCC activity and, when present in an ADC deplete regulatory T cells (Tregs) from peripheral blood mononuclear cells (PBMC). The antigen binding proteins when present in an ADC demonstrate in vitro cytotoxicity towards CD25 expressing lymphoma cells and in vivo cytotoxicity towards CD25 expressing lymphoma cell xenografts.

In some aspects, the antigen binding proteins provided, when present in an ADC, deplete Treg cells in human CD25 transgenic mice while having no effect on CD4 T cells in the mice.

Advantageously, when present in MMAE ADCs, the antigen binding proteins deplete human Tregs cells from human peripheral blood but not CD4 or CD8 cells. In contrast, when the antigen binding proteins are present, e.g., in camptothecin or anthracycline ADCs they deplete Treg cells, CD4, and CD8 T cells.

Further provided are detuned antigen binding proteins that bind to CD25 with reduced affinity. In some aspects, some of the detuned antigen binding proteins bind human and cynomolgus monkey CD25 with reduced affinity compared to non-detuned antigen binding proteins. In some aspects, the detuned antigen binding proteins provided show different extents of reduction of CD25 binding. In some aspects, the detuned antigen binding proteins provided also show different extents of reduction of in vitro cytotoxicity towards CD25 expressing lymphoma cell lines and in vivo anti-tumor activity towards CD25 expressing lymphoma xenografts.

In some aspects, the detuned antigen binding protein also demonstrates reduced Treg depletion from PBMCs and, when present in an ADC, shows reduced in vitro cytotoxicity towards purified human Treg cells and Treg cells in human CD25 transgenic mice. In some aspects, the detuned antigen binding protein also demonstrates no anti-tumor activity towards purified human CD8 T cells or CD4 T cells in the human CD25 transgenic mice.

When tested in a syngeneic colon cancer model in human CD25 transgenic mice, the detuned antigen binding protein ADC shows anti-tumor activity that is higher than the anti-tumor activity of a non-detuned antigen binding protein ADC and is comparable to a known anti-tumor PD1 antibody.

In view of some CD25 antigen binding proteins or fragments thereof having reduced binding affinity to CD25, the antigen binding proteins that are provided can be used to alter a number of important biological activities, including, for example, to deplete Treg cells while leaving other CD25 expressing T cells unaffected. In addition, in some aspects, the antigen binding proteins are used to treat the consequences, symptoms, and/or the pathology associated with Treg cell activity. In some aspects, such therapeutic uses include, but are not limited to, immunotherapy application, including treatment of cancer. In some aspects, other uses for the antigen binding proteins include, for example, diagnosis of CD25-associated disease or conditions and screening assays to determine the presence or absence of CD25.

### III. Target

CD25 (IL-2 receptor α) is part of the IL-2 receptor complex that further comprises IL2 receptor β (CD122) and IL-2 receptor γ (CD132). CD25 is present on many types of T lymphocytes. CD25 is expressed at high levels on regulatory T cells (Tregs) and is required for Treg function. The expression of CD25 was found to be elevated on tumor infiltrating lymphocytes (TILs) compared to peripheral blood mononuclear cells. And within intratumor lymphocytes, CD25 expression was found to be highest on intratumor Tregs compared to other T cells.

The sequence of human CD25 is shown in Table 4 as SEQ ID NO: 42.

### IV. Anti-CD25 Antigen Binding Proteins

A variety of antigen binding proteins are provided herein and are described in greater detail below. In some embodiments, the antigen binding proteins that are disclosed herein comprise a scaffold, such as a polypeptide or polypeptides, into which one or more (e.g., 1, 2, 3, 4, 5 or 6) hypervariable regions (HVRs) or complementarity determining regions (CDRs) are embedded, grafted, and/or joined. In some antigen binding proteins, the HVRs or CDRs are embedded, grafted or joined into a “framework” region, which orients the HVRs or CDR(s) such that the proper antigen binding properties of the HVRs or CDRs are achieved. In some aspects, the antigen binding protein comprises one or more VH and/or VL domains.

In some antigen binding proteins, the HVR or CDR sequences are embedded, grafted or joined in or into a protein scaffold or other biocompatible polymer. In some aspects, the antigen binding protein is an antibody, or is derived from an antibody. Accordingly, the antigen binding proteins that are provided include, but are not limited to, monoclonal antibodies, multispecific antibodies (e.g., bispecific antibodies), minibodies, domain antibodies, synthetic antibodies (sometimes referred to herein as “antibody mimetics”), chimeric antibodies, humanized antibodies, human antibodies, antibody fusions, ADCs, and portions or fragments of each of the foregoing. Examples of antigen binding proteins provided herein that are fragments include, but are not limited to, a Fab, a Fab′, a F(ab′)2, a scFv, and a domain antibody. In certain aspects, the antigen binding protein is a DVD-Ig, DART, or BiTE or in another format as described in greater detail below.

The CD25 antibodies SG25Ab-9 and SG25Ab-9 YH98A in some aspects bind to CD25 with an affinity (e.g., EC₅₀) of about 50 pM to about 500 nM. In some aspects, the CD25 antibodies SG25Ab-9 and SG25Ab-9 YH98A bind to CD25 with an affinity of about 75 pM to about 480 nM, about 100 pM to about 450 nM, about 150 pM to about 400 nM, about 200 pM to about 350 nM, about 250 pM to about 300 nM, about 300 pM to about 250 nM, about 350 pM to about 200 nM, about 400 pM to about 150 nM, about 450 pM to about 140 nM, about 500 pM to about 130 nM, about 525 pM to about 120 nM, about 550 pM and 110 nM, about 575 pM to about 100 nM, about 600 pM to about 90 nM, about 625 pM to about 80 nM, about 650 pM to about 70 nM, about 675 pM to about 65 nM, about 700 pM to about 60 nM, about 750 pM to about 55 nM, about 800 pM to about 50 nM, about 850 pM to about 45 nM, about 900 pM to about 40 nM, about 950 pM to about 35 nM, about 1 nM to about 30 nM, about 1.5 nM to about 25 nM, about 2 nM to about 20 nM, about 2.5 nM to about 15 nM, about 3 nM to about 10 nM; or about 50 pM to about 100 pM, about 50 pM to about 0.5 nM, about 75 pM to about 1 nM, about 0.5 nM to about 5 nM; or about 0.05, 0.1, 0.2, 0.3, 0.4, 0.5, 0.6, 0.7, 0.8, 0.9, 1, 2, 3, 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90,91, 92, 93, 94, 95, 96, 97, 98, 99, 100, 102, 104, 106, 108, 110, 112, 114, 116, 118, 120, 122, 124, 126, 128, 130, 132, 134, 136, 138, 140, 142, 144, 146, 148, 150, 175, 200, 225, 250, 275, 300, 325, 350, 375, 400, 425, 450, 475, or 500 nM. In some aspects, the binding affinity is determined according to the assay described in Example 1. In some aspects, the binding affinity is measured as binding to surface immobilized recombinant CD25 using biolayer interferometry or ELISA. In some aspects, the binding affinity is measured as binding to surface immobilized recombinant CD25 using ELISA. In some aspects, the binding affinity is measured as binding to CD25 expressed on a cell surface using flow cytometry.

In some aspects, the antigen binding proteins bind to human CD25 and cynomolgus CD25. In some aspects, the antigen binding proteins bind to human and cynomolgus CD25 with similar affinity. In some aspects, the antigen binding proteins bind to human CD25 but do not bind to cynomolgus CD25. In some aspects, the antigen binding proteins bind to human CD25 immobilized on a surface with an affinity of about 50 pM to about 0.5 nM and to cynomolgus CD25 immobilized to a surface with an affinity of about 0.8 nM to about 5 nM as measured by ELISA as shown in Example 2.

In some aspects, the antigen binding proteins bind to CD25 expressed on human cells with an affinity between about 80 pM and 3 nM, or about 100 pM and about 600 pM, or about 600 pM and about 2.4 nM as shown in Example 2.

In some aspects, the antigen binding proteins internalize into CD25 expressing cells. In some aspects, the antigen binding proteins internalize into CD25 expressing cells with an efficacy similar to daclizumab as shown in Example 2.

In some aspects, the antigen binding proteins enable ADCC activity as shown in Example 2. In some aspects, the antigen binding proteins enable ADCC activity when incubated with human NK cells and tumor target cells. In some aspects, the antigen binding proteins when present in an ADC enable ADCC activity with human NK cells and tumor target cells as exemplified in Example 2. In some aspects, the antigen binding proteins when fucosylated enable ADCC activity when incubated with human NK cells and tumor target cells. In some aspects, the antigen binding proteins when non-fucosylated enable ADCC activity when incubated with human NK cells and tumor target cells.

In some aspects, the antigen binding proteins are variant antigen binding proteins that comprise a mutation in at least one amino acid in a heavy chain variable region and bind to surface immobilized CD25 with reduced affinity compared to antigen binding proteins without a mutation. In some aspects, the antigen binding proteins bind surface immobilized CD25 with an affinity of about 100 pM to about 200 pM and the variant antigen binding proteins bind surface immobilized CD25 with an affinity of between about 250 pM and about 250 nM, or about 300 pM and about 200 nM, about 400 pM and about 180 nM, about 500 pM and about 170 nM or about 900 pM and about 150 nM as measured by ELISA as shown in Example 3.

In some aspects, the antigen binding proteins are variant antigen binding proteins that comprise a mutation in at least one amino acid in a heavy chain variable region and bind to cell surface expressed CD25 with reduced affinity compared to antigen binding proteins without a mutation. In some aspects, the antigen binding proteins bind cell surface expressed CD25 with an affinity of about 600 pM to about 700 pM and the variant antigen binding proteins bind cell surface expressed CD25 with an affinity of between about 800 pM and about 300 nM, as measured by ELISA as shown in Example 3. In some aspects, the antigen binding proteins bind cell surface expressed CD25 with an affinity of about 800 pM to about 1 nM and the variant antigen binding proteins bind cell surface expressed CD25 with an affinity of between about 1.1 nM and about 500 nM, as measured by flow cytometry as shown in Example 3.

In some aspects, the antigen binding proteins are variant antigen binding proteins that comprise a mutation in at least one amino acid in a heavy chain variable region and have reduced ADCC activity when incubated with human NK cells and tumor target cells compared to antigen binding proteins without a mutation as shown in Example 3.

In some aspects, the antigen binding proteins are variant antigen binding proteins that comprise a mutation in at least one amino acid in a heavy chain variable region and have reduced in vitro cytotoxicity when present in an ADC compared to antigen binding proteins without a mutation as shown in Example 4.

In some aspects, the antigen binding proteins are variant antigen binding proteins that comprise a mutation in at least one amino acid in a heavy chain variable region and have reduced in vivo anti-tumor activity when present in an ADC compared to antigen binding proteins without a mutation as shown in Example 5.

In some aspects, the antigen binding proteins are variant antigen binding proteins that comprise a mutation in at least one amino acid in a heavy chain variable region and have reduced Treg depleting activity compared to antigen binding proteins without a mutation as shown in Example 6.

In some aspects, the antigen binding proteins are variant antigen binding proteins that comprise a mutation in at least one amino acid in a heavy chain variable region and have no in vitro cytotoxic activity towards CD8 T cells when present in an ADC similar to antigen binding proteins without a mutation as shown in Example 6.

In some aspects, the antigen binding proteins are variant antigen binding proteins that comprise a mutation in at least one amino acid in a heavy chain variable region and have no in vitro cytotoxic activity towards CD4 T cells when present in an ADC similar to antigen binding proteins without a mutation as shown in Example 6.

In some aspects, the antigen binding proteins are variant antigen binding proteins that comprise a mutation in at least one amino acid in a heavy chain variable region and have similar in vivo anti-tumor activity in a colon cancer mouse model when present in an ADC as antigen binding proteins without a mutation as shown in Example 7.

In some aspects, the antigen binding proteins are variant antigen binding proteins that comprise a mutation in at least one amino acid in a heavy chain variable region and have reduced in vivo Treg depleting activity in non-human primates when present in an ADC compared to antigen binding proteins without a mutation as shown in Example 8.

In some aspects, the antigen binding proteins are variant antigen binding proteins that comprise a mutation in at least one amino acid in a heavy chain variable region and have similar total antibody pharmacokinetics in non-human primates when present in an ADC as antigen binding proteins without a mutation as shown in Example 8.

In some aspects, the antigen binding proteins are variant antigen binding proteins that comprise a mutation in at least one amino acid in a heavy chain variable region and have similar antibody-conjugated drug pharmacokinetics in non-human primates when present in an ADC as antigen binding proteins without a mutation as shown in Example 8.

Thus, in some aspects, the antigen binding protein has 1, 2, 3, 4, 5, 6, 7, 8, 9, 10, 11, 12, 13, 14, or 15 of the characteristics 1-15 in any combination or the antigen binding protein has 1, 2, 3, 4, 5, 6, 7, 8, 9, 10, 11, 12, 13, 14 or 15 of the characteristics 16-26 in any combination:
1. Binding to human and/or cynomolgus CD25;
2. Internalization into cells expressing CD25;
3. ADCC activity;
4. In vitro cytotoxicity towards CD25 expressing tumor cell lines when present in an ADC;
5. In vivo anti-tumor activity in CD25 expressing tumor models in mice when present in an ADC;
6. In vivo Treg cell depleting activity in a human CD25 transgenic mouse when present in an ADC;
7. Lack of in vivo CD4 T cell depleting activity in a human CD25 transgenic mouse when present in an ADC;
8. In vitro human Treg depleting activity in PBMC when present in a MMAE ADC;
9. Low level of in vitro human CD4 T cell depleting activity in PBMC when present in a MMAE ADC;
10. Lack of in vitro human CD8 T cell depleting activity in PBMC when present in a MMAE ADC;
11. In vitro human Treg cell depleting activity in PBMC when present in a Camptothecin or Anthracycline ADC;
12. In vitro human CD 8 T cell depleting activity in PBMC when present in a Camptothecin or Anthracycline ADC;
13. Anti-tumor activity in a colon cancer mouse model when present in a MMAE ADC;
14. Reduced binding to human and/or cynomolgus CD25 when comprising a mutation in a heavy chain variable region;
15. Reduced ADCC activity when comprising a mutation in a heavy chain variable region;
16. Reduced in vitro cytotoxicity towards CD25 expressing tumor cell lines when comprising a mutation in a heavy chain variable region and present in an ADC;
17. Reduced in vivo anti-tumor activity in CD25 expressing tumor models in mice when comprising a mutation in a heavy chain variable region and present in an ADC;
18. Reduced in vitro human Treg depleting activity in PBMC when and comprising a mutation in a heavy chain variable region present in an ADC;
19. Reduced in vitro cytotoxicity towards purified human Treg cells when comprising a mutation in a heavy chain variable region and present in MMAE ADC;
20. Lack of in vitro cytotoxicity towards purified human CD8 T cells when comprising a mutation in a heavy chain variable region and present in MMAE ADC;
21. Lack of in vivo CD4 T cell depletion in human CD25 transgenic mice when comprising a mutation in a heavy chain variable region and present in a MMAE ADC;
22. Reduced in vivo Treg depletion in human CD25 transgenic mice when comprising a mutation in a heavy chain variable region and present in a MMAE ADC;
23. Increased anti-tumor activity in a colon cancer mouse model when comprising a mutation in a heavy chain variable region and present in a MMAE ADC;
24. Reduced in vivo Treg depletion in non-human primates when comprising a mutation in a heavy chain variable region and present in a MMAE ADC;
25. Maintenance of total antibody pharmacokinetics in non-human primates when comprising a mutation in a heavy chain variable region and present in a MMAE ADC;
26. Maintenance of antibody-conjugated MMAE pharmacokinetics in non-human primates when comprising a mutation in a heavy chain variable region.

### A. Exemplary Antibodies and Antibody Fragments

In some aspects, the antigen binding proteins that are provided include the CD25 antibodies SG25Ab-1, SG25Ab-2, SG25Ab-3, SG25Ab-4, SG25Ab-5, SG25Ab-6, SG25Ab-7, SG25Ab-8, and SG25Ab-9, or antigen binding fragments thereof, that are described in the Examples herein.

In some aspects, the antigen binding protein is the CD25 antibody SG25Ab-9, which interacts with CD25 through heavy chain variable region amino acid residues comprising at least one of YH27, SH28, Y32, Y52, G53, D54, S55, D56, G96, YH98, YH99, A100, FH100A, or DH101. In some aspects, the antigen binding protein is the CD25 antibody SG25Ab-9 and the paratope of SG25Ab-9 comprises at least one of the heavy chain variable region amino acid residues YH27, SH28, Y32, Y52, G53, D54, S55, D56, G96, YH98, YH99, A100, FH100A, or DH101.

In some aspects, the antigen binding protein is the CD25 antibody SG25Ab-9 and comprises a mutation of any one or more of amino acid residues YH27, SH28, Y32, Y52, G53, D54, S55, D56, G96, YH98, YH99, A100, FH100A, or DH101, wherein the binding affinity of SG25Ab-9 to CD25 expressed on a cell surface as measured by flow cytometry is affected as exemplified in Example 3. In some aspects, the antigen binding protein is the CD25 antibody SG25Ab-9 and comprises a mutation of any one or more of amino acid residues YH27, SH28, Y32, Y52, G53, D54, S55, D56, G96, YH98, YH99, A100, FH100A, or DH101, wherein the binding affinity of SG25Ab-9 to CD25 on a surface as measured by ELISA is affected as exemplified in Example 3.

In some aspects, the antigen binding protein is the CD25 antibody SG25Ab-9 and a mutation of at least 1, 2, 3, 4, 5, 6, 7, 8, 9, 10, 11, 12, 13, or 14 of amino acid residues YH27, SH28, Y32, Y52, G53, D54, S55, D56, G96, YH98, YH99, A100, FH100A, or DH101 reduces the binding affinity of SG25Ab-9 to surface immobilized CD25. In some aspects, the antigen binding protein is the CD25 antibody SG25Ab-9 (SG25Ab-9) and a mutation of at least 1, 2, 3, 4, 5, 6, 7, 8, 9, 10, 11, 12, 13, or 14 of amino acid residues YH27, SH28, Y32, Y52, G53, D54, S55, D56, G96, YH98, YH99, A100, FH100A, or DH101 reduces the binding affinity of SG25Ab-9 to CD25 expressed on a cell surface.

In some aspects, the antigen binding protein is present in an ADC, wherein the antigen binding protein is the CD25 antibody SG25Ab-9 and a mutation of any one of amino acid residues YH27, SH28, Y32, Y52, G53, D54, S55, D56, G96, YH98, YH99, A100, FH100A, or DH101 in SG25Ab-9 present in an ADC reduces the in vitro cytotoxicity towards CD25 expressing tumor cell lines compared to SG25Ab-9 as exemplified in Example 4.

In some aspects, the antigen binding protein is present in an ADC, wherein the antigen binding protein is the CD25 antibody SG25Ab-9 and a mutation of any one of amino acid residues YH27, SH28, Y32, Y52, G53, D54, S55, D56, G96, YH98, YH99, A100, FH100A, or DH101 in SG25Ab-9 present in an ADC reduces the in vivo anti-tumor activity towards CD25 expressing tumor cell lines in a xenograft mouse model compared to SG25Ab-9 as exemplified in Example 5.

In some aspects, the antigen binding protein is the CD25 antibody SG25Ab-9 and a mutation of any one of amino acid residues YH27, SH28, Y32, Y52, G53, D54, S55, D56, G96, YH98, YH99, A100, FH100A, or DH101 in SG25Ab-9 reduces the in vitro Treg depleting activity compared to SG25Ab-9 as exemplified in Example 6.

In some aspects, the antigen binding protein is present in an ADC, wherein the antigen binding protein is the CD25 antibody SG25Ab-9 and a mutation of any one of amino acid residues YH27, SH28, Y32, Y52, G53, D54, S55, D56, G96, YH98, YH99, A100, FH100A, or DH101 in SG25Ab-9 present in an ADC reduces the in vitro cytotoxic activity towards purified human Treg cells compared to SG25Ab-9 as exemplified in Example 6.

In some aspects, the antigen binding protein is present in an ADC, wherein the antigen binding protein is the CD25 antibody SG25Ab-9 and a mutation of any one of amino acid residues YH27, SH28, Y32, Y52, G53, D54, S55, D56, G96, YH98, YH99, A100, FH100A, or DH101 in SG25Ab-9 present in an ADC maintains the lack of in vitro cytotoxic activity towards purified human CD8 T cells of SG25Ab-9 as exemplified in Example 6.

In some aspects, the antigen binding protein is the CD25 antibody SG25Ab-9 and a mutation of any one of amino acid residues YH27, SH28, Y32, Y52, G53, D54, S55, D56, G96, YH98, YH99, A100, FH100A, or DH101 in SG25Ab-9 maintains the lack of in vivo cytotoxic activity towards CD4 T cells of SG25Ab-9 in a human CD25 transgenic mouse as exemplified in Example 6.

In some aspects, the antigen binding protein is the CD25 antibody SG25Ab-9 and a mutation of any one of amino acid residues YH27, SH28, Y32, Y52, G53, D54, S55, D56, G96, YH98, YH99, A100, FH100A, or DH101 in SG25Ab-9 reduces the in vivo cytotoxic activity towards Treg cells of SG25Ab-9 in a human CD25 transgenic mouse as exemplified in Example 6.

In some aspects, the antigen binding protein is the CD25 antibody SG25Ab-9 and a mutation of any one of amino acid residues YH27, SH28, Y32, Y52, G53, D54, S55, D56, G96, YH98, YH99, A100, FH100A, or DH101 in SG25Ab-9 increases the anti-tumor activity of SG25Ab-9 in a colon cancer xenograft model of a human CD25 transgenic mouse as exemplified in Example 7.

In some aspects, the antigen binding protein is the CD25 antibody SG25Ab-9 and increases the in vivo cytotoxic activity towards Treg cells in PBMC in a colon cancer xenograft model of a human CD25 transgenic mouse as exemplified in Example 8.

In some aspects, the antigen binding protein is the CD25 antibody SG25Ab-9 and increases the in vivo cytotoxic activity towards Treg cells in splenocytes in a colon cancer xenograft model of a human CD25 transgenic mouse as exemplified in Example 8.

In some aspects, the antigen binding protein is the CD25 antibody SG25Ab-9 and substantially increases the in vivo cytotoxic activity towards Treg cells in the tumor of a colon cancer xenograft model of a human CD25 transgenic mouse as exemplified in Example 8.

In some aspects, the antigen binding protein is the CD25 antibody SG25Ab-9 and a mutation of any one of amino acid residues YH27, SH28, Y32, Y52, G53, D54, S55, D56, G96, YH98, YH99, A100, FH100A, or DH101 in SG25Ab-9 reduces the in vivo Treg depleting activity of SG25Ab-9 in a non-human primate model as exemplified in Example 9.

In some aspects, the antigen binding protein is the antibody SG25Ab-9 YH98A, the sequences of the CDRs, variable domains, and framework sequences of which are summarized in Tables 2, 3, and 4 below. Thus, in certain aspects, the antigen binding protein is one of the antibodies as listed in Tables 2, 3, and 4 below. Such antibodies comprise the corresponding CDR, variable domain, and heavy and light chain amino acid sequences as indicated in Tables 2, 3, and 4.

TABLE 2
CDR SEQ ID NOs for SG25Ab-9, SG25Ab-9 YH98A, SG25Ab-4

| | |
|---|---|
| Antigen Binding | CDR SEQ ID NOS: |

| Protein ID | H1 | H2 | H3 | L1 | L2 | L3 |
|---|---|---|---|---|---|---|
| SG25Ab-9 | 1 | 2 | 3 | 4 | 5 | 6 |
| SG25Ab-9 | 1 | 2 | 21 | 4 | 5 | 6 |
| YH98A | | | | | | |
| SG25Ab-4 | 25 | 26 | 27 | 28 | 29 | 30 |

TABLE 3
Variable Domain and Heavy and Light SEQ ID
NOs for SG25Ab-9, SG25Ab-9 YH98A, SG25Ab-4

| Antigen | | | | |
|---|---|---|---|---|
| Binding | | | | |
| Protein ID | VH | VL | HC | LC |
| SG25Ab-9 | 7 | 8 | 45 | 47 |
| SG25Ab-9 | 22 | 8 | 46 | 47 |
| YH98A | | | | |
| SG25Ab-4 | 31 | 32 | 43 | 44 |

TABLE 4
Antigen Binding Proteins Sequences

| | Antigen | | |
|---|---|---|---|
| SEQ | Binding | | |
| ID NO. | Protein ID | Name | SEQUENCE |
| 1 | SG25Ab-9; | CDR-H1 | SYWIG |
| | SG25Ab-9 | | |
| | YH98A | | |
| 2 | SG25Ab-9; | CDR-H2 | IIYPGDSDTRYSPSFQG |
| | SG25Ab-9 | | |
| | YH98A | | |
| 3 | SG25Ab-9 | CDR-H3 | LGSYYAFDI |
| 4 | SG25Ab-9; | CDR-L1 | TGTSSDVGAYIYVS |
| | SG25Ab-9 | | |
| | YH98A | | |
| 5 | SG25Ab-9; | CDR-L2 | DVSKRPS |
| | SG25Ab-9 | | |
| | YH98A | | |
| 6 | SG25Ab-9; | CDR-L3 | SSYTRSSTWV |
| | SG25Ab-9 | | |
| | YH98A | | |
| 7 | SG25Ab-9 | VH | QVQLVQSGAEVKKPGESLKISCKGSGYSSTSY |
| | | | WIGWVRQMPGKGLEWMGIIYPGDSDTRYSPSF |
| | | | QGQVTISADKSISTAYLQWSSLKASDTAMYYC |
| | | | ARLGSYYAFDIWGRGTMVTVSS |
| 8 | SG25Ab-9; | VL | QSALTQPASVSASPGQSITISCTGTSSDVGAYIY |
| | SG25Ab-9 | | VSWYQQLSGKPPKLILYDVSKRPSGISDRFSGS |
| | YH98A | | KSGNTASLTISGLQADDEADYYCSSYTRSSTW |
| | | | VFGGGTQLTVL |
| 9 | SG25Ab-9; | H-FR1 | QVQLVQSGAEVKKPGESLKISCKGSGYSST |
| | SG25Ab-9 | | |
| | YH98A | | |
| 10 | SG25Ab-9; | H-FR2 | WVRQMPGKGLEWMG |
| | SG25Ab-9 | | |
| | YH98A | | |
| 11 | SG25Ab-9; | H-FR3 | QVTISADKSISTAYLQWSSLKASDTAMYYCAR |
| | SG25Ab-9 | | |
| | YH98A | | |
| 12 | SG25Ab-9; | H-FR4 | WGRGTMVTVSS |
| | SG25Ab-9 | | |
| | YH98A | | |
| 13 | SG25Ab-9; | L-FR1 | QSALTQPASVSASPGQSITISC |
| | SG25Ab-9 | | |
| | YH98A | | |
| 14 | SG25Ab-9; | L-FR2 | WYQQLSGKPPKLILY |
| | SG25Ab-9 | | |
| | YH98A | | |
| 15 | SG25Ab-9; | L-FR3 | GISDRFSGSKSGNTASLTISGLQADDEADYYC |
| | SG25Ab-9 | | |
| | YH98A | | |
| 16 | SG25Ab-9; | L-FR4 | FGGGTQLTVL |
| | SG25Ab-9 | | |
| | YH98A | | |
| 17 | SG25Ab-9 | HC | mawvwtllflmaaaqgaqaQVQLVQSGAEVKKPGESL |
| | | (signal | KISCKGSGYSSTSYWIGWVRQMPGKGLEWMGI |
| | | peptide | IYPGDSDTRYSPSFQGQVTISADKSISTAYLQWS |
| | | italicized, | SLKASDTAMYYCARLGSYYAFDIWGRGTMVT |
| | | variable | VSSastkgpsvfplapsskstsggtaalgclvkdyfpepvtvswnsga |
| | | domain | ltsgvhtfpavlqssglyslssvvtvpssslgtqtyicnvnhkpsntkvdk |
| | | capitalized, | kvepkscdkthtcppcpapellggpsvflfppkpkdtlmisrtpevtcv |
| | | Y98 | vvdvshedpevkfnwyvdgvevhnaktkpreeqynstyrvvsvltvl |
| | | bolded, | hqdwlngkeykckvsnkalpapiektiskakgqprepqvytlppsrde |
| | | CDRs | ltknqvsltclvkgfypsdiavewesngqpennykttppvldsdgsffl |
| | | underlined, | yskltvdksrwqqgnvfscsvmhealhnhytqkslslspgk |
| | | constant | |
| | | domain | |
| | | lower case) | |
| 18 | SG25Ab-9 | HC | atggcttgggtgtggaccttgctattcctgatggcagctgcccaaggcgc |
| | SG25Ab-9; | nucleic | ccaagcaCAGGTCCAGCTGGTGCAGTCTGGAGC |
| | | acid | TGAGGTGAAGAAGCCTGGGGAGTCTCTGAAG |
| | | (signal | ATCTCCTGCAAGGGCTCTGGCTACTCATCCAC |
| | | peptide | TTCCTATTGGATAGGCTGGGTGCGCCAGATG |
| | | italicized, | CCTGGAAAGGGACTGGAGTGGATGGGCATCA |
| | | variable | TTTATCCTGGTGATTCTGACACACGCTACTCT |
| | | domain | CCATCTTTCCAAGGCCAGGTGACCATCTCTGC |
| | | capitalized, | AGACAAGTCCATCAGCACTGCCTATCTGCAG |
| | | Y98 | TGGAGCAGCCTGAAGGCTTCAGACACTGCCA |
| | | bolded, | TGTACTACTGTGCTAGACTGGGTTCTTACTAT |
| | | CDRs | GCCTTTGACATCTGGGGCAGAGGCACCATGG |
| | | underlined, | TCACCGTCTCCTCAgctagcaccaagggcccatctgtettccc |
| | | constant | cctggcaccctcctccaagagcacctctgggggcacagctgccctgggc |
| | | domain | tgcctggtcaaggactacttccctgaacctgtgacagtgtcctggaactca |
| | | lower case) | ggagccctgaccagcggcgtgcacaccttcccggctgtcctacagtcctc |
| | | | aggactctactccctcagcagcgtggtgaccgtgccctccagcagcttgg |
| | | | gcacccagacctacatctgcaacgtgaatcacaagcccagcaacaccaa |
| | | | ggtggacaagaaagttgagcccaaatcttgtgacaaaactcacacatgcc |
| | | | caccgtgcccagcacctgaactcctggggggaccgtcagtcttcctcttcc |
| | | | ccccaaaacccaaggacaccctcatgatctcccggacccctgaggtcac |
| | | | atgcgtggtggtggacgtgagccacgaagaccctgaggtcaagttcaact |
| | | | ggtacgtggacggcgtggaggtgcataatgccaagacaaagccgcggg |
| | | | aggagcagtacaacagcacgtaccgtgtggtcagcgtcctcaccgtcctg |
| | | | caccaggactggctgaatggcaaggagtacaagtgcaaggtctccaaca |
| | | | aagccctcccagcccccatcgagaaaaccatctccaaagccaaagggca |
| | | | gccccgagaaccacaggtgtacaccctgcccccatcccgggatgagctg |
| | | | accaagaaccaggtcagcctgacctgcctggtcaaaggcttctatcccag |
| | | | cgacatcgccgtggagtgggagagcaatgggcagccggagaacaacta |
| | | | caagaccacgcctcccgtgctggactccgacggctccttcttcctctacag |
| | | | caagctcaccgtggacaagagcaggtggcagcaggggaacgtcttctca |
| | | | tgctccgtgatgcatgaggctctgcacaaccactacacacagaagagcct |
| | | | ctccctgtctccgggtaaatga |
| 19 | SG25Ab-9 | LC | mawalllltlltqdtgswaQSALTQPASVSASPGQSITISC |
| | YH98A | (signal | TGTSSDVGAYIYVSWYQQLSGKPPKLILYDVSK |
| | | peptide | RPSGISDRFSGSKSGNTASLTISGLQADDEADY |
| | | italicized, | YCSSYTRSSTWVFGGGTQLTVLgqpkaapsvtlfppss |
| | | variable | eelqankatlvclisdfypgavtvawkadsspvkagvetttpskqsnnk |
| | | domain | yaassylsltpeqwkshrsyscqvthegstvektvaptecs |
| | | capitalized, | |
| | | CDRs | |
| | | underlined, | |
| | | constant | |
| | | domain | |
| | | lower case) | |
| 20 | SG25Ab-9; | LC | atggcctgggctctgctgctcctcactctcctcactcaggacacaggatc |
| | SG25Ab-9 | nucleic | ctgggccCAGTCTGCTCTGACACAGCCAGCTTCA |
| | YH98A | acid | GTGTCTGCATCTCCAGGACAGTCCATCACCA |
| | | (signal | TCTCCTGCACTGGAACCAGCAGTGATGTTGG |
| | | peptide | GGCATATATCTATGTCAGCTGGTACCAGCAG |
| | | italicized, | CTGTCAGGCAAACCACCAAAGCTGATCCTCT |
| | | variable | ATGATGTCTCCAAGCGGCCCTCTGGGATCTCT |
| | | domain | GACAGGTTCAGTGGCTCCAAGTCTGGGAACA |
| | | capitalized, | CAGCCTCTCTCACAATCTCTGGGCTGCAGGCT |
| | | CDRs | GATGATGAGGCAGACTATTACTGCTCTTCAT |
| | | underlined, | ATACTAGGAGCAGCACTTGGGTGTTCGGCGG |
| | | constant | AGGAACCCAGCTGACTGTCCTAggtcagcccaaggc |
| | | domain | tgccccctcggtcactctgttcccgccctcctctgaggagcttcaagccaa |
| | | lower case) | caaggccacactggtgtgtctcataagtgacttctacccgggagccgtgac |
| | | | agtggcctggaaggcagatagcagccccgtcaaggcgggagtggagac |
| | | | caccacaccctccaaacaaagcaacaacaagtacgcggccagcagctat |
| | | | ctgagcctgacgcctgagcagtggaagtcccacagaagctacagctgcc |
| | | | aggtcacgcatgaagggagcaccgtggagaagacagtggcccctacag |
| | | | aatgttcatag |
| 21 | SG25Ab-9 | YH98A | LGSAYAFDI |
| | YH98A | CDR-H3 | |
| 22 | SG25Ab-9 | YH98A | QVQLVQSGAEVKKPGESLKISCKGSGYSSTSY |
| | YH98A | VH | WIGWVRQMPGKGLEWMGIIYPGDSDTRYSPSF |
| | | | QGQVTISADKSISTAYLQWSSLKASDTAMYYC |
| | | | ARLGSAYAFDIWGRGTMVTVSS |
| 23 | SG25Ab-9 | YH98A | mawvwtllflmaaaqgaqaQVQLVQSGAEVKKPGESL |
| | YH98A | HC (signal | KISCKGSGYSSTSYWIGWVRQMPGKGLEWMGI |
| | | peptide | IYPGDSDTRYSPSFQGQVTISADKSISTAYLQWS |
| | | italicized, | SLKASDTAMYYCARLGSAYAFDIWGRGTMVT |
| | | variable | VSSastkgpsvfplapsskstsggtaalgclvkdyfpepvtvswnsga |
| | | domain | ltsgvhtfpavlqssglyslssvvtvpssslgtqtyicnvnhkpsntkvdk |
| | | capitalized, | kvepkscdkthtcppcpapellggpsvflfppkpkdtlmisrtpevtcv |
| | | YH98A | vvdvshedpevkfnwyvdgvevhnaktkpreeqynstyrvvsvltvl |
| | | bolded, | hqdwlngkeykckvsnkalpapiektiskakgqprepqvytlppsrde |
| | | CDRs | ltknqvsltclvkgfypsdiavewesngqpennykttppvldsdgsffl |
| | | underlined, | yskltvdksrwqqgnvfscsvmhealhnhytqkslslspgk |
| | | constant | |
| | | domain | |
| | | lower case) | |
| 24 | SG25Ab-9 | YH98A | atggcttgggtgtggaccttgctattcctgatggcagctgcccaaggcgc |
| | YH98A | HC nucleic | ccaagcaCAGGTCCAGCTGGTGCAGTCTGGAGC |
| | | acid (signal | TGAGGTGAAGAAGCCTGGGGAGTCTCTGAAG |
| | | peptide | ATCTCCTGCAAGGGCTCTGGCTACTCATCCAC |
| | | italicized, | TTCCTATTGGATAGGCTGGGTGCGCCAGATG |
| | | variable | CCTGGAAAGGGACTGGAGTGGATGGGCATCA |
| | | domain | TTTATCCTGGTGATTCTGACACACGCTACTCT |
| | | capitalized, | CCATCTTTCCAAGGCCAGGTGACCATCTCTGC |
| | | YH98A | AGACAAGTCCATCAGCACTGCCTATCTGCAG |
| | | bolded, | TGGAGCAGCCTGAAGGCTTCAGACACTGCCA |
| | | CDRs | TGTACTACTGTGCTAGACTGGGTTCTGCTTAT |
| | | underlined, | GCCTTTGACATCTGGGGCAGAGGCACCATGG |
| | | constant | TCACCGTCTCCTCAgctagcaccaagggcccatctgtcttccc |
| | | domain | cctggcaccctcctccaagagcacctctgggggcacagctgccctgggc |
| | | lower case) | tgcctggtcaaggactacttccctgaacctgtgacagtgtcctggaactca |
| | | | ggagccctgaccagcggcgtgcacaccttcccggctgtcctacagtcctc |
| | | | aggactctactccctcagcagcgtggtgaccgtgccctccagcagcttgg |
| | | | gcacccagacctacatctgcaacgtgaatcacaagcccagcaacaccaa |
| | | | ggtggacaagaaagttgagcccaaatcttgtgacaaaactcacacatgcc |
| | | | caccgtgcccagcacctgaactcctggggggaccgtcagtcttcctcttcc |
| | | | ccccaaaacccaaggacaccctcatgatctcccggacccctgaggtcac |
| | | | atgcgtggtggtggacgtgagccacgaagaccctgaggtcaagttcaact |
| | | | ggtacgtggacggcgtggaggtgcataatgccaagacaaagccgcggg |
| | | | aggagcagtacaacagcacgtaccgtgtggtcagcgtcctcaccgtcctg |
| | | | caccaggactggctgaatggcaaggagtacaagtgcaaggtctccaaca |
| | | | aagccctcccagcccccatcgagaaaaccatctccaaagccaaagggca |
| | | | gccccgagaaccacaggtgtacaccctgcccccatcccgggatgagctg |
| | | | accaagaaccaggtcagcctgacctgcctggtcaaaggcttctatcccag |
| | | | cgacatcgccgtggagtgggagagcaatgggcagccggagaacaacta |
| | | | caagaccacgcctcccgtgctggactccgacggctccttcttcctctacag |
| | | | caagctcaccgtggacaagagcaggtggcagcaggggaacgtcttctca |
| | | | tgctccgtgatgcatgaggctctgcacaaccactacacacagaagagcct |
| | | | ctccctgtctccgggtaaatga |
| 25 | SG25Ab-4 | CDR-H1 | RYWIA |
| 26 | SG25Ab-4 | CDR-H2 | IIYPGDSDARYSPTFEG |
| 27 | SG25Ab-4 | CDR-H3 | LGSYYAFDI |
| 28 | SG25Ab-4 | CDR-L1 | TGTSSDVGGYNYVS |
| 29 | SG25Ab-4 | CDR-L2 | DVSKRPS |
| 30 | SG25Ab-4 | CDR-L3 | SSYTSSSTWV |
| 31 | SG25Ab-4 | VH | EVQLVQSGAEVKKPGESLKISCKGPEYSFNRY |
| | | | WIAWVRQRPGKGLEWMGIIYPGDSDARYSPTF |
| | | | EGHVTLSADMSLTTAYLQWSSLRASDTAMYY |
| | | | CARLGSYYAFDIWGKGTMVTVSS |
| 32 | SG25Ab-4 | VL | QSALTQPASVSGSPGQSITIFCTGTSSDVGGYNY |
| | | | VSWYQQHPGKAPKLMIYDVSKRPSGVSNRFSG |
| | | | SKSGNTASLTISGLQAEDEADYYCSSYTSSSTW |
| | | | VFGGGTQLTVL |
| 33 | SG25Ab-4 | H-FR1 | EVQLVQSGAEVKKPGESLKISCKGPEYSFN |
| 34 | SG25Ab-4 | H-FR2 | WVRQRPGKGLEWMG |
| 35 | SG25Ab-4 | H-FR3 | HVTLSADMSLTTAYLQWSSLRASDTAMYYCAR |
| 36 | SG25Ab-4 | H-FR4 | WGKGTMVTVSS |
| 37 | SG25Ab-4 | L-FR1 | QSALTQPASVSGSPGQSITIFC |
| 38 | SG25Ab-4 | L-FR2 | WYQQHPGKAPKLMIY |
| 39 | SG25Ab-4 | L-FR3 | GVSNRFSGSKSGNTASLTISGLQAEDEADYYC |
| 40 | SG25Ab-4 | L-FR4 | FGGGTQLTVL |
| 41 | SG25Ab-9 | C domain | astkgpsvfplapsskstsggtaalgclvkdyfpepvtvswnsgaltsgv |
| | | | htfpavlqssglyslssvvtvpssslgtqtyicnvnhkpsntkvdkkvep |
| | | | kscdkthtcppcpapellggpsvflfppkpkdtlmisrtpevtcvvvdv |
| | | | shedpevkfnwyvdgvevhnaktkpreeqynstyrvvsvltvlhqd |
| | | | wlngkeykckvsnkalpapiektiskakgqprepqvytlppsrdeltk |
| | | | nqvsltclvkgfypsdiavewesngqpennykttppvldsdgsfflysk |
| | | | ltvdksrwqqgnvfscsvmhealhnhytqkslslspgk |
| 42 | human | | MDSYLLMWGLLTFIMVPGCQAELCDDDPPEIP |
| | CD25 | | HATFKAMAYKEGTMLNCECKRGFRRIKSGSLY |
| | | | MLCTGNSSHSSWDNQCQCTSSATRNTTKQVTP |
| | | | QPEEQKERKTTEMQSPMQPVDQASLPGHCREP |
| | | | PPWENEATERIYHFVVGQMVYYQCVQGYRAL |
| | | | HRGPAESVCKMTHGKTRWTQPQLICTGEMETS |
| | | | QFPGEEKPQASPEGRPESETSCLVTTTDFQIQTE |
| | | | MAATMETSIFTTEYQVAVAGCVFLLISVLLLSG |
| | | | LTWQRRQRKSRRTI |
| 43 | HC | | EVQLVQSGAEVKKPGESLKISCKGPEYSFNRY |
| | SGAb25-4 | | WIAWVRQRPGKGLEWMGIIYPGDSDARYSPTF |
| | | | EGHVTLSADMSLTTAYLQWSSLRASDTAMYY |
| | | | CARLGSYYAFDIWGKGTMVTVSSASTKGPSVF |
| | | | PLAPSSKSTSGGTAALGCLVKDYFPEPVTVSWN |
| | | | SGALTSGVHTFPAVLQSSGLYSLSSVVTVPSSSL |
| | | | GTQTYICNVNHKPSNTKVDKKVEPKSCDKTHT |
| | | | CPPCPAPELLGGPSVFLFPPKPKDTLMISRTPEV |
| | | | TCVVVDVSHEDPEVKFNWYVDGVEVHNAKTK |
| | | | PREEQYNSTYRVVSVLTVLHQDWLNGKEYKC |
| | | | KVSNKALPAPIEKTISKAKGQPREPQVYTLPPSR |
| | | | DELTKNQVSLTCLVKGFYPSDIAVEWESNGQP |
| | | | ENNYKTTPPVLDSDGSFFLYSKLTVDKSRWQQ |
| | | | GNVFSCSVMHEALHNHYTQKSLSLSPGK |
| 44 | LC | | QSALTQPASVSGSPGQSITIFCTGTSSDVGGYNY |
| | SGAb25-4 | | VSWYQQHPGKAPKLMIYDVSKRPSGVSNRFSG |
| | | | SKSGNTASLTISGLQAEDEADYYCSSYTSSSTW |
| | | | VFGGGTQLTVLSGQPKAAPSVTLFPPSSEELQA |
| | | | NKATLVCLISDFYPGAVTVAWKADSSPVKAGV |
| | | | ETTTPSKQSNNKYAASSYLSLTPEQWKSHRSYS |
| | | | CQVTHEGSTVEKTVAPTECS |
| 45 | SG25Ab-9 | HC without | QVQLVQSGAEVKKPGESLKISCKGSGYSSTSY |
| | | signal | WIGWVRQMPGKGLEWMGIIYPGDSDTRYSPSF |
| | | peptide | QGQVTISADKSISTAYLQWSSLKASDTAMYYC |
| | | (variable | ARLGSYYAFDIWGRGTMVTVSSastkgpsvfplapss |
| | | domain | kstsggtaalgclvkdyfpepvtvswnsgaltsgvhtfpavlqssglysl |
| | | capitalized, | ssvvtvpssslgtqtyicnvnhkpsntkvdkkvepkscdkthtcppcp |
| | | Y98 | apellggpsvflfppkpkdtlmisrtpevtcvvvdvshedpevkfnwy |
| | | bolded, | vdgvevhnaktkpreeqynstyrvvsvltvlhqdwlngkeykckvsn |
| | | CDRs | kalpapiektiskakgqprepqvytlppsrdeltknqvsltclvkgfyps |
| | | underlined, | diavewesngqpennykttppvldsdgsfflyskltvdksrwqqgnvf |
| | | constant | scsvmhealhnhytqkslslspgk |
| | | domain | |
| | | lower case) | |
| 46 | SG25Ab-9 | HC without | QVQLVQSGAEVKKPGESLKISCKGSGYSSTSY |
| | YH98A | signal | WIGWVRQMPGKGLEWMGIIYPGDSDTRYSPSF |
| | | peptide | QGQVTISADKSISTAYLQWSSLKASDTAMYYC |
| | | (variable | ARLGSAYAFDIWGRGTMVTVSSastkgpsvfplapss |
| | | domain | kstsggtaalgclvkdyfpepvtvswnsgaltsgvhtfpavlqssglysl |
| | | capitalized, | ssvvtvpssslgtqtyicnvnhkpsntkvdkkvepkscdkthtcppcp |
| | | YH98A | apellggpsvflfppkpkdtlmisrtpevtcvvvdvshedpevkfnwy |
| | | bolded, | vdgvevhnaktkpreeqynstyrvvsvltvlhqdwlngkeykckvsn |
| | | CDRs | kalpapiektiskakgqprepqvytlppsrdeltknqvsltclvkgfyps |
| | | underlined, | diavewesngqpennykttppvldsdgsfflyskltvdksrwqqgnvf |
| | | constant | scsvmhealhnhytqkslslspgk |
| | | domain | |
| | | lower case) | |
| 47 | SG25Ab-9; | LC without | QSALTQPASVSASPGQSITISCTGTSSDVGAYIY |
| | SG25Ab-9 | signal | VSWYQQLSGKPPKLILYDVSKRPSGISDRFSGS |
| | YH98A | peptide | KSGNTASLTISGLQADDEADYYCSSYTRSSTW |
| | | (variable | VFGGGTQLTVLgqpkaapsvtlfppsseelqankatlvclisdf |
| | | domain | ypgavtvawkadsspvkagvetttpskqsnnkyaassylsltpeqwks |
| | | capitalized, | hrsyscqvthegstvektvaptecs |
| | | Y98 | |
| | | bolded, | |
| | | CDRs | |
| | | underlined, | |
| | | constant | |
| | | domain | |
| | | lower case) | |

Certain of the antigen binding proteins disclosed herein comprise the 6 CDRs as follows:
a CDR-H1, a CDR-H2, a CDR-H3, a CDR-L1, a CDR-L2, and a CDR-L3, comprising the amino acid sequences of SEQ ID NOs: 1 to 6, respectively;
a CDR-H1, a CDR-H2, a CDR-H3, a CDR-L1, a CDR-L2, and a CDR-L3, comprising the amino acid sequences of SEQ ID NOs: 1, 2, 21, 4, 5, and 6, respectively; or
a CDR-H1, a CDR-H2, a CDR-H3, a CDR-L1, a CDR-L2, and a CDR-L3, comprising the amino acid sequences of SEQ ID NOs: 25 to 30, respectively.

Some of the antigen binding proteins disclosed herein comprise the VH and VL sequences as follows:
a VH comprising the amino acid sequence of SEQ ID NO: 7, and a VL comprising the amino acid sequence of SEQ ID NO: 8;
a VH comprising the amino acid sequence of SEQ ID NO: 22, and a VL comprising the amino acid sequence of SEQ ID NO: 8; or
a VH comprising the amino acid sequence of SEQ ID NO: 31, and a VL comprising the amino acid sequence of SEQ ID NO: 32.

In a further aspects, certain antigen binding proteins comprise an HC and an LC as follows:
a HC comprising the amino acid sequence of SEQ ID NO: 45, and a LC comprising the amino acid sequence of SEQ ID NO: 47;
a HC comprising the amino acid sequence of SEQ ID NO: 46, and a LC comprising the amino acid sequence of SEQ ID NO: 47; or
a HC comprising the amino acid sequence of SEQ ID NO: 43, and a LC comprising the amino acid sequence of SEQ ID NO: 44.

In other aspects, the antigen binding proteins that are provided include or are derived from one or more of the CDRs, variable heavy chains, variable light chains, heavy chains, and/or light chains of the antibodies listed in Tables 2, 3, and 4, or variants or derivatives thereof, such as those described below.

For example, in one aspects, the antigen binding protein comprises a CDR-H3 comprising the amino acid sequence selected from any one of SEQ ID NOs: 3, 21, and 27. In another aspects, the antigen binding protein comprises a CDR-L3 comprising the amino acid selected from any one of SEQ ID NOs: 6 and 30. In yet another aspects, the antigen binding protein comprises a CDR-H3 comprising the amino acid sequence selected from any one of SEQ ID NOs: 3, 21, and 27 and a CDR-L3 comprising the amino acid sequence selected from any one of SEQ ID NOs: 6 and 30.

In another aspects, the antigen binding protein comprises at least one, at least two, or all three of the VH CDR sequences, wherein the VH CDR sequence(s) is/are selected from (a) a CDR-H1 comprising the amino acid sequence selected from any one of SEQ ID NOs: 1 and 25; (b) a CDR-H2 comprising the amino acid sequence selected from any one of SEQ ID NOs: 2 and 26; and (c) a CDR-H3 comprising the amino acid sequence selected from any one of SEQ ID NOs: 3, 21, and 27, provided that in aspects in which the antigen binding protein comprises multiple CDRs each CDR is selected from a different group (a)-(c). The antigen binding protein in other aspects comprises (a) a CDR-H1 comprising the amino acid sequence selected from any one of SEQ ID NOs: 1 and 25; (b) a CDR-H2 comprising the amino acid sequence selected from any one of SEQ ID NOs: 2 and 26; and (c) a CDR-H3 comprising the amino acid sequence selected from any one of SEQ ID NOs: 3, 21, and 27.

In yet another aspects, the antigen binding protein comprises at least one, at least two, or all three of the VL CDR sequences, wherein the VL CDR sequence(s) is/are selected from (a) a CDR-L1 comprising the amino acid sequence selected from any one of SEQ ID NOs: 4 and 28; (b) a CDR-L2 comprising the amino acid sequence selected from any one of SEQ ID NOs: 5 and 29; and (c) a CDR-L3 comprising the amino acid sequence selected from any one of SEQ ID NOs: 6 and 30, provided that in aspects in which the antigen binding protein comprises multiple CDRs each CDR is selected from a different group (a)-(c). The antigen binding protein in other aspects comprises (a) a CDR-L1 comprising the amino acid sequence selected from any one of SEQ ID NOs: 4 and 28; (b) a CDR-L2 comprising the amino acid sequence selected from any one of SEQ ID NOs: 5 and 29; and (c) a CDR-L3 comprising the amino acid sequence selected from any one of SEQ ID NOs: 6 and 30.

In still another aspects, the antigen binding protein comprises (a) a VH domain comprising at least one, at least two, or all three of the VH CDR sequences, wherein the VH CDR sequence(s) is/are selected from (i) a CDR-H1 comprising the amino acid sequence selected from any one of SEQ ID NOs: 1 and 25; (ii) a CDR-H2 comprising the amino acid sequence selected from any one of SEQ ID NOs: 2 and 26; and (iii) a CDR-H3 comprising the amino acid sequence selected from any one of SEQ ID NOs: 3, 21, and 27; and (b) a VL domain comprising at least one, at least two, or all three of the VL CDR sequences, wherein the VL CDR sequence(s) is/are selected from (i) CDR-L1 comprising the amino acid sequence selected from any one of SEQ ID NOs: 4 and 28; (ii) CDR-L2 comprising the amino acid sequence selected from any one of SEQ ID NOs: 5 and 29; and (iii) CDR-L3 comprising the amino acid sequence selected from any one of SEQ ID NOs: 6 and 30, provided that in aspects in which the antigen binding protein comprises multiple CDRs each CDR is selected from a different group.

In another aspects, the antigen binding protein comprises at least one, two, three, four, five, or six CDRs selected from (a) a CDR-H1 comprising the amino acid sequence selected from any one of SEQ ID NOs: 1 and 25; (b) a CDR-H2 comprising the amino acid sequence selected from any one of SEQ ID NOs: 2 and 26; (c) a CDR-H3 comprising the amino acid sequence selected from any one of SEQ ID NOs: 3, 21, and 27; (d) a CDR-L1 comprising the amino acid sequence selected from any one of SEQ ID NOs: 4 and 28; (e) a CDR-L2 comprising the amino acid sequence selected from any one of SEQ ID NOs: 5 and 29; and an (f) CDR-L3 comprising the amino acid sequence selected from any one of SEQ ID NOs: 6 and 30, provided that in aspects in which the antigen binding protein comprises multiple CDRs each CDR is selected from a different group (a)-(f).

In yet another aspects, the antigen binding protein comprises (a) a CDR-H1 comprising the amino acid sequence selected from any one of SEQ ID NOs: 1 and 25; (b) a CDR-H2 comprising the amino acid sequence selected from any one of SEQ ID NOs: 2 and 26; (c) a CDR-H3 comprising the amino acid sequence selected from any one of SEQ ID NOs: 3, 21, and 27; (d) a CDR-L1 comprising the amino acid sequence selected from any one of SEQ ID NOs: 4 and 28; (e) a CDR-L2 comprising the amino acid sequence selected from any one of SEQ ID NOs: 5 and 29; and an (f) CDR-L3 comprising the amino acid sequence selected from any one of SEQ ID NOs: 6 and 30.

In a further aspects, the antigen binding protein comprises (a) a VH comprising a CDR-H1, a CDR-H2, and a CDR-H3 as contained in any one of the amino acid sequences of SEQ ID NOs: 7, 22, or 31, and (b) a VL comprising a CDR-L1, a CDR-L2, and a CDR-H3 as contained in any one of the amino acid sequences of SEQ ID NOs: 8 or 32.

Certain antigen binding proteins comprise a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence selected from any one of SEQ ID NOs: 1 and 25, the CDR-H2 reference sequence has the amino acid sequence selected from any one of SEQ ID NOs: 2 and 26, and the CDR-H3 reference sequence has the amino acid sequence selected from any one of SEQ ID NOs: 3, 21, and 27. In such aspects, the amino acid changes typically are insertions, deletions and/or substitutions. In some of these aspects, the collective number of amino acid changes are 1-3; in other aspects, the collective number of amino acid changes are 1 or 2. In certain of the foregoing aspects, the changes are conservative amino acid substitutions.

In other aspects, an antigen binding protein comprises a VL comprising a CDR-L1, a CDR-L2, and a CDR-L3, wherein the CDRs of the VL collectively have at most 1, 2, 3, 4, or 5 amino acid changes relative to a corresponding HVR reference sequence, and wherein the CDR-L1 reference sequence has the amino acid sequence selected from any one of SEQ ID NOs: 4 and 28, the CDR-L2 reference sequence has the amino acid sequence selected from any one of SEQ ID NOs: 5 and 29, and the CDR-L3 reference sequence has the amino acid sequence selected from any one of SEQ ID NOs: 6 and 30. In such aspects, the amino acid changes typically are insertions, deletions and/or substitutions. In some of these aspects, the collective number of amino acid changes are 1-3; in other aspects, the collective number of amino acid changes are 1 or 2. In certain of the foregoing aspects, the changes are conservative amino acid substitutions.

In a further aspects, an antigen binding protein comprises (a) a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence selected from any one of SEQ ID NOs: 1 and 25, the CDR-H2 reference sequence has the amino acid sequence selected from any one of SEQ ID NOs: 2 and 26, and the CDR-H3 reference sequence has the amino acid sequence selected from any one of SEQ ID NOs: 3, 21, and 27, and (b) a VL comprising a CDR-L1, a CDR-L2, and a CDR-L3, wherein the CDRs of the VL collectively have at most 1, 2, 3, 4, or 5 amino acid changes relative to a corresponding HVR reference sequence, and wherein the CDR-L1 reference sequence has the amino acid sequence selected from any one of SEQ ID NOs: 4 and 28, the CDR-L2 reference sequence has the amino acid sequence selected from any one of SEQ ID NOs: 5 and 29, and the CDR-L3 reference sequence has the amino acid sequence selected from any one of SEQ ID NOs: 6 and 30. In such aspects, the amino acid changes typically are insertions, deletions and/or substitutions. In some of these aspects, the collective number of amino acid changes are 1-3; in other aspects, the collective number of amino acid changes are 1 or 2. In certain of the foregoing aspects, the changes are conservative amino acid substitutions.

The antigen binding protein in another aspects comprises a VH domain, wherein the VH domain sequence has at least 80%, 85%, 90%, 91%, 92%, 93%, 94%, 95%, 96%, 97%, 98%, or 99% sequence identity to the amino acid sequence selected from any one of SEQ ID NOs: 7 and 31, provided the antigen binding protein retains the ability to bind to CD25. In certain aspects, such an antigen binding protein contains substitutions (e.g., conservative substitutions), insertions, and/or deletions relative to the reference sequence (i.e., one of SEQ ID NOs: 7 or 31), provided that such an antigen binding protein retains the ability to bind to CD25. In certain aspects, 1, 2, 3, 4, 5, 6, 7, 8, 9, or 10 amino acids have been substituted, inserted and/or deleted in any one of SEQ ID NOs: 7 or 31. In some aspects, 1-5 or 1-3 amino acids have been substituted, inserted and/or deleted in the VH sequence. In certain of these aspects, such substitutions, insertions, or deletions occur in regions outside the CDRs (i.e., in the FRs). In further such aspects, the VH comprises one, two or three CDRs selected from: (a) a CDR-H1 comprising the amino acid sequence selected from any one of SEQ ID NO: 1 and 25; (b) a CDR-H2 comprising the amino acid sequence selected from any one of SEQ ID NOs: 2 and 26; (c) a CDR-H3 comprising the amino acid sequence selected from any one of SEQ ID NOs: 3, 21, and 27.

The antigen binding protein in another aspects comprises a VL domain, wherein the VL domain sequence has at least 80%, 85%, 90%, 91%, 92%, 93%, 94%, 95%, 96%, 97%, 98%, or 99% sequence identity to the amino acid sequence selected from any one of SEQ ID NOs: 8 and 32, provided the antigen binding protein retains the ability to bind to CD25. In certain aspects, such an antigen binding protein contains substitutions (e.g., conservative substitutions), insertions, and/or deletions relative to the reference sequence (i.e., one of SEQ ID NOs: 8 and 32), provided that such an antigen binding protein retains the ability to bind to CD25. In certain aspects, 1, 2, 3, 4, 5, 6, 7, 8, 9, or 10 amino acids have been substituted, inserted and/or deleted in any one of SEQ ID NOs: 8 or 32. In some aspects, 1-5 or 1-3 amino acids have been substituted, inserted and/or deleted in the VL sequence. In certain of these aspects, such substitutions, insertions, or deletions occur in regions outside the CDRs (i.e., in the FRs). In further such aspects, the VL comprises one, two or three CDRs selected from: (a) a CDR-L1 comprising the amino acid sequence selected from any one of SEQ ID NOs: 4 and 28; (b) a CDR-L2 comprising the amino acid sequence selected from any one of SEQ ID NOs: 5 and 29; (c) a CDR-L3 comprising the amino acid sequence selected from any one of SEQ ID NOs: 6 and 30.

In a further aspects, the antigen binding protein comprises (a) a VH domain, wherein the VH domain sequence has at least 80%, 85%, 90%, 91%, 92%, 93%, 94%, 95%, 96%, 97%, 98%, or 99% sequence identity to the amino acid sequence selected from any one of SEQ ID NOs: 7 and 31, and (b) a VL domain, wherein the VL domain sequence has at least 80%, 85%, 90%, 91%, 92%, 93%, 94%, 95%, 96%, 97%, 98%, or 99% sequence identity to the amino acid sequence selected from any one of SEQ ID NOs: 8 and 32, provided the antigen binding protein retains the ability to bind to CD25. In certain aspects, such an antigen binding protein contains substitutions (e.g., conservative substitutions), insertions, and/or deletions relative to the reference sequence (i.e., one of SEQ ID NOs: 7 and 31 for the VH domain and one of SEQ ID NOs: 8 and 32 for the VL domain), provided that such an antigen binding protein retains the ability to bind to CD25. In certain aspects, 1, 2, 3, 4, 5, 6, 7, 8, 9, or 10 amino acids have been substituted, inserted and/or deleted in the VH and/or the VL sequence. In some aspects, 1-5 or 1-3 amino acids have been substituted, inserted and/or deleted in the VH and/or VL sequence. In other aspects, 1-5 or 1-3 amino acids have been substituted, inserted and/or deleted in the VH and VL sequence collectively. In certain of these aspects, such substitutions, insertions, or deletions occur in regions outside the CDRs (i.e., in the FRs). In further such aspects, the VH comprises one, two, or three CDRs selected from: (i) a CDR-H1 comprising the amino acid sequence selected from any one of SEQ ID NOs: 1 and 25; (ii) a CDR-H2 comprising the amino acid sequence selected from any one of SEQ ID NOs: 2 and 26; (iii) a CDR-H3 comprising the amino acid sequence selected from any one of SEQ ID NOs: 3, 21, and 27, and the VL comprises one, two, or three CDRs selected from: (i) a CDR-L1 comprising the amino acid sequence selected from any one of SEQ ID NOs: 4 and 28; (ii) a CDR-L2 comprising the amino acid sequence selected from any one of SEQ ID NOs: 5 and 29; (iii) a CDR-L3 comprising the amino acid sequence selected from any one of SEQ ID NOs 6 and 30.

The antigen binding protein in any of the foregoing aspects can be an antibody in any form. As such, the antigen binding protein described in any of the above aspects can be, for example, a monoclonal antibody, a multispecific antibody, a human, humanized or chimeric antibody, and CD25 binding fragments of any of the above, such as a single chain antibody, an Fab fragment, an F(ab′) fragment, or a fragment produced by a Fab expression library. The antibodies can be of any immunoglobulin isotype (e.g., IgG, IgE, IgM, IgD, IgA and IgY), class (e.g., IgG1, IgG2, IgG3, IgG4, IgA1 and IgA2) or subclass.

In certain aspects, an antigen binding protein with the CDR and/or variable domain sequences described herein is a fragment of an antibody and include, but are not limited to, Fab, Fab′ and F(ab′)2, Fd, single-chain Fvs (scFv), single-chain antibodies, disulfide-linked Fvs (sdFv) and fragments comprising either a VL or VH domain. Antigen binding proteins, including single-chain antibodies, may comprise the variable region(s) alone or in combination with the entirety or a portion of the following: hinge region, CH1, CH2, CH3 and CL domains. Also included in the present disclosure are antigen binding proteins comprising any combination of variable region(s) with a hinge region, CH1, CH2, CH3 and CL domains.

The antigen binding protein can be monospecific, bispecific, trispecific or of greater multi specificity. Multispecific antibodies can be specific for different epitopes of CD25 or may be specific for both CD25 as well as for a heterologous protein. See, e.g., PCT publications WO 93/17715; WO 92/08802; WO 91/00360; WO 92/05793; Tutt, et al., 1991, J. Immunol. 147:60 69; U.S. Pat. Nos. 4,474,893; 4,714,681; 4,925,648; 5,573,920; 5,601,819; and Kostelny et al., 1992, J. Immunol. 148:1547 1553.

In any of the aspects described herein, one or several amino acids (e.g., 1, 2, 3 or 4) at the amino or carboxy terminus of the light and/or heavy chain, such as the C-terminal lysine of the heavy chain, may be missing or derivatized in some or all of the molecules in a composition. One specific example of such a modification, is an antigen binding protein in which the carboxy terminal lysine of the heavy chain is missing (e.g., as part of a post-translational modification). Furthermore, it should be understood that any of the sequences described herein include post-translational modifications to the specified sequence during expression of the antigen binding protein in cell culture (e.g., a CHO cell culture).

In further aspects, the antigen binding protein is one that binds to the same epitope as one of the antigen binding proteins as described in this section A, such as those listed in Tables 2, 3, and 4.

In additional aspects, the antigen binding protein is one that competes with an antigen binding protein as described in this section A, including, for example, those listed in Tables 2, 3, and 4. Additional details on such antigen binding proteins are described in section H on competing antigen binding proteins below.

In certain aspects, in addition to having the sequence and/or binding characteristics described in this section, the antigen binding protein in addition has 1, 2, 3, 4, 5, 6, 7, 8, 9, 10, 11, 12, 13, 14 or 15 of the characteristics 1-15 in any combination or the antigen binding protein in addition has 1, 2, 3, 4, 5, 6, 7, 8, 9, 10, 11, 12, 13, 14 or 15 of characteristics 16-30 in any combination:
1. Binding to human and/or cynomolgus CD25;
2. Internalization into cells expressing CD25;
3. ADCC activity;
4. In vitro cytotoxicity towards CD25 expressing tumor cell lines when present in an ADC;
5. In vivo anti-tumor activity in CD25 expressing tumor models in mice when present in an ADC;
6. In vivo Treg cell depleting activity in a human CD25 transgenic mouse when present in an ADC;
7. Lack of in vivo CD4 T cell depleting activity in a human CD25 transgenic mouse when present in an ADC;
8. In vitro human Treg depleting activity in PBMC when present in a MMAE ADC;
9. Low level of in vitro human CD4 T cell depleting activity in PBMC when present in a MMAE ADC;
10. Lack of in vitro human CD8 T cell depleting activity in PBMC when present in a MMAE ADC;
11. In vitro human Treg cell depleting activity in PBMC when present in a Camptothecin or Anthracycline ADC;
12. In vitro human CD 8 T cell depleting activity in PBMC when present in a Camptothecin or Anthracycline ADC;
13. Anti-tumor activity in a colon cancer mouse model when present in a MMAE ADC;
14. Reduced binding to human and/or cynomolgus CD25 when comprising a mutation in a heavy chain variable region;
15. Reduced ADCC activity when comprising a mutation in a heavy chain variable region;
16. Reduced in vitro cytotoxicity towards CD25 expressing tumor cell lines when comprising a mutation in a heavy chain variable region and present in an ADC;
17. Reduced in vivo anti-tumor activity in CD25 expressing tumor models in mice when comprising a mutation in a heavy chain variable region and present in an ADC;
18. Reduced in vitro human Treg depleting activity in PBMC when and comprising a mutation in a heavy chain variable region present in an ADC;
19. Reduced in vitro cytotoxicity towards purified human Treg cells when comprising a mutation in a heavy chain variable region and present in MMAE ADC;
20. Lack of in vitro cytotoxicity towards purified human CD8 T cells when comprising a mutation in a heavy chain variable region and present in MMAE ADC;
21. Lack of in vivo CD4 T cell depletion in human CD25 transgenic mice when comprising a mutation in a heavy chain variable region and present in a MMAE ADC;
22. Reduced in vivo Treg depletion in human CD25 transgenic mice when comprising a mutation in a heavy chain variable region and present in a MMAE ADC;
23. Increased anti-tumor activity in a colon cancer mouse model when comprising a mutation in a heavy chain variable region and present in a MMAE ADC;
24. Reduced in vivo Treg depletion in non-human primates when comprising a mutation in a heavy chain variable region and present in a MMAE ADC;
25. Maintenance of total antibody pharmacokinetics in non-human primates when comprising a mutation in a heavy chain variable region and present in a MMAE ADC;
26. Maintenance of antibody-conjugated MMAE pharmacokinetics in non-human primates when comprising a mutation in a heavy chain variable region.

In other aspects, the antigen binding protein is not an antibody or fragment thereof but instead comprises a non-antibody scaffold into which one or more CDRs (e.g., 1, 2, 3, 4, 5 or 6) and/or one or more variable domains as described herein is grafted, inserted, and/or joined, such as those described in greater detail in section [IV.P] below.

### B. Antigen Binding Proteins and Related Aspects

In one aspect, the antigen binding protein comprises a CDR-H3 comprising the amino acid sequence of SEQ ID NO: 3. In another aspect, the antigen binding protein comprises a CDR-L3 comprising the amino acid selected of SEQ ID NO: 6. In yet another aspect, the antigen binding protein comprises a CDR-H3 comprising the amino acid sequence of SEQ ID NO: 3 and a CDR-L3 comprising the amino acid sequence selected of SEQ ID NO: 6.

In another aspects, the antigen binding protein comprises at least one, at least two, or all three VH CDR sequences, wherein the VH CDR sequence(s) is/are selected from (a) a CDR-H1 comprising the amino acid sequence of SEQ ID NO: 1; (b) a CDR-H2 comprising the amino acid sequence of SEQ ID NO: 2; (c) a CDR-H3 comprising the amino acid sequence of SEQ ID NO: 3, provided that in aspects in which the antigen binding protein comprises multiple CDRs each CDR is selected from a different group (a)-(c).

In certain aspects, the antigen binding protein comprises a VH and a VL domain, wherein the heavy chain comprises (a) a CDR-H1 comprising the amino acid sequence of SEQ ID NO: 1; (b) a CDR-H2 comprising the amino acid sequence of SEQ ID NO: 2; and (c) a CDR-H3 comprising the amino acid sequence of SEQ ID NO: 3, and wherein the combined heavy chain and light chain bind CD25. In other aspects, the antigen binding protein comprises a heavy chain and a light chain, wherein the heavy chain comprises (a) a CDR-H1 comprising the amino acid sequence of SEQ ID NO: 1; (b) a CDR-H2 comprising the amino acid sequence of SEQ ID NO: 2; and (c) a CDR-H3 comprising the amino acid sequence of SEQ ID NO: 3, wherein the light chain comprises a CDR-L3, and wherein the combined heavy chain and light chain bind CD25.

In yet another aspects, the antigen binding protein comprises at least one, at least two, or all three VL CDR sequences, wherein the VL HVR sequence(s) is/are selected from (a) a CDR-L1 comprising the amino acid sequence of SEQ ID NO: 4; (b) a CDR-L2 comprising the amino acid sequence of SEQ ID NO: 5; and (c) a CDR-L3 comprising the amino acid sequence of SEQ ID NO: 6, provided that in aspects in which the antigen binding protein comprises multiple CDRs each CDR is selected from a different group (a)-(c). The antigen binding protein in other aspects comprises (a) a CDR-L1 comprising the amino acid sequence of SEQ ID NO: 4; (b) a CDR-L2 comprising the amino acid sequence of SEQ ID NO: 5; and (c) a CDR-L3 comprising the amino acid sequence of SEQ ID NO: 6.

In some aspects, the antigen binding protein comprises a heavy chain and a light chain, wherein the light chain comprises (a) a CDR-L1 comprising the amino acid sequence of SEQ ID NO: 4; (b) a CDR-L2 comprising the amino acid sequence of SEQ ID NO: 5; and (c) a CDR-L3 comprising the amino acid sequence of SEQ ID NO: 6, and wherein the combined heavy chain and light chain bind CD25. In other aspects, the antigen binding protein comprises a heavy chain and a light chain, wherein the light chain comprises (a) a CDR-L1 comprising the amino acid sequence of SEQ ID NO: 4; (b) a CDR-L2 comprising the amino acid sequence of SEQ ID NO: 5; and (c) a CDR-L3 comprising the amino acid sequence of SEQ ID NO: 6, wherein the heavy chain comprises a CDR-H3 comprising the amino acid sequence of SEQ ID NO: 3, and wherein the combined heavy chain and light chain bind CD25.

In still another aspects, the antigen binding protein comprises (a) a VH domain comprising at least one, at least two, or all three VH CDR sequences, wherein the VH CDR sequence(s) is/are selected from (i) a CDR-H1 comprising the amino acid sequence of SEQ ID NO: 1; (ii) a CDR-H2 comprising the amino acid sequence of SEQ ID NO: 2; and (iii) a CDR-H3 comprising the amino acid sequence of SEQ ID NO: 3; and (b) a VL domain comprising at least one, at least two, or all three VL CDR sequences, wherein the VL CDR sequence(s) is/are selected from (i) CDR-L1 comprising the amino acid sequence of SEQ ID NO: 4; (ii) CDR-L2 comprising the amino acid sequence of SEQ ID NO: 5; and (iii) CDR-L3 comprising the amino acid sequence of SEQ ID NO: 6, provided that in aspects in which the antigen binding protein comprises multiple CDRs each CDR is selected from a different group.

In another aspects, the antigen binding protein comprises at least one, two, three, four, five, or six CDRs selected from (a) a CDR-H1 comprising the amino acid sequence of SEQ ID NO: 1; (b) a CDR-H2 comprising the amino acid sequence of SEQ ID NO: 2; (c) a CDR-H3 comprising the amino acid sequence of SEQ ID NO: 3; (d) a CDR-L1 comprising the amino acid sequence of SEQ ID NO: 4; (e) a CDR-L2 comprising the amino acid sequence of SEQ ID NO: 5; and an (f) CDR-L3 comprising the amino acid sequence of SEQ ID NO: 6, provided that in aspects in which the antigen binding protein comprises multiple CDRs each CDR is selected from a different group (a)-(f).

In yet another aspects, the antigen binding protein comprises (a) a CDR-H1 comprising the amino acid sequence of SEQ ID NO: 1; (b) a CDR-H2 comprising the amino acid sequence of SEQ ID NO: 2; (c) a CDR-H3 comprising the amino acid sequence of SEQ ID NO: 3; (d) a CDR-L1 comprising the amino acid sequence of SEQ ID NO: 4; (e) a CDR-L2 comprising the amino acid sequence of SEQ ID NO: 5; and an (f) CDR-L3 comprising the amino acid sequence of SEQ ID NO: 6.

In a further aspects, the antigen binding protein comprises (a) a VH comprising a CDR-H1, a CDR-H2, and a CDR-H3 comprising the amino acid sequences of the CDR-H1, CDR-H2, and CDR-H3 in the VH amino acid sequence set forth in SEQ ID NO: 7, and (b) a VL comprising a CDR-L1, a CDR-L2, and a CDR-H3 comprising the amino acid sequences of the CDR-L1, CDR-L2, and the CDR-L3 in the VL amino acid sequence as set forth in SEQ ID NO: 8.

Certain antigen binding proteins comprise a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence of SEQ ID NO: 1, the CDR-H2 reference sequence has the amino acid sequence of SEQ ID NO: 2, and the CDR-H3 reference sequence has the amino acid sequence of SEQ ID NO: 3. In such aspects, the amino acid changes typically are insertions, deletions and/or substitutions. In some of these aspects, the collective number of amino acid changes are 1-3; in other aspects, the collective number of amino acid changes are 1 or 2. In some of the foregoing aspects, the changes are conservative amino acid substitutions.

In other aspects, an antigen binding protein comprises a VL comprising a CDR-L1, a CDR-L2, and a CDR-L3, wherein the CDRs of the VL collectively have at most 1, 2, 3, 4, or 5 amino acid changes relative to a corresponding HVR reference sequence, and wherein the CDR-L1 reference sequence has the amino acid sequence of SEQ ID NO: 1, the CDR-L2 reference sequence has the amino acid sequence of SEQ ID NO: 2, and the CDR-L3 reference sequence has the amino acid sequence of SEQ ID NO: 3. In such aspects, the amino acid changes typically are insertions, deletions and/or substitutions. In some of these aspects, the collective number of amino acid changes are 1-3; in other aspects, the collective number of amino acid changes are 1 or 2. In certain of the foregoing aspects, the changes are conservative amino acid substitutions.

In a further aspects, an antigen binding protein comprises (a) a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence of SEQ ID NO: 1, the CDR-H2 reference sequence has the amino acid sequence of SEQ ID NO: 2, and the CDR-H3 reference sequence has the amino acid sequence of SEQ ID NO: 3, and (b) a VL comprising a CDR-L1, a CDR-L2, and a CDR-L3, wherein the CDRs of the VL collectively have at most 1, 2, 3, 4, or 5 amino acid changes relative to a corresponding HVR reference sequence, and wherein the CDR-L1 reference sequence has the amino acid sequence of SEQ ID NO: 4, the CDR-L2 reference sequence has the amino acid sequence of SEQ ID NO: 5, and the CDR-L3 reference sequence has the amino acid sequence of SEQ ID NO: 6. In such aspects, the amino acid changes typically are insertions, deletions and/or substitutions. In some of these aspects, the collective number of amino acid changes are at most 1-3 amino acid changes; in other aspects, the collective number of amino acid changes are 1 or 2 changes. In certain of the foregoing aspects, the changes are conservative amino acid substitutions.

An antigen binding protein as provided herein can comprise any suitable framework variable domain sequence, provided that the antibody retains the ability to bind CD25 (e.g., human CD25). As used herein, heavy chain framework regions are designated “HC-FR1-FR2-FR3-FR4,” and light chain framework regions are designated “LC-FR1-FR2-FR3-FR4.” In some aspects, the antigen binding protein comprises a heavy chain variable domain framework sequence of SEQ ID NO: 9, 10, 11, and 12, which correspond to amino acid sequence of HC-FR1, HC-FR2, HC-FR3, and HC-FR4, respectively. In some aspects, the antigen binding protein comprises a light chain variable domain framework sequence of SEQ ID NO: 13, 14, 15, and 16, which correspond to the amino acid sequence of LC-FR1, LC-FR2, LC-FR3, and LC-FR4, respectively. In some aspects, an antigen binding protein comprises CDR sequences as described in this section that have been inserted or grafted into their respective locations in such frameworks. In some aspects, one or more of the framework regions differs from the foregoing framework sequences by no more than 1, 2, 3, 4, 5, 6, 7, 8, 9, or 10 amino acids in the VH and/or VL framework regions. In certain of these aspects, there are at most 1, 2, 3, 4, or 5 amino acid differences in the VH and/or VL framework regions. In still other aspects, there are at most 1 or 2, 1-3, or 1-5 amino acid differences in the VH and/or VL framework regions. In certain aspects, there are at most 1 or 2, 1-3, or 1-5 amino acid differences in the VH framework regions. In some aspects, there are at most 1 or 2, 1-3, or 1-5 amino acid differences in the VL framework regions. In any of the foregoing aspects, the differences can be conservative amino acid substitutions. In some aspects, the differences correspond to backmutations.

As an example of such an aspects, certain antigen binding proteins as provided herein comprise a heavy chain variable region (VH) and a light chain variable region (VL), wherein the antibody comprises:
(a) a VH comprising:
   (1) a HC-FR1 comprising the amino acid sequence of SEQ ID NO: 9;
   (2) a CDR-H1 comprising the amino acid sequence of SEQ ID NO: 1;
   (3) a HC-FR2 comprising the amino acid sequence of SEQ ID NO: 10;
   (4) a CDR-H2 comprising the amino acid sequence of SEQ ID NO: 2;
   (5) a HC-FR3 comprising the amino acid sequence of SEQ ID NO: 11;
   (6) a CDR-H3 comprising the amino acid sequence of SEQ ID NO: 3; and
   (7) a HC-FR4 comprising the amino acid sequence of SEQ ID NO: 12, and/or
(b) a VL comprising:
   (1) a LC-FR1 comprising the amino acid sequence of SEQ ID NO: 13;
   (2) a CDR-L1 comprising the amino acid sequence of SEQ ID NO: 4;
   (3) a LC-FR2 comprising the amino acid sequence of SEQ ID NO: 14;
   (4) a CDR-L2 comprising the amino acid sequence of SEQ ID NO: 5;
   (5) a LC-FR3 comprising the amino acid sequence of SEQ ID NO: 15;
   (6) a CDR-L3 comprising the amino acid sequence of SEQ ID NO: 6; and
   (7) a LC-FR4 comprising the amino acid sequence of SEQ ID NO: 16.

In some aspects, the antigen binding protein comprises a VH comprising the amino acid sequence of SEQ ID NO: 7, and a VL comprising the amino acid sequence of SEQ ID NO: 8.

The antigen binding protein in other aspects comprises a VH domain, wherein the VH domain sequence has at least 80%, 85%, 90%, 91%, 92%, 93%, 94%, 95%, 96%, 97%, 98%, or 99% sequence identity to the amino acid sequence of SEQ ID NO: 7, provided the antigen binding protein retains the ability to bind to CD25. In certain aspects, such an antigen binding protein contains substitutions (e.g., conservative substitutions), insertions, or deletions relative to the reference sequence (i.e., SEQ ID NO: 7), provided that such an antigen binding protein retains the ability to bind to CD25. In certain aspects, 1, 2, 3, 4, 5, 6, 7, 8, 9, or 10 amino acids have been substituted, inserted and/or deleted in SEQ ID NO: 7. In some aspects, 1-5 or 1-3 amino acids have been substituted, inserted and/or deleted in the VH sequence. In certain of these aspects, such substitutions, insertions, or deletions occur in regions outside the CDRs (i.e., in the FRs). In further such aspects, the VH comprises one, two or three CDRs selected from: (a) a CDR-H1 comprising the amino acid sequence of SEQ ID NO: 1; (b) a CDR-H2 comprising the amino acid sequence of SEQ ID NO: 2; and (c) a CDR-H3 comprising the amino acid of SEQ ID NO: 3.

The antigen binding protein in another aspect comprises a VL domain, wherein the VL domain sequence has at least 80%, 85%, 90%, 91%, 92%, 93%, 94%, 95%, 96%, 97%, 98%, or 99% sequence identity to the amino acid sequence of SEQ ID NO: 8, provided the antigen binding protein retains the ability to bind to CD25. In certain aspects, such an antigen binding protein contains substitutions (e.g., conservative substitutions), insertions, or deletions relative to the reference sequence (i.e., SEQ ID NO: 8), provided that such an antigen binding protein retains the ability to bind to CD25. In certain aspects, 1, 2, 3, 4, 5, 6, 7, 8, 9, or 10 amino acids have been substituted, inserted and/or deleted in SEQ ID NO: 8. In some aspects, 1-5 or 1-3 amino acids have been substituted, inserted and/or deleted in the VL sequence. In certain of these aspects, such substitutions, insertions, or deletions occur in regions outside the CDRs (i.e., in the FRs). In further such aspects, the VL comprises one, two or three CDRs selected from: (a) a CDR-L1 comprising the amino acid sequence of SEQ ID NO: 4; (b) a CDR-L2 comprising the amino acid sequence of SEQ ID NO: 5; and (c) a CDR-L3 comprising the amino acid sequence of SEQ ID NO: 6.

In a further aspects, the antigen binding protein comprises (a) a VH domain, wherein the VH domain sequence has at least 80%, 85%, 90%, 91%, 92%, 93%, 94%, 95%, 96%, 97%, 98%, or 99% sequence identity to the amino acid sequence of SEQ ID NO: 7, and (b) a VL domain, wherein the VL domain sequence has at least 80%, 85%, 90%, 91%, 92%, 93%, 94%, 95%, 96%, 97%, 98%, or 99% sequence identity to the amino acid sequence of SEQ ID NO: 8, provided the antigen binding protein retains the ability to bind to CD25. In certain aspects, such an antigen binding protein contains substitutions (e.g., conservative substitutions), insertions, or deletions relative to the reference sequence (i.e., SEQ ID NO: 7 for the VH domain and SEQ ID NO: 8 for the VL domain), provided that such an antigen binding protein retains the ability to bind to CD25. In certain aspects, 1, 2, 3, 4, 5, 6, 7, 8, 9, or 10 amino acids have been substituted, inserted and/or deleted in the VH and/or VL sequence. In some aspects, 1-5 or 1-3 amino acids have been substituted, inserted and/or deleted in the VH and/or VL sequence. In other aspects, 1-5 or 1-3 amino acids have been substituted, inserted and/or deleted in the VL and VH sequences collectively. In certain of these aspects, such substitutions, insertions, or deletions occur in regions outside the CDRs (i.e., in the FRs). In further such aspects, the VH comprises one, two, or three CDRs selected from: (i) a CDR-H1 comprising the amino acid sequence of SEQ ID NO: 1; (ii) a CDR-H2 comprising the amino acid sequence of SEQ ID NO: 2; (iii) a CDR-H3 comprising the amino acid sequence of SEQ ID NO: 3, and the VL comprises one, two, or three CDRs selected from: (i) a CDR-L1 comprising the amino acid sequence of SEQ ID NO: 4; (ii) a CDR-L2 comprising the amino acid sequence of SEQ ID NO: 5; (iii) a CDR-L3 comprising the amino acid sequence of SEQ ID NO: 6.

In further aspects, an antigen binding protein comprises an HC comprising the amino acid sequence of SEQ ID NO: 45, and a LC comprising the amino acid sequence of SEQ ID NO: 47.

In one aspect, the antigen binding protein comprises a CDR-H3 comprising the amino acid sequence of SEQ ID NO: 3. In another aspect, the antigen binding protein comprises a CDR-L3 comprising the amino acid selected of SEQ ID NO: 6. In yet another aspect, the antigen binding protein comprises a CDR-H3 comprising the amino acid sequence of SEQ ID NO: 21 and a CDR-L3 comprising the amino acid sequence selected of SEQ ID NO: 6.

In another aspect, the antigen binding protein comprises at least one, at least two, or all three VH CDR sequences, wherein the VH CDR sequence(s) is/are selected from (a) a CDR-H1 comprising the amino acid sequence of SEQ ID NO: 1; (b) a CDR-H2 comprising the amino acid sequence of SEQ ID NO: 2; (c) a CDR-H3 comprising the amino acid sequence of SEQ ID NO: 21, provided that in aspects in which the antigen binding protein comprises multiple CDRs each CDR is selected from a different group (a)-(c).

In certain aspects, the antigen binding protein comprises a VH and a VL domain, wherein the heavy chain comprises (a) a CDR-H1 comprising the amino acid sequence of SEQ ID NO: 1; (b) a CDR-H2 comprising the amino acid sequence of SEQ ID NO: 2; and (c) a CDR-H3 comprising the amino acid sequence of SEQ ID NO: 21, and wherein the combined heavy chain and light chain bind CD25. In other aspects, the antigen binding protein comprises a heavy chain and a light chain, wherein the heavy chain comprises (a) a CDR-H1 comprising the amino acid sequence of SEQ ID NO: 1; (b) a CDR-H2 comprising the amino acid sequence of SEQ ID NO: 2; and (c) a CDR-H3 comprising the amino acid sequence of SEQ ID NO: 21, wherein the light chain comprises a CDR-L3, and wherein the combined heavy chain and light chain bind CD25.

In yet another aspects, the antigen binding protein comprises at least one, at least two, or all three VL CDR sequences, wherein the VL CDR sequence(s) is/are selected from (a) a CDR-L1 comprising the amino acid sequence of SEQ ID NO: 4; (b) a CDR-L2 comprising the amino acid sequence of SEQ ID NO: 5; and (c) a CDR-L3 comprising the amino acid sequence of SEQ ID NO: 6, provided that in aspects in which the antigen binding protein comprises multiple CDRs each CDR is selected from a different group (a)-(c). The antigen binding protein in other aspects comprises (a) a CDR-L1 comprising the amino acid sequence of SEQ ID NO: 4; (b) a CDR-L2 comprising the amino acid sequence of SEQ ID NO: 5; and (c) a CDR-L3 comprising the amino acid sequence of SEQ ID NO: 6.

In some aspects, the antigen binding protein comprises a heavy chain and a light chain, wherein the light chain comprises (a) a CDR-L1 comprising the amino acid sequence of SEQ ID NO: 4; (b) a CDR-L2 comprising the amino acid sequence of SEQ ID NO: 5; and (c) a CDR-L3 comprising the amino acid sequence of SEQ ID NO: 6, and wherein the combined heavy chain and light chain bind CD25. In other aspects, the antigen binding protein comprises a heavy chain and a light chain, wherein the light chain comprises (a) a CDR-L1 comprising the amino acid sequence of SEQ ID NO: 4; (b) a CDR-L2 comprising the amino acid sequence of SEQ ID NO: 5; and (c) a CDR-L3 comprising the amino acid sequence of SEQ ID NO: 6, wherein the heavy chain comprises a CDR-H3 comprising the amino acid sequence of SEQ ID NO: 21, and wherein the combined heavy chain and light chain bind CD25.

In still another aspects, the antigen binding protein comprises (a) a VH domain comprising at least one, at least two, or all three VH CDR sequences, wherein the VH CDR sequence(s) is/are selected from (i) a CDR-H1 comprising the amino acid sequence of SEQ ID NO: 1; (ii) a CDR-H2 comprising the amino acid sequence of SEQ ID NO: 2; and (iii) a CDR-H3 comprising the amino acid sequence of SEQ ID NO: 21; and (b) a VL domain comprising at least one, at least two, or all three VL CDR sequences, wherein the VL CDR sequence(s) is/are selected from (i) CDR-L1 comprising the amino acid sequence of SEQ ID NO: 4; (ii) CDR-L2 comprising the amino acid sequence of SEQ ID NO: 5; and (iii) CDR-L3 comprising the amino acid sequence of SEQ ID NO: 6, provided that in aspects in which the antigen binding protein comprises multiple CDRs each CDR is selected from a different group.

In another aspect, the antigen binding protein comprises at least one, two, three, four, five, or six CDRs selected from (a) a CDR-H1 comprising the amino acid sequence of SEQ ID NO: 1; (b) a CDR-H2 comprising the amino acid sequence of SEQ ID NO: 2; (c) a CDR-H3 comprising the amino acid sequence of SEQ ID NO: 21; (d) a CDR-L1 comprising the amino acid sequence of SEQ ID NO: 4; (e) a CDR-L2 comprising the amino acid sequence of SEQ ID NO: 5; and an (f) CDR-L3 comprising the amino acid sequence of SEQ ID NO: 6, provided that in aspects in which the antigen binding protein comprises multiple CDRs each CDR is selected from a different group (a)-(f).

In yet another aspect, the antigen binding protein comprises (a) a CDR-H1 comprising the amino acid sequence of SEQ ID NO: 1; (b) a CDR-H2 comprising the amino acid sequence of SEQ ID NO: 2; (c) a CDR-H3 comprising the amino acid sequence of SEQ ID NO: 21; (d) a CDR-L1 comprising the amino acid sequence of SEQ ID NO: 4; (e) a CDR-L2 comprising the amino acid sequence of SEQ ID NO: 5; and an (f) CDR-L3 comprising the amino acid sequence of SEQ ID NO: 6.

In a further aspect, the antigen binding protein comprises (a) a VH comprising a CDR-H1, a CDR-H2, and a CDR-H3 comprising the amino acid sequences of the CDR-H1, CDR-H2, and CDR-H3 in the VH amino acid sequence set forth in SEQ ID NO: 22, and (b) a VL comprising a CDR-L1, a CDR-L2, and a CDR-H3 comprising the amino acid sequences of the CDR-L1, CDR-L2, and the CDR-L3 in the VL amino acid sequence as set forth in SEQ ID NO: 8.

Certain antigen binding proteins comprise a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence of SEQ ID NO: 1, the CDR-H2 reference sequence has the amino acid sequence of SEQ ID NO: 2, and the CDR-H3 reference sequence has the amino acid sequence of SEQ ID NO: 21. In such aspects, the amino acid changes typically are insertions, deletions and/or substitutions. In some of these aspects, the collective number of amino acid changes are 1-3; in other aspects, the collective number of amino acid changes are 1 or 2. In some of the foregoing aspects, the changes are conservative amino acid substitutions.

In other aspects, an antigen binding protein comprises a VL comprising a CDR-L1, a CDR-L2, and a CDR-L3, wherein the CDRs of the VL collectively have at most 1, 2, 3, 4, or 5 amino acid changes relative to a corresponding CDR reference sequence, and wherein the CDR-L1 reference sequence has the amino acid sequence of SEQ ID NO: 1, the CDR-L2 reference sequence has the amino acid sequence of SEQ ID NO: 2, and the CDR-L3 reference sequence has the amino acid sequence of SEQ ID NO: 21. In such aspects, the amino acid changes typically are insertions, deletions and/or substitutions. In some of these aspects, the collective number of amino acid changes are 1-3; in other aspects, the collective number of amino acid changes are 1 or 2. In certain of the foregoing aspects, the changes are conservative amino acid substitutions.

In a further aspect, an antigen binding protein comprises (a) a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence of SEQ ID NO: 1, the CDR-H2 reference sequence has the amino acid sequence of SEQ ID NO: 2, and the CDR-H3 reference sequence has the amino acid sequence of SEQ ID NO: 21, and (b) a VL comprising a CDR-L1, a CDR-L2, and a CDR-L3, wherein the CDRs of the VL collectively have at most 1, 2, 3, 4, or 5 amino acid changes relative to a corresponding CDR reference sequence, and wherein the CDR-L1 reference sequence has the amino acid sequence of SEQ ID NO: 4, the CDR-L2 reference sequence has the amino acid sequence of SEQ ID NO: 5, and the CDR-L3 reference sequence has the amino acid sequence of SEQ ID NO: 6. In such aspects, the amino acid changes typically are insertions, deletions and/or substitutions. In some of these aspects, the collective number of amino acid changes are at most 1-3 amino acid changes; in other aspects, the collective number of amino acid changes are 1 or 2 changes. In certain of the foregoing aspects, the changes are conservative amino acid substitutions.

An antigen binding protein as provided herein can comprise any suitable framework variable domain sequence, provided that the antibody retains the ability to bind CD25 (e.g., human CD25). As used herein, heavy chain framework regions are designated “HC-FR1-FR2-FR3-FR4,” and light chain framework regions are designated “LC-FR1-FR2-FR3-FR4.” In some aspects, the antigen binding protein comprises a heavy chain variable domain framework sequence of SEQ ID NO: 9, 10, 11, and 12, which correspond to amino acid sequence of HC-FR1, HC-FR2, HC-FR3, and HC-FR4, respectively. In some aspects, the antigen binding protein comprises a light chain variable domain framework sequence of SEQ ID NO: 13, 14, 15, and 16, which correspond to the amino acid sequence of LC-FR1, LC-FR2, LC-FR3, and LC-FR4, respectively. In some aspects, an antigen binding protein comprises CDR sequences as described in this section that have been inserted or grafted into their respective locations in such frameworks. In some aspects, one or more of the framework regions differs from the foregoing framework sequences by no more than 1, 2, 3, 4, 5, 6, 7, 8, 9, or 10 amino acids in the VH and/or VL framework regions. In certain of these aspects, there are at most 1, 2, 3, 4, or 5 amino acid differences in the VH and/or VL framework regions. In still other aspects, there are at most 1 or 2, 1-3, or 1-5 amino acid differences in the VH and/or VL framework regions. In certain aspects, there are at most 1 or 2, 1-3, or 1-5 amino acid differences in the VH framework regions. In some aspects, there are at most 1 or 2, 1-3, or 1-5 amino acid differences in the VL framework regions. In any of the foregoing aspects, the differences can be conservative amino acid substitutions. In some aspects, the differences correspond to backmutations.

As an example of such an aspects, certain antigen binding proteins as provided herein comprise a heavy chain variable region (VH) and a light chain variable region (VL), wherein the antibody comprises:
(a) a VH comprising:
   (1) a HC-FR1 comprising the amino acid sequence of SEQ ID NO: 9;
   (2) a CDR-H1 comprising the amino acid sequence of SEQ ID NO: 1;
   (3) a HC-FR2 comprising the amino acid sequence of SEQ ID NO: 10;
   (4) a CDR-H2 comprising the amino acid sequence of SEQ ID NO: 2;
   (5) a HC-FR3 comprising the amino acid sequence of SEQ ID NO: 11;
   (6) a CDR-H3 comprising the amino acid sequence of SEQ ID NO: 21; and
   (7) a HC-FR4 comprising the amino acid sequence of SEQ ID NO: 12, and/or
(b) a VL comprising:
   (1) a LC-FR1 comprising the amino acid sequence of SEQ ID NO: 13;
   (2) a CDR-L1 comprising the amino acid sequence of SEQ ID NO: 4;
   (3) a LC-FR2 comprising the amino acid sequence of SEQ ID NO: 14;
   (4) a CDR-L2 comprising the amino acid sequence of SEQ ID NO: 5;
   (5) a LC-FR3 comprising the amino acid sequence of SEQ ID NO: 15;
   (6) a CDR-L3 comprising the amino acid sequence of SEQ ID NO: 6; and
   (7) a LC-FR4 comprising the amino acid sequence of SEQ ID NO: 16.

In some aspects, the antigen binding protein comprises a VH comprising the amino acid sequence of SEQ ID NO: 22, and a VL comprising the amino acid sequence of SEQ ID NO: 8.

The antigen binding protein in other aspects comprises a VH domain, wherein the VH domain sequence has at least 80%, 85%, 90%, 91%, 92%, 93%, 94%, 95%, 96%, 97%, 98%, or 99% sequence identity to the amino acid sequence of SEQ ID NO: 22, provided the antigen binding protein retains the ability to bind to CD25. In certain aspects, such an antigen binding protein contains substitutions (e.g., conservative substitutions), insertions, or deletions relative to the reference sequence (i.e., SEQ ID NO: 22), provided that such an antigen binding protein retains the ability to bind to CD25. In certain aspects, 1, 2, 3, 4, 5, 6, 7, 8, 9, or 10 amino acids have been substituted, inserted and/or deleted in SEQ ID NO: 22. In some aspects, 1-5 or 1-3 amino acids have been substituted, inserted and/or deleted in the VH sequence. In certain of these aspects, such substitutions, insertions, or deletions occur in regions outside the CDRs (i.e., in the FRs). In further such aspects, the VH comprises one, two or three CDRs selected from: (a) a CDR-H1 comprising the amino acid sequence of SEQ ID NO: 1; (b) a CDR-H2 comprising the amino acid sequence of SEQ ID NO: 2; and (c) a CDR-H3 comprising the amino acid of SEQ ID NO: 21.

The antigen binding protein in another aspects comprises a VL domain, wherein the VL domain sequence has at least 80%, 85%, 90%, 91%, 92%, 93%, 94%, 95%, 96%, 97%, 98%, or 99% sequence identity to the amino acid sequence of SEQ ID NO: 8, provided the antigen binding protein retains the ability to bind to CD25. In certain aspects, such an antigen binding protein contains substitutions (e.g., conservative substitutions), insertions, or deletions relative to the reference sequence (i.e., SEQ ID NO: 8), provided that such an antigen binding protein retains the ability to bind to CD25. In certain aspects, 1, 2, 3, 4, 5, 6, 7, 8, 9, or 10 amino acids have been substituted, inserted and/or deleted in SEQ ID NO: 8. In some aspects, 1-5 or 1-3 amino acids have been substituted, inserted and/or deleted in the VL sequence. In certain of these aspects, such substitutions, insertions, or deletions occur in regions outside the CDRs (i.e., in the FRs). In further such aspects, the VL comprises one, two or three CDRs selected from: (a) a CDR-L1 comprising the amino acid sequence of SEQ ID NO: 4; (b) a CDR-L2 comprising the amino acid sequence of SEQ ID NO: 5; and (c) a CDR-L3 comprising the amino acid sequence of SEQ ID NO: 6.

In a further aspect, the antigen binding protein comprises (a) a VH domain, wherein the VH domain sequence has at least 80%, 85%, 90%, 91%, 92%, 93%, 94%, 95%, 96%, 97%, 98%, or 99% sequence identity to the amino acid sequence of SEQ ID NO: 22, and (b) a VL domain, wherein the VL domain sequence has at least 80%, 85%, 90%, 91%, 92%, 93%, 94%, 95%, 96%, 97%, 98%, or 99% sequence identity to the amino acid sequence of SEQ ID NO: 8, provided the antigen binding protein retains the ability to bind to CD25. In certain aspects, such an antigen binding protein contains substitutions (e.g., conservative substitutions), insertions, or deletions relative to the reference sequence (i.e., SEQ ID NO: 22 for the VH domain and SEQ ID NO: 8 for the VL domain), provided that such an antigen binding protein retains the ability to bind to CD25. In certain aspects, 1, 2, 3, 4, 5, 6, 7, 8, 9, or 10 amino acids have been substituted, inserted and/or deleted in the VH and/or VL sequence. In some aspects, 1-5 or 1-3 amino acids have been substituted, inserted and/or deleted in the VH and/or VL sequence. In other aspects, 1-5 or 1-3 amino acids have been substituted, inserted and/or deleted in the VL and VH sequences collectively. In certain of these aspects, such substitutions, insertions, or deletions occur in regions outside the CDRs (i.e., in the FRs). In further such aspects, the VH comprises one, two, or three CDRs selected from: (i) a CDR-H1 comprising the amino acid sequence of SEQ ID NO: 1; (ii) a CDR-H2 comprising the amino acid sequence of SEQ ID NO: 2; (iii) a CDR-H3 comprising the amino acid sequence of SEQ ID NO: 21, and the VL comprises one, two, or three CDRs selected from: (i) a CDR-L1 comprising the amino acid sequence of SEQ ID NO: 4; (ii) a CDR-L2 comprising the amino acid sequence of SEQ ID NO: 5; (iii) a CDR-L3 comprising the amino acid sequence of SEQ ID NO: 6.

In a further aspect, an antigen binding protein comprises an HC comprising the amino acid sequence of SEQ ID NO: 46, and a LC comprising the amino acid sequence of SEQ ID NO: 47.

In some aspects, the antigen binding protein comprises a CDR-H3 comprising the amino acid sequence of SEQ ID NO: 27. In another aspect, the antigen binding protein comprises a CDR-L3 comprising the amino acid selected of SEQ ID NO: 30. In yet another aspect, the antigen binding protein comprises a CDR-H3 comprising the amino acid sequence of SEQ ID NO: 27 and a CDR-L3 comprising the amino acid sequence selected of SEQ ID NO: 30.

In other aspects, the antigen binding protein comprises at least one, at least two, or all three VH CDR sequences, wherein the VH CDR sequence(s) is/are selected from (a) a CDR-H1 comprising the amino acid sequence of SEQ ID NO: 25; (b) a CDR-H2 comprising the amino acid sequence of SEQ ID NO: 26; (c) a CDR-H3 comprising the amino acid sequence of SEQ ID NO: 27, provided that in aspects in which the antigen binding protein comprises multiple CDRs each CDR is selected from a different group (a)-(c).

In certain aspects, the antigen binding protein comprises a VH and a VL domain, wherein the heavy chain comprises (a) a CDR-H1 comprising the amino acid sequence of SEQ ID NO: 25; (b) a CDR-H2 comprising the amino acid sequence of SEQ ID NO: 26; and (c) a CDR-H3 comprising the amino acid sequence of SEQ ID NO: 27, and wherein the combined heavy chain and light chain bind CD25. In other aspects, the antigen binding protein comprises a heavy chain and a light chain, wherein the heavy chain comprises (a) a CDR-H1 comprising the amino acid sequence of SEQ ID NO: 25; (b) a CDR-H2 comprising the amino acid sequence of SEQ ID NO: 26; and (c) a CDR-H3 comprising the amino acid sequence of SEQ ID NO: 27, wherein the light chain comprises a CDR-L3, and wherein the combined heavy chain and light chain bind CD25.

In yet another aspects, the antigen binding protein comprises at least one, at least two, or all three VL CDR sequences, wherein the VL CDR sequence(s) is/are selected from (a) a CDR-L1 comprising the amino acid sequence of SEQ ID NO: 28; (b) a CDR-L2 comprising the amino acid sequence of SEQ ID NO: 29; and (c) a CDR-L3 comprising the amino acid sequence of SEQ ID NO: 30, provided that in aspects in which the antigen binding protein comprises multiple CDRs each CDR is selected from a different group (a)-(c). The antigen binding protein in other aspects comprises (a) a CDR-L1 comprising the amino acid sequence of SEQ ID NO: 28; (b) a CDR-L2 comprising the amino acid sequence of SEQ ID NO: 29; and (c) a CDR-L3 comprising the amino acid sequence of SEQ ID NO: 30.

In some aspects, the antigen binding protein comprises a heavy chain and a light chain, wherein the light chain comprises (a) a CDR-L1 comprising the amino acid sequence of SEQ ID NO: 28; (b) a CDR-L2 comprising the amino acid sequence of SEQ ID NO: 29; and (c) a CDR-L3 comprising the amino acid sequence of SEQ ID NO: 30, and wherein the combined heavy chain and light chain bind CD25. In other aspects, the antigen binding protein comprises a heavy chain and a light chain, wherein the light chain comprises (a) a CDR-L1 comprising the amino acid sequence of SEQ ID NO: 28; (b) a CDR-L2 comprising the amino acid sequence of SEQ ID NO: 29; and (c) a CDR-L3 comprising the amino acid sequence of SEQ ID NO: 30, wherein the heavy chain comprises a CDR-H3 comprising the amino acid sequence of SEQ ID NO: 27, and wherein the combined heavy chain and light chain bind CD25.

In still another aspects, the antigen binding protein comprises (a) a VH domain comprising at least one, at least two, or all three VH CDR sequences, wherein the VH CDR sequence(s) is/are selected from (i) a CDR-H1 comprising the amino acid sequence of SEQ ID NO: 25; (ii) a CDR-H2 comprising the amino acid sequence of SEQ ID NO: 26; and (iii) a CDR-H3 comprising the amino acid sequence of SEQ ID NO: 27; and (b) a VL domain comprising at least one, at least two, or all three VL CDR sequences, wherein the VL CDR sequence(s) is/are selected from (i) CDR-L1 comprising the amino acid sequence of SEQ ID NO: 28; (ii) CDR-L2 comprising the amino acid sequence of SEQ ID NO: 29; and (iii) CDR-L3 comprising the amino acid sequence of SEQ ID NO: 30, provided that in aspects in which the antigen binding protein comprises multiple CDRs each CDR is selected from a different group.

In another aspect, the antigen binding protein comprises at least one, two, three, four, five, or six CDRs selected from (a) a CDR-H1 comprising the amino acid sequence of SEQ ID NO: 25; (b) a CDR-H2 comprising the amino acid sequence of SEQ ID NO: 26; (c) a CDR-H3 comprising the amino acid sequence of SEQ ID NO: 27; (d) a CDR-L1 comprising the amino acid sequence of SEQ ID NO: 28; (e) a CDR-L2 comprising the amino acid sequence of SEQ ID NO: 29; and an (f) CDR-L3 comprising the amino acid sequence of SEQ ID NO: 30, provided that in aspects in which the antigen binding protein comprises multiple CDRs each CDR is selected from a different group (a)-(f).

In yet another aspect, the antigen binding protein comprises (a) a CDR-H1 comprising the amino acid sequence of SEQ ID NO: 25; (b) a CDR-H2 comprising the amino acid sequence of SEQ ID NO: 26; (c) a CDR-H3 comprising the amino acid sequence of SEQ ID NO: 27; (d) a CDR-L1 comprising the amino acid sequence of SEQ ID NO: 28; (e) a CDR-L2 comprising the amino acid sequence of SEQ ID NO: 29; and an (f) CDR-L3 comprising the amino acid sequence of SEQ ID NO: 30.

In further aspects, the antigen binding protein comprises (a) a VH comprising a CDR-H1, a CDR-H2, and a CDR-H3 comprising the amino acid sequences of the CDR-H1, CDR-H2, and CDR-H3 in the VH amino acid sequence set forth in SEQ ID NO: 31, and (b) a VL comprising a CDR-L1, a CDR-L2, and a CDR-H3 comprising the amino acid sequences of the CDR-L1, CDR-L2, and the CDR-L3 in the VL amino acid sequence as set forth in SEQ ID NO: 32.

Certain antigen binding proteins comprise a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence of SEQ ID NO: 25, the CDR-H2 reference sequence has the amino acid sequence of SEQ ID NO: 26, and the CDR-H3 reference sequence has the amino acid sequence of SEQ ID NO: 27. In such aspects, the amino acid changes typically are insertions, deletions and/or substitutions. In some of these aspects, the collective number of amino acid changes are 1-3; in other aspects, the collective number of amino acid changes are 1 or 2. In some of the foregoing aspects, the changes are conservative amino acid substitutions.

In other aspects, an antigen binding protein comprises a VL comprising a CDR-L1, a CDR-L2, and a CDR-L3, wherein the CDRs of the VL collectively have at most 1, 2, 3, 4, or 5 amino acid changes relative to a corresponding CDR reference sequence, and wherein the CDR-L1 reference sequence has the amino acid sequence of SEQ ID NO: 25, the CDR-L2 reference sequence has the amino acid sequence of SEQ ID NO: 26, and the CDR-L3 reference sequence has the amino acid sequence of SEQ ID NO: 27. In such aspects, the amino acid changes typically are insertions, deletions and/or substitutions. In some of these aspects, the collective number of amino acid changes are 1-3; in other aspects, the collective number of amino acid changes are 1 or 2. In certain of the foregoing aspects, the changes are conservative amino acid substitutions.

In further aspects, an antigen binding protein comprises (a) a VH comprising a CDR-H1, a CDR-H2, and a CDR-H3, wherein the CDRs of the VH collectively have at most 1, 2, 3, 4, or 5 amino acid changes relative to a corresponding CDR reference sequence, and wherein the CDR-H1 reference sequence has the amino acid sequence of SEQ ID NO: 25, the CDR-H2 reference sequence has the amino acid sequence of SEQ ID NO: 26, and the CDR-H3 reference sequence has the amino acid sequence of SEQ ID NO: 27, and (b) a VL comprising a CDR-L1, a CDR-L2, and a CDR-L3, wherein the CDRs of the VL collectively have at most 1, 2, 3, 4, or 5 amino acid changes relative to a corresponding CDR reference sequence, and wherein the CDR-L1 reference sequence has the amino acid sequence of SEQ ID NO: 28, the CDR-L2 reference sequence has the amino acid sequence of SEQ ID NO: 29, and the CDR-L3 reference sequence has the amino acid sequence of SEQ ID NO: 30. In such aspects, the amino acid changes typically are insertions, deletions and/or substitutions. In some of these aspects, the collective number of amino acid changes are at most 1-3 amino acid changes; in other aspects, the collective number of amino acid changes are 1 or 2 changes. In certain of the foregoing aspects, the changes are conservative amino acid substitutions.

An antigen binding protein as provided herein can comprise any suitable framework variable domain sequence, provided that the antibody retains the ability to bind CD25 (e.g., human CD25). As used herein, heavy chain framework regions are designated “HC-FR1-FR2-FR3-FR4,” and light chain framework regions are designated “LC-FR1-FR2-FR3-FR4.” In some aspects, the antigen binding protein comprises a heavy chain variable domain framework sequence of SEQ ID NO: 33, 34, 35, and 36, which correspond to amino acid sequence of HC-FR1, HC-FR2, HC-FR3, and HC-FR4, respectively. In some aspects, the antigen binding protein comprises a light chain variable domain framework sequence of SEQ ID NO: 37, 38, 39 and 40, which correspond to the amino acid sequence of LC-FR1, LC-FR2, LC-FR3, and LC-FR4, respectively. In some aspects, an antigen binding protein comprises CDR sequences as described in this section that have been inserted or grafted into their respective locations in such frameworks. In some aspects, one or more of the framework regions differs from the foregoing framework sequences by no more than 1, 2, 3, 4, 5, 6, 7, 8, 9, or 10 amino acids in the VH and/or VL framework regions. In certain of these aspects, there are at most 1, 2, 3, 4, or 5 amino acid differences in the VH and/or VL framework regions. In still other aspects, there are at most lor 2, 1-3, or 1-5 amino acid differences in the VH and/or VL framework regions. In certain aspects, there are at most 1 or 2, 1-3, or 1-5 amino acid differences in the VH framework regions. In some aspects, there are at most 1 or 2, 1-3, or 1-5 amino acid differences in the VL framework regions. In any of the foregoing aspects, the differences can be conservative amino acid substitutions. In some aspects, the differences correspond to backmutations.

As an example of such an aspect, certain antigen binding proteins as provided herein comprise a heavy chain variable region (VH) and a light chain variable region (VL), wherein the antibody comprises:
(a) a VH comprising:
   (1) a HC-FR1 comprising the amino acid sequence of SEQ ID NO: 33;
   (2) a CDR-H1 comprising the amino acid sequence of SEQ ID NO: 25;
   (3) a HC-FR2 comprising the amino acid sequence of SEQ ID NO: 34;
   (4) a CDR-H2 comprising the amino acid sequence of SEQ ID NO: 26;
   (5) a HC-FR3 comprising the amino acid sequence of SEQ ID NO: 35;
   (6) a CDR-H3 comprising the amino acid sequence of SEQ ID NO: 27; and
   (7) a HC-FR4 comprising the amino acid sequence of SEQ ID NO: 36, and/or
(b) a VL comprising:
   (1) a LC-FR1 comprising the amino acid sequence of SEQ ID NO: 37;
   (2) a CDR-L1 comprising the amino acid sequence of SEQ ID NO: 28;
   (3) a LC-FR2 comprising the amino acid sequence of SEQ ID NO: 38;
   (4) a CDR-L2 comprising the amino acid sequence of SEQ ID NO: 29;
   (5) a LC-FR3 comprising the amino acid sequence of SEQ ID NO: 39;
   (6) a CDR-L3 comprising the amino acid sequence of SEQ ID NO: 30; and
   (7) a LC-FR4 comprising the amino acid sequence of SEQ ID NO: 40.

In some aspects, the antigen binding protein comprises a VH comprising the amino acid sequence of SEQ ID NO: 31, and a VL comprising the amino acid sequence of SEQ ID NO: 32.

The antigen binding protein in other aspects comprises a VH domain, wherein the VH domain sequence has at least 80%, 85%, 90%, 91%, 92%, 93%, 94%, 95%, 96%, 97%, 98%, or 99% sequence identity to the amino acid sequence of SEQ ID NO: 31, provided the antigen binding protein retains the ability to bind to CD25. In certain aspects, such an antigen binding protein contains substitutions (e.g., conservative substitutions), insertions, or deletions relative to the reference sequence (i.e., SEQ ID NO: 31), provided that such an antigen binding protein retains the ability to bind to CD25. In certain aspects, 1, 2, 3, 4, 5, 6, 7, 8, 9, or 10 amino acids have been substituted, inserted and/or deleted in SEQ ID NO: 31. In some aspects, 1-5 or 1-3 amino acids have been substituted, inserted and/or deleted in the VH sequence. In certain of these aspects, such substitutions, insertions, or deletions occur in regions outside the CDRs (i.e., in the FRs). In further such aspects, the VH comprises one, two or three CDRs selected from: (a) a CDR-H1 comprising the amino acid sequence of SEQ ID NO: 25; (b) a CDR-H2 comprising the amino acid sequence of SEQ ID NO: 26; and (c) a CDR-H3 comprising the amino acid of SEQ ID NO: 27.

The antigen binding protein in other aspects comprises a VL domain, wherein the VL domain sequence has at least 80%, 85%, 90%, 91%, 92%, 93%, 94%, 95%, 96%, 97%, 98%, or 99% sequence identity to the amino acid sequence of SEQ ID NO: 32, provided the a antigen binding protein retains the ability to bind to CD25. In certain aspects, such an antigen binding protein contains substitutions (e.g., conservative substitutions), insertions, or deletions relative to the reference sequence (i.e., SEQ ID NO: 8), provided that such an antigen binding protein retains the ability to bind to CD25. In certain aspects, 1, 2, 3, 4, 5, 6, 7, 8, 9, or 10 amino acids have been substituted, inserted and/or deleted in SEQ ID NO: 8. In some aspects, 1-5 or 1-3 amino acids have been substituted, inserted and/or deleted in the VL sequence. In certain of these aspects, such substitutions, insertions, or deletions occur in regions outside the CDRs (i.e., in the FRs). In further such aspects, the VL comprises one, two or three CDRs selected from: (a) a CDR-L1 comprising the amino acid sequence of SEQ ID NO: 28; (b) a CDR-L2 comprising the amino acid sequence of SEQ ID NO: 29; and (c) a CDR-L3 comprising the amino acid sequence of SEQ ID NO: 30.

In further aspects, the antigen binding protein comprises (a) a VH domain, wherein the VH domain sequence has at least 80%, 85%, 90%, 91%, 92%, 93%, 94%, 95%, 96%, 97%, 98%, or 99% sequence identity to the amino acid sequence of SEQ ID NO: 31, and (b) a VL domain, wherein the VL domain sequence has at least 80%, 85%, 90%, 91%, 92%, 93%, 94%, 95%, 96%, 97%, 98%, or 99% sequence identity to the amino acid sequence of SEQ ID NO: 32, provided the antigen binding protein retains the ability to bind to CD25. In certain aspects, such an antigen binding protein contains substitutions (e.g., conservative substitutions), insertions, or deletions relative to the reference sequence (i.e., SEQ ID NO: 31 for the VH domain and SEQ ID NO: 32 for the VL domain), provided that such an antigen binding protein retains the ability to bind to CD25. In certain aspects, 1, 2, 3, 4, 5, 6, 7, 8, 9, or 10 amino acids have been substituted, inserted and/or deleted in the VH and/or VL sequence. In some aspects, 1-5 or 1-3 amino acids have been substituted, inserted and/or deleted in the VH and/or VL sequence. In other aspects, 1-5 or 1-3 amino acids have been substituted, inserted and/or deleted in the VL and VH sequences collectively. In certain of these aspects, such substitutions, insertions, or deletions occur in regions outside the CDRs (i.e., in the FRs). In further such aspects, the VH comprises one, two, or three CDRs selected from: (i) a CDR-H1 comprising the amino acid sequence of SEQ ID NO: 25; (ii) a CDR-H2 comprising the amino acid sequence of SEQ ID NO: 26; (iii) a CDR-H3 comprising the amino acid sequence of SEQ ID NO: 27, and the VL comprises one, two, or three CDRs selected from: (i) a CDR-L1 comprising the amino acid sequence of SEQ ID NO: 28; (ii) a CDR-L2 comprising the amino acid sequence of SEQ ID NO: 29; (iii) a CDR-L3 comprising the amino acid sequence of SEQ ID NO: 30.

In further aspects, an antigen binding protein comprises an HC comprising the amino acid sequence of SEQ ID NO: 43, and a LC comprising the amino acid sequence of SEQ ID NO: 44.

The antigen binding protein as described in this section can be an antibody in any form. As such, the antigen binding protein described in any of the above aspects can be monoclonal, and may be multispecific, human, humanized or chimeric antibodies, single chain antibodies, Fab fragments, F(ab′) fragments, fragments produced by a Fab expression library, and CD25 binding fragments of any of the above. The antibodies can be of any immunoglobulin isotype (e.g., IgG, IgE, IgM, IgD, IgA and IgY), class (e.g., IgG1, IgG2, IgG3, IgG4, IgA1 and IgA2) or subclass.

In certain aspects, an antigen binding protein with the CDR and/or variable domain sequences described herein is an antigen-binding fragment (e.g., human antigen-binding fragments) and include, but are not limited to, Fab, Fab′ and F(ab′)₂, Fd, single-chain Fvs (scFv), single-chain antibodies, disulfide-linked Fvs (sdFv) and fragments comprising either a VL or VH domain.

The antigen binding protein can be monospecific or part of a bispecific or trispecific antigen binding protein, or part of an antigen binding protein of greater multi-specificity. Multispecific antibodies can be specific for different epitopes of CD25 or may be specific for both CD25 and a heterologous protein.

Also provided herein are antigen binding proteins that bind to the same epitope as antigen binding protein SG25Ab-9, SG25Ab-9 YH98A, or SG25Ab-4 or other antigen binding proteins as described in this section.

Other antigen binding proteins that are provided herein compete with antigen binding protein SG25Ab-9, SG25Ab-9 YH98A, or SG25Ab-4 or other antigen binding proteins as described in this section for binding to CD25.

### C. Chimeric Antigen Binding Proteins

In certain aspects, the antigen binding protein provided herein is a chimeric antibody. In some aspects, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Certain chimeric antibodies are described, e.g., in U.S. Pat. No. 4,816,567; and Morrison et al., (1984) Proc. Nat. Acad. Sci. USA, 81:6851-6855 (1984)). Chimeric antibodies include antigen-binding fragments thereof.

Nonlimiting exemplary chimeric antibodies include chimeric antibodies comprising any of the heavy and/or light chain variable regions as described herein. In certain aspects, the heavy and/or light chain variable domains are selected from SEQ ID NOs: 7, 8, 22, 31 and 32. Additional nonlimiting exemplary chimeric antibodies include chimeric antibodies comprising heavy chain CDR sequences (e.g., CDRs) or portions thereof, and/or light chain CDR sequences (e.g., CDRs) as provided herein. For example, in some aspects, the CDR (e.g., CDR) sequences are from an antibody selected from SEQ ID NOs: 1-6, 21, and 25-30.

### D. Humanized Antigen Binding Proteins

In certain aspects, the antigen binding protein is a humanized antibody that binds CD25. Typically, a non-human antibody is humanized to reduce immunogenicity to humans, while retaining the specificity and affinity of the parental non-human antibody. A humanized antibody is a genetically engineered antibody in which the CDRs (e.g., CDRs) or portions thereof from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. No. 5,859,205; and Foote, U.S. Pat. No. 6,881,557).

The acceptor antibody sequences can be, for example, a mature human antibody sequence, a composite of such sequences, a consensus sequence of human antibody sequences, or a germline region sequence. Human acceptor sequences can be selected for a high degree of sequence identity in the variable region frameworks with donor sequences to match canonical forms between acceptor and donor CDRs or CDRs among other criteria. Thus, a humanized antibody is an antibody having CDRs or CDRs entirely or substantially from a donor antibody and variable region framework sequences and constant regions, if present, entirely or substantially from human antibody sequences. Similarly, a humanized heavy chain typically has all three CDRs or CDRs entirely or substantially from a donor antibody heavy chain, and a heavy chain variable region framework sequence and heavy chain constant region, if present, substantially from human heavy chain variable region framework and constant region sequences. Likewise, a humanized light chain usually has all three CDRs entirely or substantially from a donor antibody light chain, and a light chain variable region framework sequence and light chain constant region, if present, substantially from human light chain variable region framework and constant region sequences. A CDR or CDR in a humanized antibody is substantially from a corresponding CDR or CDR in a non-human antibody when at least 80%, 85%, 90%, 95% or 100% of corresponding residues (as defined by Kabat) are identical between the respective CDRs or CDRs. The variable region framework sequences of an antibody chain or the constant region of an antibody chain are substantially from a human variable region framework sequence or human constant region respectively when at least 80%, 85%, 90%, 95% or 100% of corresponding residues defined by Kabat are identical.

Although humanized antibodies often incorporate all six CDRs (e.g., CDRs, preferably as defined by Kabat) from a mouse antibody, they can also be made with less than all CDRs or CDRs (e.g., at least 3, 4, or 5) CDRs or CDRs from a mouse antibody (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; and Tamura et al, Journal of Immunology, 164:1432-1441, 2000).

Certain amino acids from the human variable region framework residues can be selected for substitution based on their possible influence on CDR (e.g., CDR) conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid: (1) noncovalently binds antigen directly, (2) is adjacent to a CDR or CDR region, (3) otherwise interacts with a CDR or CDR region (e.g. is within about 6 Å of such a region); (4) mediates interaction between the heavy and light chains; (5) is the result of somatic mutation in the mouse chain; or (6) is a site of glycosylation.

Framework residues from classes (1)-(3) are sometimes alternately referred to as canonical and vernier residues. Canonical residues refer to framework residues defining the canonical class of the donor CDR loops determining the conformation of a CDR loop (Chothia and Lesk, J. Mol. Biol. 196, 901-917 (1987), Thornton & Martin, J. Mol. Biol., 263, 800-815, 1996). Vernier residues refer to a layer of framework residues that support antigen-binding loop conformations and play a role in fine-tuning the fit of an antibody to antigen (Foote & Winter, 1992, J Mol Bio. 224, 487-499).

Humanized antibodies and methods of making them are reviewed, e.g., in Almagro and Fransson, (2008) Front. Biosci. 13: 1619-1633, and are further described, e.g., in Riechmann et al., (1988) Nature 332:323-329; Queen et al., (1989) Proc. Natl Acad. Sci. USA 86: 10029-10033; U.S. Pat. Nos. 5,821,337, 7,527,791, 6,982,321, and 7,087,409; Kashmiri et al., (2005) Methods 36:25-34 (describing specificity determining region (SDR) grafting); Padlan, (1991) Mol. Immunol. 28:489-498 (describing “resurfacing”); Dall′Acqua et al., (2005) Methods 36:43-60 (describing “FR shuffling”); and Osbourn et al., (2005) Methods 36:61-68 and Klimka et al., (2000) Br. J Cancer, 83:252-260 (describing the “guided selection” approach to FR shuffling).

Human framework regions that may be used for humanization include but are not limited to: framework regions selected using the “best-fit” method (see, e.g., Sims et al. (1993) J. Immunol. 151:2296); framework regions derived from the consensus sequence of human antibodies of a particular subgroup of light or heavy chain variable regions (see, e.g., Carter et al. (1992) Proc. Natl. Acad. Sci. USA, 89:4285; and Presta et al. (1993) J Immunol, 151:2623); human mature (somatically mutated) framework regions or human germline framework regions (see, e.g., Almagro and Fransson, (2008) Front. Biosci. 13:1619-1633); and framework regions derived from screening FR libraries (see, e.g., Baca et al., (1997) J Biol. Chem. 272: 10678-10684 and Rosok et al., (1996) J Biol. Chem. 271:22611-22618). Nonlimiting exemplary chimeric antibodies include chimeric antibodies comprising or derived from any of the CDR (e.g., CDR), and/or heavy and/or light chain variable regions as disclosed herein. Specific examples of such antibodies include chimeric antibodies comprising the heavy chain CDRs of SEQ ID NO: 1-3, 21, or 25-27 and/or the light chain CDRs of SEQ ID NO: 4-6, or 28-30. In some aspects, chimeric antibodies comprise VH of SEQ ID NO: 7, 22, or 31 and/or VL of SEQ ID NO: 8, or 32.

### E. Human Antigen Binding Proteins

In certain aspects, the antigen binding protein provided herein is a human antigen binding protein, such as a human antibody. Human antibodies can be produced using various techniques known in the art. Human antibodies are described generally in van Dijk and van de Winkel, (2001) Curr. Opin. Pharmacol. 5:368-374 and Lonberg, (2008) Curr. Opin. Immunol. 20:450-459. In some aspects, the antibody is not a naturally-occurring antibody.

Human antibodies can be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, (2005) Nat. Biotech. 23: 1117-1125. See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor (1984) J Immunol, 133: 3001; Brodeur et al., Monoclonal Antibody Production Techniques and Applications, pp. 51-63 (Marcel Dekker, Inc., New York, 1987); and Boerner et al, (1991) J Immunol., 147:86). Human antibodies generated via human B-cell hybridoma technology are also described in Li et al., (2006) Proc. Natl. Acad. Sci. USA, 103:3557-3562. Additional methods include those described, for example, in U.S. Pat. No. 7,189,826 (describing production of monoclonal human IgM antibodies from hybridoma cell lines) and Ni, (2006) Xiandai Mianyixue, 26(4):265-268 (describing human-human hybridomas). Human hybridoma technology (Trioma technology) is also described in Vollmers and Brandlein, (2005) Histology and Histopathology, 20(3):927-937, and Vollmers and Brandlein, (2005) Methods and Findings in Experimental and Clinical Pharmacology, 27(3): 185-191.

Human antibodies can also be generated by isolating Fv clone variable domain sequences selected from human-derived phage display libraries. Such variable domain sequences may then be combined with a desired human constant domain. Techniques for selecting human antibodies from antibody libraries are described below.

Human antibodies can also be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178: 1-37 (O'Brien et al., ed., Human Press, Totowa, NJ, 2001) and further described, e.g., in the McCafferty et al, (1990) Nature 348:552-554; Clackson et al, (1991) Nature 352: 624-628; Marks et al, (1992) J. Mol. Biol 222: 581-597; Marks and Bradbury, in Methods in Molecular Biology 248: 161-175 (Lo, ed., Human Press, Totowa, NJ, 2003); Sidhu et al, (2004) J Mol. Biol. 338(2): 299-310; Lee et al., (2004) J Mol. Biol. 340(5): 1073-1093; Fellouse, (2004) Proc. Natl. Acad. Sci. USA 101(34): 12467-12472; and Lee et al, (2004) J Immunol. Methods 284(1-2): 119-132 and PCT publication WO 99/10494.

In certain phage display methods, repertoires of V_{H} and V_{L} genes are separately cloned by polymerase chain reaction (PCR) and recombined randomly in phage libraries, which can then be screened for antigen-binding phage as described in Winter et al., (1994) Ann. Rev. Immunol., 12:433-455. Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., (1993) EMBO J 12:725-734. Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter (1992), J. Mol. Biol, 227:381-388. Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

In some aspects, the antigen binding protein is a human anti-CD25 antibody that binds to a polypeptide having the sequence of SEQ ID NO: 42 and that comprise any of the CDRs (e.g., CDRs), and/or heavy and/or light chain variable regions as disclosed herein. Specific examples of such antibodies include CD25 antibodies SG25Ab-9, SG25Ab-9 YH98A, and SG25Ab-4, which are described in greater detail in the Examples and which have the sequences shown in Table 4.

### F. Exemplary Antibody Constant Regions

For those aspects in which antigen binding proteins are antibodies, the heavy and light chain variable regions of antibodies described herein can be linked to at least a portion of a human constant region. In some aspects, the human heavy chain constant region is of an isotype selected from IgA, IgG, and IgD. In some aspects, the human light chain constant region is of an isotype selected from κ and λ. In some aspects, an antibody described herein comprises a human IgG constant region. In some aspects, an antibody described herein comprises a human IgG4 heavy chain constant region. In some of these aspects, an antibody described herein comprises an S241P mutation in the human IgG4 constant region. In some aspects, an antibody described herein comprises a human IgG4 constant region and a human κ light chain.

Throughout the present specification and claims unless explicitly stated or known to one skilled in the art, the numbering of the residues in an immunoglobulin heavy chain is that of the EU index as in Kabat et al., Sequences of Proteins of Immunological Interest, 5th Ed. Public Health Service, National Institutes of Health, Bethesda, Md. (1991), expressly incorporated herein by reference. The “EU index as in Kabat” refers to the residue numbering of the human IgG1 EU antibody.

Human constant regions show allotypic variation and isoallotypic variation between different individuals, that is, the constant regions can differ in different individuals at one or more polymorphic positions. Isoallotypes differ from allotypes in that sera recognizing an isoallotype binds to a non-polymorphic region of a one or more other isotypes. Reference to a human constant region includes a constant region with any natural allotype or any permutation of residues occupying polymorphic positions in natural allotypes. Also, up to 1, 2, 5, or 10 mutations may be present relative to a natural human constant region, such as those indicated above to reduce Fcγ receptor binding or increase binding to FcRn.

In some aspects, the human constant region comprises SEQ ID NO: 41.

In some aspects, one or several amino acids at the amino or carboxy terminus of the light and/or heavy chain, such as the C-terminal lysine of the heavy chain, may be missing or derivatized in a proportion or all of the molecules.

The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have strong complement-dependent cytotoxicity, human isotype IgG2 weak complement-dependent cytotoxicity and human IgG4 lacks complement-dependent cytotoxicity. Human IgG1 and IgG3 also induce stronger cell-mediated effector functions than human IgG2 and IgG4. Light chain constant regions can be lambda or kappa.

Furthermore, as described in greater detail below, substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004).

### G. Variants

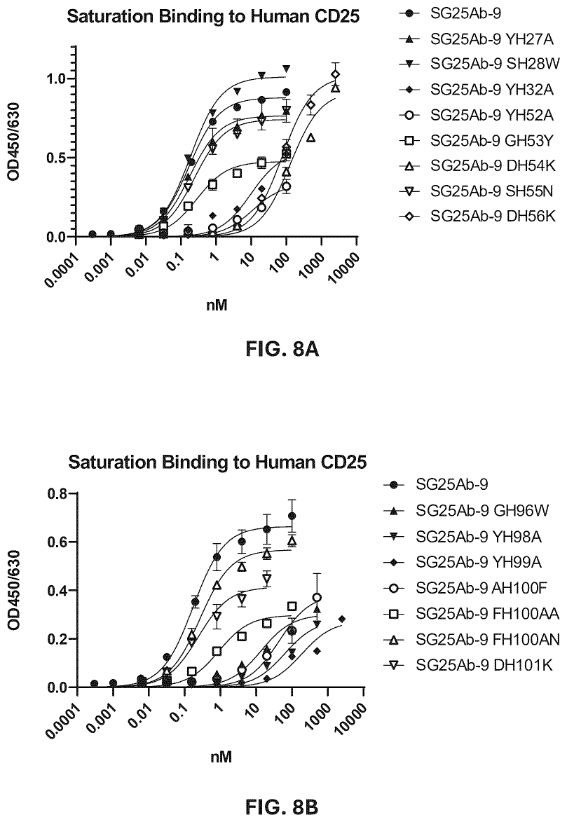

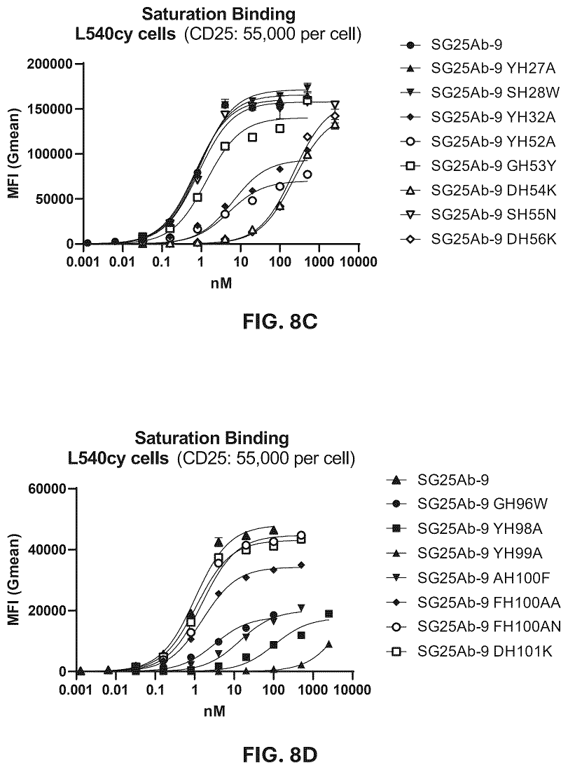

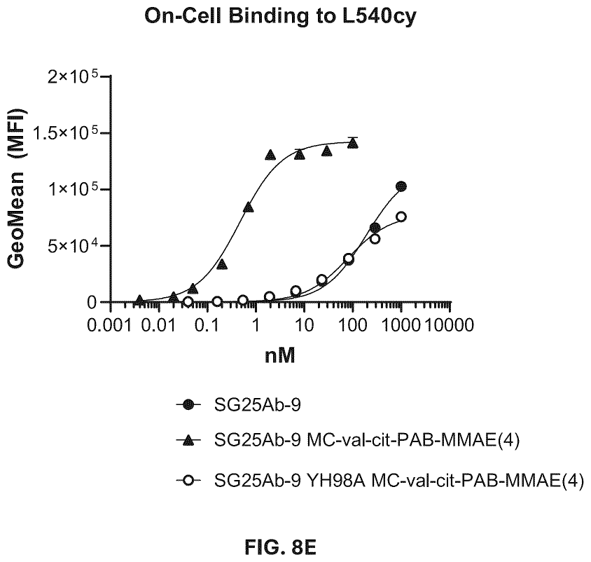

The antigen binding proteins provided herein also include amino acid sequence variants of the antigen binding proteins provided herein such as those described in Tables 2, 3, and 4 and/or FIGS. 8-20. As an example, variants with improved binding affinity and/or other biological properties of the antibody can be prepared. In some aspects, antibodies with reduced binding affinity and improved biological properties can be prepared. Amino acid sequence variants of an antigen binding protein can be prepared by introducing appropriate modifications into the nucleotide sequence encoding the antigen binding protein, or by peptide synthesis. Such modifications include, for example, deletions from, and/or insertions into and/or substitutions of residues within the amino acid sequences of the antigen binding protein. Any combination of deletion, insertion, and substitution can be made to arrive at the final construct, provided that the final construct possesses the desired characteristics, e.g., antigen-binding.

### 1. Substitution, Insertion and Deletion Variants

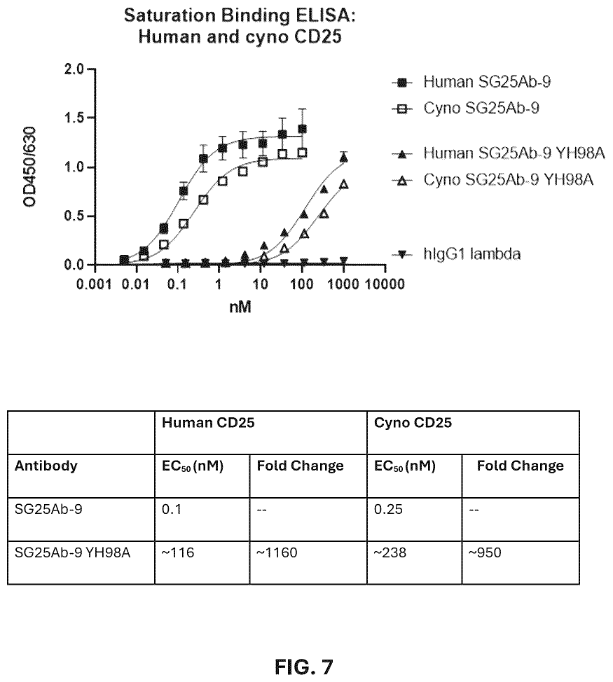

In some aspects, an antigen binding protein is a variant in that it has one or more amino acid substitutions, deletions and/or insertions relative to an antigen binding protein as described herein (e.g., an antigen binding protein having the amino acid sequences as described in Tables 2, 3, and 4, Examples 3-8, and FIGS. 7-19. In certain such aspects, the variant has one or more amino acid substitutions. In further such aspects, the substitutions are conservative amino acid substitutions.

An amino acid substitution can include but are not limited to the replacement of one amino acid in a polypeptide with another amino acid. Conservative amino acid substitutions can encompass non-naturally occurring amino acid residues, which are typically incorporated by chemical peptide synthesis rather than by synthesis in biological systems. Naturally occurring residues can be divided into classes based on common side chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Sites of interest for substitutional mutagenesis include the CDRs and FRs. Conservative substitutions are shown in Table 5 below under the heading of “Preferred Substitutions.” More substantial changes are provided in Table 5 under the heading of “Exemplary Substitutions,” and as further described below in reference to amino acid side chain classes. Amino acid substitutions may be introduced into an antibody of interest and the products screened for a desired activity, e.g., retained/improved antigen binding, decreased binding, decreased immunogenicity, improved ADCC or CDC, or improved cell specificity.

TABLE 5
Substitutions

| Original | Exemplary | Preferred |
|---|---|---|
| Residue | Substitutions | Substitutions |
| Ala | Val; Leu; Ile | Val |
| Arg | Lys; Gln; Asn | Lys |
| Asn | Gln; His; Asp; Lys; Arg | Gln |
| Asp | Glu; Asn | Glu |
| Cys | Ser; Ala | Ser |
| Gln | Asn; Glu | Asn |
| Glu | Asp; Gln | Asp |
| Gly | Pro; Ala | Ala |
| His | Asn; Gln; Lys; Arg | Arg |
| Ile | Leu; Val; Met; Ala; Phe; Norleucine | Leu |
| Leu | Norleucine; Ile; Val; Met; Ala; Phe | Ile |
| Lys | Arg; Gln; Asn | Arg |
| Met | Leu; Phe; Ile | Leu |
| Phe | Trp; Leu; Val; Ile; Ala; Tyr | Leu |
| Pro | Ala | Ala |
| Ser | Thr; Ala; Cys | Thr |
| Thr | Val; Ser | Ser |
| Trp | Tyr; Phe | Tyr |
| Tyr | Trp; Phe; Thr; Ser | Phe |
| Val | Ile; Leu; Met; Phe; Ala; Norleucine | Leu |

Non-conservative substitutions involve exchanging a member of one of these classes for another class. In some aspects, glycerin is mutated to alanine, aspartic acid is mutated to asparagine or alanine, or tyrosine is mutated to alanine.

In altering the amino acid sequence of the antigen binding protein (e.g., anti-CD25 antibody), in some aspects the hydropathic index of amino acids can be considered. Each amino acid has been assigned a hydropathic index on the basis of its hydrophobicity and charge characteristics as follows: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. (Kyte et al., 1982, J Mol. Biol., 157:105-131). It is known that certain amino acids can be substituted for other amino acids having a similar hydropathic index or score and still retain a similar biological activity. In making changes based upon the hydropathic index, in certain aspects, the substitution of amino acids whose hydropathic indices are within +2 is included. In certain aspects, those which are within +1 are included, and in certain aspects, those within +0.5 are included.

It is also understood in the art that the substitution of like amino acids can be made effectively on the basis of hydrophilicity, particularly where the biologically functional protein or peptide (e.g., antibody) thus created is intended for use in immunological aspects, as in the present case. In certain aspects, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain aspects, the substitution of amino acids whose hydrophilicity values are within ±2 is included, in certain aspects, those which are within ±1 are included, and in certain aspects, those within ±0.5 are included. One can also identify epitopes from primary amino acid sequences on the basis of hydrophilicity. These regions are also referred to as “epitopic core regions.”

Alterations (e.g., substitutions) can be made in CDRs, e.g., to modulate antibody affinity. Such alterations can be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted. In some aspects, diversity is introduced into CDRs to reduce binding affinity. In some aspects, diversity is introduced into the variable genes by any of a variety of methods (e.g., error-prone PCR, chain shuffling, or oligonucleotide-directed mutagenesis) to create a secondary library. The library is then screened to identify any antibody variants with the desired reduced binding affinity. In some aspects, diversity is introduced using CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted to reduce binding affinity. In some aspects, CD25 antibodies with reduced binding affinity to CD25 comprise a mutation in at least one heavy chain variable region residue YH27, SH28, Y32, Y52, G53, D54, 555, D56, G96, YH98A, YH99, A100, FH100A, or DH101.

In certain aspects, substitutions, insertions, or deletions can occur within one or more CDRs so long as such alterations do not substantially reduce the ability of the antibody to bind antigen. For example, conservative alterations (e.g., conservative substitutions as provided herein) that do not excessively change binding affinity may be made in CDRs. Such alterations may, for example, be outside of antigen contacting residues in the CDRs. In certain aspects of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

2. Variants with Modified Fc Region

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

In certain aspects, an antibody variant is prepared that has improved or diminished binding to FcRs are described. (See, e.g., U.S. Pat. No. 6,737,056; WO 2004/056312, and Shields et al., J. Biol. Chem. 9(2): 6591-6604 (2001).) In some aspects, an antibody variant comprises an Fc region with one or more amino acid substitutions which improve ADCC, e.g., substitutions at positions 298, 333, and/or 334 of the Fc region (EU numbering of residues). For instance, a systemic substitution of solvent-exposed amino acids of human IgG1 Fc region has generated IgG variants with altered FcγR binding affinities (Shields et al., 2001, J Biol. Chem. 276:6591-604). When compared to parental IgG1, a subset of these variants involving substitutions at Thr256/Ser298, Ser298/Glu333, Ser298/Lys334, or Ser298/Glu333/Lys334 to Ala demonstrate increased in both binding affinity toward FcγR and ADCC activity (Shields et al., 2001, J. Biol. Chem. 276:6591-604; Okazaki et al., 2004, J Mol. Biol. 336:1239-49).

In some aspects, alterations are made in the Fc region to alter (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000). For instance, complement fixation activity of antibodies (both C1q binding and CDC activity) can be improved by substitutions at Lys326 and Glu333 (Idusogie et al., 2001, J. Immunol. 166:2571-2575). The same substitutions on a human IgG2 backbone can convert an antibody isotype that binds poorly to C1q and is severely deficient in complement activation activity to one that can both bind C1q and mediate CDC (Idusogie et al., 2001, J Immunol. 166:2571-75). Several other methods have also been applied to improve complement fixation activity of antibodies. For example, the grafting of an 18-amino acid carboxyl-terminal tail piece of IgM to the carboxyl-termini of IgG greatly enhances their CDC activity. This is observed even with IgG4, which normally has no detectable CDC activity (Smith et al., 1995, J Immunol. 154:2226-36). Also, substituting Ser444 located close to the carboxy-terminal of IgG1 heavy chain with Cys induced tail-to-tail dimerization of IgG1 with a 200-fold increase of CDC activity over monomeric IgG1 (Shopes et al., 1992, J. Immunol. 148:2918-22). In addition, a bispecific diabody construct with specificity for C1q also confers CDC activity (Kontermann et al., 1997, Nat. Biotech. 15:629-31).

Complement activity can be reduced by mutating at least one of the amino acid residues 318, 320, and 322 of the heavy chain to a residue having a different side chain, such as Ala. Other alkyl-substituted non-ionic residues, such as Gly, Ile, Leu, or Val, or such aromatic non-polar residues as Phe, Tyr, Trp and Pro in place of any one of the three residues also reduce or abolish Cq binding. Ser, Thr, Cys, and Met can be used at residues 320 and 322, but not 318, to reduce or abolish C1q binding activity. Replacement of the 318 (Glu) residue by a polar residue may modify but not abolish C1q binding activity. Replacing residue 297 (Asn) with Ala results in removal of lytic activity but only slightly reduces (about three fold weaker) affinity for C1q. This alteration destroys the glycosylation site and the presence of carbohydrate that is required for complement activation. Any other substitution at this site also destroys the glycosylation site. The following mutations and any combination thereof also reduce C1q binding: D270A, K322A, P329A, and P311S (see WO 06/036291).

The half-life of an antibody as provided herein can be increased or decreased to modify its therapeutic activities. FcRn is a receptor that is structurally similar to MHC Class I antigen that non-covalently associates with β2-microglobulin. FcRn regulates the catabolism of IgGs and their transcytosis across tissues (Ghetie and Ward, 2000, Annu. Rev. Immunol. 18:739-766; Ghetie and Ward, 2002, Immunol. Res. 25:97-113). The IgG-FcRn interaction takes place at pH 6.0 (pH of intracellular vesicles) but not at pH 7.4 (pH of blood); this interaction enables IgGs to be recycled back to the circulation (Ghetie and Ward, 2000, Ann. Rev. Immunol. 18:739-766; Ghetie and Ward, 2002, Immunol. Res. 25:97-113). The region on human IgG₁ involved in FcRn binding has been mapped (Shields et al., 2001, J. Biol. Chem. 276:6591-604). Alanine substitutions at positions Pro238, Thr256, Thr307, Gln311, Asp312, Glu380, Glu382, or Asn434 of human IgG1 enhance FcRn binding (Shields et al., 2001, J. Biol. Chem. 276:6591-604). IgG1 molecules harboring these substitutions have longer serum half-lives. Consequently, these modified IgG1 molecules may be able to carry out their effector functions, and hence exert their therapeutic efficacies, over a longer period of time compared to unmodified IgG₁. Other exemplary substitutions for increasing binding to FcRn include a Gln at position 250 and/or a Leu at position 428. Other studies have shown that binding of the Fc region to FcRn can be improved by introducing one or more substitutions at one or more the following Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (see, e.g., U.S. Pat. Nos. 7,371,826; and 7,361,740).

3. Antibody Variants with Modified Glycosylation

In certain aspects, an antibody as provided herein includes one or more modifications so as to increase or decrease the extent to which the antibody is glycosylated. Addition or deletion of glycosylation sites to an antibody can be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure.

Engineering of this glycoform on IgG can significantly improve IgG-mediated ADCC. Addition of bisecting N-acetylglucosamine modifications (Umana et al., 1999, Nat. Biotechnol. 17:176-180; Davies et al., 2001, Biotech. Bioeng. 74:288-94) to this glycoform or removal of fucose (Shields et al., 2002, J. Biol. Chem. 277:26733-40; Shinkawa et al., 2003, J. Biol. Chem. 278:6591-604; Niwa et al., 2004, Cancer Res. 64:2127-33) from this glycoform are two examples of IgG Fc engineering that improves the binding between IgG Fc and FcγR, thereby enhancing Ig-mediated ADCC activity. Antibodies including such substitutions or engineering are included in some of the aspects provided herein.

In certain aspects, antibodies are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g., complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about +3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Patent Application No. US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Other antibodies are further provided which contain bisected oligosaccharides, e.g., in which a biantennary oligosaccharide attached to the Fc region of the antibody is bisected by GlcNAc. Such antibodies may have reduced fucosylation and/or improved ADCC function. Examples of such antibodies are described, e.g., in WO 2003/011878 (Jean-Mairet et al.); U.S. Pat. No. 6,602,684 (Umana et al.); and US 2005/0123546 (Umana et al.). Antibodies with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

### 4. Cysteine Engineered Antibody Variants

In some aspects, an antibody variant as provided herein includes a substitution of the native amino acid to a cysteine residue at amino acid position 234, 235, 237, 239, 267, 298, 299, 326, 330, or 332, preferably an S239C mutation (substitutions of the constant regions are according to the EU index) in a human IgG1 isotype. The presence of an additional cysteine residue allows interchain disulfide bond formation. Such interchain disulfide bond formation can cause steric hindrance, thereby reducing the affinity of the Fc region-FcγR binding interaction. The cysteine residue(s) introduced in or in proximity to the Fc region of an IgG constant region can also serve as sites for conjugation to therapeutic agents (e.g., coupling cytotoxic drugs using thiol specific reagents such as maleimide derivatives of drugs). The presence of a therapeutic agent causes steric hindrance, thereby further reducing the affinity of the Fc region-FcγR binding interaction. Other substitutions at any of positions 234, 235, 236 and/or 237 reduce affinity for Fcγ receptors, particularly FcγRI receptor (see, e.g., U.S. Pat. Nos. 6,624,821, 5,624,821.)

In other cysteine engineered antibody variants, one or more reactive thiol groups are positioned at accessible sites of the antibody and can be used to conjugate the antibody to other moieties, such as drug moieties or linker-drug moieties, to create an immunoconjugate, as described further herein. In certain aspects, any one or more of the following residues may be substituted with cysteine: V205 (Kabat numbering) of the light chain; A118 (EU numbering) of the heavy chain; and 5400 (EU numbering) of the heavy chain Fc region. Generating of cysteine engineered antibodies are described, e.g., in U.S. Pat. No. 7,521,541.

### 5. Exemplary Fc Variants

Certain of the antigen binding proteins that are provided include modifications to the constant region from e.g., Liu et al., Antibodies, 9: 64, 2020, Tables 1 and 2, included herein by reference in its entirety. Other modifications include afucosylation or other mutations to enhance Fc gamma receptor binding; Fc null such as LALAPG, FcRn enhancing or deleting, knobs in holes or Fab arm exchange mutations.

### H. Competing Antigen Binding Proteins

The antigen binding proteins provided herein include those that compete with one of the exemplified antigen binding proteins described above for specific binding to CD25 (e.g., human CD25 of SEQ ID NO: 42). In some of these aspects, the test and reference antigen binding proteins cross-compete with one another. Such antigen binding proteins may bind to the same epitope as one of the antigen binding proteins described herein, or to an overlapping epitope. Antigen binding proteins including fragments that compete with the exemplified antigen binding proteins are expected to show similar functional properties (e.g., one or more of the activities described above). The exemplified antigen binding proteins and fragments include those described above, including those with: 1) the heavy and/or light chains, 2) VHs and/or VLs, and/or 3) that comprise one or more of the CDRs included in Table 4.

Thus, in some aspects, the antigen binding proteins that are provided include those that compete with an antibody having: (a) all 6 of the CDRs listed for the same antibody listed in Table 4; (b) a VH and a VL listed for the same antibody listed in Table 4; or (c) two light chains and two heavy chains as specified for the same antibody listed in Table 4.

In some aspects, competition or cross-competition is determined by surface plasmon resonance analysis (e.g., BIACORE®) (see, e.g., Abdiche, et al., 2009, Anal. Biochem. 386:172-180; Abdiche, et al., 2012, J Immunol Methods 382:101-116; and Abdiche, et al., 2014 PLoS One 9:e92451). In some aspects, competition or cross-competition is determined by biolayer interferometry (BLI).

I. Antigen Binding Proteins that Bind the Same Epitope

In another aspects, the antigen binding proteins that are provided include those that bind the same epitope as any of the antigen binding proteins described herein. A variety of techniques are available to identify antigen binding proteins that bind to the same epitope as one or more of the antigen binding proteins described herein. Such methods include, for instance, competition assays such as described herein, screening of peptide fragments, MS-based protein footprinting, alanine or glutamine scanning approaches, and via x-ray analysis of crystals of antigen:antigen binding protein complexes which provides atomic resolution of the epitope.

One approach for determining the epitope or epitope region (an “epitope region” is a region comprising the epitope or overlapping with the epitope) bound by a specific antibody involves assessing binding of an antigen binding protein to peptides comprising fragments of CD25, e.g., non-denatured or denatured fragments. A series of overlapping peptides encompassing the sequence of CD25 (e.g., human CD25) can be prepared and screened for binding, e.g. in a direct ELISA, a competitive ELISA (where the peptide is assessed for its ability to prevent binding of an antibody to CD25 bound to a well of a microtiter plate), or on a chip. Such peptide screening methods may not be capable of detecting some discontinuous functional epitopes, i.e. functional epitopes that involve amino acid residues that are not contiguous along the primary sequence of the CD25 polypeptide chain.

In other aspects, the region(s) containing residues that are in contact with or are buried by an antibody can be identified by mutating specific residues in CD25 and determining whether the antigen binding protein can bind the mutated or variant CD25 protein. By making a number of individual mutations, residues that play a direct role in binding or that are in sufficiently close proximity to the antibody such that a mutation can affect binding between the antigen binding protein and antigen can be identified. From a knowledge of these amino acids, the domain(s) or region(s) of the antigen that contain residues in contact with the antigen binding protein or covered by the antibody can be elucidated. Such a domain can include the binding epitope of an antigen binding protein. The general approach for such scanning techniques involves substituting arginine and/or glutamic acid residues (typically individually) for an amino acid in the wild-type polypeptide. These two amino acids are typically used in such scanning techniques because they are charged and bulky and thus have the potential to disrupt binding between an antigen binding protein and the CD25 in the region of the CD25 where the mutation is introduced. Arginines that exist in the wild-type antigen are replaced with glutamic acid. A variety of such individual mutants are obtained and the collected binding results analyzed to determine what residues affect binding (see, e.g., Nanevicz, T., et al., 1995, J. Biol. Chem., 270:37, 21619-21625 and Zupnick, A., et al., 2006, J. Biol. Chem., 281:29, 20464-20473).

An alternative approach for identifying an epitope is by MS-based protein footprinting, such as hydrogen/deuterium exchange mass spectrometry (HDX-MS) and Fast Photochemical Oxidation of Proteins (FPOP). Methods for conducting HDX-MS are described, for example, in Wei et al. (2014) Drug Discovery Today 19:95. Methods for performing FPOP are described, for instance, in Hambley and Gross (2005) J. American Soc. Mass Spectrometry 16:2057.

The epitope bound by an antigen binding protein can also be determined by structural methods, such as an X-ray crystal structure determination, molecular modeling, and nuclear magnetic resonance (NMR) spectroscopy, including NMR determination of the H-D exchange rates of labile amide hydrogens in the antigen when free and when bound in a complex with an antigen binding protein (see, e.g., Zinn-Justin et al. (1992) Biochemistry 31, 11335-11347; and Zinn-Justin et al. (1993) Biochemistry 32, 6884-6891).

X-ray crystallography analyses can be accomplished using any of the known methods in the art. Examples of crystallization methods are described, for instance, by Giege et al. (1994) Acta Crystallogr. D50:339-350; and McPherson (1990) Eur. J. Biochem. 189:1-23). Such crystallization approaches include microbatch (e.g. Chayen (1997) Structure 5:1269-1274), hanging-drop vapor diffusion (e.g. McPherson (1976) J. Biol. Chem. 251:6300-6303), seeding and dialysis. Once formed, the antigen binding protein:antigen crystals themselves can be studied using well-known X-ray diffraction techniques and can be refined using computer software such as X-PLOR (Yale University, 1992, distributed by Molecular Simulations, Inc.; see e.g. Blundell & Johnson (1985) Meth. Enzymol. 114 & 115, H. W. Wyckoff et al., eds., Academic Press; U.S. Patent Application Publication No. 2004/0014194), and BUSTER (Bricogne (1993) Acta Cryst. D49:37-60; Bricogne (1997) Meth. Enzymol. 276A:361-423, Carter & Sweet, eds.; Roversi et al. (2000) Acta Cryst. D56:1313-1323).

In some aspects, the antigen binding protein binds an epitope of CD25 that is continuous. In some aspects, the antigen binding protein binds an epitope of CD25 that is discontinuous.

In some aspects, the antigen binding protein binds an epitope that is bound by any one of the antigen binding proteins described herein and in Tables 2, 3, and 4.

### J. Exemplary Functional Activities

### 1. Affinity

The antigen binding proteins in some aspects bind to CD25 with an affinity (e.g., EC₅₀) of about 50 pM to about 500 nM. In some aspects, the antigen binding protein binds to CD25 with an affinity of about 75 pM to about 480 nM, about 100 pM to about 450 nM, about 150 pM to about, 400 nM, about 200 pM to about 350 nM, about 250 pM to about 300 nM, about 300 pM to about 250 nM, about 350 pM to about 200 nM, about 400 pM to about 150 nM, about 450 pM to about 140 nM, about 500 pM to about 130 nM, about 525 pM to about 120 nM, about 550 pM and 110 nM, about 575 pM to about 100 nM, about 600 pM to about 90 nM, about 625 pM to about 80 nM, about 650 pM to about 70 nM, about 675 pM to about 65 nM, about 700 pM to about 60 nM, about 750 pM to about 55 nM, about 800 pM to about 50 nM, about 850 pM to about 45 nM, about 900 pM to about 40 nM, about 950 pM to about 35 nM, about 1 nM to about 30 nM, about 1.5 nM to about 25 nM, about 2 nM to about 20 nM, about 2.5 nM to about 15 nM, about 3 nM to about 10 nM; or about 1, 2, 3, 4, 5, 6, 7, 8, 9, 10, 11, 12 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 102, 104, 106, 108, 110, 112, 114, 116, 118, 120, 122, 124, 126, 128, 130, 132, 134, 136, 138, 140, 142, 144, 146, 148, 150, 175, 200, 225, 250, 275, 300, 325, 350, 375, 400, 425, 450, 475, 500 nM. In some aspects, the binding affinity is determined according to the assay described in Example 1.

### 2. Species Cross-Reactivity

In certain aspects, antigen binding proteins that are provided (e.g., those with sequences as described in Tables 2, 3 and 4 bind to human CD25 of SEQ ID NO: 42 and cynomolgus CD25.

In certain aspects, the antigen binding protein binds human CD25 and cynomolgus CD25 with similar affinity (0.1-0.7 nM) as determined by ELISA.

### 3. Additional Functional Activities

In some aspects, the antigen binding protein binds human CD25. In some aspects, the antigen binding proteins bind to human CD25 and cynomolgus CD25. In some aspects, the antigen binding proteins bind to human and cynomolgus CD25 with similar affinity. In some aspects, the antigen binding proteins bind to human CD25 but do not bind to cynomolgus CD25. In some aspects, the antigen binding proteins bind to human CD25 immobilized on a surface with an affinity of about 50 pM to about 0.5 nM and to cynomolgus CD25 immobilized to a surface with an affinity of about 0.8 nM to about 5 nM as measured by ELISA as shown in Example 2.

In some aspects, the antigen binding proteins bind to CD25 expressed on human cells with an affinity between about 80 pM and 3 nM, or about 100 pM and about 600 pM, or about 600 pM and about 2.4 nM as shown in Example 2.

In some aspects, the antigen binding proteins internalize into CD25 expressing cells. In some aspects, the antigen binding proteins internalize into CD25 expressing cells with an efficacy similar to Daclizumab as shown in Example 2.

In some aspects, the antigen binding proteins enable ADCC activity as shown in Example 2. In some aspects, the antigen binding proteins enable ADCC activity when incubated with human NK cells and tumor target cells. In some aspects, the antigen binding proteins when present in an ADC enable ADCC activity with human NK cells and tumor target cells. In some aspects, the antigen binding proteins when fucosylated enable ADCC activity when incubated with human NK cells and tumor target cells. In some aspects, the antigen binding proteins when non-fucosylated enable ADCC activity when incubated with human NK cells and tumor target cells.

In some aspects, the antigen binding proteins are variant antigen binding proteins that comprise a mutation in at least one amino acid in a heavy chain variable region and bind to surface immobilized CD25 with reduced affinity compared to antigen binding proteins without a mutation. In some aspects, the antigen binding proteins bind surface immobilized CD25 with an affinity of about 100 pM to about 200 pM and the variant antigen binding proteins bind surface immobilized CD25 with an affinity of between about 250 pM and about 250 nM, or about 300 pM and about 200 nM, about 400 pM and about 180 nM, about 500 pM and about 170 nM or about 900 pM and about 150 nM as measured by ELISA as shown in Example 3.

In some aspects, the antigen binding proteins are variant antigen binding proteins that comprise a mutation in at least one amino acid in a heavy chain variable region and bind to cell surface expressed CD25 with reduced affinity compared to antigen binding proteins without a mutation. In some aspects, the antigen binding proteins bind cell surface expressed CD25 with an affinity of about 600 pM to about 700 pM and the variant antigen binding proteins bind cell surface expressed CD25 with an affinity of between about 800 pM and about 300 nM, as measured by ELISA as shown in Example 3. In some aspects, the antigen binding proteins bind cell surface expressed CD25 with an affinity of about 800 pM to about 1 nM and the variant antigen binding proteins bind cell surface expressed CD25 with an affinity of between about 1.1 nM and about 500 nM, as measured by flow cytometry as shown in Example 3.

In some aspects, the antigen binding proteins are variant antigen binding proteins that comprise a mutation in at least one amino acid in a heavy chain variable region and have reduced ADCC activity when incubated with human NK cells and tumor target cells compared to antigen binding proteins without a mutation as shown in Example 3.

In some aspects, the antigen binding proteins are variant antigen binding proteins that comprise a mutation in at least one amino acid in a heavy chain variable region and have reduced in vitro cytotoxicity when present in an ADC compared to antigen binding proteins without a mutation as shown in Example 4.

In some aspects, the antigen binding proteins are variant antigen binding proteins that comprise a mutation in at least one amino acid in a heavy chain variable region and have reduced in vivo anti-tumor activity when present in an ADC compared to antigen binding proteins without a mutation as shown in Example 5.

In some aspects, the antigen binding proteins are variant antigen binding proteins that comprise a mutation in at least one amino acid in a heavy chain variable region and have reduced Treg depleting activity compared to antigen binding proteins without a mutation as shown in Example 6.

In some aspects, the antigen binding proteins are variant antigen binding proteins that comprise a mutation in at least one amino acid in a heavy chain variable region and have reduced in vitro cytotoxic activity towards Treg cells when present in an ADC compared to antigen binding proteins without a mutation as shown in Example 6.

In some aspects, the antigen binding proteins are variant antigen binding proteins that comprise a mutation in at least one amino acid in a heavy chain variable region and have no in vitro cytotoxic activity towards CD8 T cells when present in an ADC similar to antigen binding proteins without a mutation as shown in Example 6.

In some aspects, the antigen binding proteins are variant antigen binding proteins that comprise a mutation in at least one amino acid in a heavy chain variable region and have no in vitro cytotoxic activity towards CD4 T cells when present in an ADC similar to antigen binding proteins without a mutation as shown in Example 6.

In some aspects, the antigen binding proteins are variant antigen binding proteins that comprise a mutation in at least one amino acid in a heavy chain variable region and have similar in vivo anti-tumor activity in a colon cancer mouse model when present in an ADC as antigen binding proteins without a mutation as shown in Example 7.

In some aspects, the antigen binding proteins are variant antigen binding proteins that comprise a mutation in at least one amino acid in a heavy chain variable region and have reduced in vivo Treg depleting activity in non-human primates when present in an ADC compared to antigen binding proteins without a mutation as shown in Example 8.

In some aspects, the antigen binding proteins are variant antigen binding proteins that comprise a mutation in at least one amino acid in a heavy chain variable region and have similar total antibody pharmacokinetics in non-human primates when present in an ADC as antigen binding proteins without a mutation as shown in Example 9.

In some aspects, the antigen binding proteins are variant antigen binding proteins that comprise a mutation in at least one amino acid in a heavy chain variable region and have similar antibody-conjugated drug pharmacokinetics in non-human primates when present in an ADC as antigen binding proteins without a mutation as shown in Example 9.

Thus, in some aspects, an antigen binding protein has 1, 2, 3, 4, 5, 6, 7, 8, 9, 10, 11, 12, 13, 14 or 15 of the characteristics 1-15 in any combination or the antigen binding protein in addition has 1, 2, 3, 4, 5, 6, 7, 8, 9, 10, 11, 12, 13, 14 or 15 of characteristics 16-26 in any combination:
1. Binding to human and/or cynomolgus CD25;
2. Internalization into cells expressing CD25;
3. ADCC activity;
4. In vitro cytotoxicity towards CD25 expressing tumor cell lines when present in an ADC;
5. In vivo anti-tumor activity in CD25 expressing tumor models in mice when present in an ADC;
6. In vivo Treg cell depleting activity in a human CD25 transgenic mouse when present in an ADC;
7. Lack of in vivo CD4 T cell depleting activity in a human CD25 transgenic mouse when present in an ADC;
8. In vitro human Treg depleting activity in PBMC when present in a MMAE ADC;
9. Low level of in vitro human CD4 T cell depleting activity in PBMC when present in a MMAE ADC;
10. Lack of in vitro human CD8 T cell depleting activity in PBMC when present in a MMAE ADC;
11. In vitro human Treg cell depleting activity in PBMC when present in a Camptothecin or Anthracycline ADC;
12. In vitro human CD 8 T cell depleting activity in PBMC when present in a Camptothecin or Anthracycline ADC;
13. Anti-tumor activity in a colon cancer mouse model when present in a MMAE ADC;
14. Reduced binding to human and/or cynomolgus CD25 when comprising a mutation in a heavy chain variable region;
15. Reduced ADCC activity when comprising a mutation in a heavy chain variable region;
16. Reduced in vitro cytotoxicity towards CD25 expressing tumor cell lines when comprising a mutation in a heavy chain variable region and present in an ADC;
17. Reduced in vivo anti-tumor activity in CD25 expressing tumor models in mice when comprising a mutation in a heavy chain variable region and present in an ADC;
18. Reduced in vitro human Treg depleting activity in PBMC when and comprising a mutation in a heavy chain variable region present in an ADC;
19. Reduced in vitro cytotoxicity towards purified human Treg cells when comprising a mutation in a heavy chain variable region and present in MMAE ADC;
20. Lack of in vitro cytotoxicity towards purified human CD8 T cells when comprising a mutation in a heavy chain variable region and present in MMAE ADC;
21. Lack of in vivo CD4 T cell depletion in human CD25 transgenic mice when comprising a mutation in a heavy chain variable region and present in a MMAE ADC;
22. Reduced in vivo Treg depletion in human CD25 transgenic mice when comprising a mutation in a heavy chain variable region and present in a MMAE ADC;
23. Increased anti-tumor activity in a colon cancer mouse model when comprising a mutation in a heavy chain variable region and present in a MMAE ADC;
24. Reduced in vivo Treg depletion in non-human primates when comprising a mutation in a heavy chain variable region and present in a MMAE ADC;
25. Maintenance of total antibody pharmacokinetics in non-human primates when comprising a mutation in a heavy chain variable region and present in a MMAE ADC;
26. Maintenance of antibody-conjugated MMAE pharmacokinetics in non-human primates when comprising a mutation in a heavy chain variable region.

### K. Mimetics

Mimetics (e.g., “peptide mimetics” or “peptidomimetics”) based upon the variable region domains and CDRs that are described herein are also provided. These analogs can be peptides, non-peptides or combinations of peptide and non-peptide regions. Fauchere, 1986, Adv. Drug Res. 15:29; Veber and Freidinger, 1985, TINS p. 392; and Evans et al., 1987, J. Med. Chem. 30:1229. Peptidomimetics are proteins that are structurally similar to an antibody displaying a desired biological activity, such as here the ability to specifically bind CD25, but have one or more peptide linkages optionally replaced by a linkage selected from: —CH₂NH—, —CH₂S—, —CH₂—CH₂—, —CH—CH-(cis and trans), —COCH₂—, —CH(OH)CH₂—, and —CH₂SO—. Systematic substitution of one or more amino acids of a consensus sequence with a D-amino acid of the same type (e.g., D-lysine in place of L-lysine) may be used in certain aspects to generate more stable proteins. In addition, constrained peptides comprising a consensus sequence or a substantially identical consensus sequence variation may be generated by methods known in the art (Rizo and Gierasch, 1992, Ann. Rev. Biochem. 61:387), for example, by adding internal cysteine residues capable of forming intramolecular disulfide bridges which cyclize the peptide.

### L. Derivatives

In some aspects, the antigen binding protein is a derivative of an antigen binding protein, such as those described herein (see, e.g., Tables 2, 3, and 4). The derivatized antigen binding proteins can comprise any molecule or substance that imparts a desired property to the antigen binding protein (e.g., antibody or fragment), such as increased half-life in a particular use. The derivatized antigen binding protein can comprise, for example, a detectable (or labeling) moiety (e.g., a radioactive, colorimetric, antigenic or enzymatic molecule, or a detectable bead (such as a magnetic or electrodense (e.g., gold) bead); a molecule that binds to another molecule (e.g., biotin or streptavidin); a therapeutic or diagnostic moiety (e.g., a radioactive, cytotoxic, or pharmaceutically active moiety); or a molecule that increases the suitability of the antigen binding protein for a particular use (e.g., administration to a subject, such as a human subject, or other in vivo or in vitro uses). Examples of molecules that can be used to derivatize an antigen binding protein include albumin (e.g., human serum albumin) and polyethylene glycol (PEG). Albumin-linked and PEGylated derivatives of antigen binding proteins can be prepared using techniques well known in the art.

Other derivatives include covalent or aggregative conjugates of antigen binding proteins with other proteins or polypeptides, such as by expression of recombinant fusion proteins comprising heterologous polypeptides fused to the N-terminus or C-terminus of the antigen binding protein. For example, the conjugated peptide may be a heterologous signal (or leader) polypeptide, e.g., the yeast alpha-factor leader, or a peptide such as an epitope tag. Antigen binding protein-containing fusion proteins can comprise peptides added to facilitate purification or identification of the antigen binding protein (e.g., poly-His, or a FLAG peptide).

### M. Oligomers

Oligomers that contain one or more antigen binding proteins are also provided. Oligomers can be in the form of covalently-linked or non-covalently-linked dimers, trimers, or higher oligomers. In an aspects, oligomers comprising two or more antigen binding proteins are provided, with one example being a homodimer. Other oligomers include heterodimers, homotrimers, heterotrimers, homotetramers, heterotetramers and the like.

In some aspects, oligomers comprise multiple CD25 antigen binding polypeptides joined via covalent or non-covalent interactions between peptide moieties fused to the CD25 antigen binding proteins. Such peptides may be peptide linkers (spacers), or peptides that have the property of promoting oligomerization. Leucine zippers and certain polypeptides derived from antibodies are among the peptides that can promote oligomerization of antigen binding proteins attached thereto, as described in more detail below.

In particular aspects, the oligomers comprise from two to four CD25 antigen binding proteins. The CD25 antigen binding protein moieties of the oligomer may be in any of the forms described above, e.g., variants or fragments.

In some aspects, an oligomer is prepared using polypeptides derived from immunoglobulins. Preparation of fusion proteins comprising certain heterologous polypeptides fused to various portions of antibody-derived polypeptides (including the Fc domain) has been described, e.g., by Ashkenazi et al., 1991, Proc. Natl. Acad. Sci. USA 88:10535; Byrn et al., 1990, Nature 344:677; and Hollenbaugh et al., 1992 “Construction of Immunoglobulin Fusion Proteins”, in Current Protocols in Immunology, Suppl. 4, pages 10.19.1-10.19.11.

In some aspects, the antigen binding protein is a dimer created by fusing a CD25 antigen binding protein to the Fc region of an antibody. The dimer can be made by, for example, inserting a gene fusion encoding the fusion protein into an appropriate expression vector, expressing the gene fusion in host cells transformed with the recombinant expression vector, and allowing the expressed fusion protein to assemble much like antibody molecules, whereupon interchain disulfide bonds form between the Fc moieties to yield the dimer.

Alternatively, the oligomer is a fusion protein comprising multiple CD25 antigen binding proteins, with or without peptide linkers (spacer peptides). Among the suitable peptide linkers are those described in U.S. Pat. Nos. 4,751,180 and 4,935,233.

In some aspects, oligomeric CD25 antigen binding protein oligomers are prepared using a leucine zipper. Leucine zipper domains are peptides that promote oligomerization of the proteins in which they are found (Landschulz et al., 1988, Science 240:1759). Among the known leucine zippers are naturally-occurring peptides and derivatives thereof that dimerize or trimerize. Examples of leucine zipper domains suitable for producing soluble oligomeric proteins are described in PCT application WO 94/10308, and the leucine zipper derived from lung surfactant protein D (SPD) described in Hoppe et al., 1994, FEBS Letters 344:191. The use of a modified leucine zipper that allows for stable trimerization of a heterologous protein fused thereto is described in Fanslow et al., 1994, Semin. Immunol. 6:267-278. In some aspects, recombinant fusion proteins comprising a CD25 antigen binding protein fragment or derivative fused to a leucine zipper peptide are expressed in suitable host cells, and the soluble oligomeric CD25 antigen binding protein fragments or derivatives that form are recovered from the culture supernatant.

### N. Multispecific Antigen Binding Proteins

In a further aspect, the antigen binding protein can be a multispecific antigen binding protein, e.g., a multispecific antibody such as a bispecific antibody. In certain aspects, a multispecific antigen binding protein is a multispecific antibody that has binding specificity for at least two different targets. In some of these aspects, one of the binding specificities is for CD25 and the other is for a different antigen. In other aspects, the bispecific antibody binds to two different epitopes of CD25. In some aspects, the bispecific antibody binds an antigen on a target cells and can be used to localize cytotoxic agents to cells expressing CD25. Bispecific antibodies can be prepared as full-length antibodies or antibody fragments.

A variety of techniques for making multispecific antibodies can be utilized, including for example, recombinant co-expression of two immunoglobulin heavy chain-light chain pairs having different specificities (see Milstein and Cuello, Nature 305: 537 (1983)), WO 93/08829, and Traunecker et al., EMBO J 10: 3655 (1991)), and “knob-in-hole” engineering (see, e.g., U.S. Pat. No. 5,731,168). Multi-specific antigen binding proteins (e.g., antibodies) can also be made by engineering electrostatic steering effects for making antibody Fc-heterodimeric molecules (WO 2009/089004A1); cross linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); and using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Exemplary bispecific antibody molecules as provided herein comprise (i) two antibodies one with a specificity to CD25 and another to a second target that are conjugated together, (ii) a single antibody that has one chain specific to CD25 and a second chain specific to a second molecule, and (iii) a single chain antibody that has specificity to CD25 and a second molecule. In certain aspects, the second target/second molecule is a target other than CD25. In another aspects, however, the second target is a different region or epitope on CD25 such that the bispecific antibody binds two different epitopes on CD25.

### O. Other Exemplary Formats

An antigen binding protein (e.g., an antibody or antigen-binding fragment thereof) can be a single polypeptide, or can include two, three, four, five, six, seven, eight, nine, or ten (the same or different) polypeptides. In some aspects where the antibody or antigen-binding fragment thereof is a single polypeptide, the antibody or antigen-binding fragment can include a single antigen-binding domain or two antigen-binding domains. In some aspects where the antibody or antigen-binding fragment is a single polypeptide and includes two antigen-binding domains, the first and second antigen-binding domains can be identical or different from each other (and can specifically bind to the same or different antigens or epitopes).

The different parts of the antigen binding proteins described herein, such as the variable domains of the antibodies described above in Tables 2, 3, and 4, can be arranged in various configurations to obtain additional antigen binding proteins. For example, in some aspects where the antibody or the antigen-binding fragment is a single polypeptide, the first antigen-binding domain and the second antigen-binding domain (if present) can each be independently selected from the group of a VH domain, a V_{H}H domain, a V_{NAR} domain, and a scFv. In some aspects where the antibody or the antigen-binding fragment is a single polypeptide, the antibody or antigen-binding fragment can be a BiTE®, a (scFv)₂, a nanobody, a nanobody-HSA, a DART, a TandAb, a scDiabody, a scDiabody-CH3, scFv-CH-CL-scFv, a HSAbody, scDiabody-HAS, a tandem-scFv, an Adnectin, a DARPin, a fibronectin, and a DEP conjugate. Additional examples of antigen-binding domains that can be used when the antibody or antigen-binding fragment is a single polypeptide are known in the art.

A V_{H}H domain is a single monomeric variable antibody domain that can be found in camelids. A V_{NAR} domain is a single monomeric variable antibody domain that can be found in cartilaginous fish. Non-limiting aspects of V_{H}H domains and V_{NAR} domains are described in, e.g., Cromie et al., Curr. Top. Med. Chem. 15:2543-2557, 2016; De Genst et al., Dev Comp. Immunol. 30:187-198, 2006; De Meyer et al., Trends Biotechnol. 32:263-270, 2014; Kijanka et al., Nanomedicine 10:161-174, 2015; Kovaleva et al., Expert. Opin. Biol. Ther. 14:1527-1539, 2014; Krah et al., Immunopharmacol. Immunotoxicol. 38:21-28, 2016; Mujic-Delic et al., Trends Pharmacol. Sci. 35:247-255, 2014; Muyldermans, J. Biotechnol. 74:277-302, 2001; Muyldermans et al., Trends Biochem. Sci. 26:230-235, 2001; Muyldermans, Ann. Rev Biochem. 82:775-797, 2013; Rahbarizadeh et al., Immunol. Invest. 40:299-338, 2011; Van Audenhove et al., EBioMedicine 8:40-48, 2016; Van Bockstaele et al., Curr. Opin. Investig. Drugs 10:1212-1224, 2009; Vincke et al., Methods Mol. Biol. 911:15-26, 2012; and Wesolowski et al., Med. Microbiol. Immunol. 198:157-174, 2009.

In some aspects where the antibody or antigen-binding fragment is a single polypeptide and includes two antigen-binding domains, the first antigen-binding domain and the second antigen-binding domain can both be V_{H}H domains, or at least one antigen-binding domain can be a V_{H}H domain. In some aspects where the antibody or antigen-binding fragment is a single polypeptide and includes two antigen-binding domains, the first antigen-binding domain and the second antigen-binding domain are both V_{NAR} domains, or at least one antigen-binding domain is a V_{NAR} domain. In some aspects where the antibody or antigen-binding domain is a single polypeptide, the first antigen-binding domain is a scFv domain. In some aspects where the antibody or antigen-binding fragment is a single polypeptide and includes two antigen-binding domains, the first antigen-binding domain and the second antigen-binding domain can both be scFv domains, or at least one antigen-binding domain can be a scFv domain.

In some aspects, the antibody or antigen-binding fragment can include two or more polypeptides (e.g., two, three, four, five, six, seven, eight, nine, or ten polypeptides). In some aspects where the antibody or antigen-binding fragment includes two or more polypeptides, two, three, four, five or six of the polypeptides of the two or more polypeptides can be identical.

In some aspects where the antibody or antigen-binding fragment includes two or more polypeptides (e.g., two, three, four, five, six, seven, eight, nine, or ten polypeptides), two or more of the polypeptides of the antibody or antigen-binding fragment can assemble (e.g., non-covalently assemble) to form one or more antigen-binding domains, e.g., an antigen-binding fragment of an antibody (e.g., any of the antigen-binding fragments of an antibody described herein), a V_{H}H-scAb, a V_{H}H-Fab, a Dual scFab, a F(ab′)₂, a diabody, a crossMab, a DAF (two-in-one), a DAF (four-in-one), a DutaMab, a DT-IgG, a knobs-in-holes common light chain, a knobs-in-holes assembly, a charge pair, a Fab-arm exchange, a SEEDbody, a LUZ-Y, a Fcab, a rck-body, an orthogonal Fab, a DVD-IgG, a IgG(H)-scFv, a scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, Zybody, DVI-IgG, Diabody-CH3, a triple body, a miniantibody, a minibody, a TriBi minibody, scFv-CH3 KIH, Fab-scFv, a F(ab′)2-scFv2, a scFv-KIH, a Fab-scFv-Fc, a tetravalent HCAb, a scDiabody-Fc, a Diabody-Fe, a tandem scFv-Fc, a V_{H}H-Fc, a tandem V_{H}H-Fc, a V_{H}H-Fc KiH, a Fab-V_{H}H-Fc, an Intrabody, a dock and lock, an ImmTAC, an IgG-IgG conjugate, a Cov-X-Body, a scFv1-PEG-scFv2, an Adnectin, a DARPin, a fibronectin, and a DEP conjugate. See, e.g., Spiess et al., Mol. Immunol. 67:95-106, 2015, incorporated in its entirety herewith, for a description of these elements.

In some aspects, the antigen binding protein is based upon a non-immunoglobulin scaffold. Examples of other scaffolds into which the binding domains (e.g., CDRs or CDRs) such as described herein can be inserted or grafted include, but are not limited to, human fibronectin (e.g., the 10^{th} extracellular domain of human fibronectin III), neocarzinostatin CBM4-2, anticalins derived from lipocalins, designed ankyrin repeat domains (DARPins), protein-A domain (protein Z), Kunitz domains, Im9, TPR proteins, zinc finger domains, pVIII, GC4, transferrin, B-domain of SPA, Sac7d, A-domain, SH3 domain of Fyn kinase, and C-type lectin-like domains (see, e.g., Gebauer and Skerra (2009) Curr. Opin. Chem. Biol., 13:245-255; Binz et al. (2005) Nat. Biotech. 23:1257-1268; and Yu et al. (2017) Annu Rev Anal Chem 10:293-320, each of which is incorporated herein by reference in its entirety).

### V. Antigen Binding Protein Expression and Production

### A. Nucleic Acid Molecules Encoding Antigen Binding Proteins

Nucleic acid molecules that encode for the antigen binding proteins described herein, or portions thereof, are also provided. Such nucleic acids include, for example: 1) those encoding an antigen binding protein (e.g., an antibody or a fragment thereof), or a derivative, or variant thereof; 2) polynucleotides encoding a heavy and/or light chain, VH and/or VL domains, or 1 or more of the CDRs or CDRs located within a variable domain (e.g., 1, 2 or all 3 of the VH CDRs or CDRs or 1, 2 or all 3 of the VL CDRs or CDRs); 3) polynucleotides sufficient for use as hybridization probes, PCR primers or sequencing primers for identifying, analyzing, mutating or amplifying such encoding polynucleotides; 4) anti-sense nucleic acids for inhibiting expression of such encoding polynucleotides, and 5) complementary sequences of the foregoing. The nucleic acids can be any length. They can be, for example, 5, 10, 15, 20, 25, 30, 35, 40, 45, 50, 75, 100, 125, 150, 175, 200, 250, 300, 350, 400, 450, 500, 750, or 1,000 or more nucleotides in length, and/or can comprise one or more additional sequences, for example, regulatory sequences, and/or be part of a larger nucleic acid, for example, a vector. The nucleic acids can be single-stranded or double-stranded.

The nucleic acid molecules can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., other chromosomal DNA, e.g., the chromosomal DNA that is linked to the isolated DNA in nature) or proteins, by standard techniques, including alkaline/SDS treatment, CsCl banding, column chromatography, restriction enzymes, agarose gel electrophoresis and others well known in the art. See, F. Ausubel, et al., ed. (1987) Current Protocols in Molecular Biology, Greene Publishing and Wiley Interscience, New York. A nucleic acid described herein can be, for example, DNA or RNA and may or may not contain intronic sequences. In certain aspects, the nucleic acid is a cDNA molecule.

Exemplary nucleic acid molecules that encode the VH and VL sequences of the antibodies that are provided herein are shown in Table 4.

Thus, nucleic acid molecules comprising polynucleotides that encode one or more chains of an antigen binding protein, such as anti-CD25 antibodies, are provided. In some aspects, a nucleic acid molecule comprises a polynucleotide that encodes a heavy chain or a light chain of an antigen binding protein (e.g., an anti-CD25 antibody). In some aspects, a nucleic acid molecule comprises both a polynucleotide sequence that encodes a heavy chain and a polynucleotide sequence that encodes a light chain, of an antigen binding protein (e.g., an anti-CD25 antibody). In some aspects, a first nucleic acid molecule comprises a first polynucleotide sequence that encodes a heavy chain and a second nucleic acid molecule comprises a second polynucleotide sequence that encodes a light chain.

In certain aspects, the nucleic acid molecule comprises a polynucleotide encoding one or more of the amino acid sequences selected from the group consisting of: SEQ ID NOs: 1-17, 19, 21-23, 25-41, and 43-47.

In a particular aspects, the nucleic acid molecule comprises a polynucleotide encoding a VH amino acid sequence of selected from the group consisting of: SEQ ID NOs: 7, 22, and 31.

In another particular aspects, the nucleic acid molecule comprises a polynucleotide encoding one or more of the CDR-H3 amino acid sequences selected from the group consisting of: SEQ ID NOs: 3, 21, and 27.

In yet another aspects, the nucleic acid molecule comprises a polynucleotide encoding one or more of the VL amino acid sequences selected from the group consisting of: SEQ ID NOs: 8 and 32.

In still another aspects, the nucleic acid molecule comprises a polynucleotide encoding one or more of the CDR-L3 amino acid sequences selected from the group consisting of: SEQ ID NOs: 6 and 30.

In some aspects, the nucleic acid molecule comprises a polynucleotide encoding the VH of one of the antibodies provided herein. In some aspects, the nucleic acid comprises a polynucleotide encoding the VL of one of the antibodies provided herein. In still other aspects, the nucleic acid encodes both the VH and the VL of one of the antibodies provided herein.

In some aspects, the nucleic acid encodes a variant of one or more of the above amino acid sequences (e.g., the heavy chain and/or light chain amino acid sequences, or the VH and/or VL amino acid sequences disclosed herein), wherein the variants has at most 25 amino acid modifications, such as at most 20, such as at most 15, 14, 13, 12 or 11 amino acid modifications, such as 10, 9, 8, 7, 6, 5, 4, 3, 2 or 1 amino-acid modifications, such as deletions or insertions, preferably substitutions, such as conservative substitutions.

Also provided are nucleic acid molecules that have at least 80%, 85%, 90% (e.g., 95%, 96%, 97%, 98%, or 99%) sequence identity to any of the aforementioned sequences, including those listed in Table 4. Thus, for example, in certain aspects, the nucleic comprises a nucleotide sequence that encodes the heavy and/or light chain sequence or the VH and/or VL sequence of one of the antigen binding proteins disclosed herein in Table 4.

Once nucleic acids encoding VH and VL segments are obtained, these nucleic acids can be further manipulated by standard recombinant DNA techniques, for example to convert the variable region genes to full-length antibody chain genes, to Fab fragment genes or to a scFv gene. In these manipulations, a VL- or VH-encoding nucleic acid is operatively linked to another nucleic acid encoding another polypeptide, such as an antibody constant region or a flexible linker.

The isolated nucleic acid encoding the VH region can be converted to a full-length heavy chain gene by operatively linking the VH-encoding nucleic acid to another nucleic acid molecule encoding heavy chain constant regions (hinge, CH1, CH2 and/or CH3). The sequences of human heavy chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and nucleic acid fragments encompassing these regions can be obtained by standard PCR amplification. The heavy chain constant region can be an IgG1, IgG2, IgG3, IgG4, IgA, IgE, IgM or IgD constant region, for example, an IgG1 region. For a Fab fragment heavy chain gene, the VH-encoding nucleic can be operatively linked to another nucleic acid molecule encoding only the heavy chain CH1 constant region.

The isolated nucleic acid molecule encoding the VL region can be converted to a full-length light chain gene (as well as a Fab light chain gene) by operatively linking the VL-encoding nucleic acid molecule to another nucleic acid molecule encoding the light chain constant region, CL. The sequences of human light chain constant region genes are known in the art (see e.g., Kabat, E. A., et al. (1991) Sequences of Proteins of Immunological Interest, Fifth Edition, U.S. Department of Health and Human Services, NIH Publication No. 91-3242) and nucleic acid fragments encompassing these regions can be obtained by standard PCR amplification. The light chain constant region can be a kappa or lambda constant region.

To create a scFv gene, the VH- and VL-encoding nucleic acid fragments are operatively linked to another fragment encoding a flexible linker, e.g., encoding the amino acid sequence (Gly4-Ser)₃, such that the VH and VL sequences can be expressed as a contiguous single-chain protein, with the VL and VH regions joined by the flexible linker (see e.g., Bird et al. (1988) Science 242:423-426; Huston et al. (1988) Proc. Natl. Acad. Sci. USA 85:5879-5883; McCafferty et al., (1990) Nature 348:552-554).

In another aspect, nucleic acid molecules that are suitable for use as primers or hybridization probes for the detection of nucleic acid sequences are also provided. A nucleic acid molecule can comprise only a portion of a nucleic acid sequence encoding a full-length polypeptide, for example, a fragment that can be used as a probe or primer or a fragment encoding an active portion (e.g., CD25 binding portion) of a polypeptide.

Probes based on the sequence of a nucleic acid can be used to detect the nucleic acid or similar nucleic acids, for example, transcripts encoding a polypeptide. The probe can comprise a label group, e.g., a radioisotope, a fluorescent compound, an enzyme, or an enzyme co-factor. Such probes can be used to identify a cell that expresses the polypeptide.

Vectors, including expression vectors, comprising one or more nucleic acids encoding one or more components of the antigen binding proteins (e.g. VH and/or VL; and light chains, and/or heavy chains) are also provided. An expression vector can include, but is not limited to, sequences that affect or control transcription, translation, and, if introns are present, affect RNA splicing of a coding region operably linked thereto. Nucleic acid sequences necessary for expression in prokaryotes include a promoter, optionally an operator sequence, a ribosome binding site and possibly other sequences. Eukaryotic cells are known to utilize promoters, enhancers, and termination and polyadenylation signals.

The expression vector can also include a secretory signal peptide sequence that is operably linked to the coding sequence of interest, such that the expressed polypeptide can be secreted by the recombinant host cell, for more facile isolation of the polypeptide of interest from the cell, if desired. For instance, in some aspects, signal peptide sequences can be appended/fused to the amino terminus of any of the variable region polypeptide sequences listed in Table 4 or any of the full chain polypeptide sequences listed in Table 4. In certain aspects, a signal peptide is fused to the amino terminus of any of the variable region polypeptide sequences in Table 4 or full chain polypeptide sequences in Table 4. Other signal or secretory peptides are known to those of skill in the art and may be fused to any of the variable region polypeptide chains, for example, to facilitate or optimize expression in particular host cells.

Expression and cloning vectors of the invention will typically contain a promoter that is recognized by the host organism and operably linked to the molecule encoding the polypeptide. A large number of promoters, recognized by a variety of potential host cells, are well known. A suitable promoter is operably linked to the DNA encoding e.g., heavy chain, light chain, or other component of the antibodies and antigen-binding fragments of the invention, by removing the promoter from the source DNA by restriction enzyme digestion and inserting the desired promoter sequence into the vector. Suitable promoters for use with yeast hosts are also well known in the art. Yeast enhancers are advantageously used with yeast promoters. Suitable promoters for use with mammalian host cells are well known and include, but are not limited to, those obtained from the genomes of viruses such as polyoma virus, fowlpox virus, adenovirus (such as Adenovirus serotypes 2, 8, or 9), bovine papilloma virus, avian sarcoma virus, cytomegalovirus, retroviruses, hepatitis-B virus and Simian Virus 40 (SV40). Other suitable mammalian promoters include heterologous mammalian promoters, for example, heat-shock promoters and the actin promoter.

Additional specific promoters that can be utilized include, but are not limited to: SV40 early promoter (Benoist and Chambon, 1981, Nature 290:304-310); CMV promoter (Thornsen et al., 1984, Proc. Natl. Acad. U.S.A. 81:659-663); the promoter contained in the 3′ long terminal repeat of Rous sarcoma virus (Yamamoto et al., 1980, Cell 22:787-797); herpes thymidine kinase promoter (Wagner et al., 1981, Proc. Natl. Acad. Sci. U.S.A. 78: 1444-1445); promoter and regulatory sequences from the metallothionine gene (Prinster et al., 1982, Nature 296:39-42); and prokaryotic promoters such as the beta-lactamase promoter (Villa-Kamaroff et al., 1978, Proc. Natl. Acad. Sci. U.S.A. 75:3727-3731); or the tac promoter (DeBoer et al., 1983, Proc. Natl. Acad. Sci. U.S.A. 80:21-25).

In certain aspects, nucleic acids encoding the different components of the antigen binding protein can be inserted into the same expression vector. For instance, the nucleic acid encoding an anti-CD25 antibody light chain or variable region can be cloned into the same vector as the nucleic acid encoding an anti-CD25 antibody heavy chain or variable region. In such aspects, the two nucleic acids may be separated by an internal ribosome entry site (IRES) and under the control of a single promoter such that the light chain and heavy chain are expressed from the same mRNA transcript. Alternatively, the two nucleic acids can be under the control of two separate promoters such that the light chain and heavy chain are expressed from two separate mRNA transcripts. In some aspects, the nucleic acid encoding the anti-CD25 antibody light chain or variable region is cloned into one expression vector and the nucleic acid encoding the anti-CD25 antibody heavy chain or variable region is cloned into a second expression vector. In such aspects, a host cell may be co-transfected with both expression vectors to produce complete antibodies or antigen-binding fragments of the invention.

### B. Host Cells

After the vector has been constructed and the one or more nucleic acid molecules encoding the components of the antigen binding proteins described herein has been inserted into the proper site(s) of the vector or vectors, the completed vector(s) may be inserted into a suitable host cell for amplification and/or polypeptide expression.

Thus, in another aspect, host cells comprising nucleic acid molecules or vectors such as described herein are also provided. In various aspects, antigen binding protein heavy chains and/or light chains can be expressed in prokaryotic cells, such as bacterial cells, or in eukaryotic cells, such as fungal cells (such as yeast), plant cells, insect cells, and mammalian cells. The selection of an appropriate host cell depends upon various factors, such as desired expression levels, polypeptide modifications that are desirable or necessary for activity (such as glycosylation or phosphorylation) and ease of folding into a biologically active molecule.

Introduction of one or more nucleic acids into a desired host cell can be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

Exemplary prokaryotic host cells include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacillus, such as B. subtilis and B. licheniformis, Pseudomonas, and Streptomyces.

Yeast can also be used as host cells including, but not limited to, S. cerevisae, S. pombe; or K. lactis.

A variety of mammalian cell lines can be used as hosts and include, but are not limited to, immortalized cell lines available from the American Type Culture Collection (ATCC), including but not limited to Chinese hamster ovary (CHO) cells, including CHOK1 cells (ATCC CCL61), DXB-11, DG-44, and Chinese hamster ovary cells/-DHFR (CHO, Urlaub et al., Proc. Natl. Acad. Sci. USA 77: 4216, 1980); Expi CHO (Thermo Fisher), monkey kidney CV1 line transformed by SV40 (COS-7, ATCC CRL 1651); human embryonic kidney line (293 or 293 cells subcloned for growth in suspension culture, (Graham et al., J. Gen Virol. 36: 59, 1977); Expi HEK (Thermo Fisher), baby hamster kidney cells (BHK, ATCC CCL 10); mouse sertoli cells (TM4, Mather, Biol. Reprod. 23: 243-251, 1980); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human hepatoma cells (Hep G2, HB 8065); mouse mammary tumor (MMT 060562, ATCC CCL51); TM cells (Mather et al., Annals N.Y Acad. Sci. 383: 44-68, 1982); MRC 5 cells or FS4 cells; mammalian myeloma cells, and a number of other cell lines.

Once a suitable host cell has been prepared, it can be used to express the desired antigen binding protein. Thus, in a further aspect, methods for producing an antigen binding protein as described herein are also provided. In general, such methods comprise culturing a host cell comprising one or more expression vectors as described herein in a culture medium under conditions permitting expression of the antigen binding protein as encoded by the one or more expression vectors; and recovering the antigen binding protein from the culture medium.

In some aspects, the antigen binding protein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

### VI. Antigen Binding Protein Conjugates

The antigen binding protein that are provided herein can be conjugated to cytotoxic or cytostatic moieties (including pharmaceutically compatible salts thereof) to form a conjugate, such as an ADC. Particularly suitable moieties for conjugation to antigen binding proteins (e.g., antibodies) are cytotoxic agents (e.g., chemotherapeutic agents), prodrug converting enzymes, radioactive isotopes or compounds, or toxins (these moieties being collectively referred to as a therapeutic agent). For example, an antigen binding protein (e.g., an anti-CD25 antibody) can be conjugated to a cytotoxic agent such as a chemotherapeutic agent, or a toxin (e.g., a cytostatic or cytocidal agent such as, for example, abrin, ricin A, pseudomonas exotoxin, or diphtheria toxin). Examples of useful classes of cytotoxic agents include, for example, DNA minor groove binders, DNA alkylating agents, and tubulin inhibitors. Exemplary cytotoxic agents include, for example, auristatins, camptothecins, calicheamicins, duocarmycins, etoposides, maytansinoids (e.g., DM1, DM2, DM3, DM4) taxanes, benzodiazepines (e.g., pyrrolo[1,4]benzodiazepines, indolinobenzodiazepines, and oxazolidinobenzodiazepines) and vinca alkaloids.

In one aspect, an antigen binding protein (e.g., an anti-CD25 antibody) is conjugated to a pro-drug converting enzyme. The pro-drug converting enzyme can be recombinantly fused to the antibody or chemically conjugated thereto using known methods. Exemplary pro-drug converting enzymes are carboxypeptidase G2, betaglucuronidase, penicillin-V-amidase, penicillin-G-amidase, β-lactamase, β-glucosidase, nitroreductase and carboxypeptidase A.

Techniques for conjugating therapeutic agents to proteins, and in particular to antibodies, are well-known. (See, e.g., Alley et al., Current Opinion in Chemical Biology 2010 14:1-9; Senter, Cancer J, 2008, 14(3):154-169.) The therapeutic agent can be conjugated in a manner that reduces its activity unless it is cleaved off the antibody (e.g., by hydrolysis, by proteolytic degradation, or by a cleaving agent). In some aspects, the therapeutic agent is attached to the antibody with a cleavable linker that is sensitive to cleavage in the intracellular environment of the CD25 expressing cancer cell but is not substantially sensitive to the extracellular environment, such that the conjugate is cleaved from the antibody when it is internalized by the CD25-expressing cancer cell (e.g., in the endosomal or, for example by virtue of pH sensitivity or protease sensitivity, in the lysosomal environment or in the caveolear environment). In some aspects, the therapeutic agent can also be attached to the antibody with a non-cleavable linker.

Typically, the ADC comprises a linker region between the therapeutic agent and the antigen binding protein (e.g., anti-CD25 antibody). The linker generally is cleavable under intracellular conditions, such that cleavage of the linker releases the therapeutic agent from the antibody in the intracellular environment (e.g., within a lysosome or endosome or caveolea). The linker can be, e.g., a peptidyl linker that is cleaved by an intracellular peptidase or protease enzyme, including a lysosomal or endosomal protease. Cleaving agents can include cathepsins B and D and plasmin (see, e.g., Dubowchik and Walker, Pharm. Therapeutics 83:67-123, 1999). Most typical are peptidyl linkers that are cleavable by enzymes that are present in CD25-expressing cells. For example, a peptidyl linker that is cleavable by the thiol-dependent protease cathepsin-B, which is highly expressed in cancerous tissue, can be used (e.g., a linker comprising a Phe-Leu or a Val-Cit peptide).

The cleavable linker can be pH-sensitive, i.e., sensitive to hydrolysis at certain pH values. Typically, the pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; 5,622,929; Dubowchik and Walker, Pharm. Therapeutics 83:67-123, 1999; Neville et al., Biol. Chem. 264:14653-14661, 1989.) Such linkers are relatively stable under neutral pH conditions, such as those in the blood, but are unstable at below pH 5.5 or 5.0, the approximate pH of the lysosome.

Other linkers are cleavable under reducing conditions (e.g., a disulfide linker). Disulfide linkers include those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT. (See, e.g., Thorpe et al., Cancer Res. 47:5924-5931, 1987; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

The linker can also be a malonate linker (Johnson et al., Anticancer Res. 15:1387-93, 1995), a maleimidobenzoyl linker (Lau et al., Bioorg-Med-Chem. 3:1299-1304, 1995), or a 3′-N-amide analog (Lau et al., Bioorg-Med-Chem. 3:1305-12, 1995).

In other aspects, the linker is a non-cleavable linker, such as a maleimido-alkylene- or maleimide-aryl linker that is directly attached to the therapeutic agent and released by proteolytic degradation of the antibody.

Typically, the linker is not substantially sensitive to the extracellular environment, meaning that no more than about 20%, typically no more than about 15%, more typically no more than about 10%, and even more typically no more than about 5%, no more than about 3%, or no more than about T % of the linkers in a sample of the ADC is cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or therapeutic agent (the “control sample”) for a predetermined time period (e.g., 2, 4, 8, 16, or 24 hours) and then comparing the amount of unconjugated antibody or therapeutic agent present in the ADC sample with that present in control sample, as measured, for example, by high performance liquid chromatography.

The linker can also promote cellular internalization. The linker can promote cellular internalization when conjugated to the therapeutic agent (i.e., in the milieu of the linker-therapeutic agent moiety of the ADC or ADC derivative as described herein). Alternatively, the linker can promote cellular internalization when conjugated to both the therapeutic agent and the antigen binding protein (e.g., anti-CD25 antibody) (i.e., in the milieu of the ADC as described herein).

Exemplary ADCs include auristatin based ADCs meaning that the drug component is an auristatin drug. Auristatins bind tubulin, have been shown to interfere with microtubule dynamics and nuclear and cellular division, and have anticancer activity. Typically the auristatin based ADC comprises a linker between the auristatin drug and the antigen binding protein (e.g., anti-CD25 antibody). The linker can be, for example, a cleavable linker (e.g., a peptidyl linker) or a non-cleavable linker (e.g., linker released by degradation of the antibody).

In some aspects, the linker is selected from alkyl, substituted alkyl, alkenyl, substituted alkenyl, alkynyl, substituted alkynyl, alkoxy, substituted alkoxy, amino, substituted amino, carboxyl, carboxyl ester, acyl amino, alkylamide, substituted alkylamide, aryl, substituted aryl, heteroaryl, substituted heteroaryl, cycloalkyl, substituted cycloalkyl, heterocyclyl, and substituted heterocyclyl. In some aspects, the linker can be a polymer and may include a polyalkylene glycol and derivatives thereof, including polyethylene glycol, methoxypolyethylene glycol, polyethylene glycol homopolymers, polypropylene glycol homopolymers, copolymers of ethylene glycol with propylene glycol (e.g., where the homopolymers and copolymers are unsubstituted or substituted at one end with an alkyl group), polyvinyl alcohol, polyvinyl ethyl ethers, polyvinylpyrrolidone, combinations thereof, and the like. In some aspects, the polymer is a polyalkylene glycol. In certain embodiments, the polymer is a polyethylene glycol.

In some aspects, the ADC is an auristatin based ADC. The auristatin can be auristatin E or a derivative thereof. In some aspects, the auristatin can be monomethyl auristatin E (MMAE), or dolostatin 10/auristatin. In some aspects, the auristatin can be an ester formed between auristatin E and a keto acid. For example, auristatin E can be reacted with paraacetyl benzoic acid or benzoylvaleric acid to produce AEB and AEVB, respectively. Another typical auristatin includes MMAE. The synthesis and structure of exemplary auristatins are described in U.S. Publication Nos. 7,659,241, 7,498,298, 2009-0111756, 2009-0018086, and 7,968,687 each of which is incorporated herein by reference in its entirety and for all purposes.

Exemplary auristatin based ADCs include valine-citrulline peptide linked MMAE (vcMMAE), ADCs as shown below wherein Ab is an antigen binding protein (e.g., an anti-CD25 antibody as described herein) and val-cit represents the valine-citrulline (vc) dipeptide and me represents maleimide-caproic acid:

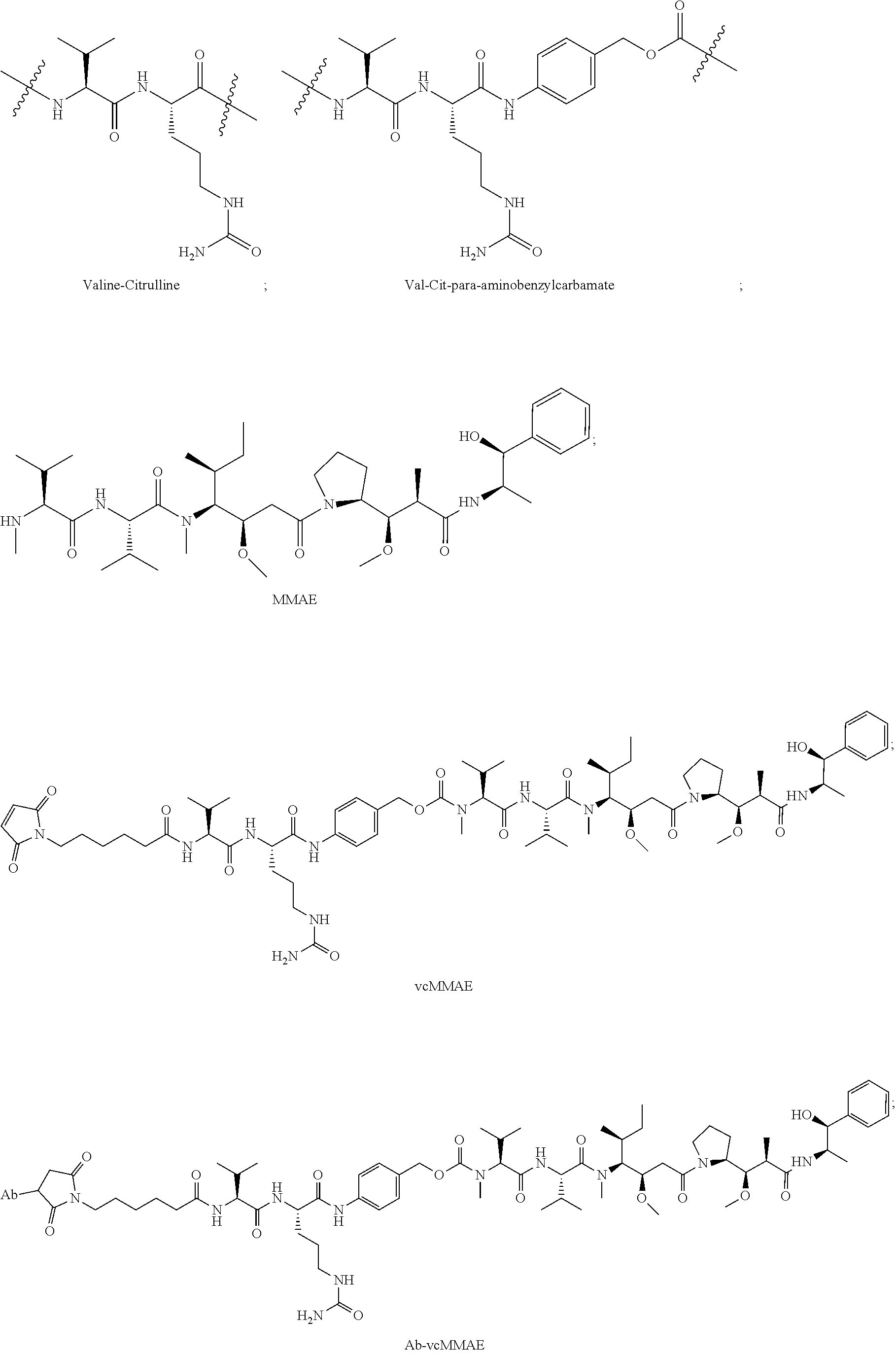

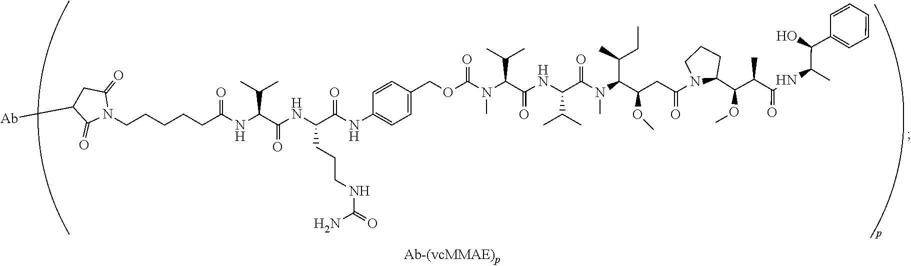

or a pharmaceutically acceptable salt thereof. The drug loading is represented by p, the number of drug-linker molecules per antibody. Depending on the context, p can represent the average number of drug-linker molecules per antibody in a composition of antibodies, also referred to the average drug loading. P ranges from 1 to 20 and is preferably from 1 to 8. In some preferred aspects, when p represents the average drug loading, p ranges from about 2 to about 5. In some aspects, p is about 2. In some aspects, p is about 3. In some aspects, p is about 4. In some aspects, p is about 5.

In some aspects, the auristatin-based ADC comprises one auristatin molecule conjugated to the antibody. In some aspects, the auristatin-based ADC comprises more than one auristatin molecule conjugated to the antibody. In some aspects, the ADC comprises 1, 2, 3, 4, 5, 6, 7, 8, 9, or 10 auristatin molecules.

In one embodiment, the ADC comprises:

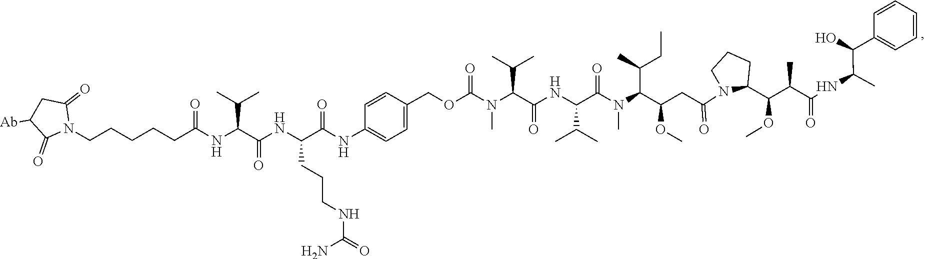

wherein Ab is an antigen binding protein that comprises (a) a CDR-H1 comprising the amino acid sequence of SEQ ID NO: 1; a CDR-H2 comprising the amino acid sequence of SEQ ID NO:2; (c) a CDR-H3 comprising the amino acid sequence of SEQ ID NO: 21; (d) a CDR-L1 comprising the amino acid sequence of SEQ ID NO: 4; (e) a CDR-L2 comprising the amino acid sequence of SEQ ID NO: 5; and (f) a CDR-L3 comprising the amino acid sequence of SEQ ID NO: 6. In some embodiments, the antigen binding protein is an antibody. In some embodiments, the antigen binding protein comprises a VH that has an amino acid sequence that has at least 80%, 85%, 90%, 95% or 99% amino acid sequence identity to the amino acid sequence of SEQ ID NO: 22. In some embodiments, the VH comprises the amino acid sequence of SEQ ID NO: 22. In some embodiments, the antigen binding protein comprises a VL that has an amino acid sequence that has at least 80%, 85%, 90%, 95% or 99% amino acid sequence identity to the amino acid sequence of SEQ ID NO: 8. In some embodiments, the VL comprises the amino acid sequence of SEQ ID NO: 8. In some embodiments, the antigen binding protein comprises a VH that has an amino acid sequence that has at least 80%, 85%, 90%, 95% or 99% amino acid sequence identity to the amino acid sequence of SEQ ID NO: 22 and a VL that has an amino acid sequence that has at least 80%, 85%, 90%, 95% or 99% amino acid sequence identity to the amino acid sequence of SEQ ID NO: 8. In some embodiments, the antigen binding protein comprises a VH that comprises the amino acid sequence of SEQ ID NO: 22 and a VL that comprises the amino acid sequence of SEQ ID NO: 8. In some embodiments, the antigen binding protein comprises a HC that has an amino acid sequence that has at least 80%, 85%, 90%, 95% or 99% amino acid sequence identity to the amino acid sequence of SEQ ID NO: 46. In some embodiments, the HC comprises the amino acid sequence of SEQ ID NO: 46. In some embodiments, the antigen binding protein comprises a LC that has an amino acid sequence that has at least 80%, 85%, 90%, 95% or 99% amino acid sequence identity to the amino acid sequence of SEQ ID NO: 47. In some embodiments, the LC comprises the amino acid sequence of SEQ ID NO: 47. In some embodiments, the antigen binding protein comprises a HC that has an amino acid sequence that has at least 80%, 85%, 90%, 95% or 99% amino acid sequence identity to the amino acid sequence of SEQ ID NO: 46 and a LC that has an amino acid sequence that has at least 80%, 85%, 90%, 95% or 99% amino acid sequence identity to the amino acid sequence of SEQ ID NO: 47. In some embodiments, the antigen binding protein comprises a HC that comprises the amino acid sequence of SEQ ID NO: 46 and a LC that comprises the amino acid sequence of SEQ ID NO: 47.

In one embodiment, the ADC comprises:

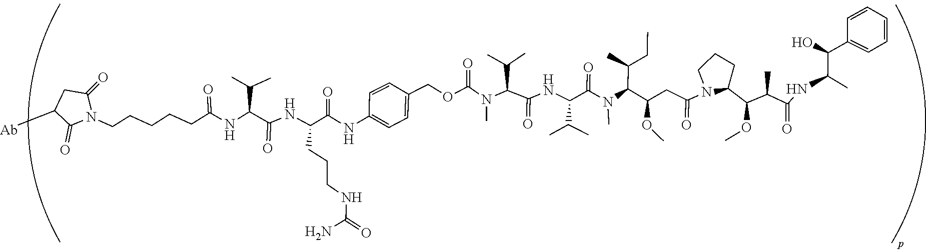

wherein the Ab is an antigen binding protein comprising (a) a CDR-H1 comprising the amino acid sequence of SEQ ID NO: 1; a CDR-H2 comprising the amino acid sequence of SEQ ID NO:2; (c) a CDR-H3 comprising the amino acid sequence of SEQ ID NO: 21; (d) a CDR-L1 comprising the amino acid sequence of SEQ ID NO: 4; (e) a CDR-L2 comprising the amino acid sequence of SEQ ID NO: 5; and (f) a CDR-L3 comprising the amino acid sequence of SEQ ID NO: 6, and p ranges from 1 to 20, preferably from 1 to 8, and in some preferred aspects, when p represents the average drug loading, p ranges from about 2 to about 5, and in some aspects, p is about 4. In some embodiments, the antigen binding protein is an antibody. In some embodiments, the antigen binding protein comprises a VH that has an amino acid sequence that has at least 80%, 85%, 90%, 95% or 99% amino acid sequence identity to the amino acid sequence of SEQ ID NO: 22. In some embodiments, the VH comprises the amino acid sequence of SEQ ID NO: 22. In some embodiments, the antigen binding protein comprises a VL that has an amino acid sequence that has at least 80%, 85%, 90%, 95% or 99% amino acid sequence identity to the amino acid sequence of SEQ ID NO: 8. In some embodiments, the VL comprises the amino acid sequence of SEQ ID NO: 8. In some embodiments, the antigen binding protein comprises a VH that has an amino acid sequence that has at least 80%, 85%, 90%, 95% or 99% amino acid sequence identity to the amino acid sequence of SEQ ID NO: 22 and a VL that has an amino acid sequence that has at least 80%, 85%, 90%, 95% or 99% amino acid sequence identity to the amino acid sequence of SEQ ID NO: 8. In some embodiments, the antigen binding protein comprises a VH that comprises the amino acid sequence of SEQ ID NO: 22 and a VL that comprises the amino acid sequence of SEQ ID NO: 8. In some embodiments, the antigen binding protein comprises a HC that has an amino acid sequence that has at least 80%, 85%, 90%, 95% or 99% amino acid sequence identity to the amino acid sequence of SEQ ID NO: 46. In some embodiments, the HC comprises the amino acid sequence of SEQ ID NO: 46. In some embodiments, the antigen binding protein comprises a LC that has an amino acid sequence that has at least 80%, 85%, 90%, 95% or 99% amino acid sequence identity to the amino acid sequence of SEQ ID NO: 47. In some embodiments, the LC comprises the amino acid sequence of SEQ ID NO: 47. In some embodiments, the antigen binding protein comprises a HC that has an amino acid sequence that has at least 80%, 85%, 90%, 95% or 99% amino acid sequence identity to the amino acid sequence of SEQ ID NO: 46 and a LC that has an amino acid sequence that has at least 80%, 85%, 90%, 95% or 99% amino acid sequence identity to the amino acid sequence of SEQ ID NO: 47. In some embodiments, the antigen binding protein comprises a HC that comprises the amino acid sequence of SEQ ID NO: 46 and a LC that comprises the amino acid sequence of SEQ ID NO: 47.

In some embodiments, the ADC comprises an antigen binding protein that binds CD25 (e.g., an anti-CD25 antibody) and the drug is MMAE, wherein the antigen binding protein comprises a CDR-H1, CDR-H2, and CDR-H3 having amino acid sequences SEQ ID NOs: 1, 2 and 21, respectively, and a CDR-L1, CDR-L2, and CDR-L3 having amino acid sequences SEQ ID NO: 4, 5 and 6, respectively. In some embodiments, the antigen binding protein comprises a VH that comprises the amino acid sequence of SEQ ID NO: 22 and a VL that comprises the amino acid sequence of SEQ ID NO: 8. In some embodiments, the antigen binding protein comprises a HC that comprises the amino acid sequence of SEQ ID NO: 46 and a LC that comprises the amino acid sequence of SEQ ID NO: 47. In some embodiments, the antigen binding protein is linked to MMAE by maleimidocaproyl valine citrulline p-amino-benzyloxy (mc-vc-pAB).

In some embodiments, the ADC is represented by formula Ab-(L-U)n, wherein Ab is an anti-CD25 antibody, L is a linker between the cytotoxic molecule and the anti-CD25 antibody, U is the conjugated cytotoxic molecule, and n is an integer from 1 to 8 (for example, from 2 to 6, or about 2, about 3, about 4, about 5, or about 6), representing the number of cytotoxic molecules bound to the antibody. In some embodiments, the anti-CD25 antibody comprises a CDR-H1, CDR-H2, and CDR-H3 having amino acid sequences SEQ ID NOs: 1, 2 and 21, respectively, and a CDR-L1, CDR-L2, and CDR-L3 having amino acid sequences SEQ ID NO: 4, 5 and 6, respectively, wherein the linker is mc-vc-pAB, and the cytotoxic molecule is MMAE. In some embodiments, the anti-CD25 antibody comprises a VH that comprises the amino acid sequence of SEQ ID NO: 22 and a VL that comprises the amino acid sequence of SEQ ID NO: 8. In some embodiments, the anti-CD25 antibody comprises a HC that comprises the amino acid sequence of SEQ ID NO: 46 and a LC that comprises the amino acid sequence of SEQ ID NO: 47.

In some aspects, the antigen binding protein is an anti-CD25 antibody and is conjugated to a camptothecin-based drug.

In some aspects, the cytostatic or cytotoxic agent is a cytostatic or cytotoxic agent disclosed in U.S. Provisional Application No. 63/397,776, U.S. Provisional Application No. 63/321,105, U.S. Provisional Application No. 63/407,609, WO 2019/195665, WO 2019/236954, or WO 2021/067861.

In some aspects, the linker is a linker disclosed in WO 2021/055865, WO 2022/198232, WO 2022/198231, or WO 2015/057699.

The average number of drugs per antibody in a preparation may be characterized by conventional means such as mass spectroscopy, HIC, ELISA assay, and HPLC. In some aspects, the antigen binding protein (e.g., anti-CD25 antibody) is attached to the drug-linker through a cysteine residue of the antibody. In some aspects, the cysteine residue is one that is engineered into the antibody. In other aspects, the cysteine residue is an interchain disulfide cysteine residue.

### VI. Therapeutic Applications

### A. Methods of Treating Diseases

In another aspect, methods of treating disorders associated with cells that express CD25, e.g., cancers, are provided. The cells may or may not express elevated levels of CD25 relative to cells that are not associated with a disorder of interest. Thus, certain aspects involve the use of the antigen binding proteins described herein (e.g., anti-CD25 antibodies), either as a naked antibody or as a conjugate (e.g., an ADC) to treat a subject, for example a subject with a cancer. In some of these aspects, the method comprises administering an effective amount of an antigen binding protein (e.g., an anti-CD25 antibody), or ADC (e.g., antibody-MMAE conjugate), or a composition comprising such an antigen binding protein or ADC to a subject in need thereof. In certain exemplary aspects, the method comprises treating cancer in a cell, tissue, organ, animal or patient. Most typically, the treatment method comprises treating a cancer in a human. In some aspects, the treatment involves monotherapy. In other methods, the antigen binding protein is administered as part of a combination therapy with one or more other therapeutic agents, surgery and/or radiation.

In some aspects, methods of treating disorders associated with Treg cells are provided. In some aspects, the antigen binding proteins bind CD25 and target CD25 expressing cells. In some aspects, the CD25 expressing cells are tumor cells. In some aspects, the CD25 expressing cells are T cells. In some aspects, the CD25 expressing cells are Treg cells.

In some aspects, the antigen binding proteins are “detuned” antigen binding proteins with reduced binding affinity to their antigen compared to non detuned antigen binding proteins. In some aspects, the antigen binding proteins are detuned CD25 binding proteins with reduced binding affinity to CD25 compared to non detuned CD25 binding proteins. In some aspects, the methods of treating a Treg associated condition or disease comprise administering to a subject in need thereof a detuned CD25 binding protein as disclosed herein.

In some aspects, a detuned CD25 binding protein as disclosed herein binds CD25 on high CD25 expressing Treg cells but does not bind or binds to a lower extent non-Treg cell populations including CD4 and CD8 T cells that express lower levels of CD25 compared to Treg cells.

In some aspects, when present in an ADC a detuned CD25 binding protein as disclosed herein has a therapeutic effect on Treg cells but has no effect or minimal effects on non-Treg cells including CD4 and CD8 T cells.

In some aspects, a method of treating a Treg associated condition or disease comprise administering a detuned CD25 ADC that comprises a MMAE. In some aspects, the detuned CD25 MMAE ADC is characterized by an enhanced cytotoxic effect on Treg cells compared to non-Treg CD4 or CD8 T cells. Without wanting to be bound by theory, it is hypothesized that this is caused by an increased binding of the detuned CD25 ADC to high CD25 expressing Treg cells compared to lower CD25 expressing CD4 and CD8 cells combined with a greater sensitivity of Treg cells to MMAE due to their reduced expression of MDR1 compared to CD4 or CD8 T cells. Advantageously, the combined use of detuned CD25 binding protein and MMAE provide a high efficacy Treg targeting therapy.

In some aspects, the method comprises administering an effective amount of an antigen binding protein (e.g., an anti-CD25 antibody), or ADC (e.g., antibody-MMAE conjugate), or a composition comprising such an antigen binding protein or ADC to a subject in need thereof. In some aspects, the subject in need thereof is a subject having cancer. In some aspects, the cancer expresses CD25. In some aspects, the cancer does not express CD25. In some aspects, the cancer comprises Treg cells. In some aspects, the intra-cancer Treg cells express CD25. In some aspects, the effective amount is an amount that reduces the number of Treg cells in a cancer such anti-cancer immunity is enhanced.

In some aspects, the methods comprise administering an effective amount of CD25 ADC that effectively reduces intra-tumor Treg cells and does not effectively reduce intra-tumor CD4 T cells and/or intra-tumor CD8 T cells. In some aspects, the methods comprise administering an effective amount of CD25 ADC that effectively reduces intra-tumor Treg cells but does not effectively reduce systemic Treg cells.

Positive therapeutic effects in cancer can be measured in a number of ways (See, e.g., W. A. Weber, J. Null. Med. 50:15-10S (2009); and Eisenhauer et al., Eur. J Cancer 45:228-247 (2009)). In some aspects, response to treatment with an antigen binding protein or conjugate is assessed using RECIST 1.1 criteria. In some aspects, the treatment achieved by a therapeutically effective amount is any of inhibition of further tumor growth, inducement of tumor regression, a partial response (PR), a complete response (CR), progression free survival (PFS), disease free survival (DFS), objective response (OR) or overall survival (OS). In some aspects, treatment delays or prevents the onset of metastasis. Progress in treatment can be monitored using various methods. For instance, inhibition can result in reduced tumor size and/or a decrease in metabolic activity within the tumor. Both of these parameters can be measured by MRI or PET scans, for example. Inhibition can also be monitored by biopsy to ascertain the level of necrosis, tumor cell death and the level of vascularity within the tumor. The dosage regimen of a therapy described herein that is effective to treat a cancer patient may vary according to factors such as the disease state, age, and weight of the patient, and the ability of the therapy to elicit an anti-cancer response in the subject. While an aspects of the treatment method, medicaments and uses of the present invention may not be effective in achieving a positive therapeutic effect in every subject, it should do so in a statistically significant number of subjects as determined by any statistical test known in the art such as the Student's t-test, the chi2-test, the U-test according to Mann and Whitney, the Kruskal-Wallis test (H-test), Jonckheere-Terpstra-test and the Wilcoxon-test.

“RECIST 1.1 Response Criteria” as used herein means the definitions set forth in Eisenhauer et al., Eur. J Cancer 45:228-247 (2009) for target lesions or non-target lesions, as appropriate, based on the context in which response is being measured.

The dosage of the antigen binding protein (e.g., anti-CD25 antibody) or antibody-drug conjugate (ADC) administered can vary depending upon known factors, such as the pharmacodynamic characteristics of the particular agent, and its mode and route of administration; the age, health, and weight of the recipient; the type and extent of disease or indication to be treated, the nature and extent of symptoms, kind of concurrent treatment, frequency of treatment, and the effect desired. The initial dosage can be increased beyond the upper level in order to rapidly achieve the desired blood-level or tissue-level. Alternatively, the initial dosage can be smaller than the optimum, and the daily dosage may be progressively increased during the course of treatment.

The frequency of administration depends on the half-life of the antigen binding protein or ADC in the circulation, the condition of the patient and the route of administration among other factors.

Accordingly, a method of treating cancer in a subject in need thereof can comprises administering an antigen binding protein or ADC disclosed herein. For example, the antigen binding protein (e.g., an anti-CD25 antibody) or ADC (e.g., anti-CD25 ADC) may be administered at least once every four weeks, such as at least once every three weeks. For example, the antigen binding protein (e.g., an anti-CD25 antibody) or ADC (e.g., anti-CD25 ADC) is administered 1, 2, or 3 times every four weeks or 1 or 2 times every three weeks. In some embodiments, the antigen binding protein (e.g., an anti-CD25 antibody) or ADC (e.g., anti-CD25 ADC) is administered weekly, every two weeks (biweekly), every three weeks or monthly. The antigen binding protein (e.g., an anti-CD25 antibody) or ADC (e.g., anti-CD25 ADC) may be administered weekly, every two weeks (biweekly), every three weeks or monthly.

In another embodiment, the antigen binding protein (e.g., an anti-CD25 antibody) or ADC (e.g., anti-CD25 ADC) is administered twice every three weeks, such as two weekly doses every three weeks (e.g., Days 1 and 8 every 21 days). In one embodiment, the antibody or ADC is administered three times every four weeks, such as three weekly doses every four weeks (e.g., Days 1, 8, 15 every 28 days). In another embodiment, the antibody or ADC is administered twice every four weeks, such as two biweekly doses every four weeks (e.g., Days 1, 15 every 28 days). In another embodiment, the antibody or ADC is administered once every three weeks (e.g., Day 1 every 21 days).

The dose of the antigen binding protein (e.g., an anti-CD25 antibody) or ADC (e.g., anti-CD25 ADC) may be about 0.1 mg/kg to about 3.0 mg/kg, such as about 0.25 to about 2.0 mg/kg, for example, about 0.375 mg/kg, about 0.5 mg/kg, about 0.563 mg/kg, about 0.625 mg/kg, about 0.667 mg/kg, about 0.75 mg/kg, about 0.833 mg/kg, about 0.875 mg/kg, about 0.937 mg/kg, about 1.0 mg/kg, about 1.167 mg/kg, about 1.25 mg/kg, about 1.312 mg/kg, about 1.33 mg/kg, about 1.5 mg/kg, about 1.75 mg/kg or about 2.0 mg/kg. The dose may be based on AIBW (Adjusted Ideal Body Weight). For example, the dose may be about 0.1 mg/kg to about 3.0 mg/kg, such as about 0.25 mg/kg to about 2.0 mg/kg, or about 0.375 mg/kg, about 0.5 mg/kg, about 0.563 mg/kg, about 0.625 mg/kg, about 0.667 mg/kg, about 0.75 mg/kg, about 0.833 mg/kg, about 0.875 mg/kg, about 0.937 mg/kg, about 1.0 mg/kg, about 1.167 mg/kg, about 1.25 mg/kg, about 1.312 mg/kg, about 1.33 mg/kg, about 1.5 mg/kg, about 1.75 mg/kg or about 2.0 mg/kg AIBW. In some embodiments, the dose is administered intravenously (IV). The administration, such as by IV, may be over about 5, 10, 15, 20, 25, 30, 35, 40, 45, 50, 55, or 60 minutes.

In some embodiments, the dose may be about 0.1 mg/kg to about 3.0 mg/kg, such as about 0.25 mg/kg to about 2.0 mg/kg, and is administered at least once every four weeks. In some embodiments, the dose is administered 1, 2 or 3 times every four weeks, or 1 or 2 times every three weeks. The dose of the antigen binding protein (e.g., an anti-CD25 antibody) or ADC (e.g., anti-CD25 ADC) can be about 0.75 mg/kg, about 1.0 mg/kg, about 1.25 mg/kg, about 1.5 mg/kg, about 1.75 mg/kg or about 2.0 mg/kg, for example, when administered on a dosing schedule of two doses every four weeks, such as two biweekly doses every four weeks (e.g., Days 1, 15 every 28 days). In some embodiments, the dose may be about 0.375 mg/kg, about 0.5 mg/kg, about 0.625 mg/kg, about 0.75 mg/kg, about 0.875 mg/kg or about 1.0 mg/kg, for example, when administered on a dosing schedule of two doses every four weeks, such as two biweekly doses every four weeks (e.g., Days 1, 15 every 28 days). The dose may also be about 0.563 mg/kg, about 0.75 mg/kg, about 0.937 mg/kg, about 1.125 mg/kg, about 1.312 mg/kg or about 1.5 mg/kg, example, when administered on a dosing schedule of three doses every four weeks, such as three weekly doses every four weeks (e.g., Days 1, 8, 15 every 28 days). The dose may also be about 0.563 mg/kg, about 0.75 mg/kg, about 0.937 mg/kg, about 1.125 mg/kg, about 1.312 mg/kg or about 1.5 mg/kg, example, when administered on a dosing schedule of once every three weeks (e.g., Day 1 every 21 days). The dose may also be about 0.5 mg/kg, about 0.667 mg/kg, about 0.833 mg/kg, about 1.0 mg/kg, about 1.167 mg/kg or about 1.33 mg/kg, example, when administered on a dosing schedule of two doses every three weeks, such as two weekly doses every three weeks (e.g., Days 1 and 8 every 21 days).

Any cancer comprising Tregs can be treated with the antigen binding protein or ADC described herein. The cancer may contain CD25-positive and CD25-negative cells. In some aspects, cancers suitable for treatment with the antigen binding proteins provided herein are those that possess CD25 expression in a cancerous cell or tissue (i.e., “CD25-expressing cancers”). Examples of cancers that can be treated with an antigen binding protein or ADC thereof include, but are not limited to, hematopoietic tumors, hematopoietic tumors that give rise to solid tumors, solid tumors, soft tissue tumors, and metastatic lesions.

In some aspects, cancers suitable for treatment with the antigen binding proteins provided herein are those that possess Treg cells in a cancerous microenvironment, including non-CD25 expressing cancers. Examples of cancers that can be treated with an antigen binding protein or ADC thereof include, but are not limited to, hematopoietic tumors, solid tumors, soft tissue tumors, and metastatic lesions.

Examples of hematopoietic tumors that have the potential to give rise to solid tumors include, but are not limited to, diffuse large B-cell lymphomas (DLBCL), follicular lymphoma, myelodysplastic syndrome (MDS), a lymphoma, Hodgkin's disease, a malignant lymphoma, non-Hodgkin's lymphoma, Burkitt's lymphoma, multiple myeloma, Richter's Syndrome (Richter's Transformation) and the like. In some aspects, a hematopoietic tumor that has the potential to give rise to solid tumors is a diffuse large B-cell lymphoma (DLBCL). In some aspects, a hematopoietic tumor that has the potential to give rise to solid tumors is a follicular lymphoma. In some aspects, a hematopoietic tumor that has the potential to give rise to solid tumors is a myelodysplastic syndrome (MDS). In some aspects, a hematopoietic tumor that has the potential to give rise to solid tumors is a lymphoma. In some aspects, a hematopoietic tumor that has the potential to give rise to solid tumors is Hodgkin's disease. In some aspects, a hematopoietic tumor that has the potential to give rise to solid tumors is a malignant lymphoma. In some aspects, a hematopoietic tumor that has the potential to give rise to solid tumors is a non-Hodgkin's lymphoma. In some aspects, a hematopoietic tumor that has the potential to give rise to solid tumors is a Burkitt's lymphoma. In some aspects, a hematopoietic tumor that has the potential to give rise to solid tumors is a multiple myeloma. In some aspects, a hematopoietic tumor that has the potential to give rise to solid tumors is Richter's Syndrome (Richter's Transformation). In certain aspects, the cancer is selected from, but not limited to, leukemia's such as acute lymphoblastic leukemia (ALL), chronic lymphocytic leukemia (CLL), acute myelogenous leukemia (AML), chronic myelogenous leukemia (CML), hairy cell leukemia (HCL), T-cell prolymphocytic leukemia (T-PLL), large granular lymphocytic leukemia, adult T-cell leukemia, and acute monocytic leukemia (AML).

In some aspects, the cancer is another hematological cancer, including, but are not limited to, non-Hodgkin lymphoma (e.g., diffuse large B cell lymphoma, mantle cell lymphoma, B lymphoblastic lymphoma, peripheral T cell lymphoma and Burkitt's lymphoma), B-lymphoblastic lymphoma; B-cell chronic lymphocytic leukemia/small lymphocytic lymphoma; lymphoplasmacytic lymphoma; splenic marginal zone B-cell lymphoma (±villous lymphocytes); plasma cell myeloma/plasmacytoma; extranodal marginal zone B-cell lymphoma of the MALT type; nodal marginal zone B-cell lymphoma (monocytoid B cells); follicular lymphoma; diffuse large B-cell lymphomas; Burkitt's lymphoma; precursor T-lymphoblastic lymphoma; T adult T-cell lymphoma (HTLV 1-positive); extranodal NK/T-cell lymphoma, nasal type; enteropathy-type T-cell lymphoma; hepatosplenic γ-δ T-cell lymphoma; subcutaneous panniculitis-like T-cell lymphoma; mycosis fungoides/sezary syndrome; anaplastic large cell lymphoma, T/null cell, primary cutaneous type; anaplastic large cell lymphoma, T-/null-cell, primary systemic type; peripheral T-cell lymphoma, not otherwise characterized; angioimmunoblastic T-cell lymphoma, multiple myeloma, polycythemia vera or myelofibrosis, cutaneous T-cell lymphoma, small lymphocytic lymphoma (SLL), marginal zone lymphoma, CNS lymphoma, immunoblastic large cell lymphoma, precursor B-lymphoblastic lymphoma and the like.

Exemplary solid tumors that can be treated include, but are not limited to, malignancies, e.g., sarcomas (including soft tissue sarcoma and osteosarcoma), adenocarcinomas, and carcinomas, of the various organ systems, such as those affecting head and neck (including pharynx), thyroid, lung (small cell lung carcinoma (SCLC) or non-small cell lung carcinoma (NSCLC)), breast, lymphoid, gastrointestinal tract (e.g., oral, esophageal, stomach, liver, pancreas, small intestine, colon and rectum, anal canal), genitals and genitourinary tract (e.g., renal, urothelial, bladder, ovarian, uterine, cervical, endometrial, prostate, testicular), central nervous system (e.g., neural or glial cells, e.g., neuroblastoma or glioma), skin (e.g., melanoma) and the like. In some aspects, the solid tumor is an NMDA receptor positive teratom, breast cancer, colon cancer, pancreatic cancer (e.g., a pancreatic neuroendocrine tumors (PNET) or a pancreatic ductal adenocarcinoma (PDAC)), stomach cancer, uterine cancer, and ovarian cancer. In some aspects, the cancer includes a squamous cell cancer, lung squamous cell carcinoma, pituitary cancer, esophageal carcinoma, astrocytoma, soft tissue sarcoma, non-small cell lung cancer, lung adenocarcinoma, squamous carcinoma of the lung, mesothelioma, cancer of the peritoneum, hepatocellular cancer, gastrointestinal cancer, stomach adenocarcinoma, pancreatic cancer, pancreatic adenocarcinoma, glioblastoma, cervical cancer, ovarian cancer, ovarian serous cystadenocarcinoma, liver cancer, bladder cancer, hepatoma, breast cancer, breast invasive carcinoma, colon cancer, colorectal cancer, endometrial or uterine carcinoma, uterine corpus endometrial carcinoma, cervical squamous cell carcinoma and endocervical adenocarcinoma, salivary gland carcinoma, kidney cancer, kidney renal clear cell carcinoma, renal cancer, bladder urothelial carcinoma, liver cancer, prostate cancer, testicular germ cell tumor, rectum adenocarcinoma, colon adenocarcinoma, sarcoma, vulval cancer, thyroid cancer, thymoma, hepatic carcinoma, brain cancer, endometrial cancer, testis cancer, cholangiocarcinoma, gallbladder carcinoma, gastric cancer, melanoma, skin cutaneous melanoma, various types of head and neck cancer, head neck squamous cell carcinoma, acute myeloid leukemia, ALL, lymphoid neoplasm diffuse large B-cell lymphoma, LCML, chronic lymphocytic leukemia, anaplastic large cell lymphoma, and Hodgkin lymphoma.

The methods, the antigen binding proteins and ADCs described herein can be used in combination with other therapeutic agents and/or modalities. In such combination therapeutic methods, two (or more) different treatments are delivered to the subject during the course of the subject's affliction with the disorder, such that the effects of the treatments on the patient overlap at a point in time. In certain aspects, the delivery of one treatment is still occurring when the delivery of the second begins, so that there is overlap in terms of administration. This is sometimes referred to herein as “simultaneous” or “concurrent delivery.” In other aspects, the delivery of one treatment ends before the delivery of the other treatment begins. In some aspects of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some aspects, delivery is such that the reduction in a symptom, or other parameter related to the disorder is greater than what would be observed with one treatment delivered in the absence of the other. The effect of the two treatments can be partially additive, wholly additive, or greater than additive (i.e., a synergistic response). The delivery can be such that an effect of the first treatment delivered is still detectable when the second is delivered.

In certain aspects, the methods provided herein include administering to the subject an antigen binding protein (e.g., an anti-CD25 antibody) or ADC (e.g., anti-CD25 ADC) as described herein, e.g., a composition or preparation, in combination with one or more additional therapies, e.g., surgery, radiation therapy, or administration of another therapeutic preparation. For example, in some aspects, the antigen binding protein is combined with chemotherapy (e.g., a cytotoxic agent), a targeted therapy (e.g., an antibody against a cancer antigen), an angiogenesis inhibitor, and/or an immunomodulatory agent, such as an inhibitor of an immune checkpoint molecule. In other aspects, the additional therapy is an anti-inflammatory (e.g., methotrexate), or an anti-fibrotic agent. The antigen binding protein (e.g., an anti-CD25 antibody) or ADC (e.g., anti-CD25 ADC) and the additional therapy can be administered simultaneously or sequentially.

Exemplary cytotoxic agents that can be used in combination with the antigen binding protein (e.g., an anti-CD25 antibody) or ADC (e.g., anti-CD25 ADC) in some aspects include anti-microtubule agents, topoisomerase inhibitors, antimetabolites, protein synthesis and degradation inhibitors, mitotic inhibitors, alkylating agents, platinating agents, inhibitors of nucleic acid synthesis, histone deacetylase inhibitors (HDAC inhibitors, e.g., vorinostat (SAHA, MK0683), entinostat (MS-275), panobinostat (LBH589), trichostatin A (TSA), mocetinostat (MGCD0103), belinostat (PXD101), romidepsin (FK228, depsipeptide)), DNA methyltransferase inhibitors, nitrogen mustards, nitrosoureas, ethylenimines, alkyl sulfonates, triazenes, folate analogs, nucleoside analogs, ribonucleotide reductase inhibitors, vinca alkaloids, taxanes, epothilones, intercalating agents, agents capable of interfering with a signal transduction pathway, agents that promote apoptosis and radiation, or antibody molecule conjugates that bind surface proteins to deliver a toxic agent. In one aspects, the cytotoxic agent that can be administered with an antigen binding protein described herein is a platinum-based agent (such as cisplatin), cyclophosphamide, dacarbazine, methotrexate, fluorouracil, gemcitabine, capecitabine, hydroxyurea, topotecan, irinotecan, azacytidine, vorinostat, ixabepilone, bortezomib, taxanes (e.g., paclitaxel or docetaxel), cytochalasin B, gramicidin D, ethidium bromide, emetine, mitomycin, etoposide, tenoposide, vincristine, vinblastine, vinorelbine, colchicin, anthracyclines (e.g., doxorubicin or epirubicin) daunorubicin, dihydroxy anthracin dione, mitoxantrone, mithramycin, actinomycin D, adriamycin, 1-dehydrotestosterone, glucocorticoids, procaine, tetracaine, lidocaine, propranolol, puromycin, ricin, or maytansinoids.

In some aspects, the antigen binding protein (e.g., an anti-CD25 antibody) or ADC (e.g., anti-CD25 ADC) is administered as part of a chemotherapeutic regimen such as CHOP (cyclophosphamide, doxorubicin, vincristine, and prednisone); CVP (cyclophosphamide, vincristine, and prednisone); RCVP (rituximab+CVP); RCHOP (rituximab+CHOP); RCHP (rituximab, cyclophosphamide, doxorubicin, and prednisone); RICE (Rituximab+ifosamide, carboplatin, etoposide); RDHAP, (Rituximab+dexamethasone, cytarabine, cisplatin); RESHAP (rituximab+etoposide, methylprednisolone, cytarabine, cisplatin); R-BENDA (rituximab and Bendamustine), RGDP (rituximab, gemcitabine, dexamethasone, cisplatin). In an aspects, one of CHOP, CVP, RCVP, RCHOP, RCHP, RICE, RDHAP, RESHAP, R-BENDA, and RGDP is administered in a combination therapy with an antigen binding protein or conjugate as described herein.

Examples of targeted therapies that can be combined with an antigen binding protein (e.g., an anti-CD25 antibody) or ADC (e.g., anti-CD25 ADC) in certain aspects include, but are not limited to, use of therapeutic antibodies. Exemplary antibodies include, but are not limited to, those which bind to cell surface proteins such as Her2, CDC20, CDC33, mucin-like glycoprotein, and epidermal growth factor receptor (EGFR) present on tumor cells, and optionally induce a cytostatic and/or cytotoxic effect on tumor cells displaying these proteins. Exemplary antibodies also include HERCEPTIN® (trastuzumab), which may be used to treat breast cancer and other forms of cancer, and RITUXAN® (rituximab), ZEVALIN® (ibritumomab tiuxetan), GLEEVEC® and LYMPHOCIDE® (epratuzumab), which may be used to treat non-Hodgkin's lymphoma and other forms of cancer. Other exemplary antibodies include panitumumab (VECTIBIX®), ERBITUX® (IMC-C225); ertinolib (Iressa); BEXXAR® (iodine 131 tositumomab); KDR (kinase domain receptor) inhibitors; anti VEGF antibodies and antagonists (e.g., Avastin®, motesanib, and VEGAF-TRAP); anti VEGF receptor antibodies and antigen binding regions; anti-Ang-1 and Ang-2 antibodies and antigen binding regions; antibodies to Tie-2 and other Ang-1 and Ang-2 receptors; Tie-2 ligands; antibodies against Tie-2 kinase inhibitors; inhibitors of Hif-1a, and Campath® (Alemtuzumab). In certain aspects, cancer therapy agents are polypeptides which selectively induce apoptosis in tumor cells, including, but not limited to, the TNF-related polypeptide TRAIL.

In certain aspects, an antigen binding protein as provided herein is used in combination with one or more anti-angiogenic agents that decrease angiogenesis. Such agents include, but are not limited to, IL-8 antagonists; Campath®, B-FGF; FGF antagonists; Tek antagonists (Cerretti et al., U.S. Publication No. 2003/0162712; Cerretti et al., U.S. Pat. No. 6,413,932, and Cerretti et al., U.S. Pat. No. 6,521,424); anti-TWEAK agents (which include, but are not limited to, antibodies and antigen binding regions); soluble TWEAK receptor antagonists (Wiley, U.S. Pat. No. 6,727,225); an ADAM distintegrin domain to antagonize the binding of integrin to its ligands (Fanslow et al., U.S. Publication No. 2002/0042368); anti-eph receptor and anti-ephrin antibodies, antigen binding regions, or antagonists (U.S. Pat. Nos. 5,981,245; 5,728,813; 5,969,110; 6,596,852; 6,232,447; 6,057,124); anti-VEGF agents (e.g., antibodies or antigen binding regions that specifically bind VEGF, or soluble VEGF receptors or a ligand binding regions thereof) such as Avastin® or VEGF-TRAP™, and anti-VEGF receptor agents (e.g., antibodies or antigen binding regions that specifically bind thereto), EGFR inhibitory agents (e.g., antibodies or antigen binding regions that specifically bind thereto) such as panitumumab, IRESSA® (gefitinib), TARCEVA® (erlotinib), anti-Ang-1 and anti-Ang-2 agents (e.g., antibodies or antigen binding regions specifically binding thereto or to their receptors, e.g., Tie-2/TEK), and anti-Tie-2 kinase inhibitory agents (e.g., antibodies or antigen binding regions that specifically bind and inhibit the activity of growth factors, such as antagonists of hepatocyte growth factor (HGF, also known as Scatter Factor), and antibodies or antigen binding regions that specifically bind its receptor “c-met”; anti-PDGF-BB antagonists; antibodies and antigen binding regions to PDGF-BB ligands; and PDGFR kinase inhibitors.

Other anti-angiogenic agents that can be used in combination with an antigen binding protein include agents such as MMP-2 (matrix-metalloproteinase 2) inhibitors, MMP-9 (matrix-metalloproteinase 9) inhibitors, and COX-II (cyclooxygenase II) inhibitors. Examples of useful COX-II inhibitors include CELEBREX® (celecoxib), valdecoxib, and rofecoxib.

An “immune checkpoint molecule,” as used herein, refers to a molecule in the immune system that either turns up a signal (a stimulatory molecule) and/or turns down a signal (an inhibitory molecule). Many cancers evade the immune system by inhibiting T cell signaling. Exemplary immune checkpoint molecules that can be used with an antigen binding protein in certain aspects include, but are not limited to, programmed cell death protein 1 (PD-1), programmed death-ligand 1 (PD-L1), PD-L2, cytotoxic T lymphocyte-associated protein 4 (CTLA-4), T cell immunoglobulin and mucin domain containing 3 (TIM-3), lymphocyte activation gene 3 (LAG-3), carcinoembryonic antigen related cell adhesion molecule 1 (CEACAM-1), CEACAM-5, V-domain Ig suppressor of T cell activation (VISTA), B and T lymphocyte attenuator (BTLA), T cell immunoreceptor with Ig and ITIM domains (TIGIT), leukocyte-associated immunoglobulin-like receptor 1 (LAIR1), CD160, TGFR, adenosine 2A receptor (A2AR), B7-H3 (also known as CD276), B7-H4 (also called VTCN1), indoleamine 2,3-dioxygenase (IDO), 2B4, killer cell immunoglobulin-like receptor (KIR), OX40, 4-1BB, 4-1BBL, B7-H3, Inducible T-cell Co-stimulator (ICOS/ICOS-L), CD27/CD70, Glucocorticoid-Induced TNF Receptor (GITR), CD47/Signal-Regulatory Protein alpha (SIRPα), and Indoleamine-2,3-Dioxygenase (IDO).

Specific examples of immune checkpoint inhibitors that can be used in combination with the antigen binding protein (e.g., an anti-CD25 antibody) or ADC (e.g., anti-CD25 ADC) in certain aspects include, but are not limited to, the following monoclonal antibodies: PD-1 inhibitors such as pembrolizumab (Keytruda®, Merck) and nivolumab (Opdivo®, Bristol-Myers Squibb); PD-L1 inhibitors such as atezolizumab (Tecentriq®, Genentech), avelumab (Bavencio®, Pfizer), durvalumab (Imfinzi®, AstraZeneca); and CTLA-1 inhibitors such as ipilimumab (Yervoy®, Bristol-Myers Squibb) and tremelimumab (AstraZeneca). In some aspects, the antigen binding protein (e.g., an anti-CD25 antibody) or ADC (e.g., anti-CD25 ADC) described herein is used in combination with PD-1 inhibitor pembrolizumab (Keytruda®, Merck). In some aspects, the antigen binding protein described herein is used in combination with PD-1 inhibitor nivolumab (Opdivo®, Bristol-Myers Squibb). In some aspects, the antigen binding protein (e.g., an anti-CD25 antibody) or ADC (e.g., anti-CD25 ADC) described herein is used in combination with PD-1 inhibitor sasanlimab (Pfizer). In some aspects, the antigen binding protein (e.g., an anti-CD25 antibody) or ADC (e.g., anti-CD25 ADC) described herein is used in combination with PD-L1 inhibitor atezolizumab (Tecentriq®, Genentech). In some aspects, the antigen binding protein (e.g., an anti-CD25 antibody) or ADC (e.g., anti-CD25 ADC) described herein is used in combination with PD-L1 inhibitor avelumab (Bavencio®, Pfizer). In some aspects, the antigen binding protein (e.g., an anti-CD25 antibody) or ADC (e.g., anti-CD25 ADC) described herein is used in combination with PD-L1 inhibitor durvalumab (Imfinzi®, AstraZeneca).

In some aspects, the antigen binding protein (e.g., an anti-CD25 antibody) or ADC (e.g., anti-CD25 ADC) described herein is used in combination with CTLA-1 inhibitor ipilimumab (Yervoy®, Bristol-Myers Squibb). In some aspects, the antigen binding protein described herein is used in combination with CTLA-1 inhibitor tremelimumab (AstraZeneca).

In some aspects, the antigen binding protein (e.g., an anti-CD25 antibody) or ADC (e.g., anti-CD25 ADC) described herein is used in combination with a TIGIT binding agent.

In some aspects, the antigen binding protein (e.g., an anti-CD25 antibody) or ADC (e.g., anti-CD25 ADC) described herein is used in combination with brentuximab vedotin.

In some aspects, the antigen binding protein (e.g., an anti-CD25 antibody) or ADC (e.g., anti-CD25 ADC) described herein is used in combination with an ADC in which the antibody is a CD30 antibody. In some aspects, the CD30 antibody is conjugated to camptothecin. In some aspects, the CD30 antibody is conjugated to MMAE.

In some aspects, the antigen binding protein (e.g., an anti-CD25 antibody) or ADC (e.g., anti-CD25 ADC) described herein is used in combination with a CD40 binding agent.

In some aspects, the antigen binding protein (e.g., an anti-CD25 antibody) or ADC (e.g., anti-CD25 ADC) described herein is used in combination with a bispecific 41BB-CD228 binding agent.

In some aspects, the antigen binding protein (e.g., an anti-CD25 antibody) or ADC (e.g., anti-CD25 ADC) described herein is used in combination with a Trop2 binding agent.

In some aspects, the antigen binding protein (e.g., an anti-CD25 antibody) or ADC (e.g., anti-CD25 ADC) described herein is used in combination with a gamma delta T cell engager.

In one aspect, a PD-1 inhibitor, such as sasanlimab, is administered with the antigen binding protein (e.g., an anti-CD25 antibody) or ADC (e.g., anti-CD25 ADC). Sasanlimab may be administered at a fixed dose, such as 225 mg or 300 mg. Sasanlimab may be administered within a week, within a day (e.g., 24 hours), or within 5, 10, 15, 20, 25, 30, 35, 40, 45, 50, 55, or 60 minutes of administration of the antibody or ADC. For example, sasanlimab may be administered at least about 5, 10, 15, 20, 25, 30, 35, 40, 45, 50, 55, or 60 minutes prior to administration of the antibody or ADC, or at least about 5, 10, 15, 20, 25, 30, 35, 40, 45, 50, 55, or 60 minutes after administration of the antibody or ADC. Sasanlimab may be administered once per treatment cycle, such as once every three weeks or once every four weeks.

For example, a method of treating a cancer (for example, a lymphoma or a solid tumor, such as peripheral T-cell lymphoma (PTCL), diffuse large B-cell lymphoma (DLBCL), classical Hodgkin lymphoma (cHL), head and neck squamous cell carcinoma (HNSCC), melanoma, or non-small cell lung cancer (NSCLC)) in a subject in need thereof may comprise a dose of the antigen binding protein (e.g., an anti-CD25 antibody) or ADC (e.g., anti-CD25 ADC) of about 0.1 mg/kg to about 3.0 mg/kg, such as about 0.25 mg/kg to about 2.0 mg/kg, wherein the dose is administered at least once every four weeks, such as 1, 2 or 3 times every four weeks, or 1 or 2 times every three weeks, and the method further comprises administering sasanlimab. Sasanlimab may be administered at a fixed dose. In some embodiments, sasanlimab is administered at least once every three or four weeks, for example, administered within 24 hours of administration of the antigen binding protein or the antibody-drug conjugate.

In some embodiments, the dose of the antigen binding protein (e.g., an anti-CD25 antibody) or ADC (e.g., anti-CD25 ADC) may be about 0.75 mg/kg, about 1.0 mg/kg, about 1.25 mg/kg, about 1.5 mg/kg, about 1.75 mg/kg or about 2.0 mg/kg and administered on a dosing schedule of two doses every four weeks (e.g., Days 1, 15 every 28 days), and sasanlimab is administered at a fixed dose of 300 mg once every 28 days (e.g., on Day 1 and at least 30 minutes prior to administration of the antibody or ADC).

In one embodiment, the dose of the antigen binding protein (e.g., an anti-CD25 antibody) or ADC (e.g., anti-CD25 ADC) may be about 0.375 mg/kg, about 0.5 mg/kg, about 0.625 mg/kg, about 0.75 mg/kg, about 0.875 mg/kg or about 1.0 mg/kg and administered on a dosing schedule of two doses every four weeks (e.g., Days 1, 15 every 28 days) and sasanlimab is administered at a fixed dose of 300 mg once every 28 days (e.g., on Day 1 and at least 30 minutes prior to administration of the antibody or ADC).

In another embodiment, the dose of the antigen binding protein (e.g., an anti-CD25 antibody) or ADC (e.g., anti-CD25 ADC) may be about 0.563 mg/kg, about 0.75 mg/kg, about 0.937 mg/kg, about 1.125 mg/kg, about 1.312 mg/kg or about 1.5 mg/kg, example, when administered on a dosing schedule of three doses every four weeks, such as three weekly doses every four weeks (e.g., Days 1, 8, 15 every 28 days), and sasanlimab is administered at a fixed dose of 300 mg once every 28 days (e.g., on Day 1 and at least 30 minutes prior to administration of the antibody or ADC).

In yet another embodiment, the dose of the antigen binding protein (e.g., an anti-CD25 antibody) or ADC (e.g., anti-CD25 ADC) may be about 0.563 mg/kg, about 0.75 mg/kg, about 0.937 mg/kg, about 1.125 mg/kg, about 1.312 mg/kg or about 1.5 mg/kg, example, when administered on a dosing schedule of once every three weeks (e.g., Day 1 every 21 days) and sasanlimab is administered at a fixed dose of 225 mg once every 21 days (e.g., on Day 1 and at least 30 minutes prior to administration of the antibody or ADC).

In some embodiments, the dose of the antigen binding protein (e.g., an anti-CD25 antibody) or ADC (e.g., anti-CD25 ADC) may be about 0.5 mg/kg, about 0.667 mg/kg, about 0.833 mg/kg, about 1 mg/kg, about 1.167 mg/kg or about 1.33 mg/kg, example, when administered on a dosing schedule of two doses every three weeks, such as two weekly doses every three weeks (e.g., Days 1 and 8 every 21 days) and sasanlimab is administered at a fixed dose of 225 mg once every 21 days (e.g., on Day 1 and at least 30 minutes prior to administration of the antibody or ADC).

### VIII. Diagnostic Applications

In another aspect, the antigen binding protein (e.g., an anti-CD25 antibody or fragment thereof), polypeptides, and nucleic acids as provided herein can be used in methods for detecting, diagnosing and monitoring of a disease, disorder or condition associated with CD25.

In some aspects, the method comprises detecting the expression of CD25 in a sample, e.g., a tissue sample or peritoneal fluid or pleural fluid sample, obtained from a subject suspected of having a disorder, e.g., a cancer, comprising cancer cells and CD25-expressing regulatory T cells. In some aspects, the method of detection comprises contacting the sample with an antibody, polypeptide, or polynucleotide as described herein and determining whether the level of binding differs from that of a reference or comparison sample. In some aspects, such methods are useful to determine whether CD25-positive Treg cells are present in a subject's tumor and to determine whether antibodies or antibody-conjugates described herein are an appropriate treatment for the subject.

For example, in some aspects, the cells or cell/tissue lysates are contacted with an anti-CD25 antibody and the binding between the antibody and the cell or antigen is determined. When the test cells show binding activity as compared to a reference cell of the same tissue type, it may indicate presence of a disease or condition associated with CD25-positive Treg cells. In some aspects, the test cells are from human tissues.

Various methods known in the art for detecting specific antibody-antigen binding can be used. Exemplary immunoassays which can be conducted according to the invention include fluorescence polarization immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA).

Diagnostic applications provided herein include use of an antigen binding protein (e.g., an anti-CD25 antibody or fragment thereof) to detect expression of CD25 and binding of the ligands to CD25. For diagnostic applications, the antigen binding protein typically is labeled with a detectable labeling group. Suitable labeling groups include, but are not limited to, the following: radioisotopes or radionuclides (e.g., ³H, ¹⁴C, ¹⁵N, 35S ⁹⁰Y, ⁹⁹Tc, ¹¹¹In, ¹²⁵I, ¹³¹I), fluorescent groups (e.g., FITC, rhodamine, lanthanide phosphors), enzymatic groups (e.g., horseradish peroxidase, 0-galactosidase, luciferase, alkaline phosphatase), chemiluminescent groups, biotinyl groups, or predetermined polypeptide epitopes recognized by a secondary reporter (e.g., leucine zipper pair sequences, binding sites for secondary antibodies, metal binding domains, epitope tags). In some aspects, the labeling group is coupled to the antigen binding protein via spacer arms of various lengths to reduce potential steric hindrance. Various methods for labeling proteins are known in the art and may be used. Examples of methods useful in the detection of the presence of CD25 include immunoassays such as those described above.

In another aspect, an antigen binding protein can be used to identify a cell or cells that express CD25. In a specific aspects, the antigen binding protein is labeled with a labeling group and the binding of the labeled antigen binding protein to CD25 is detected. In a further specific aspects, the binding of the antigen binding protein to CD25 is detected in vivo.

An antigen binding protein (e.g., an anti-CD25 antibody or fragment thereof) also can be used as staining reagent in pathology using techniques well known in the art.

### IX. Pharmaceutical Compositions and Formulations

Pharmaceutical compositions that comprise an antigen binding protein (e.g., an anti-CD25 antibody or fragment thereof) are also provided and can be utilized in any of the therapeutic applications disclosed herein. In certain aspects, the pharmaceutical composition comprises a therapeutically effective amount of one or a plurality of the antigen binding protein, together with pharmaceutically acceptable diluent or carrier. In other aspects, the pharmaceutical composition comprises a therapeutically effective amount of one or a plurality of the antigen binding proteins, a pharmaceutically acceptable diluent, carrier, solubilizer, emulsifier, preservative, and/or adjuvant. Acceptable formulation materials are nontoxic to recipients at the dosages and concentrations employed. The pharmaceutical compositions can be formulated as liquid, frozen or lyophilized compositions.

In certain aspects, the pharmaceutical composition can contain formulation materials for modifying, maintaining or preserving, for example, the pH, osmolarity, viscosity, clarity, color, isotonicity, odor, sterility, stability, rate of dissolution or release, adsorption or penetration of the composition. Suitable formulation materials include, but are not limited to, amino acids; antimicrobials; antioxidants; buffers; bulking agents; chelating agents; complexing agents; fillers; carbohydrates such as monosaccharides or disaccharides; proteins; coloring, flavoring and diluting agents; emulsifying agents; hydrophilic polymers; low molecular weight polypeptides; salt-forming counterions (such as sodium); preservatives; solvents (such as glycerin, propylene glycol or polyethylene glycol); sugar alcohols; suspending agents; surfactants or wetting agents; stability enhancing agents; tonicity enhancing agents; delivery vehicles; and/or pharmaceutical adjuvants. Additional details and options for suitable agents that can be incorporated into pharmaceutical compositions are provided in, for example, Remington's Pharmaceutical Sciences, 22^{nd} Edition, (Loyd V. Allen, ed.) Pharmaceutical Press (2013); Ansel et al., Pharmaceutical Dosage Forms and Drug Delivery Systems, 7^{th} ed., Lippencott Williams and Wilkins (2004); and Kibbe et al., Handbook of Pharmaceutical Excipients, 3^{rd} ed., Pharmaceutical Press (2000).

The components of the pharmaceutical composition are selected depending upon, for example, the intended route of administration, delivery format and desired dosage. See, for example, Remington's Pharmaceutical Sciences, 22^{nd} Edition, (Loyd V. Allen, ed.) Pharmaceutical Press (2013). The compositions are selected to influence the physical state, stability, rate of in vivo release and rate of in vivo clearance of the antigen binding proteins disclosed. The primary vehicle or carrier in a pharmaceutical composition can be either aqueous or non-aqueous in nature. For example, a suitable vehicle or carrier can be water for injection or physiological saline solution. In certain aspects, antigen binding protein compositions can be prepared for storage by mixing the selected composition having the desired degree of purity with optional formulation agents in the form of a lyophilized cake or an aqueous solution. Further, in certain aspects, the antigen binding protein can be formulated as a lyophilizate using appropriate excipients.

Free amino acids or proteins are used in some compositions as bulking agents, stabilizers, and/or antioxidants. As an example, lysine, proline, serine, and alanine can be used for stabilizing proteins in a formulation. Glycine is useful in lyophilization to ensure correct cake structure and properties. Arginine may be useful to inhibit protein aggregation, in both liquid and lyophilized formulations. Methionine is useful as an antioxidant. Glutamine and asparagine are included in some aspects. An amino acid is included in some formulations because of its buffering capacity. Such amino acids include, for instance, alanine, glycine, arginine, betaine, histidine, glutamic acid, aspartic acid, cysteine, lysine, leucine, isoleucine, valine, methionine, phenylalanine, aspartame, and the like. Certain formulations also include a protein excipient such as serum albumin (e.g., human serum albumin (HSA) and recombinant human albumin (rHA)), gelatin, casein, and the like.

Some compositions include a polyol. Polyols include sugars (e.g., mannitol, sucrose, trehalose, and sorbitol) and polyhydric alcohols such as, for instance, glycerol and propylene glycol, and polyethylene glycol (PEG) and related substances. Polyols are kosmotropic. They are useful stabilizing agents in both liquid and lyophilized formulations to protect proteins from physical and chemical degradation processes. Polyols also are useful for adjusting the tonicity of formulations.

Certain compositions include mannitol as a stabilizer. It is generally used with a lyoprotectant, e.g., sucrose. Sorbitol and sucrose are useful for adjusting tonicity and as stabilizers to protect against freeze-thaw stresses during transport or the preparation of bulk product during the manufacturing process. PEG is useful to stabilize proteins and as a cryoprotectant and can be used in the invention in this regard.

Sugars, including monosaccharides, di-, tri-, tetra-, and oligosaccharides; derivatized sugars such as alditols, aldonic acids, esterified sugars and the like; and polysaccharides or sugar polymers can be included in some formulations. For example, suitable carbohydrate excipients include, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like.

Surfactants can be included in certain formulations. Surfactants are typically used to prevent, minimize, or reduce protein adsorption to a surface and subsequent aggregation at air-liquid, solid-liquid, and liquid-liquid interfaces, and to control protein conformational stability. Suitable surfactants include, for example, polysorbate 20, polysorbate 80, other fatty acid esters of sorbitan esters, Triton surfactants, lechithin, tyloxapal, and poloxamer 188.

In some aspects, one or more antioxidants are included in the pharmaceutical composition. Antioxidant excipients can be used to prevent oxidative degradation of proteins. Reducing agents, oxygen/free-radical scavengers, and chelating agents are useful antioxidants in this regard. Antioxidants typically are water-soluble and maintain their activity throughout the shelf life of a product. EDTA is another useful antioxidant.

Certain formulations include metal ions that are protein co-factors and that are necessary to form protein coordination complexes. Metal ions also can inhibit some processes that degrade proteins. For example, magnesium ions (10-120 mM) can be used to inhibit isomerization of aspartic acid to isoaspartic acid.

A tonicity enhancing agent can also be included in certain formulations. Examples of such agents include alkali metal halides, preferably sodium or potassium chloride, mannitol, and sorbitol.

One or more preservatives can be included in certain formulations. Preservatives are necessary when developing multi-dose parenteral formulations that involve more than one extraction from the same container. Their primary function is to inhibit microbial growth and ensure product sterility throughout the shelf-life or term of use of the drug product. Suitable preservatives include phenol, m-cresol, p-cresol, o-cresol, chlorocresol, benzyl alcohol, phenylmercuric nitrite, phenoxyethanol, phenyl alcohol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate, thimerosal, benzoic acid, salicylic acid, chlorhexidine, or mixtures thereof in an aqueous diluent.

A pharmaceutical composition is formulated to be compatible with its intended route of administration. Examples of routes of administration are intravenous (IV), intradermal, inhalation, transdermal, topical, transmucosal, and rectal administration. A preferred route of administration for an antigen binding protein (e.g., an antibody) is IV infusion. In another preferred aspects, the preparation is administered by intramuscular or subcutaneous injection.

Formulation components suitable for parenteral administration (e.g., intravenous, subcutaneous, intraocular, intraperitoneal, intramuscular) include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and should be preserved against microorganisms. The carrier can be a solvent or dispersion medium containing, for example, water, ethanol, polyol (for example, glycerol, propylene glycol, and liquid polyethylene glycol), and suitable mixtures thereof.

Further guidance on appropriate formulations depending upon the form of delivery is provided, for example, in Remington's Pharmaceutical Sciences, 22^{nd} Edition, (Loyd V. Allen, ed.) Pharmaceutical Press (2013).

Pharmaceutical formulations are preferably sterile. Sterilization can be accomplished by any suitable method, e.g., filtration through sterile filtration membranes. Where the composition is lyophilized, filter sterilization can be conducted prior to or following lyophilization and reconstitution.

### X. Kits/Articles of Manufacture

Kits containing an antigen binding protein or ADC as described herein are also provided. In one aspect, such kits comprise one or more containers comprising an antigen binding protein (e.g., an anti-CD25 antibody) or ADC, or unit dosage forms and/or articles of manufacture. In some aspects, a unit dosage is provided wherein the unit dosage contains a predetermined amount of a composition comprising an antigen binding protein or ADC, with or without one or more additional agents. In some aspects, such a unit dosage is supplied in a single-use prefilled syringe for injection. In various aspects, the composition contained in the unit dosage may comprise: saline; a buffer, other formulation components, and/or be formulated within a stable and effective pH range as described herein. Alternatively, in some aspects, the composition is provided as a lyophilized powder that can be reconstituted upon addition of an appropriate liquid, for example, sterile water.

Some kits as provided herein further comprise instructions for use in the treatment of a disease associated with CD25-expressing Treg cells, such as cancer in accordance with any of the methods described herein. The kit can further comprise a description of how to select or identify an individual suitable for treatment. Instructions supplied in the kits of the invention are typically written instructions on a label or package insert (e.g., a paper sheet included in the kit), but machine-readable instructions (e.g., instructions carried on a magnetic or optical storage disk) are also acceptable. In some aspects, the kit further comprises another therapeutic agent, such as those described above as suitable for use in combination with the antigen binding protein.

In a further aspect, kits for detecting the presence of CD25, or a cell expressing CD25, in a sample are provided. Such kits typically comprise an antigen binding protein as described herein and instructions for use of the kit.

Certain kits, for example, are for diagnosis of cancer and comprises a container comprising an antigen binding protein (e.g., an anti-CD25 antibody), and one or more reagents for detecting binding of the antigen binding protein to CD25. Such reagents can include, for example, fluorescent tags, enzymatic tags, or other detectable tags. The reagents can also include secondary or tertiary antibodies or reagents, e.g., for use in enzymatic reactions that produce a product that can be visualized. In some aspects, a diagnostic kit comprises one or more antigen binding proteins in labeled or unlabeled form in suitable container(s), reagents for the incubations for an indirect assay, and substrates or derivatizing agents for detection in such an assay, depending on the nature of the label.

Kits such as provided herein can be used for in situ detection. Some methods utilizing such kits comprise removing a histological specimen from a patient and then combining the labeled antigen binding protein (e.g., an anti-CD25 antibody) with the biological sample. With such methods, it is possible to determine not only the presence of CD25 or CD25-fragments but also the distribution of such peptides and the cells expressing the same in the examined tissue (e.g., in the context of assessing the spread of cancer cells or in the context of assessing the amount of Treg cells present in a cancer).

In another aspect, an anti-idiotypic antibody (Id) which binds to an antigen binding protein (e.g., an anti-CD25 antibody) is provided. An Id antibody can be prepared by immunizing an animal of the same species and genetic type as the source of an anti-CD25 mAb with the mAb to which an anti-Id is being prepared. The immunized animal typically can recognize and respond to the idiotypic determinants of the immunizing antibody by producing an antibody to these idiotypic determinants (the anti-Id antibody).

The following examples, including the experiments conducted and the results achieved, are provided for illustrative purposes only and are not to be construed as limiting the scope of the appended claims.

### XI. Examples

### Example 1—Materials and Methods

### Antibody Expression, Purification, and Analysis

IgG1 antibodies were expressed via transient transfection of Expi HEK or Expi CHO cells or stable transfection of CHO-DG44 and purified using MabSelect SuRe columns (GE Healthcare). The identity and purity of each antibody was confirmed using liquid chromatography-mass spectrometry, hydrophobic interaction chromatography and size-exclusion chromatography.

### Size Exclusion Chromatography

The extent of aggregation of the conjugates was determined by SEC using an analytical SEC column (Sepax SRT-C 300 7.8 mm ID×30 cm, 5 μm) on a Waters 2695 HPLC system. The injected material was eluted using an isocratic mixture of 92.5% 25 mM sodium phosphate (pH 6.8), 350 mM NaCl, and 7.5% isopropyl alcohol at a flow rate of 1 m/min.

### Reverse-Phase Liquid Chromatography-Mass Spectrometry

Protein LC-MS data were acquired on a Waters Xevo GS-S QTOF coupled to a Waters Acquity H-Class UPLC system. Samples were reduced with 10 mM dithiothreitol (DTT) for 10 min at 37° C. and then chromatographed over an analytical reversed-phase column (Agilent Technologies, PLRP-S, 300 Å, 2.1 mm ID×50 mm, 3 μm) at 80° C. and eluted with a linear gradient of 0.01% TFA in acetonitrile from 25% to 65% in 0.05% aqueous TFA over 5 minutes, followed by isocratic 65% 0.01% TFA in acetonitrile for 0.5 min at a flow rate of 1.0 mL/min. Mass spectrometry data was acquired in ESI+ mode using a mass range of 500-4000 m/z and were deconvoluted using MaxEnt1 to determine masses of the resulting conjugates.

### Saturation Binding Analysis by ELISA

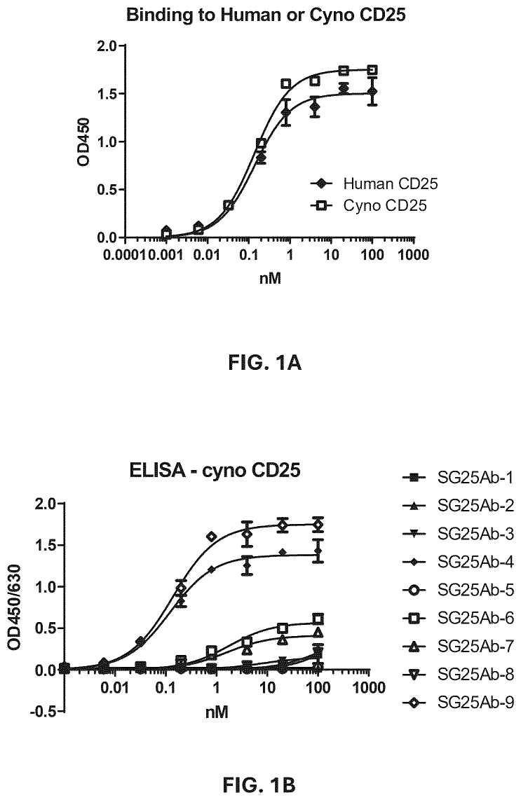

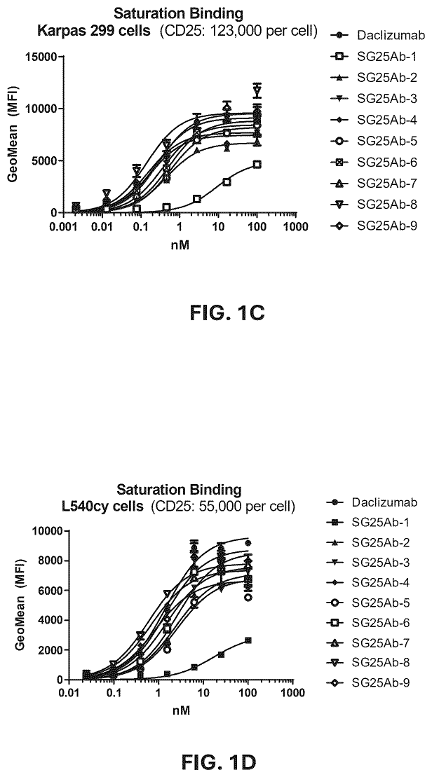

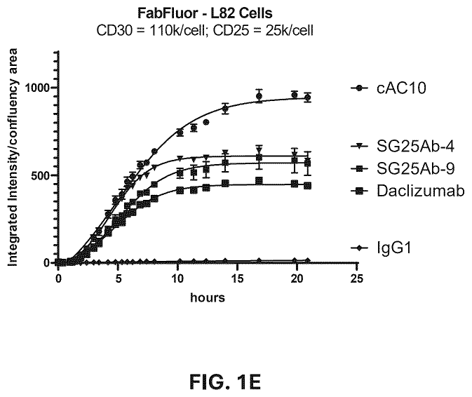

Saturation binding ELISA experiments were performed using soluble recombinant antigens, which were diluted to an appropriate concentration in 50 mM carbonate buffer, pH 9.6. To each well of a 96- or 384-well Maxisorb ELISA plate was added 100 μL of soluble antigen. The plate was sealed and stored at 4° C. overnight. The plate was then washed 3-5 times with PBS-T was buffer (PBS, pH 7.4+0.05% Tween-20). The wells were blocked using 300 μL/well of PBS-T buffer containing BSA for 1 hour at room temperature, then washed 3-5 times with PBS-T. Dilutions of antibodies were prepared in blocking buffer and added to each well in a volume of 100 μL. The antibodies were incubated for 1 hour at room temperature, and then washed 3-5 times with PBS-T. HRP-conjugated secondary antibodies (anti-human Fc) were then added and incubated for 1 hour at room temperature. The plate was washed 3-5 times with PBS-T. The ELISA was developed by adding 100 μL of TMB solution and incubating for 3-15 min at room temperature. To stop the reaction, 100 μL of 1 N sulfuric acid was added to each well. The absorbance at 450 nm was determined using a Spectramax 190 plate reader (Molecular Biosciences) and the data plotted using GraphPad Prism 6. Saturation binding analysis of SG25Ab-9 IgG1 antibody had similar binding to recombinant human or cyno CD25 (FIG. 1A).

### Saturation Binding Analysis by Flow Cytometry

For cellular binding analysis by FACS, cells expressing the target protein of interest (2×10⁵) were treated with a serial dilution of indicated antibody in staining buffer (PBS, 5% FBS, 0.2% NaN₃). Samples were incubated for 1 hour on ice and washed twice with ice-cold staining buffer. Cells were resuspended with anti-human IgG-AF488 or IgG-AF647 (JacksonImmunoResearch), 1:200 dilution in staining buffer) for 1 hour on ice. Cells were washed twice with ice cold staining buffer and resuspended in staining buffer. Labeled cells were examined by flow cytometry on an Invitrogen Attune NxT flow cytometer gated to exclude nonviable cells, and the data analyzed using FlowJo10 software. When determined, the K_{D} was calculated using GraphPad Prism 6 using non-linear regression.

### Antibody-Drug Conjugate Preparation

ADCs were prepared by reduction of antibody interchain disulfides followed by addition of a 25-100% excess maleimide as described previously (Lyon et al., Nature Biotechnology, 10: 1059-65, 2014). Partial reduction of 4 thiols per antibody was accomplished by addition of 2.1 equivalents of tris(2-carboxyethyl)-phosphine (TCEP) to an antibody solution (1-10 mg/mL in PBS, pH 7.4). Full reduction of 8 thiols per antibody was accomplished by addition of 10-12 equivalents of TCEP. The extent of antibody reduction was monitored by reverse-phase LC-MS and additional TCEP was added as needed to complete the reaction. TCEP was then removed by ultrafiltration (3×, 10-fold dilution into PBS, pH 7.4 containing 1 mM EDTA, centrifugation at 4000×g through a 30-kDa MWCO filter). Reduced antibodies in PBS-EDTA were conjugated with 25-100% excess of drug-linker as a 10 mM DMSO stock. The resulting solution was vortexed and left at room temperature for 10-20 minutes. The extent of conjugation was assessed by reverse-phase LC-MS as described above, and additional drug-linker or drug-carrier was added as needed. Once all available Cys thiols were alkylated, the crude ADC solution was purified by buffer exchange into PBS using either a PD-10 or Nap-5 desalting column (GE Healthcare) or through 3-5 rounds of ultrafiltration. The final ADC concentration was determined spectrophotometrically.

### Antibody-Dependent Cellular Cytotoxicity

NK cell mediated antibody dependent cellular cytotoxicity (ADCC) was examined in both fucosylated and non-fucosylated antibody forms. Human NK cells (158 V/V) were purified from a single human PBMC donor (Astarte) using an EasySep Human NK cell enrichment kit (STEMCELL Technologies) according to the manufacturer's instructions. Antibodies were incubated with human NK cells and CD25-expressing L540cy tumor cells for 4 hours, at which time cell-mediated cytotoxicity was assessed using a CytoTox 96 Non-Radioactive Cytotoxicity Assay (Promega) according to manufacturer's instructions.

### In Vitro Cytotoxicity Towards CD25-Expressing Lymphoma Cells

Cells were incubated with ADCs for 96 hours at 37° C. A non-binding ADC was included as a negative control. Cell viability was measured using CellTiter-Glo (Promega) according to the manufacturer's instructions. Cells (ca. 500-1000 cells/well) were seeded onto a 96-well culture plate with a clear bottom in culture medium. Dilutions of ADC were added to each well (2000 ng/mL to 0 ng/mL) and the samples were incubated for 96 hours at 37° C. Luminescence was measured using a plate reader (Envision 4605). The data was fit using Graph Pad software and an IC₅₀ was calculated.

### In Vitro Cytotoxicity Towards Purified Human Treg and CD8 T Cells

Purified human Tregs or CD8 T cells (acquired from either Astarte, Bothell, WA or STEMCELL Technologies, Vancouver, BC, Canada) were cultured in the presence of anti-CD3/CD28 beads (ThermoFisher) and incubated with ADCs for 96 hours. Cell viability was assessed using Cell Titer-Glo (Promega, Madison, WI).

In vivo Efficacy in Cell Line Xenograft Models

The in vivo efficacy of CD25 ADCs was evaluated in three xenograft models that express CD25 (DEL, L82, L540cy) in either SCID (DEL, L540cy) or NSG mice (L82). To conduct the efficacy experiments, 5×10⁶ cells were injected subcutaneously into 5 female mice per group. Mice were randomly divided to study groups and dosed with test article via intraperitoneal injection once the tumors reached approximately 100 mm³. For the L82 model in NSG mice, all mice were pre-treated with 10 mg/kg human IVIG 24 hours prior to treatment. Animals were euthanized when tumor volumes reach 800-1000 mm³. Tumor volume was calculated with the formula (volume=1/2×length×width×width). Mice showing durable regressions were terminated around day 60.

### In Vivo T Cell Depletion and Anti-Tumor Activity in a Syngeneic Tumor Model

To assess whether non-fucosylated CD25 antibody SG25Ab-9 and non-fucosylated detuned CD25 antibody SG25Ab-9 YH98A displayed differential systemic Treg depletion in vivo, human CD25 transgenic mice (C57BL/6-Il2ratml(IL2RA), Biocytogen, Wakefield, MA) were administered an intravenous dose of 0.1 mg/kg antibody test article. At 72 or 168 hours post-dose, the mice were sacrificed and the spleens harvested for flow cytometry analysis of T cell populations.

To test ADC-mediated antitumor activity and Treg depletion in a syngeneic tumor model, the C57BL/6-Il2ratml(IL2RA) mice were implanted with subcutaneous MC38 murine colon carcinoma tumors. When tumors reached ˜80-100 mm³, ADCs were administered at a dose of 1 mg/kg every three days for a total of 3 doses. ADC activity was compared to an anti-PD1 control and IgG1 ADC control. To assess the extent of Treg depletion in tumors compared to spleen and PBMC, mice were administered anti-CD25 ADC at a dose of either 3, or 6 mg/kg every three days for a total of 3 doses and compared to an IgG1 ADC control. The impact to Tregs was assessed in tumors, spleen, and PBMC by flow cytometry at 2 days post the second dose.

### Regulatory T Cell Depletion in Non-Human Primates

To evaluate and compare Treg depletion of CD25 antibody clone 123 MMAE ADC, detuned CD25 antibody SG25Ab-9 MMAE ADC and a non-binding IgG1 MMAE ADC, male cynomolgus macacques were administered an intravenous bolus dose of 6 mg/kg ADC on days 1, 22, and 43. Exploratory FACS analysis was performed on peripheral blood samples to evaluate effects on leukocyte subpopulations and potential toxicity.

Samples used for flow cytometric analysis were collected in EDTA-coated tubes twice during acclimation on days −12 and −7, and at 48, 96, 168, and 336 hrs post-Day 1 dose, prior to dosing on Days 22 and 43, and at 48, 96, and 168 hrs post-Day 43 dose. The EDTA-treated whole blood samples were placed on wet ice immediately following collection, checked for clots, then processed in ACK lysis buffer and cryopreserved in FBS and 10% DMSO.

To prepare cells for FACS analysis, one vial of each sample was thawed at 37° C. and transferred into 1 ml of RPMI 1640+10% FBS (R10) media in a deep well 96-well plate. Following centrifugation (350×g, for 5 min, at RT) supernatant was aspirated, an additional 2 mLs of R10 was added to each well, the plate was centrifuged, the wells were aspirated again, and cells were then transferred to a 96-well round bottom plate. The cells were then centrifuged again and stained with Zombie Aqua Fixable Viability dye according to manufacturer's protocol. Following the viability stain, cells were washed 1× in cell staining buffer and Fc-gamma receptors were blocked for 10 minutes. Cells were then stained in cell staining buffer for 30 min at 4° C. with antibodies for detection of surface antigens. For CD25 staining, antibody clone 4E3 (ThermoFisher) was used. Following two more washes in cell staining buffer, cell pellets were fixed, permeabilized and stained with Foxp3 PE using the True Nuclear Transcription Factor Buffer Set (Biolegend) according to the manufacturer's protocol. Cells were then resuspended in 150 μL staining buffer and analyzed on an NXT Attune flow cytometer. Flow cytometry data was analyzed using FlowJo software. Flow cytometry summary data were presented as a percent change from the baseline (day −7) in the absolute number or frequency among live lymphocytes, or as frequency of the parent population. Absolute numbers were calculated by multiplying the total lymphocyte count acquired from hematology (cells/L×109) on fresh whole blood with the frequency of each lymphocyte subpopulation within the live/single cell/lymphocyte gate determined through flow cytometry.

### Example 2—Generation and Functional Testing of Human Anti-CD25 Antibodies and Antibody-Drug Conjugates

A phage-displayed naïve human single-chain fragment variable antibody (ScFv) library (bacteriophage antibody presentation system from Creative Biolabs, 45-1 Ramsey Road, Shirley, NY, 11967) was screened to identify high affinity fully human anti-CD25 antibodies that bound to human CD25. The primary screen comprised three positive-selection panning steps against recombinant human CD25 coated plates, and two rounds of negative selection by non-specific panning against non-coated plates. Following three positive selection rounds of panning, monoclonal phage ELISA against recombinant human CD25 was used to identify 21 positive clones. DNA sequencing for all 21 positive clones identified 9 unique sequences.

Nine unique clones were recombinantly expressed in IgG1 format from HEK-293 cells for further characterization. CD25 antibody SG25Ab-9 demonstrated similar binding to human and cynomolgus CD25/IL2RA (FIG. 1A) and SG25Ab-4 also efficiently bound cynomolgus CD25 (FIG. 1B). The nine CD25 antibodies bound to human CD25 expressing cells (FIG. 1B-1D). CD25 antibody SG25Ab-9 (SG25Ab-9) and CD25 antibody SG25Ab-4 efficiently internalized into CD25 expressing cells (FIG. 1E).

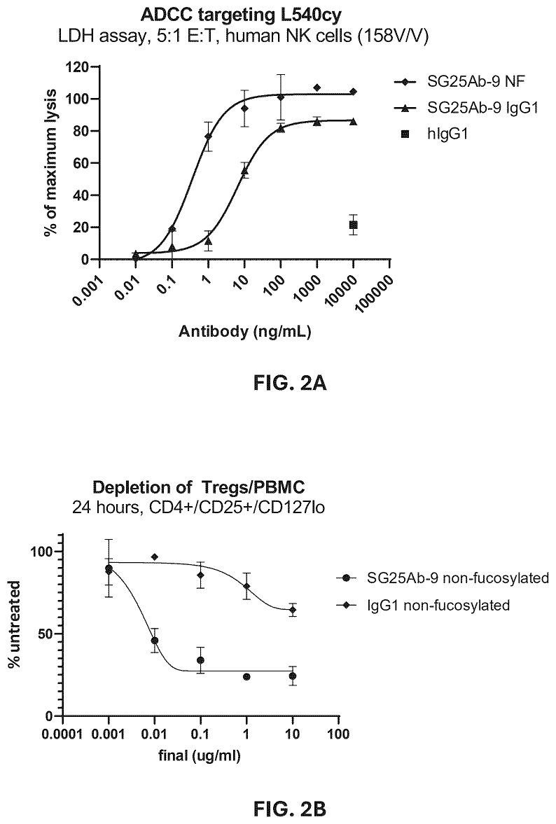

To determine whether CD25 ADCs showed antibody dependent cellular anti-tumor activity (ADCC)-mediated killing, NK cell mediated ADCC was examined on L540cy target cells. CD25 antibody SG25Ab-9 and non-fucosylated CD25 antibody SG25Ab-9 (SG25Ab-9 NF) displayed robust NK-mediated ADCC activity towards CD25 expressing L540cy cancer cells (FIG. 2A).

Further, SG25Ab-9 NF depleted Tregs from peripheral blood mononuclear cells (PBMC) (FIG. 2B). Frozen human PBMCs (Astarte) were thawed and plated at a density of 400,000 cells per well. 5-fold dilutions of antibodies were added and incubated at 37° C. for 24 hours. Samples were run by flow cytometry and Tregs were gated by labeling CD3+/CD4+/CD25+ and CD127low/neg. The extent of depletion was determined by comparison to an untreated sample.

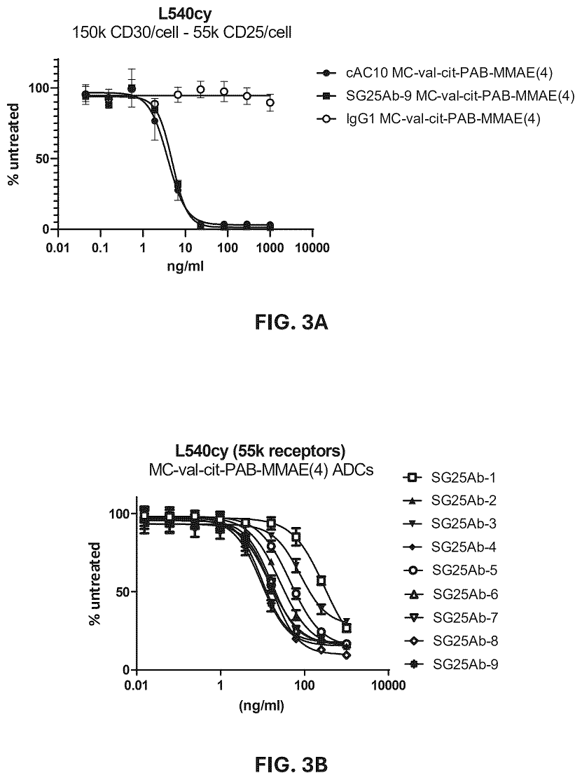

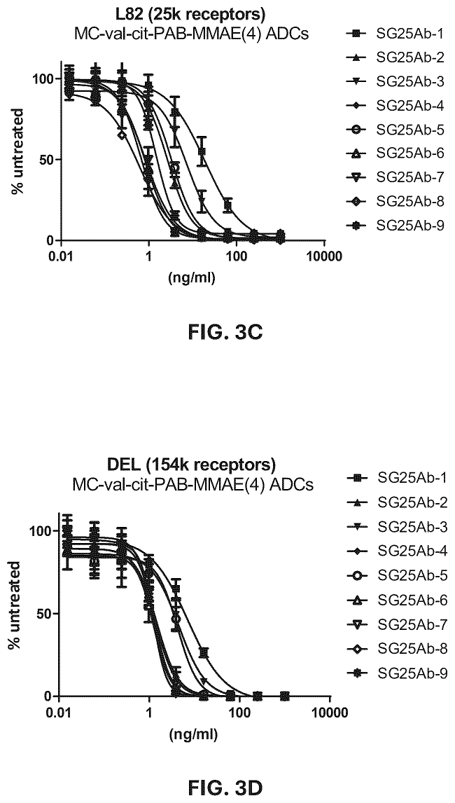

In vitro cytotoxicity was tested on several CD25 expressing cell lines. CD25 antibody SG25Ab-9 MMAE ADC showed anti-tumor activity towards L540cy cells similar to CD30 antibody cAC10 MMAE ADC (FIG. 3A). The nine CD25 antibodies when present in MMAE ADCs showed in vitro cytotoxicity towards L540cy cells, L82 cells, and DEL cells (FIGS. 3B, 3C, and 3D).

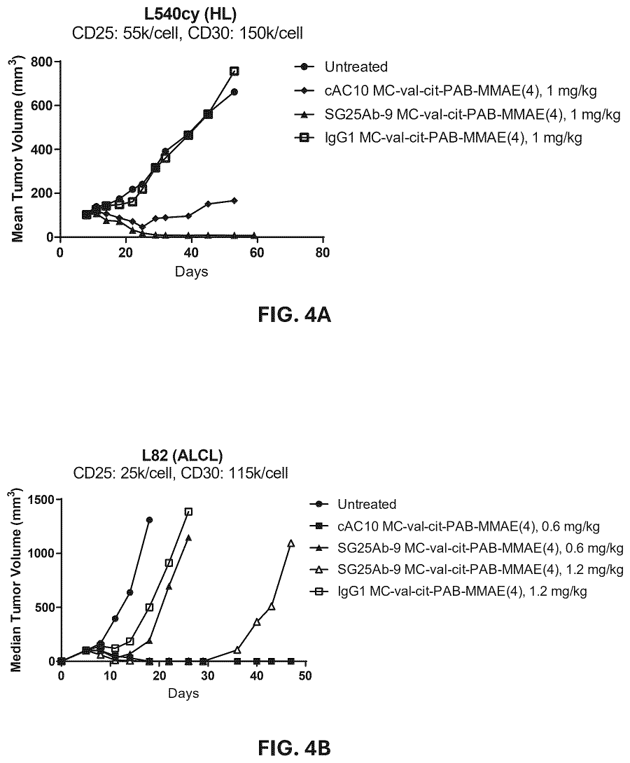

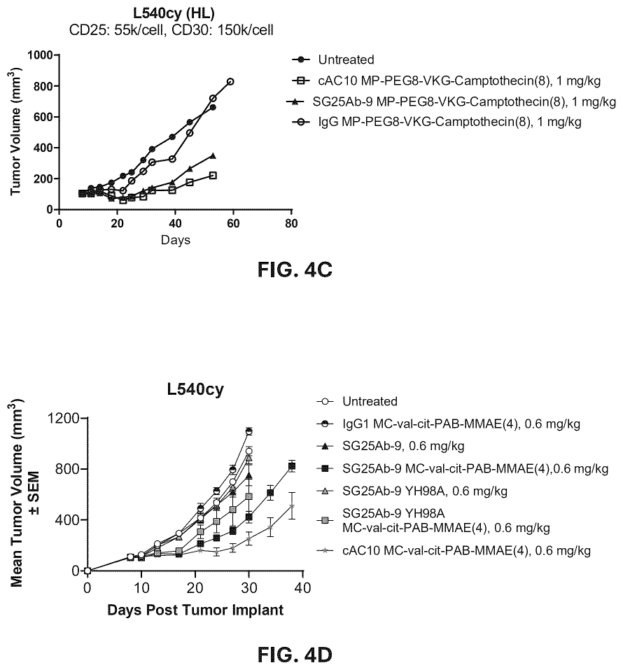

In vivo anti-tumor activity was tested in xenograft mouse models. CD25 antibody SG25Ab-9 MMAE ADC showed in vivo anti-tumor activity towards L540cy and L82 cell line-derived xenografts (FIGS. 4A and 4B). Further, CD25 antibody SG25Ab-9 when conjugated with a camptothecin payload also showed activity towards L540cy cells (FIG. 4C). Anti-tumor activity of CD25 antibody SG25Ab-9 and its MMAE ADC, SG25Ab-9 MMAE ADC (CD25 antibody SG25Ab-9 conjugated to MMAE via the linker MC-val-cit-PAB-MMAE), detuned CD25 antibody SG25Ab-9 YH98A and its MMAE ADC, detuned CD25 antibody SG25Ab-9 YH98A MMAE ADC (detuned CD25 antibody SG25Ab-9 YH98A conjugated to MMAE via the linker MC-val-cit-PAB-MMAE), CD30 antibody cAC10 and its MMAE ADC was tested in a L540cy cell line-derived xenograft and showed detuned CD25 antibody SG25Ab-9 YH98A MMAE ADC maintained a dose dependent in vivo efficacy in a human CD25+ lymphoma xenograft, which was comparable to the high affinity molecule at the higher dose (FIG. 4D).

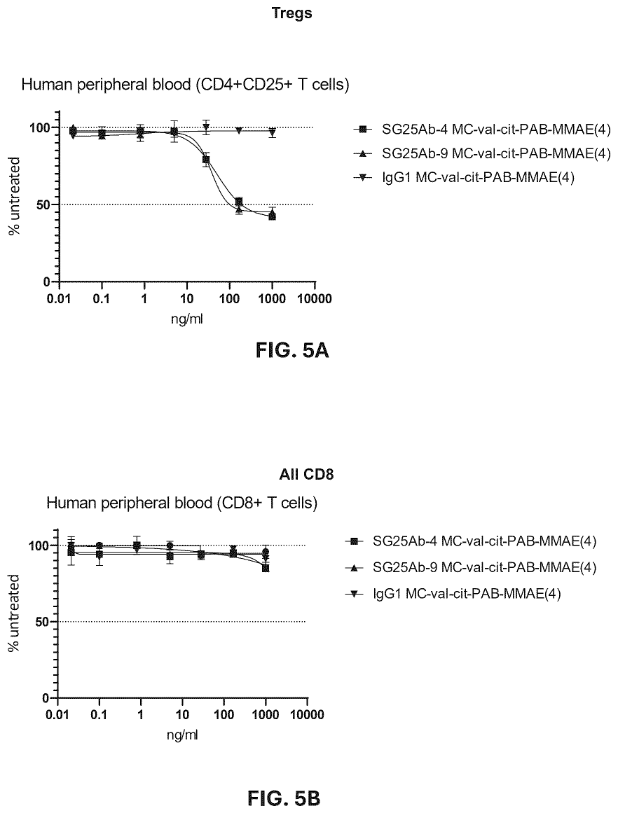

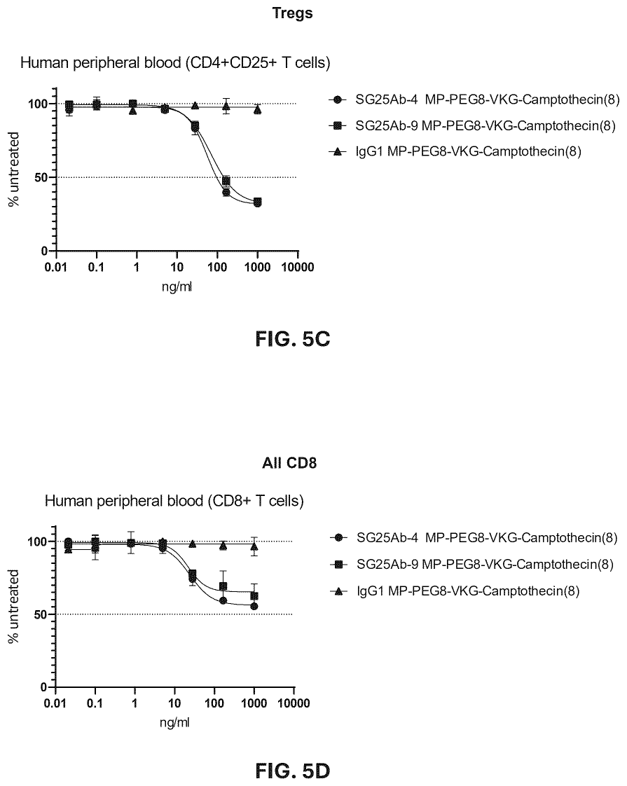

In vitro cytotoxicity of purified human T cell populations from human peripheral blood in vitro was determined. CD25 antibody SG25Ab-9 MMAE ADC and CD25 antibody SG25Ab-4 MMAE ADC killed Tregs (FIG. 5A), but did not impact CD8 T cells (FIG. 5B). In contrast, when CD25 antibody SG25Ab-9 was conjugated with a camptothecin ADC, Treg cells and CD8 T cells were depleted from human peripheral blood (FIGS. 5C and 5D).

### Example 3—Generation of Affinity Tuned Anti-CD25 Antibodies

To generate a series of SG25Ab-9 antibodies with different affinity profiles, site-directed mutagenesis of heavy chain CDR residues was performed. A series of point mutations in the heavy chain CDR residues of SG25Ab-9 were incorporated to create a panel of antibodies with differing affinities to CD25. A point mutation was introduced into the heavy chain variable domain in CDR3 (Y98 Å) of SG25Ab-9 to reduce binding to CD25.

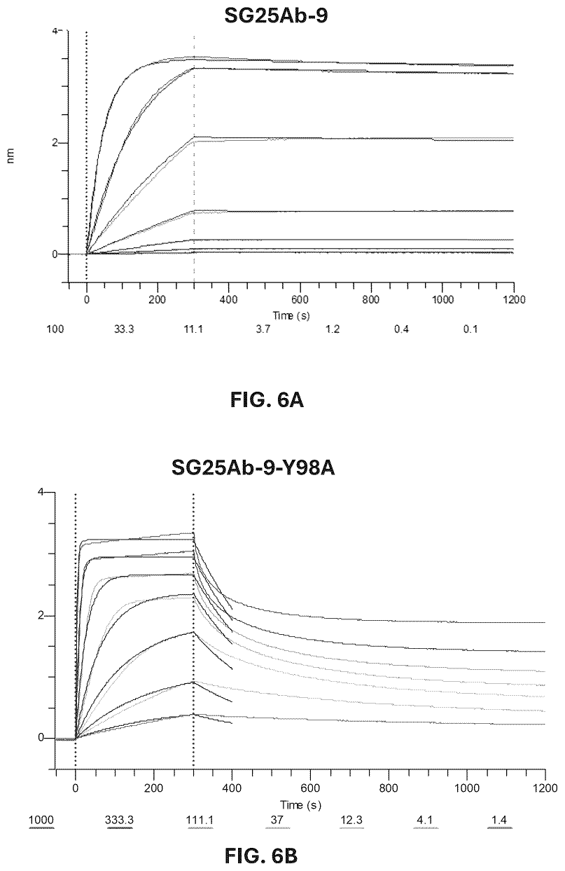

The variant, containing a Tyr to Ala modification at position 102 (YH98 Å) was ultimately identified as having reduced binding affinity to CD25 as demonstrated in kinetic binding assays. Biotinylated recombinant human CD25 was obtained from Acro Biosystems. Biotinylated hCD25 protein was loaded on SAX (High sensitivity streptavidin) tips at 0.8 nM loading density. Affinity measurements were run using a ForteBio Octet RED384 instrument in the kinetic buffer comprising 1×PBS, 0.1% BSA, 0.02% Tween20, pH 7.4. Association measurements were performed for 300 seconds and disassociation measurements were performed for 900 seconds. Each curve was reference subtracted and modeled using a 1:1 global fit. K_{D} results are reported as kₐ divided by k_{d}. The binding curves demonstrated efficient CD25 binding of CD25 antibody SG25Ab-9 (FIG. 6A) and a faster off rate for detuned CD25 antibody SG25Ab-9 YH98A (FIG. 6B).

Saturation binding analysis of CD25 antibody SG25Ab-9 and detuned CD25 antibody SG25Ab-9 YH98A on recombinant human and cyno CD25 was assessed by ELISA. CD25 antibody SGAb25-9 YH98A resulted in a decrease in affinity to both human and cyno CD25 compared to CD25 antibody SG25Ab-9 (FIG. 7).

Saturation binding analysis of CD25 antibody SG25Ab-9 and 15 CD25 antibody SG25Ab-9 variants on recombinant human CD25 was assessed by ELISA. The SG25Ab-9 variants showed a range of binding to human CD25 (FIGS. 8A and 8B), with reductions in affinity up to several hundred-fold compared to the parent SG25Ab-9 antibody (Table 6):

TABLE 6
Binding Affinity for SG25Ab-9 and its Variants

| | K_{D} | kₐ | k_{d} |
|---|---|---|---|
| Antibody | (nM) | (1/Ms) | (1/s) |
| SG25Ab-9 | 0.1 | 2.1E+05 | 2.7E−05 |
| YH27A | 0.1 | 2.4E+05 | 2.6E−05 |
| SH28W | 0.1 | 3.0E+05 | 3.8E−05 |
| YH32A | 0.8 | 3.0E+05 | 2.3E−04 |
| YH52A | 1.3 | 7.8E+05 | 1.0E−03 |
| GH53Y | 0.1 | 1.6E+06 | 8.1E−05 |
| DH54K | 24.2 | 3.3E+05 | 7.9E−03 |
| SH55N | 0.1 | 2.2E+05 | 2.8E−05 |
| DH56K | 150.9 | 2.6E+04 | 4.0E−03 |
| GH96W | 0.5 | 1.9E+06 | 1.0E−03 |
| YH98A | 14.0 | 3.1E+05 | 4.3E−03 |
| YH99A | 21.5 | 3.9E+05 | 8.5E−03 |
| AH100F | 3.9 | 3.1E+05 | 1.2E−03 |
| FH100AA | 0.6 | 2.9E+05 | 1.7E−04 |
| FH100AN | 0.3 | 2.0E+05 | 5.7E−05 |
| DH101K | 0.2 | 4.2E+05 | 1.0E−04 |

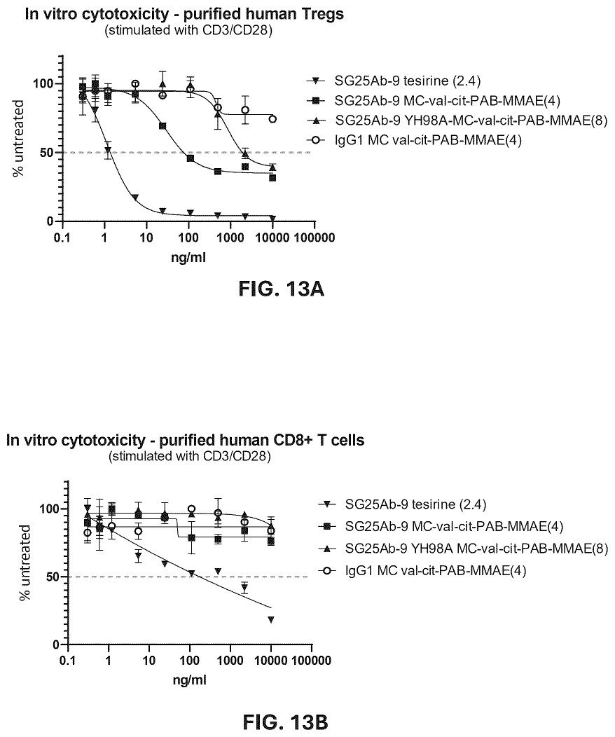

Saturation binding analysis was also performed using high CD25 expressing L540cy cells. This analysis showed that 13 of 15 CD25 antibody SG25Ab-9 variants had reduced binding affinity to L540cy cells but to differing extents (FIGS. 8C and 8D). In addition, the saturation binding analysis was performed with the CD25 antibody, SG25Ab-9, and the detuned SG25Ab-9 antibody, SG25Ab-9 YH98 Å, as MMAE-based ADCs. Both the affinity-detuned CD25 antibody ADC (SG25Ab-9 YH98A MC-val-cit-PAB-MMAE(4); “SG25Ab-9 YH98A MMAE ADC”) and CD25 antibody ADC (SG25Ab-9 MC-val-cit-PAB-MMAE(4); “SG25Ab-9 MMAE ADC”) showed ˜100-fold reduction in binding affinity versus the CD25 antibody, SG25Ab-9 (FIG. 8E), and there was a corresponding reduction in potency against purified human Tregs stimulated with CD3/CD28 beads, however, detuned CD25 antibody SG25Ab-9 YH98A MMAE ADC still effectively depleted activated Tregs at higher concentrations, as described in Example 6 (e.g., FIG. 13A).

### Example 4—In Vitro Cytotoxicity of Detuned CD25 Antibodies and Antibody-Drug Conjugates

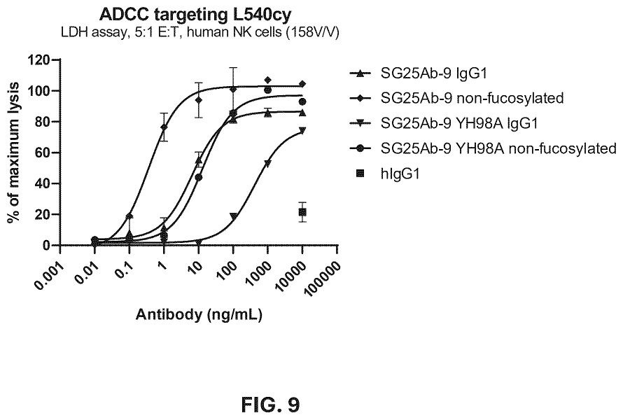

Antibody dependent cellular cytotoxicity (ADCC) mediated by human NK cells in the presence of detuned CD25 antibody SG25Ab-9 YH98A was assessed towards CD25-positive L540cy target cells. Detuned CD25 antibody SG25Ab-9 YH98A displayed reduced ADCC activity towards CD25-expressing L540cy cancer cells compared to CD25 antibody SG25Ab-9; a difference that was observed with both fucosylated and non-fucoslyated antibodies (FIG. 9).

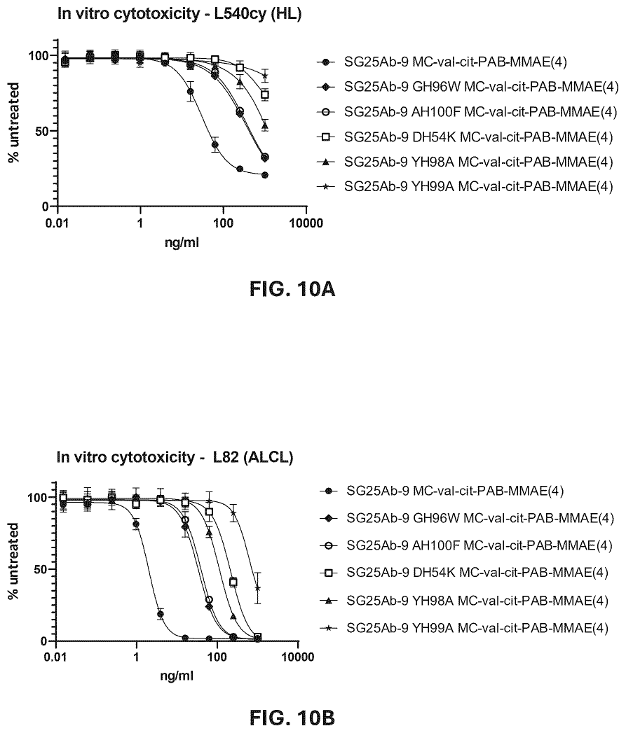

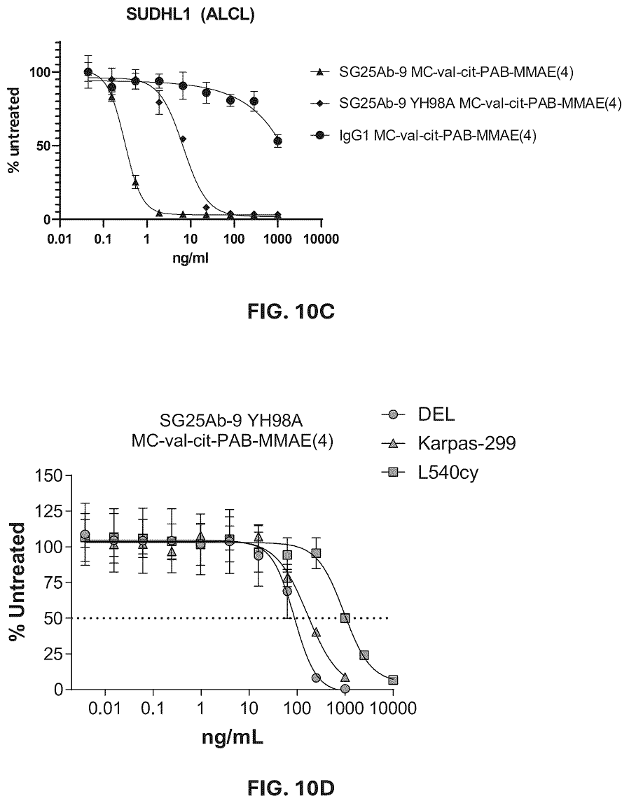

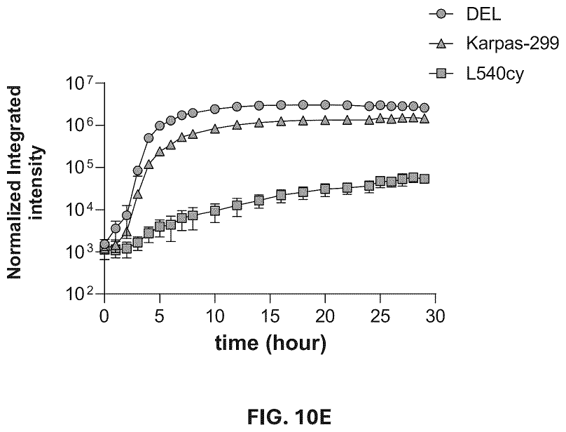

In vitro cytotoxicity of CD25 antibody SG25Ab-9 and detuned SG25Ab-9 YH98A antibodies as MMAE-based ADCs was tested on different CD25 expressing human cell lines. Detuned CD25 antibody SG25Ab-9 YH98A MMAE ADC demonstrated reduced in vitro cytotoxicity towards L540cy (HL) cells, L82 (ALCL) cells, and SUDHL1 (ALCL) cells compared to CD25 antibody SG25Ab-9 MMAE ADCs (FIGS. 10A, 10B, and 10C, respectively). In vitro cytotoxicity of detuned CD25 antibody SG25Ab-9 YH98A MMAE ADC towards DEL, cells, Karpas-299 cells, and L540cy cells, and the internalization of detuned CD25 antibody SG25Ab-9 YH98A MMAE ADC in these cells is shown in (FIGS. 10D, and 10E, respectively).

### Example 5—In Vivo Anti-Tumor Activity of Detuned CD25 Antibody-Drug Conjugates

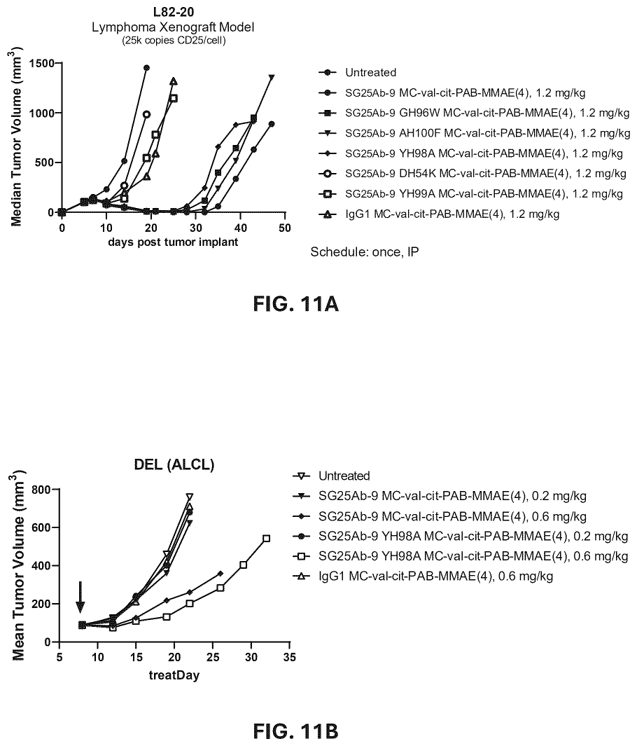

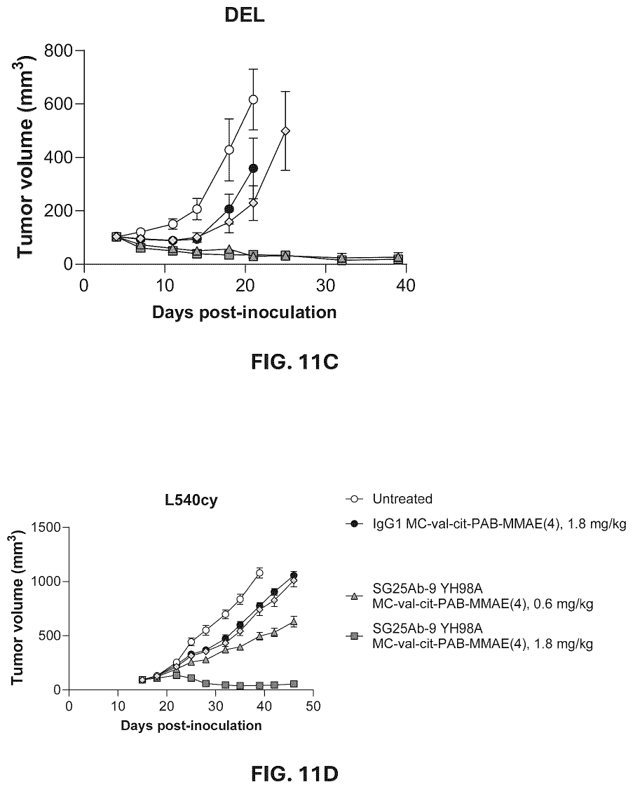

In vivo anti-tumor activity of CD25 antibody SG25Ab-9 and detuned SG25Ab-9 variant MMAE ADCs was tested in L82 and DEL xenograft mouse models. Detuned CD25 antibody SG25Ab-9 variant MMAE ADCs demonstrated different levels of reduction in anti-tumor efficacy in L82 xenografts compared to CD25 antibody SG25Ab-9 MMAE ADCs (FIG. 11A). This experiment showed that YH98A was the variant with lowest affinity that retained in vivo activity comparable to SG25Ab-9 (FIG. 11A). In a DEL lymphoma xenograft mouse model, detuned CD25 antibody SG25Ab-9 variant MMAE ADC demonstrated a comparable level of anti-tumor activity as CD25 antibody SG25Ab-9 MMAE ADCs when dosed at 0.6 mg/kg (FIG. 11B). Antitumor activity of detuned CD25 antibody SG25Ab-9 variant MMAE ADC in a DEL and a L540cy xenograft mouse model at 0.6 mg/kg and 1.8 mg/kg was also determined (FIGS. 11C and 11D, respectively).

### Example 6—Regulatory T Cell Depleting Activity of Detuned CD25 Antibodies and Antibody-Drug Conjugates

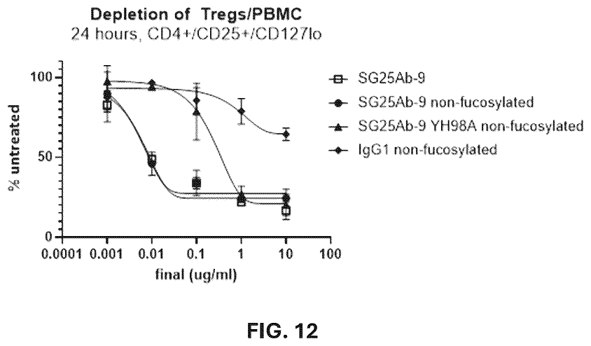

Treg depletion from PBMC in the presence of the CD25 antibody SG25Ab-9 and detuned the CD25 antibody SG25Ab-9 variant YH98A was measured. CD25 antibody SG25Ab-9 readily depleted Tregs while the detuned CD25 antibody SG25Ab-9 YH98A had reduced Treg depleting activity in vitro (FIG. 12).

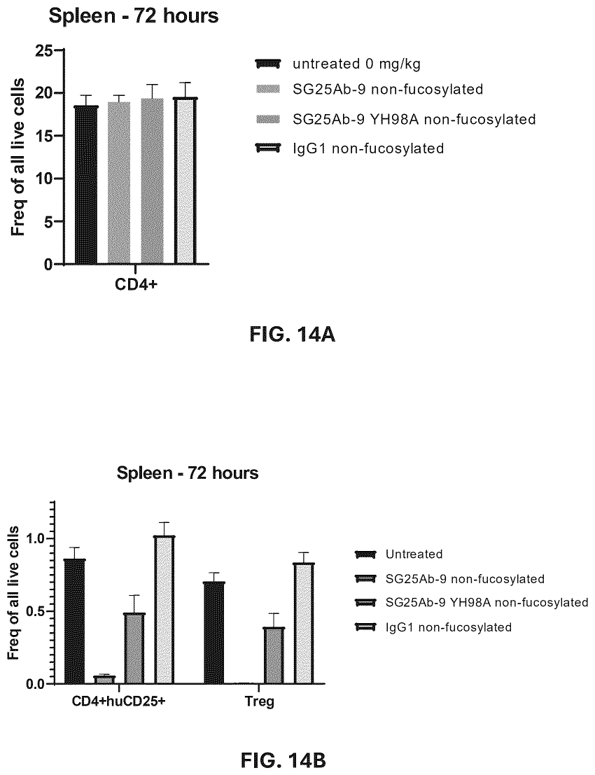

In vitro cytotoxicity towards purified human Treg cells with ADCs was also assessed. Detuned CD25 antibody SG25Ab-9 YH98A MMAE ADC showed less in vitro cytotoxicity towards purified human Treg cells than CD25 antibody SG25Ab-9 MMAE ADC. But, both CD25 antibody SG25Ab-9 MMAE ADC and detuned CD25 antibody SG25Ab-9 YH98A MMAE ADC were less cytotoxic towards purified human Treg cells than CD25 antibody SG25Ab-9 tesirine ADC (FIG. 13A). Furthermore, both CD25 antibody SG25Ab-9 MMAE ADC and detuned CD25 antibody SG25Ab-9 YH98A MMAE ADC were not cytotoxic towards purified human CD8 T cells, while the CD25 antibody SG25Ab-9 tesirine ADC was (FIG. 13B). In vivo effects of CD25 antibody SG25Ab-9 and detuned CD25 antibody SG25Ab-9 YH98A antibodies were measured in human CD25 transgenic mice and showed that neither antibody depleted splenic CD4 cells (FIG. 14A). The detuned CD25 antibody SG25Ab-9 YH98A demonstrated reduced depletion of human CD25 expressing mouse CD4 cells and human CD25 expressing mouse Treg cells than CD25 antibody SG25Ab-9 72 hours after administration (FIG. 14B).

### Example 7—Anti-Tumor Activity of Detuned CD25 Antibodies and Antibody-Drug Conjugates in a Human CD25 Mouse

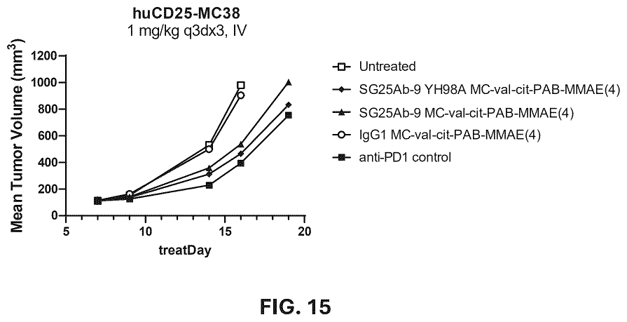

The anti-tumor activity of CD25 antibody SG25Ab-9 MMAE ADC and detuned CD25 antibody SG25Ab-9 YH98A MMAE ADC was measured in a colon cancer (MC38) xenograft model of a human CD25 transgenic mice. A transgenic mouse model expressing human CD25 (C57Bl/6-Il2ratml(IL2RA)/Bcgen) and implanted with MC38 murine colorectal carcinoma cells that lacks CD25 expression. CD25 antibody SG25Ab-9 MMAE ADC and detuned CD25 antibody SG25Ab-9 YH98A MMAE ADC provided similar tumor growth inhibition, and the extent of tumor growth delay was comparable to an anti-PD1 antibody control (FIG. 15).

### Example 8—Intratumor Treg Depletion by CD25 Antibody-Drug Conjugate, SG25Ab-9 MMAE ADC, in a Colon Cancer Xenograft Human CD25 Transgenic Mouse Model

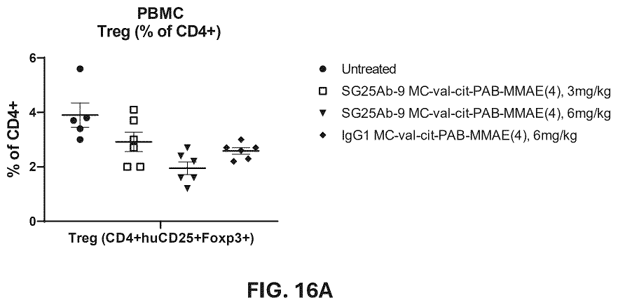

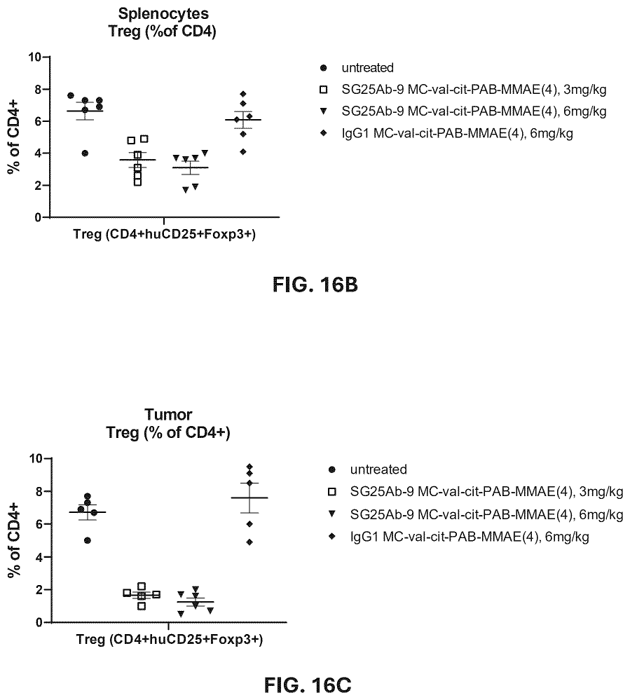

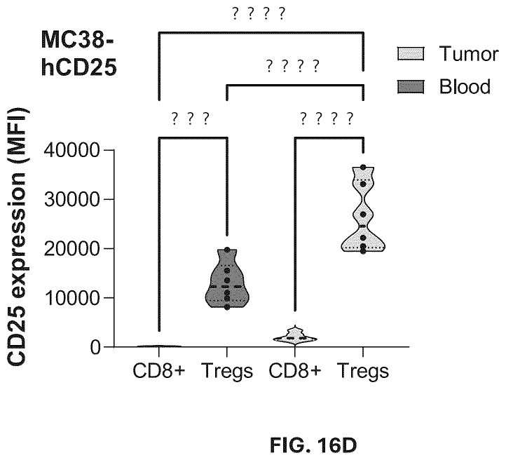

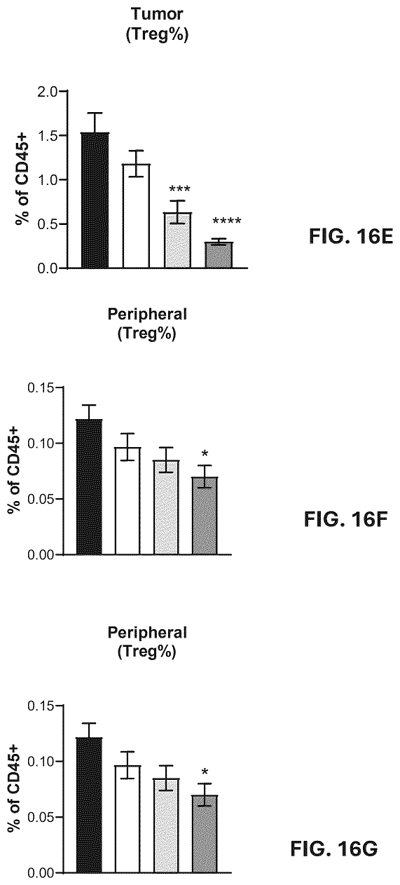

The Treg depletion by CD25 antibody SG25Ab-9 MMAE ADC was measured in a colon cancer (MC38) xenograft model of a human CD25 transgenic mouse. CD25 antibody SG25Ab-9 MMAE ADC reduced Treg cells in PBMC and splenocytes of MC38 xenograft human CD25 transgenic mice (FIGS. 16A and 16B, respectively). CD25 antibody SG25Ab-9 MMAE ADC substantially reduced Treg cells in the tumor of MC38 xenograft human CD25 transgenic mice (FIG. 16C). To demonstrate the preferential depletion of tumoral Treg by detuned CD25 antibody SG25Ab-9 YH98A MMAE ADC, the MC38 xenograft human CD25 transgenic mice received either 3 or 6 mg/kg of detuned CD25 antibody SG25Ab-9 YH98A MMAE ADC, an isotype control hIgG1-V, or no treatment (N=6 per group). Two days after the third dose, untreated mice were analyzed for CD25 expression by flow cytometry. Intratumoral Tregs had significantly higher CD25 expression compared to other T cell populations, confirming the model's translatability (FIG. 16D). After treatment, both doses of detuned CD25 antibody SG25Ab-9 YH98A MMAE ADC led to a significant reduction in intratumoral Tregs aligning with their antitumor activity, while only the higher dose (6 mg/kg) resulted in a slight decrease in peripheral Tregs (FIGS. 16E and 16F, respectively). The treatments did not have any reduction on intratumoral CD8+ T cells (FIG. 16G).

### Example 9—T Reg Depletion in Non-Human Primates

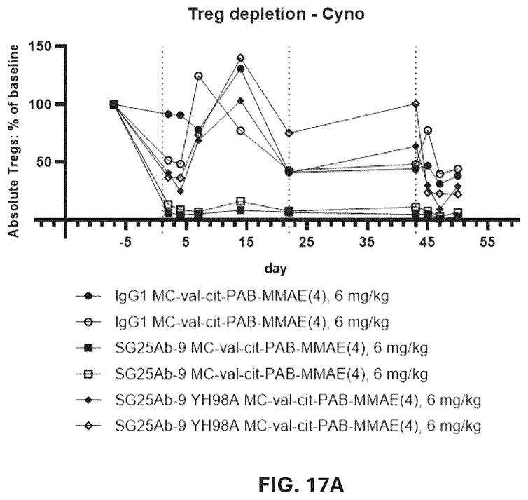

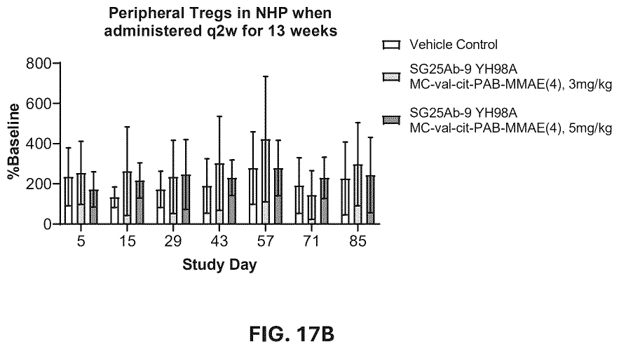

To evaluate the safety profile of detuned CD25 antibody SG25Ab-9 YH98A MMAE ADC and its impact on peripheral Tregs, repeat-dose toxicology studies in cynomolgus monkeys was performed. Detuned CD25 antibody SG25Ab-9 YH98A MMAE ADC, high-affinity CD25 antibody SG25Ab-9 MMAE ADC, or hIgG1-V were administered as bolus intravenous injections at a dose of 6 mg/kg once every three weeks, for a total of three doses (N=2 males/group). All the test articles were well tolerated in cynomolgus monkeys with consistent safety profiles to other MMAE-conjugated ADCs. Treatment with CD25 antibody SG25Ab-9 MMAE ADC led to a marked decrease in Treg cells compared to baseline (day −7) at all time points, while treatment with a non-specific IgG1 MMAE ADC had no effect (FIG. 17A). Detuned CD25 antibody SG25Ab-9 YH98A MMAE ADC treatment led to a moderate decrease in Tregs following the initial dose which generally rebounded by day 7 but following the third dose there was a larger decrease. Thus, while the high-affinity CD25 antibody SG25Ab-9 MMAE ADC elicited significant and prolonged reductions in Treg frequency with no recovery at any time points following treatment compared to baseline, detuned CD25 antibody SG25Ab-9 YH98A MMAE ADC treatment resulted in a moderate and reversible decrease in peripheral Tregs after each dose, which rebounded within a week to levels similar to the non-specific hIgG1-V. No consistent effects of the ADCs were seen in B cell, NK cell, or CD4+/CD8+ T cell subset numbers or activation. In a 13-week GLP-compliant repeat-dose toxicology in male and female cynomolgus monkeys, no effect on Treg was observed with detuned CD25 antibody SG25Ab-9 YH98A MMAE ADC at doses ≤5 mg/kg, the highest tested dose given every 2 weeks for 3 months (q2wx7), suggesting that affinity detuning enhances safety and therapeutic index (FIG. 17B).

### Example 10—T Reg Suppression by CD25 Antibody-Drug Conjugates

A Treg suppression assay was performed using the human Treg Suppression Inspector Kit (Miltenyi #130-092-909) according to manufacturer instructions. Cryopreserved human peripheral blood CD8+ T cells (Stem Cell Technologies, 200-0164) and CD4+CD25+CD127low Tregs (Stem Cell Technologies, 200-0123) from 2 normal donors were thawed and CD8+ T cells were labeled with Cell Trace™ CFSE dye (Invitrogen). Cells were mixed at ratio of Tregs to CD8+ T cells (1:2) in 96-well round-bottom plates, stimulated with CD3/CD28 beads (Treg suppression inspector kit, Miltenyi #130-092-909), and treated with CD25 antibody SG25Ab-9 or detuned CD25 antibody SG25Ab-9 YH98A MMAE ADC, at 1, 3 or 10 μg/mL, as well as non-targeting control (Isotype ADC) at 10 mg/mL. After 4 days of incubation at 37C and 5% CO2, cells were collected, washed in PBS, and stained with a Zombie Aqua viability dye (Biolegend #423102) for 15 minutes at room temperature. Cells were then washed in PBS with 2% FBS and stained with anti-hCD25-PE (Biolegend #302606), anti-hCD3-PE/Cy7 (Biolegend #300316), anti-hCD8-AF700 (Biolegend (#344724), and anti-CD4-APC Fire 750 (Biolegend #344638) for 30 minutes at 4C. Cells were washed twice, resuspended in cell staining buffer and acquired on an Attune NxT flow cytometer and Ultracomp ebeads Compensation Beads (ThermoFisher #01-2222-42) were used to compensate multi-fluorochromes.

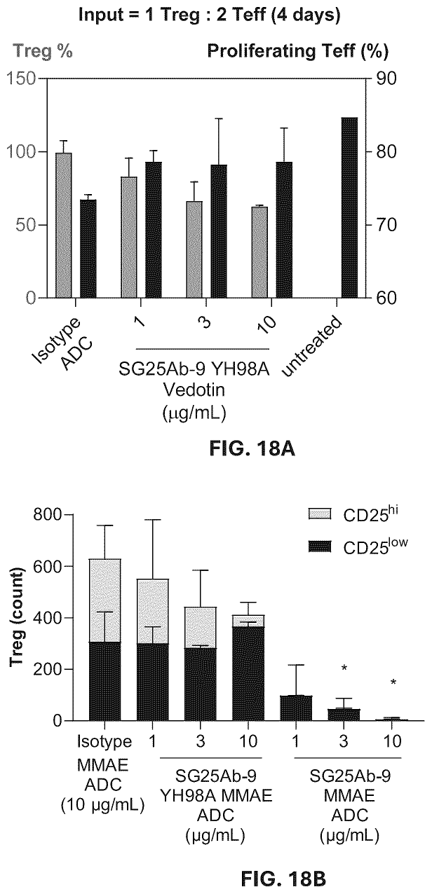

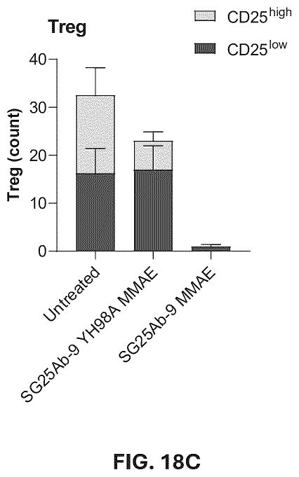

The effect of detuned CD25 antibody SG25Ab-9 YH98A MMAE ADC on normalized Treg counts (shown a percent of isotype control) and proliferating CSFElo CD8+T effector cells (shown as a percent of total CD8+ Teff cells) are shown in FIG. 18A. The activity of detuned CD25 antibody SG25Ab-9 YH98A MMAE ADC and CD25 antibody SG25Ab-9 MMAE ADC at 1, 3 or 10 g/mL on CD25hi Tregs and CD25lo Tregs counts is shown in FIG. 18B. The effect of treating PBMCs instead of a CD8 Treg co-culture with detuned CD25 antibody SG25Ab-9 YH98A MMAE ADC and CD25 antibody SG25Ab-9 MMAE ADC at 1 μg/mL on CD25hi Tregs and CD25lo Tregs counts is shown in FIG. 18C.

The data shows higher affinity CD25 ADC (CD25 antibody SG25Ab-9 MMAE ADC) kills Tregs, regardless of CD25 expression levels, while a dose dependent preferential decrease is observed in CD25hi Tregs by detuned CD25 antibody SG25Ab-9 YH98A MMAE ADC, in which low expressing Tregs were spared. This confirmed that detuned CD25 antibody SG25Ab-9 YH98A MMAE ADC preferentially targets CD25hi Tregs, which generally represent the most immunosuppressive fraction and are typically enriched in the tumor microenvironment.

### Example 11—CD25 Antibody PC61 and Detuned PC61 Binding Assay

The rat anti-mouse CD25 antibody PC61 (Huss, et al. Immunology, 2016, 148, 276-286) was reformatted with a mouse IgG2a backbone (PC61 mIgG2a), and a de-tuned variant (PC61 mIgG2a FH100BA) was identified by site-directed mutagenesis of CDR resides, as was done for SG25Ab (see Example 2).

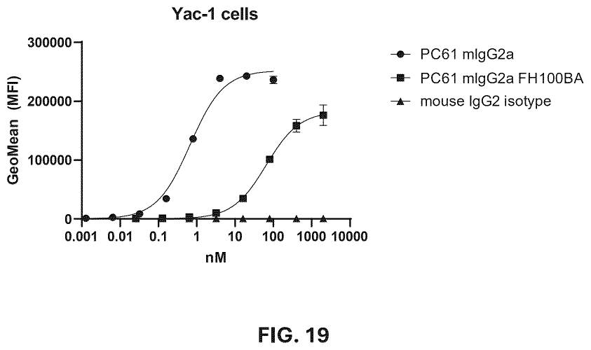

Saturation binding of PC61 mIgG2a and PC61 mIgG2a FH100BA was assessed by flow cytometry on mouse CD25 positive Yac-1 mouse lymphoma cells (ATCC, TIB-160) and showed that mutation of the F100B residue in the heavy chain of PC61 results in an approximately 100-fold decrease in affinity compared to the parent PC61 antibody, K_{D} of 64.6 vs. 0.7 nM, respectively (FIG. 19; Table 7).

TABLE 7
Binding Affinity for PC61, PC61 FH100BA

| | K_{D} |
|---|---|
| Antibody | (nM) |
| PC61 mIgG2a | 0.7 |
| PC61 mIgG2a FH100BA | 64.6 |

### Example 12—Anti-Tumor Activity of CD25 Antibody-Drug Conjugate, PC61 FH100BA MMAE ADC, in a Syngeneic Renal Adenocarcinoma Mouse Model

PC61 FH100BA-MC-VC-PABC-MMAE(4) was tested in a syngeneic renal adenocarcinoma CD25-negative Renca model. 2×10⁶ Renca cells were implanted into the flank of each BALB/c mouse. When the average tumor volume reached ˜100 mm³, mice were randomized and treated intravenously (IV) with PC61 FH100BA-mc-val-cit-PABC-MMAE(4) at 1, 3 or 6 mg/kg every three days for a total of three doses (Q3dx3) or left untreated (N=5 per group).

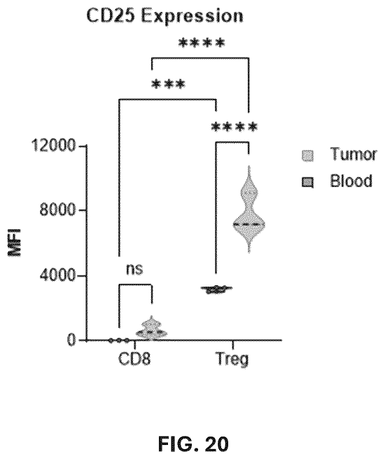

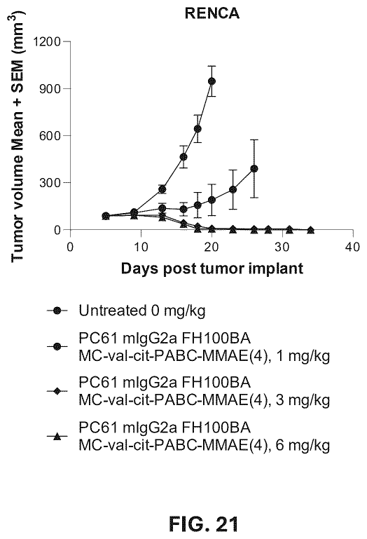

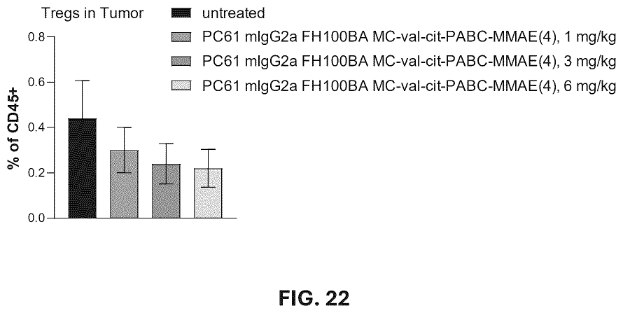

Untreated satellite mice were euthanized to profile T cells by flow cytometry (N=3). CD25 expression levels were significantly elevated on intratumoral Tregs relative to other T cell populations including peripheral Tregs and peripheral and intratumoral CD8+ T cells (FIG. 20), which recapitulates the CD25 expression profile in human Tregs. In the efficacy study, PC61 mIgG2a FH100BA MC-val-cit-PABC-MMAE(4) showed a dose-dependent response (FIG. 21), and the percentage of intratumoral Tregs to total tumoral CD45+ T cells decreased with treatment across all doses tested relative to the untreated group, supporting the MOA of Treg suppression promoting antitumor activity (FIG. 22).

### Example 13—Anti-Tumor Activity of CD25 Antibody-Drug Conjugate, PC61 FH100BA MMAE ADC with an Anti-PD-1 Antibody

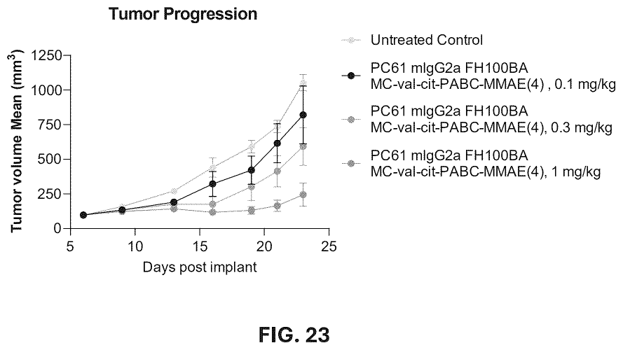

To assess combination with an anti-PD-1 agent, a sub-efficacious dose of PC61 mIgG2a FH100BA MC-val-cit-PABC-MMAE(4) was optimized in the Renca model. Renca tumor-bearing mice were treated with 0.1, 0.3 or 1 mg/kg PC61 mIgG2a FH100BA MC-val-cit-PABC-MMAE(4) or a non-binding isotype control m00 mIgG2aK (1 mg/kg) by IV q3dx3 (FIG. 23). Renca tumor-bearing mice were then treated with 0.3 mg/kg PC61 mIgG2a FH100BA MC-val-cit-PABC-MMAE(4) by IV q3dx3, 1 mg/kg anti-PD-1 (Clone 29F.1 Å12, BioXCell, Lebanon, NH) by IP q3dx3, or both. Additionally, mice were pre-treated with anti-CD8 depleting antibody (Clone 2.43, BioXCell, Lebanon, NH) by IP, 125 g, q2dx3, one week before combination treatment to assess the dependency of the observed effect on cytotoxic T cells.

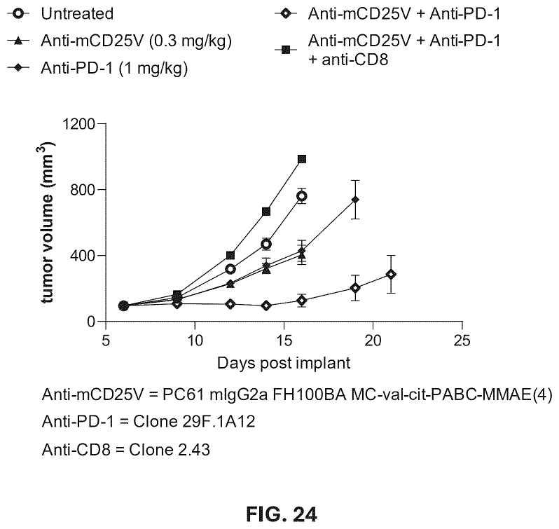

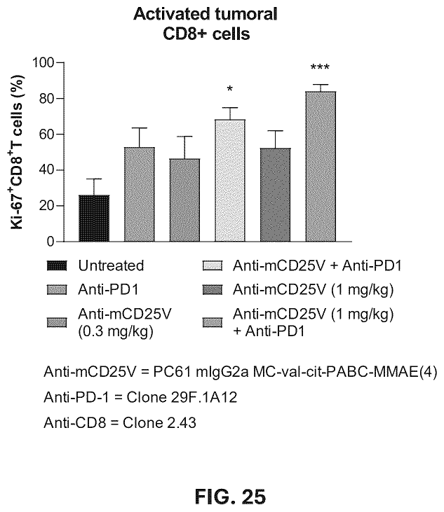

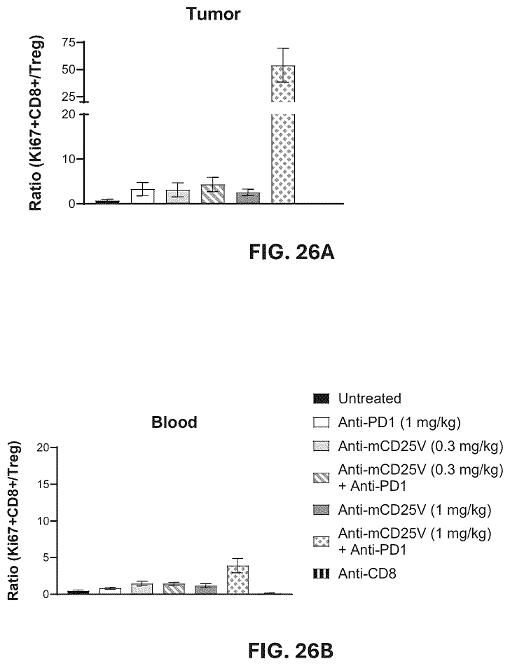

The combination of PC61 mIgG2a FH100BA MC-val-cit-PABC-MMAE(4) and anti-PD-1 demonstrated a combinatory effect relative to either single agent. Depletion of CD8+ T cells attenuated the antitumor effect of the combination therapy to a similar level as the untreated group. (FIG. 24) In parallel, the impact of the combination treatment on CD8+ T cell activation was assessed by flow cytometry using Ki67 as a marker of proliferation. Renca tumor-bearing mice were treated with mCD25 FH100BA-1006 (IV, 0.3 or 1 mg/kg q3dx3) and/or anti-PD-1 (IP, 1 mg/kg q3dx3) (N=5 mice per group). Tumor and blood samples were collected two days after the third dose to profile T cells. Both the 0.3 and 1 mg/kg doses of PC61 mIgG2a FH100BA MC-val-cit-PABC-MMAE(4)+anti-PD-1 combination treatment elicited a significant increase in proliferative tumoral Ki67+CD8+ T cells versus untreated (FIG. 25). In addition, the 1 mg/kg dose of PC61 mIgG2a FH100BA MC-val-cit-PABC-MMAE(4)+anti-PD-1 combination treatment significantly increased the tumor (FIG. 26A) and blood ratio of Ki67+CD8+ T cells to Tregs (FIG. 26B).

### Example 14: An Open-Label Phase 1 Study to Evaluate CD25 Antibody-Drug Conjugate as a Monotherapy and Part of a Combination Therapy in Advanced Malignancies

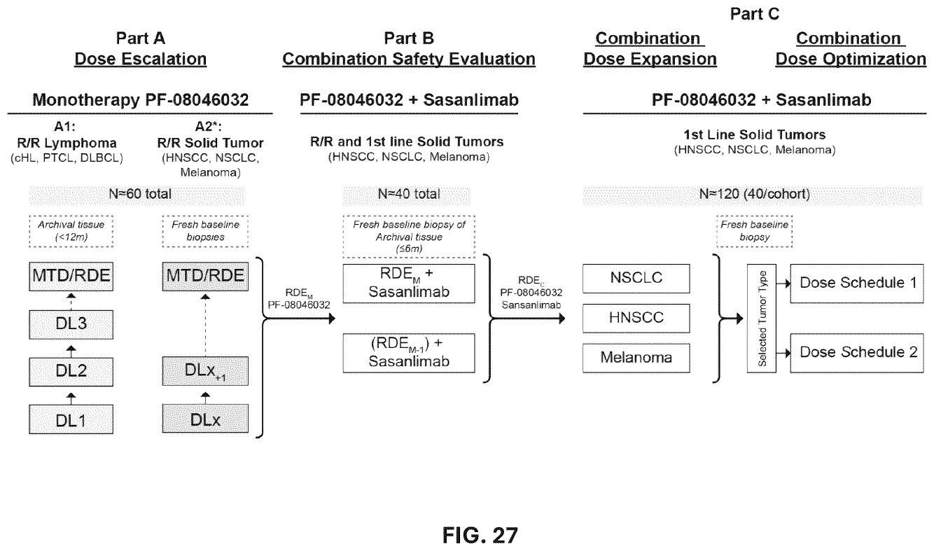

A Phase 1, open-label, multicenter, dose escalation and dose expansion study to evaluate the safety, pharmacokinetics (PK), pharmacodynamics (PD), and preliminary antitumor activity of detuned CD25 antibody SG25Ab-9 YH98A MMAE ADC as a single agent and in combination with sasanlimab in subjects with lymphomas and solid tumors advanced relapsed/refractory (r/r) lymphomas and advanced/metastatic solid tumors is conducted in three Parts: Part A, Part B, and Part C (FIG. 27).

Part A consists of two monotherapy dose escalation cohorts: Part A1—subjects with r/r lymphomas (classic Hodgkin lymphoma [cHL], peripheral T cell lymphoma [PTCL], and diffuse large B cell lymphoma [DLBCL]) and Part A2—subjects with r/r solid tumors (including but not limited to non-small cell lung cancer [NSCLC], head and neck squamous cell carcinoma [HNSCC], or melanoma), to assess the safety, tolerability and activity via direct killing of the lymphoma cells expressing CD25 of detuned CD25 antibody SG25Ab-9 YH98A MMAE ADC, whose disease has progressed on standard of care therapy. Dose escalation uses the Bayesian Optimal Interval (BOIN) design to evaluate safety and tolerability and to identify the maximum tolerated dose (MTD) of detuned CD25 antibody SG25Ab-9 YH98A MMAE ADC. Based on the totality of data and after a monotherapy recommended dose for expansion (RDEM) is identified, Part B is activated.

Part B consists of combination safety evaluation cohorts in subjects with advanced solid tumors (NSCLC, HNSCC, and melanoma), including subjects in the first line (1L) setting as well as r/r to prior immunotherapy, including but not limited to PD-(L)1 inhibitors, and CTLA-4 inhibitors. The safety, tolerability, and preliminary antitumor activity of detuned CD25 antibody SG25Ab-9 YH98A MMAE ADC in combination with sasanlimab is evaluated.

Part C is initiated after identification of the combination therapy MTD (MTD_{C}) or combination RDE (RDE_{C}) in Part B and based on totality of data. Part C consists of combination dose expansion cohorts in subjects with select advanced solid tumors (NSCLC, HNSCC, melanoma) in 1L, naïve to any immunotherapy, including but not limited to PD-(L)1 inhibitors, and CTLA-4 inhibitors. The goal of Part C is to further evaluate the safety, tolerability, and antitumor activity of the combination therapy (detuned CD25 antibody SG25Ab-9 YH98A MMAE ADC+sasanlimab) in these subjects.

### Key Inclusion Criteria

1. Tumor Types: All subjects have histologically or cytologically confirmed metastatic or unresectable malignancy.
   (a) Part A1—Relapsed/Refractory lymphomas:
      (i) Subjects with cHL, PTCL, large B-cell lymphoma/DLBCL (defined by the WHO criteria) and should have disease progression on or after treatment with standard therapies (prior treatment with brentuximab vedotin and polatuzumab vedotin is allowed; prior allogenic bone marrow transplantation is not allowed), or no standard therapy available or subject is not tolerant to available therapies.
      (ii) Subject with cHL: have had at least 2 prior systemic therapies (including but not limited to chemotherapy, brentuximab vedotin, ICIs, autologous SCT) and have no available therapies with known clinical benefit.
      (iii) Subject with PTCL: have had at least 1 prior systemic therapy and have received or have been ineligible to receive the combination of CHOP or CHOP-like therapy (must have also received autologous SCT unless ineligible).
      (iv) Subject with Large B-cell lymphoma/DLBCL (defined by WHO-HAEM5 criteria): must have received at least 2 prior systemic chemo-immunotherapy regimens, including an anti-CD20 agent and combination chemotherapy (unless contraindicated subjects should have had disease that has relapsed after or be refractory to intensive salvage chemotherapy, including autologous SCT; prior CAR T-cell therapy is allowed).
   (b) Part A2—solid tumors (r/r):
      (i) Subjects with advanced or metastatic NSCLC, HNSCC or melanoma. Limited other tumor types (e.g., gastric, cervical, microsatellite instability-high colorectal cancer [MSI-high CRC]) may be permitted. All subjects must have progressive disease following at least 1 prior approved systemic therapy; including immunotherapy (i.e., PD-(L)1 inhibitors, CTLA-4 inhibitors) if indicated and available, and should have no appropriate standard therapy at the time of enrollment.
      (ii) Subjects with tumor genomic aberrations for which there are approved and available targeted agents, as applicable for the tumor type, must have received approved mutation-specific targeted therapies and experienced PD, unless contraindicated or the subject experienced intolerance due to toxicity.
      (iii) Subjects with NSCLC and HNSCC must have received anti-PD-(L)1 or CTLA-4 inhibitors. therapy, if available, and a platinum-based chemotherapy regimen, either concurrently or sequentially and progressed/relapsed unless therapy is contraindicated or the subject experienced intolerance due to toxicity.
      (iv) Subjects with tumor previously tested for PD-(L)1 expression should have results available based on historical testing (a positive result is not required for enrollment).
      (v) 5. NSCLC (2L+): Both SCC (squamous cell carcinoma) and non-SCC histology are eligible; neuroendocrine component or histology are not eligible.
      (vi) HNSCC (2L+): Primary tumor site must arise from the oral cavity, oropharynx, hypopharynx, or larynx.
      (vii) Melanoma (2L+): Subjects with specific, targetable mutations (such as BRAF) should have received at least 1 therapy targeting that mutation, unless contraindicated.
   (c) Part B—solid tumors (r/r and 1L settings)
      (i) Subjects with NSCLC, HNSCC, or melanoma (histologically or cytologically confirmed advanced or metastatic NSCLC, HNSCC or melanoma) who either: 1. have not received prior anti-PD-(L)1 therapy or other immunotherapy agents for the tumor type; or 2. are r/r after prior anti-PD-(L)1 (all subjects must have progressive disease following at least 1 prior approved systemic therapy including PD(L)-1 inhibitor if indicated and should have no appropriate standard therapy at the time of enrollment) or other immunotherapy (last dose at least 90 days before C1D1) may be eligible.
      (ii) Subjects with tumor genomic aberrations for which there are approved and available targeted agents, as applicable for the tumor type, must have received approved mutation-specific targeted therapies and experienced PD, unless contraindicated or the subject experienced intolerance due to toxicity.
   (d) Part C—1L solid tumors
      (i) Subjects must have advanced or metastatic NSCLC, HNSCC, or melanoma and must not have received prior systemic therapy, including PD-(L)1 inhibitors, in the advanced/metastatic setting for the specific tumor type.
2. All subjects with solid tumors with genomic aberrations for which there are approved, and available targeted agents must have received approved mutation-specific targeted therapies and experienced PD, unless contraindicated or the subject experienced intolerance due to toxicity.
3. Measurable disease as defined by Lugano Classification for lymphomas and RECIST v1.1 for solid tumors.
4. Part A2: Subjects should agree to mandatory paired pre- and on-treatment de novo tumor biopsies.
5. An Easter Cooperative Oncology Group (ECOG) Performance Status score 0 or 1.
6. Resolution of acute effects of any prior therapy to either baseline severity or Common Terminology Criteria for Adverse Event (CTCAE) Grade ≤1.

### Key Exclusion Criteria

Subjects with any of the following characteristics/conditions are excluded:
1. Pre-existing peripheral neuropathy Grade ≥2, on-going at baseline, per NCI CTCAE v5.0.
2. History of Grade ≥3 immune-mediated AE that was considered related to prior immune modulatory therapy.
3. Known or suspected autoimmune disease that is currently active.
4. Known or suspected hypersensitivity to excipient contained in the drug formulation of detuned CD25 antibody SG25Ab-9 YH98A MMAE ADC or sasanlimab.
5. Subjects who have had a severe allergic reaction or anaphylactic reaction to any humanized monoclonal antibodies.

### Study Arms and Duration

For Part A (Dose Escalation): detuned CD25 antibody SG25Ab-9 YH98A MMAE ADC is administered intravenously (IV) at a defined dose level based on cohorts (lymphoma or solid tumors). The initial dosing schedule is on Day 1 and Day 15 of a 28-day cycle (2Q4W). An alternative dosing schedule may be initiated with additional subjects. Dosing continues in sequential dose escalation cohorts until a MTD or maximum administered dose (MAD) is reached and the RDEM is identified.

In Part A1, detuned CD25 antibody SG25Ab-9 YH98A MMAE ADC is initially administered by IV infusion on Day 1 and Day 15 of 28-day cycles at the planned doses listed in Table 8:

TABLE 8
Initial Dosing

| | Dose |
|---|---|
| Cohort | (mg/kg) |
| 1 | 0.75 |
| 2 | 1.0 |
| 3 | 1.25 |
| 4 | 1.5 |
| 5 | 1.75 |
| 6 | 2.0 |

Alternative schedules of detuned CD25 antibody SG25Ab-9 YH98A MMAE ADC may be evaluated in this study, such as in Table 9:

TABLE 9
Alternative Dosing Schedules

| | Dose intensity at planned dose levels for each schedule | | | |
|---|---|---|---|---|
| | (mg/kg/week) | | | |
| Dose Levels | Initial Schedule | Alternative Schedules | | |
| (mg/kg) | 2Q4W | 3Q4W | Q3W | 2Q3W |
| 0.75 | 0.375 | 0.563 | 0.25 | 0.5 |
| 1.0 | 0.5 | 0.75 | 0.333 | 0.667 |
| 1.25 | 0.625 | 0.937 | 0.417 | 0.833 |
| 1.5 | 0.75 | 1.125 | 0.5 | 1 |
| 1.75 | 0.875 | 1.312 | 0.583 | 1.167 |
| 2.0 | 1 | 1.5 | 0.667 | 1.33 |

2Q3W=Days 1, 8 every 21 days; 3Q4W=Days 1, 8, 15 every 28 days;
2Q4W=Days 1, 15 every 28 days; Q3W=Day 1 every 21 days.

The choice of which schedule(s) to open for enrollment is made by the sponsor in consultation with dose level review meeting (DLRM) members, based on available safety, DLTs, PK, pharmacodynamics, and initial antitumor activity. The starting dose level in the alternative schedule is no higher, in terms of dose intensity (mg/kg/week), than the highest dose level of the initial schedule deemed tolerable by the DLRM review. One or more dosing schedules may be evaluated sequentially or in parallel in separate cohorts. The BOIN dose escalation rules are applied separately to each dosing schedule. DLRM members may recommend investigation of lower and/or intermediate dose levels, in which case the BOIN dose escalation rules continue to be applied.

In Part A2, the initial dose of detuned CD25 antibody SG25Ab-9 YH98A MMAE ADC administered is based on the previously cleared dose and schedule, totality of data as determined by the DLRM members. The provisional dose escalation levels in Part A2 would follow the same guidance and schema as in Table 8, with initial dose selected at or below RDE for monotherapy in Part A1.

For Part B (Combination Safety Evaluation), subjects receive detuned CD25 antibody SG25Ab-9 YH98A MMAE ADC in combination with sasanlimab (administered subcutaneously [SC] at the established, fixed dose of 300 mg Q4W or 225 mg Q3W). The starting dose level of detuned CD25 antibody SG25Ab-9 YH98A MMAE ADC in Part B is one level lower than RDE_{M}, (RDE_{M}−1). The dose level at RDE_{M} may be evaluated if RDE_{M}−1 is deemed as tolerable. Additional cohorts may be considered according to the totality of data.

In a 21-day cycle, sasanlimab may be administered Q3W at a fixed dose of 225 mg on Day 1 of each cycle (e.g., Cycle 1 Day 1, then Cycle 2 Day 1) and at least 30 minutes prior to administration of detuned CD25 antibody SG25Ab-9 YH98A MMAE ADC when administered together.

In a 28-day cycle, sasanlimab may be administered Q4W at a fixed dose of 300 mg Q4W (e.g., Cycle 1 Day 1, Cycle 2 Day 1) and at least 30 minutes prior to administration of detuned CD25 antibody SG25Ab-9 YH98A MMAE ADC, when administered together.

For Part C (Combination Dose Expansion and Optimization), subjects receive detuned CD25 antibody SG25Ab-9 YH98A MMAE ADC at the RDE_{C} in combination with sasanlimab. Dose and schedules may differ between tumor-specific cohorts.

Based on emerging data, to inform the selection of optimal regimen, subjects in selected expansion cohorts may be randomized in a 1:1 ratio to receive one of the two doses and/or schedules selected based on the totality of data of the combination therapy.

In all parts, subjects may continue on treatment until disease progression, unacceptable toxicity, withdrawal of consent, initiation of subsequent therapy, or study termination, whichever occurs first.

### INCORPORATION BY REFERENCE

All references cited herein, including patents, patent applications, scientific papers, textbooks and the like are incorporated by reference in their entirety.

What is claimed is:

1. An antigen binding protein that binds CD25 comprising:
(a) a CDR-H1 comprising the amino acid sequence of SEQ ID NO: 1;
(b) a CDR-H2 comprising the amino acid sequence of SEQ ID NO: 2;
(c) a CDR-H3 comprising the amino acid sequence of SEQ ID NO: 21;
(d) a CDR-L1 comprising the amino acid sequence of SEQ ID NO: 4;
(e) a CDR-L2 comprising the amino acid sequence of SEQ ID NO: 5; and
(f) a CDR-L3 comprising the amino acid sequence of SEQ ID NO: 6.

2. The antigen binding protein of claim 1, wherein the antigen binding protein comprises an amino acid modification in one or more of CDR-H1, CDR-H2, CDR-H3, CDR-L1, CDR-L2, and CDR-L3, wherein the modifications collectively total 1, 2 or 3 conservative amino acid modifications.

3. The antigen binding protein of claim 1, wherein the antigen binding protein comprises a VH, wherein the VH comprises an amino acid sequence that has at least 80%, 85%, 90%, 95% or 99% amino acid sequence identity to the amino acid sequence of SEQ ID NO: 22.

4. The antigen binding protein of claim 1, wherein the antigen binding protein comprises a VL, wherein the VL comprises an amino acid sequence that has at least 80%, 85%, 90%, 95% or 99% amino acid sequence identity to the amino acid sequence of SEQ ID NO: 8.

5. The antigen binding protein of claim 1, wherein the antigen binding protein comprises a VH and a VL, wherein the VH comprises at least 80%, 85%, 90%, 95% or 99% amino acid sequence identity to the amino acid sequence of SEQ ID NO: 22, and the VL comprises an amino acid sequence that has at least 80%, 85%, 90%, 95% or 99% amino acid sequence identity to the amino acid sequence of SEQ ID NO: 8.

6. The antigen binding protein of claim 1, wherein the antigen binding comprises a HC comprising an amino acid sequence that has at least 80%, 85%, 90%, 95% or 99% amino acid sequence identity to the amino acid sequence of SEQ ID NO: 46.

7. The antigen binding protein of claim 1, wherein the antigen binding comprises a LC comprising an amino acid sequence that has at least 80%, 85%, 90%, 95% or 99% amino acid sequence identity to the amino acid sequence of SEQ ID NO: 47.

8. The antigen binding protein of claim 1, wherein the antigen binding comprises a HC and LC, wherein the HC comprises an amino acid sequence that has at least 80%, 85%, 90%, 95% or 99% amino acid sequence identity to the amino acid sequence of SEQ ID NO: 46 and the LC comprises an amino acid sequence that has at least 80%, 85%, 90%, 95% or 99% amino acid sequence identity to the amino acid sequence of SEQ ID NO: 47.

9. The antigen binding protein of claim 1, wherein the antigen binding protein is a monoclonal antibody.

10. The antigen binding protein of claim 1, wherein the antigen binding protein is a human, humanized or chimeric antibody.

11. The antigen binding protein of claim 1, wherein the antigen binding protein is a Fab, Fab′, Fv, scFv or (Fab′)₂ fragment.

12. An antibody-drug conjugate comprising the antigen binding protein of claim 1, wherein the antigen binding protein is conjugated to a cytotoxic or cytostatic agent.

13. The antibody-drug conjugate of claim 12, wherein the cytotoxic or cytostatic agent is conjugated to the antigen binding protein using a linker, a spacer, or both.

14. The antibody-drug conjugate of claim 13, wherein the spacer comprises a para-aminobenzylcarbamate.

15. The antibody-drug conjugate of claim 13, wherein the linker comprises a valine-citrulline dipeptide.

16. The antibody-drug conjugate of claim 12, wherein the antibody-drug conjugate comprises a linker-spacer of formula (I):

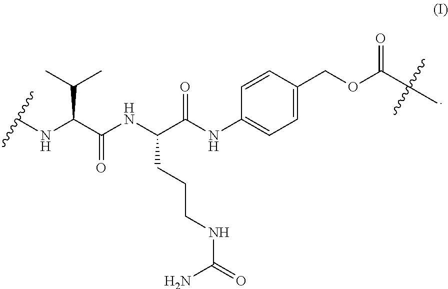 [SMILES: *N[C@H](C(=O)N[C@@H](CCCNC(N)=O)C(=O)Nc1ccc(COC(*)=O)cc1)C(C)C]

17. The antibody-drug conjugate of claim 12, wherein the antibody-drug conjugate comprises a maleimide-caproic acid attachment group.

18. The antibody-drug conjugate of claim 12, wherein the cytotoxic or cytostatic agent is an auristatin.

19. The antibody-drug conjugate of claim 18, wherein the cytotoxic agent is monomethyl auristatin E (MMAE).

20. The antibody-drug conjugate of claim 19, wherein the antibody-drug conjugate comprises 2 to 10 molecules of MMAE.

21. An antibody-drug conjugate comprising: 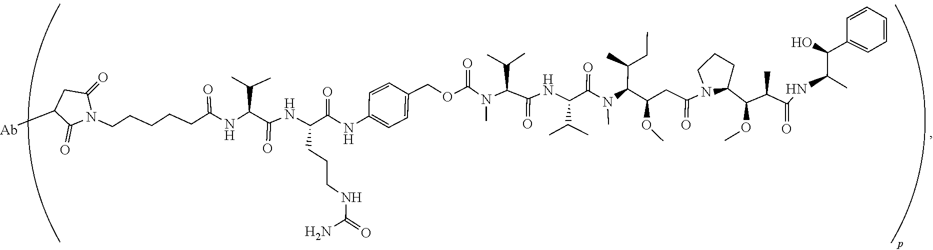
wherein Ab is an antigen binding protein that binds CD25 and comprises:
(a) a CDR-H1 comprising the amino acid sequence of SEQ ID NO: 1;
(b) a CDR-H2 comprising the amino acid sequence of SEQ ID NO: 2;
(c) a CDR-H3 comprising the amino acid sequence of SEQ ID NO: 21;
(d) a CDR-L1 comprising the amino acid sequence of SEQ ID NO: 4;
(e) a CDR-L2 comprising the amino acid sequence of SEQ ID NO: 5; and
(f) a CDR-L3 comprising the amino acid sequence of SEQ ID NO: 6; and
wherein p is from 1 to 8.

22. The antibody-drug conjugate of claim 21, wherein the antigen binding protein comprises a VH comprising the amino acid sequence of SEQ ID NO: 22 and a VL comprising the amino acid sequence of SEQ ID NO: 8.

23. The antibody-drug conjugate of claim 21, wherein the antigen binding protein comprises a HC comprising the amino acid sequence of SEQ ID NO: 46 and a LC comprising the amino acid sequence of SEQ ID NO: 47.

24. The antibody-drug conjugate of claim 21, wherein p is about 4.

25. An antibody-drug conjugate comprising the formula Ab-(L-U)n, wherein Ab is an anti-CD25 antibody, L is a linker between the cytotoxic or cytostatic molecule and the anti-CD25 antibody, U is the conjugated cytotoxic or cytostatic molecule, and n is an integer from 1 to 8, wherein the anti-CD25 antibody comprises a CDR-H1, CDR-H2, and CDR-H3 having amino acid sequences SEQ ID NOs: 1, 2 and 21, respectively, and a CDR-L1, CDR-L2, and CDR-L3 having amino acid sequences SEQ ID NO: 4, 5 and 6, respectively, wherein the linker is maleimidocaproyl valine citrulline p-amino-benzyloxy (mc-vc-pAB), and the cytotoxic or cytostatic molecule is MMAE.

26. An isolated nucleic acid encoding the antigen binding protein of claim 1.

27. A vector comprising the nucleic acid of claim 26.

28. A host cell comprising the vector of claim 27.

29. A method of producing an antigen binding protein that binds to CD25, wherein the method comprises: a) culturing the host cell of claim 28 under conditions suitable for expression of the polynucleotide encoding the antigen binding protein; and b) isolating the antigen binding protein.

30. A method of producing an antibody-drug conjugate comprising an antigen binding protein that binds to CD25, wherein the method comprises: a) culturing the host cell of any one of claim 28 under conditions suitable for expression of the polynucleotide encoding the antigen binding protein; b) isolating the antigen binding protein; and c) conjugating the antigen binding protein to a cytotoxic or cytostatic agent, wherein each unit of the cytotoxic or cytostatic agent is conjugated via a linker.

31. A method of treating cancer in a subject in need thereof comprising administering to the subject an effective amount of an antigen binding protein of claim 1 or an antibody-drug conjugate comprising the antigen binding protein of claim 1.

32. The method of claim 31, wherein the cancer is a solid tumor or lymphoma.

33. The method of claim 31, further comprising administration of radiation, a chemotherapeutic agent, a PD-1 inhibitor, a PD-L1 inhibitor, or a CTLA-1 inhibitor.

34. The method of claim 33, wherein the PD-1 inhibitor is sasanlimab, pembrolizumab or nivolumab.

35. A method of treating cancer in a subject in need thereof comprising administering to the subject a dose of about 0.1 mg/kg to about 0.3 mg/kg of the antibody-drug conjugate, wherein the dose is administered at least once every four weeks.

36. The method of claim 35, wherein the dose is administered 1, 2 or 3 times every four weeks, or 1 or 2 times every three weeks.

37. The method of claim 35, further comprising administering sasanlimab.

38. The method of claim 35, wherein the cancer is a lymphoma or solid tumor.


SEQUENCE LISTING (47 sequences)

SEQ ID NO: 1 - protein, 5 aa, synthetic construct
SYWIG

SEQ ID NO: 2 - protein, 17 aa, synthetic construct
IIYPGDSDTRYSPSFQG

SEQ ID NO: 3 - protein, 9 aa, synthetic construct
LGSYYAFDI

SEQ ID NO: 4 - protein, 14 aa, synthetic construct
TGTSSDVGAYIYVS

SEQ ID NO: 5 - protein, 7 aa, synthetic construct
DVSKRPS

SEQ ID NO: 6 - protein, 10 aa, synthetic construct
SSYTRSSTWV

SEQ ID NO: 7 - protein, 118 aa, synthetic construct
QVQLVQSGAEVKKPGESLKISCKGSGYSSTSYWIGWVRQMPGKGLEWMGIIYPGDSDTRYSPSFQGQVTISADKSISTAYLQWSSLKASDTAMYYCARLGSYYAFDIWGRGTMVTVSS

SEQ ID NO: 8 - protein, 110 aa, synthetic construct
QSALTQPASVSASPGQSITISCTGTSSDVGAYIYVSWYQQLSGKPPKLILYDVSKRPSGISDRFSGSKSGNTASLTISGLQADDEADYYCSSYTRSSTWVFGGGTQLTVL

SEQ ID NO: 9 - protein, 30 aa, synthetic construct
QVQLVQSGAEVKKPGESLKISCKGSGYSST

SEQ ID NO: 10 - protein, 14 aa, synthetic construct
WVRQMPGKGLEWMG

SEQ ID NO: 11 - protein, 32 aa, synthetic construct
QVTISADKSISTAYLQWSSLKASDTAMYYCAR

SEQ ID NO: 12 - protein, 11 aa, synthetic construct
WGRGTMVTVSS

SEQ ID NO: 13 - protein, 22 aa, synthetic construct
QSALTQPASVSASPGQSITISC

SEQ ID NO: 14 - protein, 15 aa, synthetic construct
WYQQLSGKPPKLILY

SEQ ID NO: 15 - protein, 32 aa, synthetic construct
GISDRFSGSKSGNTASLTISGLQADDEADYYC

SEQ ID NO: 16 - protein, 10 aa, synthetic construct
FGGGTQLTVL

SEQ ID NO: 17 - protein, 467 aa, synthetic construct
MAWVWTLLFLMAAAQGAQAQVQLVQSGAEVKKPGESLKISCKGSGYSSTSYWIGWVRQMPGKGLEWMGIIYPGDSDTRYSPSFQGQVTISADKSISTAYLQWSSLKASDTAMYYCARLGSYYAFDIWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - DNA, 1404 bp, synthetic construct
  source 1..1404: mol_type = other DNA
atggcttgggtgtggaccttgctattcctgatggcagctgcccaaggcgcccaagcacaggtccagctggtgcagtctggagctgaggtgaagaagcctggggagtctctgaagatctcctgcaagggctctggctactcatccacttcctattggataggctgggtgcgccagatgcctggaaagggactggagtggatgggcatcatttatcctggtgattctgacacacgctactctccatctttccaaggccaggtgaccatctctgcagacaagtccatcagcactgcctatctgcagtggagcagcctgaaggcttcagacactgccatgtactactgtgctagactgggttcttactatgcctttgacatctggggcagaggcaccatggtcaccgtctcctcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatga

SEQ ID NO: 19 - protein, 235 aa, synthetic construct
MAWALLLLTLLTQDTGSWAQSALTQPASVSASPGQSITISCTGTSSDVGAYIYVSWYQQLSGKPPKLILYDVSKRPSGISDRFSGSKSGNTASLTISGLQADDEADYYCSSYTRSSTWVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 20 - DNA, 708 bp, synthetic construct
  source 1..708: mol_type = other DNA
atggcctgggctctgctgctcctcactctcctcactcaggacacaggatcctgggcccagtctgctctgacacagccagcttcagtgtctgcatctccaggacagtccatcaccatctcctgcactggaaccagcagtgatgttggggcatatatctatgtcagctggtaccagcagctgtcaggcaaaccaccaaagctgatcctctatgatgtctccaagcggccctctgggatctctgacaggttcagtggctccaagtctgggaacacagcctctctcacaatctctgggctgcaggctgatgatgaggcagactattactgctcttcatatactaggagcagcacttgggtgttcggcggaggaacccagctgactgtcctaggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttcatag

SEQ ID NO: 21 - protein, 9 aa, synthetic construct
LGSAYAFDI

SEQ ID NO: 22 - protein, 118 aa, synthetic construct
QVQLVQSGAEVKKPGESLKISCKGSGYSSTSYWIGWVRQMPGKGLEWMGIIYPGDSDTRYSPSFQGQVTISADKSISTAYLQWSSLKASDTAMYYCARLGSAYAFDIWGRGTMVTVSS

SEQ ID NO: 23 - protein, 467 aa, synthetic construct
MAWVWTLLFLMAAAQGAQAQVQLVQSGAEVKKPGESLKISCKGSGYSSTSYWIGWVRQMPGKGLEWMGIIYPGDSDTRYSPSFQGQVTISADKSISTAYLQWSSLKASDTAMYYCARLGSAYAFDIWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - DNA, 1404 bp, synthetic construct
  source 1..1404: mol_type = other DNA
atggcttgggtgtggaccttgctattcctgatggcagctgcccaaggcgcccaagcacaggtccagctggtgcagtctggagctgaggtgaagaagcctggggagtctctgaagatctcctgcaagggctctggctactcatccacttcctattggataggctgggtgcgccagatgcctggaaagggactggagtggatgggcatcatttatcctggtgattctgacacacgctactctccatctttccaaggccaggtgaccatctctgcagacaagtccatcagcactgcctatctgcagtggagcagcctgaaggcttcagacactgccatgtactactgtgctagactgggttctgcttatgcctttgacatctggggcagaggcaccatggtcaccgtctcctcagctagcaccaagggcccatctgtcttccccctggcaccctcctccaagagcacctctgggggcacagctgccctgggctgcctggtcaaggactacttccctgaacctgtgacagtgtcctggaactcaggagccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacacagaagagcctctccctgtctccgggtaaatga

SEQ ID NO: 25 - protein, 5 aa, synthetic construct
RYWIA

SEQ ID NO: 26 - protein, 17 aa, synthetic construct
IIYPGDSDARYSPTFEG

SEQ ID NO: 27 - protein, 9 aa, synthetic construct
LGSYYAFDI

SEQ ID NO: 28 - protein, 14 aa, synthetic construct
TGTSSDVGGYNYVS

SEQ ID NO: 29 - protein, 7 aa, synthetic construct
DVSKRPS

SEQ ID NO: 30 - protein, 10 aa, synthetic construct
SSYTSSSTWV

SEQ ID NO: 31 - protein, 118 aa, synthetic construct
EVQLVQSGAEVKKPGESLKISCKGPEYSFNRYWIAWVRQRPGKGLEWMGIIYPGDSDARYSPTFEGHVTLSADMSLTTAYLQWSSLRASDTAMYYCARLGSYYAFDIWGKGTMVTVSS

SEQ ID NO: 32 - protein, 110 aa, synthetic construct
QSALTQPASVSGSPGQSITIFCTGTSSDVGGYNYVSWYQQHPGKAPKLMIYDVSKRPSGVSNRFSGSKSGNTASLTISGLQAEDEADYYCSSYTSSSTWVFGGGTQLTVL

SEQ ID NO: 33 - protein, 30 aa, synthetic construct
EVQLVQSGAEVKKPGESLKISCKGPEYSFN

SEQ ID NO: 34 - protein, 14 aa, synthetic construct
WVRQRPGKGLEWMG

SEQ ID NO: 35 - protein, 32 aa, synthetic construct
HVTLSADMSLTTAYLQWSSLRASDTAMYYCAR

SEQ ID NO: 36 - protein, 11 aa, synthetic construct
WGKGTMVTVSS

SEQ ID NO: 37 - protein, 22 aa, synthetic construct
QSALTQPASVSGSPGQSITIFC

SEQ ID NO: 38 - protein, 15 aa, synthetic construct
WYQQHPGKAPKLMIY

SEQ ID NO: 39 - protein, 32 aa, synthetic construct
GVSNRFSGSKSGNTASLTISGLQAEDEADYYC

SEQ ID NO: 40 - protein, 10 aa, synthetic construct
FGGGTQLTVL

SEQ ID NO: 41 - protein, 330 aa, synthetic construct [sequence omitted]

SEQ ID NO: 42 - protein, 272 aa, synthetic construct
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 43 - protein, 448 aa, synthetic construct
EVQLVQSGAEVKKPGESLKISCKGPEYSFNRYWIAWVRQRPGKGLEWMGIIYPGDSDARYSPTFEGHVTLSADMSLTTAYLQWSSLRASDTAMYYCARLGSYYAFDIWGKGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 217 aa, synthetic construct
QSALTQPASVSGSPGQSITIFCTGTSSDVGGYNYVSWYQQHPGKAPKLMIYDVSKRPSGVSNRFSGSKSGNTASLTISGLQAEDEADYYCSSYTSSSTWVFGGGTQLTVLSGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 45 - protein, 448 aa, synthetic construct
QVQLVQSGAEVKKPGESLKISCKGSGYSSTSYWIGWVRQMPGKGLEWMGIIYPGDSDTRYSPSFQGQVTISADKSISTAYLQWSSLKASDTAMYYCARLGSYYAFDIWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 448 aa, synthetic construct
QVQLVQSGAEVKKPGESLKISCKGSGYSSTSYWIGWVRQMPGKGLEWMGIIYPGDSDTRYSPSFQGQVTISADKSISTAYLQWSSLKASDTAMYYCARLGSAYAFDIWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 216 aa, synthetic construct
QSALTQPASVSASPGQSITISCTGTSSDVGAYIYVSWYQQLSGKPPKLILYDVSKRPSGISDRFSGSKSGNTASLTISGLQADDEADYYCSSYTRSSTWVFGGGTQLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS